(19) United States
(12) Patent Application Publication
(10) Pub. No.: US 2016/0369295 A1
(43) Pub. Date: Dec. 22, 2016

(54) DROUGHT TOLERANT PLANTS AND RELATED CONSTRUCTS AND METHODS INVOLVING GENES ENCODING DTP4 POLYPEPTIDES

(71) Applicants: E.I. DU PONT DE NEMOURS AND COMPANY, Wilmington, DE (US); PIONEER HI-BRED INTERNATIONAL INC, Johnston, IA (US)

(72) Inventors: Stephen M. Allen, Wilmington, DE (US); Bindu Andreuzza, Pradesh (IN); Norbert Brugiere, Johnston, IA (US); Zhenglin Hou, Ankeny, IA (US); Ratna Kumria, Hyderabad (IN); H. Renee Lafitte, Davis, CA (US); Xiao-Yi Li, Wilmington, DE (US); Cheng Lu, Newark, DE (US); Stanley Luck, Wilmington, DE (US); Amitabh Mohanty, Hyderabad (IN); Jeffery Mullen, Minnetrista, MN (US); Rupa Raja, Secunderabad (IN); Hajime Sakai, Newark, DE (US); Scott V. Tingey, Rockdale, TX (US); Robert W. Williams, Hockessin, DE (US)

(73) Assignees: Pioneer Hi-Bred International Inc., Johnston, IA (US); E. I. du Pont de Nemours and Company, Wilmington, DE (US)

(21) Appl. No.: 15/107,126

(22) PCT Filed: Dec. 22, 2014
(86) PCT No.: PCT/US14/71897

§ 371 Date: Jun. 22, 2016

Related U.S. Application Data
(60) Provisional application No. 61/921,754, filed on Dec. 30, 2013.

(51) Int. Cl. C12N 15/82; C12N 9/18

(52) U.S. Cl. CPC C12N 15/8273; C12N 9/18; C12Y 301/01001; C12N 15/8271; C12N 15/8261

(57) ABSTRACT

Isolated polynucleotides and polypeptides and recombinant DNA constructs useful for conferring stress tolerance are presented herein, along with compositions (such as plants or seeds) comprising these recombinant DNA constructs, and methods utilizing these recombinant DNA constructs. The recombinant DNA construct comprises a polynucleotide operably linked to a promoter that is functional in a plant, wherein said polynucleotide encodes a DTP4 polypeptide.

### CROSS REFERENCE TO RELATED APPLICATIONS

This application claims the benefit of U.S. Provisional Application No. 61/921,754, filed Dec. 30, 2013, the entire content of which is herein incorporated by reference.

### REFERENCE TO SEQUENCE LISTING SUBMITTED ELECTRONICALLY

The official copy of the sequence listing is submitted electronically via EFS-Web as an ASCII formatted sequence listing with a file named 20141218_BB1672PCT_SequenceListing created on Dec. 18, 2014 and having a size of 1,461 kilobytes and is filed concurrently with the specification. The sequence listing contained in this ASCII formatted document is part of the specification and is herein incorporated by reference in its entirety.

### FIELD

The field relates to plant breeding and genetics and, in particular, relates to recombinant DNA constructs useful in plants for conferring tolerance to drought.

### BACKGROUND

Abiotic stress is the primary cause of crop loss worldwide, causing average yield losses of more than 50% for major crops (Boyer, J. S. (1982) Science 218:443-448; Bray, E. A. et al. (2000) In Biochemistry and Molecular Biology of Plants, Edited by Buchannan, B. B. et al., Amer. Soc. Plant Biol., pp. 1158-1203). Among the various abiotic stresses, drought is the major factor that limits crop productivity worldwide. Exposure of plants to a water-limiting environment during various developmental stages appears to activate various physiological and developmental changes. Understanding of the basic biochemical and molecular mechanism for drought stress perception, transduction and tolerance is a major challenge in biology. Reviews on the molecular mechanisms of abiotic stress responses and the genetic regulatory networks of drought stress tolerance have been published (Valliyodan, B., and Nguyen, H. T., (2006) Curr. Opin. Plant Biol. 9:189-195; Wang, W., et al. (2003) Planta 218:1-14); Vinocur, B., and Altman, A. (2005) Curr. Opin. Biotechnol. 16:123-132; Chaves, M. M., and Oliveira, M. M. (2004) J. Exp. Bot. 55:2365-2384; Shinozaki, K., et al. (2003) Curr. Opin. Plant Biol. 6:410-417; Yamaguchi-Shinozaki, K., and Shinozaki, K. (2005) Trends Plant Sci. 10:88-94).

Another abiotic stress that can limit crop yields is low nitrogen stress. The adsorption of nitrogen by plants plays an important role in their growth (Gallais et al., J. Exp. Bot. 55(396):295-306 (2004)). Plants synthesize amino acids from inorganic nitrogen in the environment. Consequently, nitrogen fertilization has been a powerful tool for increasing the yield of cultivated plants, such as maize and soybean. If the nitrogen assimilation capacity of a plant can be increased, then increases in plant growth and yield increase are also expected. In summary, plant varieties that have better nitrogen use efficiency (NUE) are desirable.

### SUMMARY

The present disclosure includes:

One embodiment of the current disclosure is a plant comprising in its genome a recombinant DNA construct comprising a polynucleotide operably linked to at least one heterologous regulatory element, wherein said polynucleotide encodes a polypeptide having an amino acid sequence of at least 80% sequence identity, when compared to SEQ ID NO:18, 39, 43, 45, 47, 49, 51, 55, 59, 61,64, 65, 66, 95, 97, 101, 103, 107, 111, 113, 117, 119, 121, 123, 127, 129, 130, 131, 132, 135, 627 or 628, and wherein said plant exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, when compared to a control plant not comprising said recombinant DNA construct. In one embodiment said plant exhibits an increase in yield, biomass, or both, when compared to a control plant not comprising said recombinant DNA construct. In one embodiment, said plant exhibits said increase in yield, biomass, or both when compared, under water limiting conditions, to said control plant not comprising said recombinant DNA construct.

One embodiment of the current disclosure also includes seed of the plants disclosed herein, wherein said seed comprises in its genome a recombinant DNA construct comprising a polynucleotide operably linked to at least one heterologous regulatory element, wherein said polynucleotide encodes a polypeptide having an amino acid sequence of at least 80% sequence identity, when compared to SEQ ID NO:18, 39, 43, 45, 47, 49, 51,55, 59, 61,64, 65, 66, 95, 97, 101, 103, 107, 111, 113, 117, 119, 121,123, 127, 129, 130, 131,132, 135, 627 or 628, and wherein a plant produced from said seed exhibits an increase in at least one phenotype selected from the group consisting of: drought stress tolerance, triple stress tolerance, osmotic stress tolerance, nitrogen stress tolerance, tiller number, yield and biomass, when compared to a control plant not comprising said recombinant DNA construct.

One embodiment of the current disclosure is a method of increasing stress tolerance in a plant, wherein the stress is selected from a group consisting of: drought stress, triple stress, nitrogen stress and osmotic stress, the method comprising: (a) introducing into a regenerable plant cell a recombinant DNA construct comprising a polynucleotide operably linked to at least one heterologous regulatory sequence, wherein the polynucleotide encodes a polypeptide having an amino acid sequence of at least 80% sequence identity, when compared to SEQ ID NO:18, 39, 43, 45, 47, 49, 51,55, 59, 61,64, 65, 66, 95, 97, 101, 103, 107, 111, 113, 117, 119, 121, 123, 127, 129, 130, 131, 132, 135, 627 or 628; (b) regenerating a transgenic plant from the regenerable plant cell of (a), wherein the transgenic plant comprises in its genome the recombinant DNA construct; and (c) obtaining a progeny plant derived from the transgenic plant of (b), wherein said progeny plant comprises in its genome the recombinant DNA construct and exhibits increased tolerance to at least one stress selected from the group consisting of drought stress, triple stress, nitrogen stress and osmotic stress, when compared to a control plant not comprising the recombinant DNA construct.

The current disclosure also encompasses a method of selecting for increased stress tolerance in a plant, wherein the stress is selected from a group consisting of: drought stress, triple stress, nitrogen stress and osmotic stress, the method comprising: (a) obtaining a transgenic plant, wherein the transgenic plant comprises in its genome a recombinant DNA construct comprising a polynucleotide operably linked to at least one heterologous regulatory element, wherein said polynucleotide encodes a polypeptide having an amino acid sequence of at least 80% sequence identity, when compared to SEQ ID NO:18, 39, 43, 45, 47, 49, 51, 55, 59, 61, 64, 65, 66, 95, 97, 101, 103, 107, 111, 113, 117, 119, 121, 123, 127, 129, 130, 131, 132, 135, 627 or 628; (b) growing the transgenic plant of part (a) under conditions wherein the polynucleotide is expressed; and (c) selecting the transgenic plant of part (b) with increased stress tolerance, wherein the stress is selected from the group consisting of: drought stress, triple stress, nitrogen stress and osmotic stress, when compared to a control plant not comprising the recombinant DNA construct.

One embodiment of the current disclosure is a method of selecting for an alteration of yield, biomass, or both in a plant, comprising: (a) obtaining a transgenic plant, wherein the transgenic plant comprises in its genome a recombinant DNA construct comprising a polynucleotide operably linked to at least one regulatory element, wherein said polynucleotide encodes a polypeptide having an amino acid sequence of at least 80% sequence identity, when compared to SEQ ID NO:18, 39, 43, 45, 47, 49, 51, 55, 59, 61,64, 65, 66, 95, 97, 101, 103, 107, 111, 113, 117, 119, 121, 123, 127, 129, 130, 131, 132, 135, 627 or 628; (b) growing the transgenic plant of part (a) under conditions wherein the polynucleotide is expressed; and (c) selecting the transgenic plant of part (b) that exhibits an alteration of yield, biomass or both when compared to a control plant not comprising the recombinant DNA construct. In one embodiment, said selecting step (c) comprises determining whether the transgenic plant of (b) exhibits an alteration of yield, biomass or both when compared, under water limiting conditions, to a control plant not comprising the recombinant DNA construct. In one embodiment, said alteration is an increase.

The current disclosure also encompasses an isolated polynucleotide comprising: (a) a nucleotide sequence encoding a polypeptide with stress tolerance activity, wherein the stress is selected from a group consisting of drought stress, triple stress, nitrogen stress and osmotic stress, and wherein the polypeptide has an amino acid sequence of at least 95% sequence identity when compared to SEQ ID NO:18, 39, 43, 45, 47, 49, 51,55, 59, 61,64, 65, 66, 95, 97, 101, 103, 107, 111, 113, 117, 119, 121, 123, 127, 129, 130, 131, 132, 135, 627 or 628; or (b) the full complement of the nucleotide sequence of (a). The amino acid sequence of the polypeptide comprises SEQ ID NO:18, 39, 43, 45, 47, 49, 51, 55, 59, 61, 64, 65, 66, 95, 97, 101, 103, 107, 111, 113, 117, 119, 121, 123, 127, 129, 130, 131, 132, 135, 627 or 628. In one embodiment, the nucleotide sequence comprises SEQ ID NO:16, 17, 19, 38, 42, 44, 46, 48, 50, 54, 58, 60, 62, 63, 94, 96, 100, 102, 106, 110, 112, 116, 118, 120 or 122.

The current disclosure also encompasses a plant or seed comprising a recombinant DNA construct, wherein the recombinant DNA construct comprises any of the polynucleotides disclosed herein, wherein the polynucleotide is operably linked to at least one heterologous regulatory sequence.

In another embodiment, a plant comprising in its genome an endogenous polynucleotide operably linked to at least one heterologous regulatory element, wherein said endogenous polynucleotide encodes a polypeptide having an amino acid sequence of at least 80% sequence identity, when compared to SEQ ID NO:18, 39, 43, 45, 47, 49, 51, 55, 59, 61,64, 65, 66, 95, 97, 101, 103, 107, 111, 113, 117, 119, 121, 123, 127, 129, 130, 131, 132, 135, 627 or 628, and wherein said plant exhibits at least one phenotype selected from the group consisting of increased triple stress tolerance, increased drought stress tolerance, increased nitrogen stress tolerance, increased osmotic stress tolerance, altered ABA response, altered root architecture, increased tiller number, when compared to a control plant not comprising the heterologous regulatory element.

One embodiment is a method of increasing in a crop plant at least one phenotype selected from the group consisting of: triple stress tolerance, drought stress tolerance, nitrogen stress tolerance, osmotic stress tolerance, ABA response, tiller number, yield and biomass, the method comprising increasing the expression of a carboxylesterase in the crop plant. In one embodiment, the crop plant is maize. In one embodiment, the carboxylesterase has at least 80% sequence identity, when compared to SEQ ID NO:18, 39, 43, 45, 47, 49, 51, 55, 59, 61, 64, 65, 66, 95, 97, 101, 103, 107, 111, 113, 117, 119, 121, 123, 127, 129, 130, 131, 132, 135, 627 or 628. In one embodiment, the carboxylesterase gives an E-value score of 1E-15 or less when queried using a Profile Hidden Markov Model prepared using SEQ ID NOS:18, 29, 33, 45, 47, 53, 55, 61, 64, 65, 77, 78, 101, 103, 105, 107, 111, 115, 131, 132, 135, 137, 139, 141, 144, 433, 559 and 604, the query being carried out using the hmmsearch algorithm wherein the Z parameter is set to 1 billion.

Another embodiment is a method of making a plant that exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, when compared to a control plant, the method comprising the steps of introducing into a plant a recombinant DNA construct comprising a polynucleotide operably linked to at least one heterologous regulatory element, wherein said polynucleotide encodes a polypeptide having an amino acid sequence of at least 80% sequence identity, when compared to SEQ ID NO:18, 39, 43, 45, 47, 49, 51, 55, 59, 61,64, 65, 66, 95, 97, 101, 103, 107, 111, 113, 117, 119, 121, 123, 127, 129, 130, 131, 132, 135, 627 or 628.

Another embodiment is a method of producing a plant that exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, wherein the method comprises growing a plant from a seed comprising a recombinant DNA construct, wherein the recombinant DNA construct comprises a polynucleotide operably linked to at least one heterologous regulatory element, wherein the polynucleotide encodes a polypeptide having an amino acid sequence of at least 80% sequence identity, when compared to SEQ ID NO:18, 39, 43, 45, 47, 49, 51, 55, 59, 61, 64, 65, 66, 95, 97, 101, 103, 107, 111, 113, 117, 119, 121, 123, 127, 129, 130, 131, 132, 135, 627 or 628, wherein the plant exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, when compared to a control plant not comprising the recombinant DNA construct.

Another embodiment is a method of producing a seed, the method comprising the following: (a) crossing a first plant with a second plant, wherein at least one of the first plant and the second plant comprises a recombinant DNA construct, wherein the recombinant DNA construct comprises a polynucleotide operably linked to at least one heterologous regulatory element, wherein the polynucleotide encodes a polypeptide having an amino acid sequence of at least 80% sequence identity, when compared to SEQ ID NO:18, 39, 43, 45, 47, 49, 51, 55, 59, 61,64, 65, 66, 95, 97, 101, 103, 107, 111, 113, 117, 119, 121, 123, 127, 129, 130, 131, 132, 135, 627 or 628; and (b) selecting a seed of the crossing of step (a), wherein the seed comprises the recombinant DNA construct. A plant grown from the seed of part (b) exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, when compared to a control plant not comprising the recombinant DNA construct.

In one embodiment, a method of producing oil or a seed by-product, or both, from a seed, the method comprising extracting oil or a seed by-product, or both, from a seed that comprises a recombinant DNA construct, wherein the recombinant DNA construct comprises a polynucleotide operably linked to at least one heterologous regulatory element, wherein the polynucleotide encodes a polypeptide having an amino acid sequence of at least 80% sequence identity, when compared to SEQ ID NO:18, 39, 43, 45, 47, 49, 51,55, 59, 61, 64, 65, 66, 95, 97, 101, 103, 107, 111, 113, 117, 119, 121, 123, 127, 129, 130, 131, 132, 135, 627 or 628. In one embodiment, the seed is obtained from a plant that comprises the recombinant DNA construct and exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, when compared to a control plant not comprising the recombinant DNA construct. In one embodiment, the oil or the seed by-product, or both, comprises the recombinant DNA construct.

In another embodiment, the present disclosure includes any of the methods of the present disclosure wherein the plant is selected from the group consisting of: Arabidopsis, maize, soybean, sunflower, sorghum, canola, wheat, alfalfa, cotton, rice, barley, millet, sugar cane and switchgrass.

In another embodiment, the present disclosure concerns a recombinant DNA construct comprising any of the isolated polynucleotides of the present disclosure operably linked to at least one heterologous regulatory sequence, and a cell, a microorganism, a plant, and a seed comprising the recombinant DNA construct. The cell may be eukaryotic, e.g., a yeast, insect or plant cell, or prokaryotic, e.g., a bacterial cell.

In another embodiment, a plant comprising in its genome a recombinant DNA construct comprising a polynucleotide operably linked to at least one heterologous regulatory element, wherein said polynucleotide encodes a polypeptide having an amino acid sequence of at least 95% sequence identity, when compared to SEQ ID NO:18, and wherein said plant exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, when compared to a control plant not comprising said recombinant DNA construct.

In another embodiment, a method of making a plant that exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, when compared to a control plant, the method comprising the steps of introducing into a plant a recombinant DNA construct comprising a polynucleotide operably linked to at least one heterologous regulatory element, wherein said polynucleotide encodes a polypeptide having an amino acid sequence of at least 95% sequence identity, when compared to SEQ ID NO:18.

### BRIEF DESCRIPTION OF THE DRAWINGS AND SEQUENCE LISTING

The disclosure can be more fully understood from the following detailed description and the accompanying drawings and Sequence Listing which form a part of this application.

FIG. 1A-FIG. 1G show the alignment of the DTP4 polypeptides which were tested in ABA sensitivity assays (SEQ ID NOS:18, 39, 43, 45, 47, 49, 51, 55, 59, 61, 64, 65, 66, 95, 97, 99, 101, 103, 107, 111, 113, 117, 119, 121,123, 127, 129, 130, 131, 132, 135, 627 and 628). Residues that are identical to the residue of consensus sequence (SEQ ID NO:630) at a given position are enclosed in a box. A consensus sequence (SEQ ID NO:630) is presented where a residue is shown if identical in all sequences, otherwise, a period is shown.

FIG. 1C shows the conserved key residues for an oxyanion hole (represented by asterisks), FIG. 1D shows the conserved nucleophile elbow, FIGS. 1D, 1F and 1G also show the catalytic triad of Ser-His-Asp in shaded boxes. These come together in the tertiary structure of the polypeptide.

FIG. 2 shows the percent sequence identity and the divergence values for each pair of amino acids sequences of DTP4 polypeptides displayed in FIG. 1A-1G.

FIG. 3 shows the treatment schedule for screening plants with enhanced drought tolerance.

FIG. 4 shows the percentage germination response of the pBC-yellow-At5g62180 transgenic and wt col-0 Arabidopsis line in an ABA-response assay, at 1 μM ABA.

FIG. 5 shows the yield analysis of maize lines transformed with pCV-DTP4 encoding the Arabidopsis lead gene At5g62180.

FIG. 6A and FIG. 6B show the % germination, % greenness and % true leaf emergence in a 10-day assay, respectively for the wt Arabidopsis plants and At5g62180 transgenic line (Line ID 64) at different quad concentrations. 0% quad is indicated as GM (Growth media).

FIG. 7 shows a graph showing % Germination for the wt and At5g62180 transgenic line, after 48 h at 60%, 65% and 70% quad concentrations.

FIG. 8 shows the schematic of the ABA-Root assay.

FIG. 9 shows an effect of different ABA concentrations on the wt and At5g62180 lines.

FIG. 10 shows the yield analysis of maize lines transformed with pCV-DTP4ac encoding the Arabidopsis lead gene At5g62180, in 1^{st} year field testing, under drought stress.

FIG. 10A shows the yield analysis in 7 different locations that are categorized according to the stress experienced in these locations.

FIG. 10B shows the yield analysis across locations, grouped by stress levels.

FIG. 11 shows the analysis of the agronomic characteristics of maize lines transformed with pCV-DTP4ac encoding the Arabidopsis lead gene At5g62180.

FIG. 11A shows the analysis of ear height (EARHT) and plant height (PLANTHT) in maize lines transformed with pCV-DTP4ac encoding the Arabidopsis lead gene At5g62180.

FIG. 11B shows the analysis of thermal time to shed (TTSHD), root lodging or stalk lodging in maize lines transformed with pCV-DTP4ac encoding the Arabidopsis lead gene At5g62180.

FIG. 12 shows the percentage germination response of the transgenic Arabidopsis plants overexpressing some of the DTP4 polypeptides disclosed herein, compared with wt col-0 Arabidopsis line in an ABA-response assay, at 1 μM ABA (FIG. 12A) and 2 μM ABA (FIG. 12B). FIG. 12 C shows the percentage germination response at 1 μM ABA for some more DTP4 polypeptides, as explained in Table 8.

FIG. 13 shows the percentage green cotyledon response of the transgenic Arabidopsis plants overexpressing some of the DTP4 polypeptides disclosed herein, compared with wt col-0 Arabidopsis line in an ABA-response assay, at 1 μM ABA, as explained in Table 9.

FIG. 14 shows the yield analysis of maize lines transformed with pCV-DTP4ac encoding the Arabidopsis lead gene At5g62180, in 2^{nd} year field testing, under drought stress.

FIG. 14A shows the yield analysis in 8 “no stress” locations.

FIG. 14B shows the yield analysis in 5 “medium stress” locations.

FIG. 14C shows the yield analysis in 5 “severe stress” locations.

FIG. 14 D shows the yield analysis across locations, grouped by drought stress levels, and the last column shows the yield analysis across all locations, regardless of stress level.

FIG. 15 shows the yield analysis of maize lines transformed with pCV-DTP4ac encoding the Arabidopsis lead gene At5g62180, under low nitrogen stress.

FIG. 16A shows the yield analysis of maize lines transformed with pCV-CXE8ac encoding the DTP4 polypeptide, AT-CXE8 (At2g45600; SEQ ID NO:64), under different drought stress locations.

FIG. 16B shows the yield analysis of maize lines transformed with pCV-CXE8ac encoding the DTP4 polypeptide, AT-CXE8 (At2g45600; SEQ ID NO:64), across locations, grouped by different drought stress levels.

FIG. 17 shows the detection of DTP4 protein in transgenic maize leaves by mass spectrometry, at growth stage V9. Values are means and standard errors of 4 field plot replications.

FIG. 18 shows the tiller number in maize plants transformed with pCV-DTP4ac encoding the Arabidopsis lead gene AT-DTP4 (At5g62180), under no stress and drought stress conditions, compared to maize plants not comprising the Arabidopsis gene.

FIG. 19 shows the root and shoot growth response to ABA in maize plants transformed with pCV-DTP4ac encoding the Arabidopsis lead gene AT-DTP4 (At5g62180), under 0 μM and 10 μM ABA. The graphs represent two different experiments done on two different days.

FIG. 20 shows the leaf area in response to triple stress in maize plants transformed with pCV-DTP4ac encoding the Arabidopsis lead gene AT-DTP4 (At5g62180). The graphs represent leaf area 0, 3 and 6 days after treatment (DAT).

FIG. 21 shows the percentage germination response to osmotic stress in maize plants transformed with pCV-DTP4ac encoding the Arabidopsis lead gene AT-DTP4 (At5g62180). The graphs represent two different experiments done on two different days.

FIG. 22 shows shoot growth response in maize plants transformed with pCV-DTP4ac encoding the Arabidopsis lead gene AT-DTP4 (At5g62180), in the tall clear tube assay.

FIG. 23 shows esterase activity of AT-DTP4 fusion protein expressed in E. coli, with p-nitrophenyl acetate as substrate.

FIG. 24 shows the phylogenetic tree showing DTP4 polypeptides.

SEQ ID NO:1 is the nucleotide sequence of the 4×35S enhancer element from the pHSbarENDs2 activation tagging vector.

SEQ ID NO:2 is the nucleotide sequence of the attP1 site.

SEQ ID NO:3 is the nucleotide sequence of the attP2 site.

SEQ ID NO:4 is the nucleotide sequence of the attL1 site.

SEQ ID NO:5 is the nucleotide sequence of the attL2 site.

SEQ ID NO:6 is the nucleotide sequence of the ubiquitin promoter with 5′ UTR and first intron from Zea mays.

SEQ ID NO:7 is the nucleotide sequence of the PinII terminator from Solanum tuberosum.

SEQ ID NO:8 is the nucleotide sequence of the attR1 site.

SEQ ID NO:9 is the nucleotide sequence of the attR2 site.

SEQ ID NO:10 is the nucleotide sequence of the attB1 site.

SEQ ID NO:11 is the nucleotide sequence of the attB2 site.

SEQ ID NO:12 is the nucleotide sequence of the At5g62180-5′attB forward primer, containing the attB1 sequence, used to amplify the At5g62180 protein-coding region.

SEQ ID NO:13 is the nucleotide sequence of the At5g62180-3′attB reverse primer, containing the attB2 sequence, used to amplify the At5g62180 protein-coding region.

SEQ ID NO:14 is the nucleotide sequence of the VC062 primer, containing the T3 promoter and attB1 site, useful to amplify cDNA inserts cloned into a BLUESCRIPT® II SK(+) vector (Stratagene).

SEQ ID NO:15 is the nucleotide sequence of the VC063 primer, containing the T7 promoter and attB2 site, useful to amplify cDNA inserts cloned into a BLUESCRIPT® II SK(+) vector (Stratagene).

SEQ ID NO:16 corresponds to NCBI GI No. 30697645, which is the cDNA sequence from locus At5g62180 encoding an Arabidopsis DTP4 polypeptide.

SEQ ID NO:17 corresponds to the CDS sequence from locus At5g62180 encoding an Arabidopsis DTP4 polypeptide.

SEQ ID NO:18 corresponds to the amino acid sequence of At5g62180 encoded by SEQ ID NO:17.

SEQ ID NO:19 corresponds to a sequence of At5g62180 with alternative codons.

Table 1 presents SEQ ID NOs for the nucleotide sequences obtained from cDNA clones encoding DTP4 polypeptides from Zea mays, Dennstaedtia punctilobula, Sesbania bispinosa, Artemisia tridentata, Lamium amplexicaule, 10 Eschscholzia californica, Linum perenne, Delosperma nubigenum, Peperomia caperata, Triglochin maritime, Chlorophytum comosum, Canna×generalis.

The SEQ ID NOs for the corresponding amino acid sequences encoded by the cDNAs are also presented.

Table 2 presents SEQ ID NOs for more DTP4 polypeptides from public databases.

TABLE 1
cDNAs Encoding DTP4 Polypeptides

| | | SEQ ID NO: | SEQ ID NO: |
|---|---|---|---|
| Plant | Clone Designation* | (Nucleotide) | (Amino Acid) |
| Corn | cfp2n.pk010.p21 | 20 | 21 |
| Corn | cfp2n.pk070.m7 | 22 | 23 |
| Corn | cfp3n.pk007.i9 | 24 | 25 |
| Corn | pco524093 | 26 | 27 |
| Corn | Maize_DTP4-1 | 28 | 29 |
| Corn | Maize_DTP4-2 | 30 | 31 |
| Corn | Maize_DTP4-3 | 32 | 33 |
| Dennstaedtia punctilobula | ehsf2n.pk140.e11 | 34 | 35 |
| Dennstaedtia punctilobula | ehsf2n.pk147.p21 | 36 | 37 |
| Sesbania bispinosa | sesgr1n.pk117.j17 | 38 | 39 |
| Sesbania bispinosa | sesgr1n.pk129.m19 | 40 | 41 |
| Sesbania bispinosa | sesgr1n.pk062.h8 | 42 | 43 |
| Sesbania bispinosa | sesgr1n.pk107.c11 | 44 | 45 |
| Sesbania bispinosa | sesgr1n.pk079.h12 | 46 | 47 |
| Artemisia tridentata | arttr1n.pk125.i16 | 48 | 49 |
| Artemisia tridentata | arttr1n.pk029.e11 | 50 | 51 |
| Artemisia tridentata | arttr1n.pk222.b19 | 52 | 53 |
| Artemisia tridentata | arttr1n.pk120.m9 | 54 | 55 |
| Lamium amplexicaule | hengr1n.pk028.m4 | 56 | 57 |
| Delosperma nubigenum | icegr1n.pk156.e13 | 58 | 59 |
| Peperomia caperata (Emerald | pepgr1n.pk128.o15 | 60 | 61 |
| ripple Peperomia) | | | |
| Peperomia caperata (Emerald | pepgr1n.pk190.l24 | 94 | 95 |
| ripple Peperomia) | | | |
| Peperomia caperata (Emerald | pepgr1n.pk082.c4 | 96 | 97 |
| ripple Peperomia) | | | |
| Linum perenne | lpgr1n.pk005.f19 | 98 | 99 |
| Lamium amplexicaule | hengr1n.pk014.d12 | 100 | 101 |
| Eschscholzia californica | ecalgr1n.pk137.m22 | 102 | 103 |
| Eschscholzia californica | ecalgr1n.pk130.b16 | 104 | 105 |
| Amaranthus hypochondriacus | ahgr1c.pk108.k16 | 106 | 107 |
| Sesbania bispinosa | sesgr1n.pk022.n10_short | 108 | 109 |
| Artemisia tridentata | arttr1n.pk193.a17 | 110 | 111 |
| Artemisia tridentata | arttr1n.pk090.l10 | 112 | 113 |
| Abutilon theophrasti | abtgr1na.pk050.o13 | 150 | 151 |
| Abutilon theophrasti | abtgr1na.pk056.o14 | 152 | 153 |
| Abutilon theophrasti | abtgr1na.pk067.p20 | 154 | 155 |
| Amaranthus hypochondriacus | ahgr1c.pk004.k17 | 156 | 157 |
| Amaranthus hypochondriacus | ahgr1c.pk206.b6 | 158 | 159 |
| Amaranthus hypochondriacus | ahgr1c.pk239.c17 | 160 | 161 |
| Amaranthus hypochondriacus | ahgr1c.pk101.a18 | 162 | 163 |
| Amaranthus hypochondriacus | ahgr1c.pk101.b2 | 164 | 165 |
| Amaranthus hypochondriacus | ahgr1c.pk108.m2 | 166 | 167 |
| Amaranthus hypochondriacus | ahgr1c.pk200.a3.r | 168 | 169 |
| Amaranthus hypochondriacus | ahgr1c.pk228.f18 | 170 | 171 |
| Artemisia tridentata | arttr1n.pk011.m19 | 172 | 173 |
| Artemisia tridentata | arttr1n.pk025.j17 | 174 | 175 |
| Artemisia tridentata | arttr1n.pk030.b19 | 176 | 177 |
| Artemisia tridentata | arttr1n.pk042.k20 | 178 | 179 |
| Artemisia tridentata | arttr1n.pk123.i19 | 180 | 181 |
| Artemisia tridentata | arttr1n.pk183.a10 | 182 | 183 |
| Artemisia tridentata | arttr1n.pk101.f15 | 184 | 185 |
| Artemisia tridentata | arttr1n.pk195.e16 | 186 | 187 |
| Artemisia tridentata | arttr1n.pk047.j22 | 188 | 189 |
| Artemisia tridentata | arttr1n.pk050.l17 | 190 | 191 |
| Artemisia tridentata | arttr1n.pk006.b12.r | 192 | 193 |
| Artemisia tridentata | arttr1n.pk085.i10 | 194 | 195 |
| Artemisia tridentata | arttr1n.pk144.e19 | 196 | 197 |
| Artemisia tridentata | arttr1n.pk147.k17 | 198 | 199 |
| Artemisia tridentata | arttr1n.pk014.h9 | 200 | 201 |
| Artemisia tridentata | arttr1n.pk029.d9 | 202 | 203 |
| Artemisia tridentata | arttr1n.pk187.n1 | 204 | 205 |
| Artemisia tridentata | arttr1n.pk019.g5 | 206 | 207 |
| Artemisia tridentata | arttr1n.pk027.i2 | 208 | 209 |
| Artemisia tridentata | arttr1n.pk029.e6 | 210 | 211 |
| Artemisia tridentata | arttr1n.pk029.p23 | 212 | 213 |
| Artemisia tridentata | arttr1n.pk046.a17 | 214 | 215 |
| Artemisia tridentata | arttr1n.pk138.c10 | 216 | 217 |
| Artemisia tridentata | arttr1n.pk152.i9 | 218 | 219 |
| Artemisia tridentata | arttr1n.pk155.a16 | 220 | 221 |
| Artemisia tridentata | arttr1n.pk158.k23 | 222 | 223 |
| Artemisia tridentata | arttr1n.pk160.h6 | 224 | 225 |
| Artemisia tridentata | arttr1n.pk165.c21 | 226 | 227 |
| Artemisia tridentata | arttr1n.pk165.h5 | 228 | 229 |
| Artemisia tridentata | arttr1n.pk197.d11 | 230 | 231 |
| Artemisia tridentata | arttr1n.pk199.d13 | 232 | 233 |
| Artemisia tridentata | arttr1n.pk214.l5 | 234 | 235 |
| Artemisia tridentata | arttr1n.pk218.l1 | 236 | 237 |
| Artemisia tridentata | arttr1n.pk062.b18 | 238 | 239 |
| Artemisia tridentata | arttr1n.pk104.g4 | 240 | 241 |
| Artemisia tridentata | arttr1n.pk136.n10 | 242 | 243 |
| Artemisia tridentata | arttr1n.pk136.p12 | 244 | 245 |
| Artemisia tridentata | arttr1n.pk175.o6 | 246 | 247 |
| Artemisia tridentata | arttr1n.pk185.f17 | 248 | 249 |
| Artemisia tridentata | arttr1n.pk206.d14 | 250 | 251 |
| Artemisia tridentata | arttr1n.pk212.n16 | 252 | 253 |
| Artemisia tridentata | arttr1n.pk218.n13 | 254 | 255 |
| Artemisia tridentata | arttr1n.pk248.n3 | 256 | 257 |
| Artemisia tridentata | arttr1n.pk203.b15 | 258 | 259 |
| Canna × generalis | cannagr1n306.pk070.m16 | 260 | 261 |
| Canna × generalis | cannagr1n306.pk021.c13 | 262 | 263 |
| Chlorophytum comosum | ccgr1n308l56.pk005.i7 | 264 | 265 |
| Chlorophytum comosum | ccgr1n.pk045.c6 | 266 | 267 |
| Chlorophytum comosum | ccgr1n308l56.pk011.c6 | 268 | 269 |
| Delosperma nubigenum | icegr1n.pk047.c2 | 270 | 271 |
| Delosperma nubigenum | icegr1n.pk197.c3 | 272 | 273 |
| Delosperma nubigenum | icegr1n.pk213.k16 | 274 | 275 |
| Delosperma nubigenum | icegr1n.pk014.l3.r | 276 | 277 |
| Delosperma nubigenum | icegr1n.pk116.d7 | 278 | 279 |
| Delosperma nubigenum | icegr1n.pk035.p22.r | 280 | 281 |
| Delosperma nubigenum | icegr1n.pk073.g5.r | 282 | 283 |
| Delosperma nubigenum | icegr1n.pk162.b18 | 284 | 285 |
| Delosperma nubigenum | icegr1n.pk219.c22 | 286 | 287 |
| Dennstaedtia punctilobula | ehsf2n.pk203.m17 | 288 | 289 |
| Dennstaedtia punctilobula | ehsf2n.pk123.n16 | 290 | 291 |
| Dennstaedtia punctilobula | ehsf2n.pk148.p1 | 292 | 293 |
| Dennstaedtia punctilobula | ehsf2n.pk124.a11 | 294 | 295 |
| Dennstaedtia punctilobula | ehsf2n.pk221.a15 | 296 | 297 |
| Dennstaedtia punctilobula | ehsf2n.pk233.n18 | 298 | 299 |
| Dennstaedtia punctilobula | ehsf2n.pk049.b14 | 300 | 301 |
| Dennstaedtia punctilobula | ehsf2n.pk171.m4 | 302 | 303 |
| Eschscholzia californica | ecalgr1n.pk193.p13.r | 304 | 305 |
| Eschscholzia californica | ecalgr1n.pk130.g3 | 306 | 307 |
| Eschscholzia californica | ecalgr1n.pk016.p16 | 308 | 309 |
| Eschscholzia californica | ecalgr1n.pk042.h15 | 310 | 311 |
| Eschscholzia californica | ecalgr1n.pk128.h17 | 312 | 313 |
| Eschscholzia californica | ecalgr1n.pk132.f19 | 314 | 315 |
| Eschscholzia californica | ecalgr1n.pk008.m5 | 316 | 317 |
| Eschscholzia californica | ecalgr1n.pk063.d23 | 318 | 319 |
| Eschscholzia californica | ecalgr1n.pk070.g7 | 320 | 321 |
| Eschscholzia californica | ecalgr1n.pk121.e22 | 322 | 323 |
| Eschscholzia californica | ecalgr1n.pk132.f20 | 324 | 325 |
| Eschscholzia californica | ecalgr1n.pk140.c5 | 326 | 327 |
| Eschscholzia californica | ecalgr1n.pk145.e6 | 328 | 329 |
| Eschscholzia californica | ecalgr1n.pk172.m18 | 330 | 331 |
| Eschscholzia californica | ecalgr1n.pk194.e7 | 332 | 333 |
| Eschscholzia californica | ecalgr1n.pk152.p24 | 334 | 335 |
| Eschscholzia californica | ecalgr1n.pk007.a21 | 336 | 337 |
| Eschscholzia californica | ecalgr1n.pk028.m20 | 338 | 339 |
| Eschscholzia californica | ecalgr1n.pk049.n17 | 340 | 341 |
| Eschscholzia californica | ecalgr1n.pk086.l10 | 342 | 343 |
| Eschscholzia californica | ecalgr1n.pk092.n18.r | 344 | 345 |
| Eschscholzia californica | ecalgr1n.pk095.l21 | 346 | 347 |
| Eschscholzia californica | ecalgr1n.pk111.h1 | 348 | 349 |
| Eschscholzia californica | ecalgr1n.pk142.b14 | 350 | 351 |
| Eschscholzia californica | ecalgr1n.pk169.l22 | 352 | 353 |
| Eschscholzia californica | ecalgr1n.pk192.l15 | 354 | 355 |
| Lamium amplexicaule | hengr1n.pk056.e14 | 356 | 357 |
| Lamium amplexicaule | hengr1n.pk015.c10 | 358 | 359 |
| Lamium amplexicaule | hengr1n.pk019.g3 | 360 | 361 |
| Lamium amplexicaule | hengr1n.pk169.h24 | 362 | 363 |
| Lamium amplexicaule | hengr1n.pk019.a8 | 364 | 365 |
| Lamium amplexicaule | hengr1n.pk042.e4 | 366 | 367 |
| Lamium amplexicaule | hengr1n.pk106.i3 | 368 | 369 |
| Lamium amplexicaule | hengr1n.pk183.g9 | 370 | 371 |
| Lamium amplexicaule | hengr1n.pk006.e14 | 372 | 373 |
| Lamium amplexicaule | hengr1n.pk139.k22.r | 374 | 375 |
| Lamium amplexicaule | hengr1n.pk205.e4 | 376 | 377 |
| Lamium amplexicaule | hengr1n.pk083.p6.r | 378 | 379 |
| Lamium amplexicaule | hengr1n.pk099.i9 | 380 | 381 |
| Lamium amplexicaule | hengr1n.pk132.n2 | 382 | 383 |
| Lamium amplexicaule | hengr1n.pk166.h13 | 384 | 385 |
| Lamium amplexicaule | hengr1n.pk191.p1 | 386 | 387 |
| Lamium amplexicaule | hengr1n.pk252.o11 | 388 | 389 |
| Lamium amplexicaule | hengr1n.pk007.p2 | 390 | 391 |
| Lamium amplexicaule | hengr1n.pk121.a23 | 392 | 393 |
| Lamium amplexicaule | hengr1n.pk062.j19 | 394 | 395 |
| Lamium amplexicaule | hengr1n.pk104.j11 | 396 | 397 |
| Lamium amplexicaule | hengr1n.pk124.a20 | 398 | 399 |
| Lamium amplexicaule | hengr1n.pk182.c11 | 400 | 401 |
| Lamium amplexicaule | hengr1n.pk252.b18 | 402 | 403 |
| Linum perenne | lpgr1n.pk122.d12 | 404 | 405 |
| Linum perenne | lpgr1n.pk049.d20 | 406 | 407 |
| Linum perenne | lpgr1n.pk023.c23.r | 408 | 409 |
| Linum perenne | lpgr1n.pk008.f18 | 410 | 411 |
| Linum perenne | lpgr1n.pk085.m11 | 412 | 413 |
| Linum perenne | lpgr1n.pk102.p22 | 414 | 415 |
| Linum perenne | lpgr1n.pk055.f13.r | 416 | 417 |
| Linum perenne | lpgr1n.pk059.i18.r | 418 | 419 |
| Linum perenne | lpgr1n.pk074.m24.r | 420 | 421 |
| Linum perenne | lpgr1n.pk016.a14 | 422 | 423 |
| Linum perenne | lpgr1n.pk030.p21 | 424 | 425 |
| Linum perenne | lpgr1n.pk035.j14 | 426 | 427 |
| Linum perenne | lpgr1n.pk060.a17 | 428 | 429 |
| Peperomia caperata | pepgr1n.pk053.k21 | 430 | 431 |
| Peperomia caperata | pepgr1n.pk070.b11 | 432 | 433 |
| Peperomia caperata | pepgr1n.pk098.f11 | 434 | 435 |
| Peperomia caperata | pepgr1n.pk048.n2 | 436 | 437 |
| Peperomia caperata | pepgr1n.pk240.d2 | 438 | 439 |
| Peperomia caperata | pepgr1n.pk075.j19 | 440 | 441 |
| Peperomia caperata | pepgr1n.pk143.g17 | 442 | 443 |
| Peperomia caperata | pepgr1n.pk224.n19 | 444 | 445 |
| Peperomia caperata | pepgr1n.pk236.p10 | 446 | 447 |
| Sesbania bispinosa | sesgr1n.pk067.o14 | 448 | 449 |
| Sesbania bispinosa | sesgr1n.pk069.p21 | 450 | 451 |
| Sesbania bispinosa | sesgr1n.pk140.i18 | 452 | 453 |
| Sesbania bispinosa | sesgr1n.pk119.d14 | 454 | 455 |
| Sesbania bispinosa | sesgr1n.pk059.f22 | 456 | 457 |
| Sesbania bispinosa | sesgr1n.pk108.j9 | 458 | 459 |
| Sesbania bispinosa | sesgr1n.pk019.p14 | 460 | 461 |
| Sesbania bispinosa | sesgr1n.pk117.d15 | 462 | 463 |
| Sesbania bispinosa | sesgr1n.pk132.p20 | 464 | 465 |
| Sesbania bispinosa | sesgr1n.pk142.e7 | 466 | 467 |
| Sesbania bispinosa | sesgr1n.pk151.n5 | 468 | 469 |
| Sesbania bispinosa | sesgr1n.pk154.p5 | 470 | 471 |
| Sesbania bispinosa | sesgr1n.pk172.f15 | 472 | 473 |
| Sesbania bispinosa | sesgr1n.pk120.c11 | 474 | 475 |
| Sesbania bispinosa | sesgr1n.pk007.h12 | 476 | 477 |
| Sesbania bispinosa | sesgr1n.pk024.h4 | 478 | 479 |
| Sesbania bispinosa | sesgr1n.pk028.i7 | 480 | 481 |
| Sesbania bispinosa | sesgr1n.pk034.p15 | 482 | 483 |
| Sesbania bispinosa | sesgr1n.pk041.p8 | 484 | 485 |
| Sesbania bispinosa | sesgr1n.pk080.f8 | 486 | 487 |
| Sesbania bispinosa | sesgr1n.pk083.d4 | 488 | 489 |
| Sesbania bispinosa | sesgr1n.pk126.e15 | 490 | 491 |
| Sesbania bispinosa | sesgr1n.pk172.d10 | 492 | 493 |
| Triglochin maritima | tmgr2n.pk038.g19 | 494 | 495 |
| Triglochin maritima | tmgr2n308l56.pk045.l21 | 496 | 497 |
| Triglochin maritima | tmgr2n.pk042.m4.r | 498 | 499 |
| Triglochin maritima | tmgr2n.pk009.b15 | 500 | 501 |
| Triglochin maritima | tmgr2n.pk020.a24 | 502 | 503 |
| Triglochin maritima | tmgr2n.pk036.i19 | 504 | 505 |
| Triglochin maritima | tmgr2n.pk048.f6 | 506 | 507 |
| Triglochin maritima | tmgr2n308l56.pk031.p21 | 508 | 509 |

*The “Full-Insert Sequence” (“FIS”) is the sequence of the entire cDNA insert.

SEQ ID NO:62 is the nucleotide sequence encoding AT-CXE8 polypeptide; corresponding to At2g45600 locus (Arabidopsis thaliana).

SEQ ID NO:63 is the AT-CXE8 nucleotide sequence with alternative codons.

SEQ ID NO:64 is the amino acid sequence corresponding to NCBI GI No. 75318485 (AT-CXE8), encoded by the sequence given in SEQ ID NO:62 and 63; (Arabidopsis thaliana).

SEQ ID NO:65 is the amino acid sequence corresponding to NCBI GI No. 75318486 (AT-CXE9), encoded by the locus At2g45610.1 (Arabidopsis thaliana).

SEQ ID NO:66 is the amino acid sequence corresponding to NCBI GI No. 75335430 (AT-CXE18), encoded by the locus At5g23530.1 (Arabidopsis thaliana).

SEQ ID NO:67 is the amino acid sequence corresponding to the locus LOC_Os08g43430.1, a rice (japonica) predicted protein from the Michigan State University Rice Genome Annotation Project Osa1 release 6.

SEQ ID NO:68 is the amino acid sequence corresponding to the locus LOC_Os03g14730.1, a rice (japonica) predicted protein from the Michigan State University Rice Genome Annotation Project Osa1 release 6.

SEQ ID NO:69 is the amino acid sequence corresponding to the locus LOC_Os07g44890.1, a rice (japonica) predicted protein from the Michigan State University Rice Genome Annotation Project Osa1 release 6.

SEQ ID NO:70 is the amino acid sequence corresponding to the locus LOC_Os07g44860.1, a rice (japonica) predicted protein from the Michigan State University Rice Genome Annotation Project Osa1 release 6.

SEQ ID NO:71 is the amino acid sequence corresponding to the locus LOC_Os07g44910.1, a rice (japonica) predicted protein from the Michigan State University Rice Genome Annotation Project Osa1 release 6.

SEQ ID NO:72 is the amino acid sequence corresponding to Sb07g025010.1, a sorghum (Sorghum bicolor) predicted protein from the Sorghum JGI genomic sequence version 1.4 from the US Department of energy Joint Genome Institute.

SEQ ID NO:73 is the amino acid sequence corresponding to Sb01g040930.1, a sorghum (Sorghum bicolor) predicted protein from the Sorghum JGI genomic sequence version 1.4 from the US Department of energy Joint Genome Institute.

SEQ ID NO:74 is the amino acid sequence corresponding to Glyma20g29190.1, a soybean (Glycine max) predicted protein from predicted coding sequences from Soybean JGI Glyma1.01 genomic sequence from the US Department of energy Joint Genome Institute.

SEQ ID NO:75 is the amino acid sequence corresponding to Glyma20g29200.1, a soybean (Glycine max) predicted protein from predicted coding sequences from Soybean JGI Glyma1.01 genomic sequence from the US Department of energy Joint Genome Institute.

SEQ ID NO:76 is the amino acid sequence corresponding to Glyma16g32560.1, a soybean (Glycine max) predicted protein from predicted coding sequences from Soybean JGI Glyma1.01 genomic sequence from the US Department of energy Joint Genome Institute.

SEQ ID NO:77 is the amino acid sequence corresponding to Glyma07g09040.1, a soybean (Glycine max) predicted protein from predicted coding sequences from Soybean JGI Glyma1.01 genomic sequence from the US Department of energy Joint Genome Institute.

SEQ ID NO:78 is the amino acid sequence corresponding to Glyma07g09030.1, a soybean (Glycine max) predicted protein from predicted coding sequences from Soybean JGI Glyma1.01 genomic sequence from the US Department of energy Joint Genome Institute.

SEQ ID NO:79 is the amino acid sequence corresponding to Glyma03g02330.1, a soybean (Glycine max) predicted protein from predicted coding sequences from Soybean JGI Glyma1.01 genomic sequence from the US Department of energy Joint Genome Institute.

SEQ ID NO:80 is the amino acid sequence corresponding to Glyma09g27500.1, a soybean (Glycine max) predicted protein from predicted coding sequences from Soybean JGI Glyma1.01 genomic sequence from the US Department of energy Joint Genome Institute.

SEQ ID NO:81 the amino acid sequence presented in SEQ ID NO:12 of U.S. Pat. No. 7,915,050 (Arabidopsis thaliana).

SEQ ID NO:82 is the amino acid sequence corresponding to NCBI GI No. 194704970 (Zea mays).

SEQ ID NO:83 the amino acid sequence presented in SEQ ID NO:260345 of US Patent Publication No. US20120216318 (Zea mays).

SEQ ID NO:84 is the amino acid sequence corresponding to NCBI GI No. 195636334 (Zea mays).

SEQ ID NO:85 the amino acid sequence presented in SEQ ID NO:331675 of US Patent Publication No. US20120216318.

SEQ ID NO:86 is the amino acid sequence corresponding to NCBI GI No. 194707422 (Zea mays).

SEQ ID NO:87 the amino acid sequence presented in SEQ ID NO:7332 of U.S. Pat. No. 8,343,764 (Zea mays).

SEQ ID NO:88 is the amino acid sequence corresponding to NCBI GI No. 223948401 (Zea mays).

SEQ ID NO:89 the amino acid sequence presented in SEQ ID NO:16159 of U.S. Pat. No. 7,569,389 (Zea mays).

SEQ ID NO:90 is the amino acid sequence corresponding to NCBI GI No. 23495723 (Oryza sativa).

SEQ ID NO:91 the amino acid sequence presented in SEQ ID NO:50819 of US Patent Publication No. US20120017292 (Zea mays).

SEQ ID NO:92 is the amino acid sequence corresponding to NCBI GI No. 215768720 (Oryza sativa).

SEQ ID NO:93 the amino acid sequence presented in SEQ ID NO:10044 of U.S. Pat. No. 8,362,325 (Sorghum bicolor).

SEQ ID NO:114 is the nucleotide sequence of a DTP4 polypeptide from Carica papaya.

SEQ ID NO:115 is the amino acid sequence of a polypeptide, encoded by the nucleotide sequence presented in SEQ ID NO:114 (Carica papaya).

SEQ ID NO:116 is the nucleotide sequence of a polypeptide from Eutrema salsugineum.

SEQ ID NO:117 is the amino acid sequence of a polypeptide, encoded by the nucleotide sequence presented in SEQ ID NO:116 (Eutrema salsugineum).

SEQ ID NO:118 is the nucleotide sequence of an assembled contig from Brassica napus and Brassica oleracea sequences(Bn-Bo).

SEQ ID NO:119 is the amino acid sequence of a polypeptide, encoded by the nucleotide sequence presented in SEQ ID NO:118.

SEQ ID NO:120 is the nucleotide sequence of an assembled contig from Brassica napus and Brassica oleracea sequences (Bole-someBnap).

SEQ ID NO:121 is the amino acid sequence of a polypeptide, encoded by the nucleotide sequence presented in SEQ ID NO:120.

SEQ ID NO:122 is the nucleotide sequence of an assembled contig of ESTs from Brassica napus.

SEQ ID NO:123 is the amino acid sequence of a polypeptide, encoded by the nucleotide sequence presented in SEQ ID NO:122.

SEQ ID NO:124 is the nucleotide sequence of an assembled contig of ESTs from Citrus sinensis and Citrus clementina.

SEQ ID NO:125 is the amino acid sequence of a DTP4 polypeptide from Citrus sinensis and Citrus clementina.

SEQ ID NO:126 is the amino acid sequence of a DTP4 polypeptide from Raphanus sativus.

SEQ ID NO:127 is the amino acid sequence of a DTP4 polypeptide from Arabidopsis lyrata.

SEQ ID NO:128 is the amino acid sequence of a DTP4 polypeptide from Olimarabidopsis pumila.

SEQ ID NO:129 is the amino acid sequence of a DTP4 polypeptide from Capsella rubella.

SEQ ID NO:130 is the amino acid sequence of a DTP4 polypeptide from Capsella rubella.

SEQ ID NO:131 is the amino acid sequence of a DTP4 polypeptide from Brassica rapa subsp. pekinensis.

SEQ ID NO:132 is the amino acid sequence of a DTP4 polypeptide from Brassica rapa subsp. pekinensis.

SEQ ID NO:133 is the amino acid sequence of a DTP4 polypeptide from Prunus persica.

SEQ ID NOS:134 and 135 are the amino acid sequences of 2 DTP4 homologs from Vitis vinifera.

SEQ ID NO:136 is the nucleotide sequence of a Vitis vinifera DTP4 polypeptide named GSVIVT01027568001 (unique_1).

SEQ ID NO:137 is the amino acid sequence of the DTP4 polypeptide sequence of a Vitis vinifera DTP4 polypeptide (GSVIVT01027568001; unique_1).

SEQ ID NO:138 is the nucleotide sequence of a Vitis vinifera DTP4 homolog named GSVIVT01027566001 (unique_2).

SEQ ID NO:139 is the amino acid sequence of the DTP4 polypeptide sequence of a Vitis vinifera DTP4 polypeptide (GSVIVT01027566001; unique_2).

SEQ ID NO:140 is the nucleotide sequence of a Vitis vinifera DTP4 homolog named GSVIVT01027569001 (unique_3).

SEQ ID NO:141 is the amino acid sequence of the DTP4 polypeptide sequence of a Vitis vinifera DTP4 polypeptide (GSVIVT01027569001; unique_3).

SEQ ID NOS:142-149 are the amino acid sequences of DTP4 polypeptides from Populus trichocarpa.

SEQ ID NO:627 is the amino acid sequence encoded by the locus At1g49660 (AT-CXE5) (Arabidopsis thaliana).

SEQ ID NO:628 is the amino acid sequence encoded by the locus At5g16080 (AT-CXE17) (Arabidopsis thaliana).

SEQ ID NO:629 is the sequence of the fusion protein of AT-DTP4 overexpressed in E. coli.

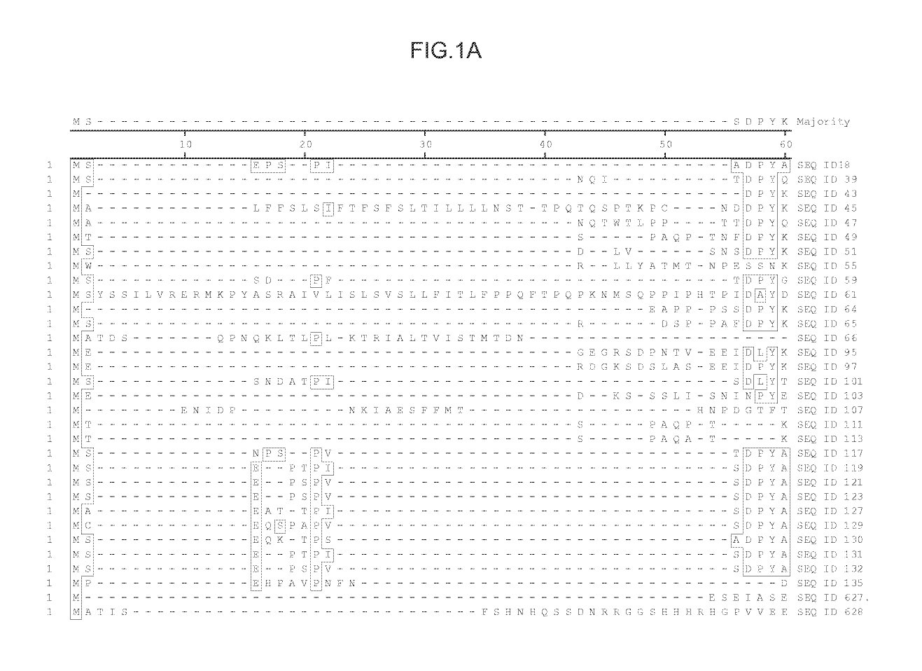

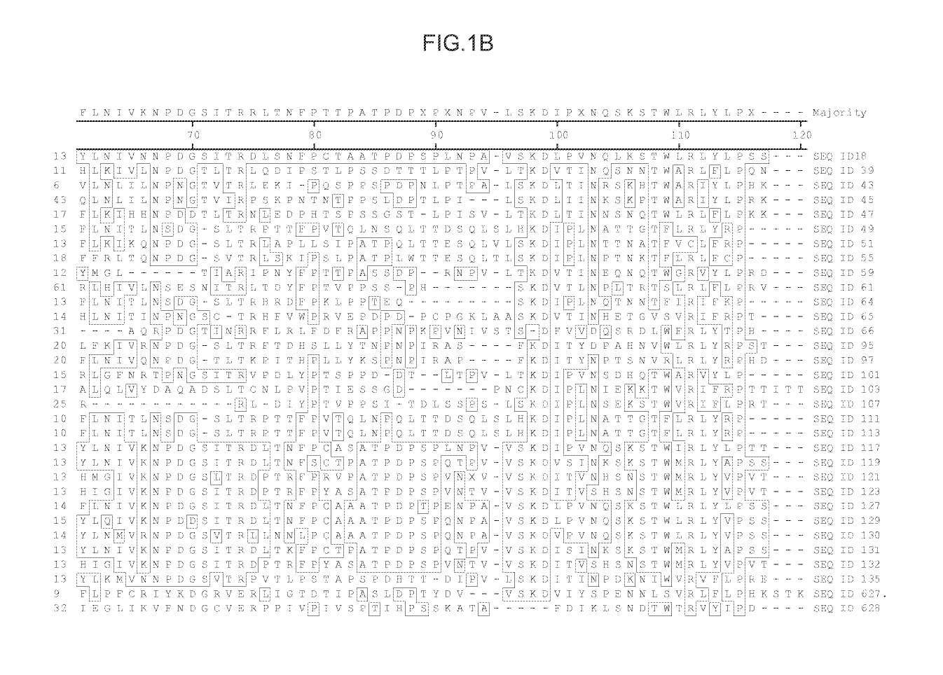

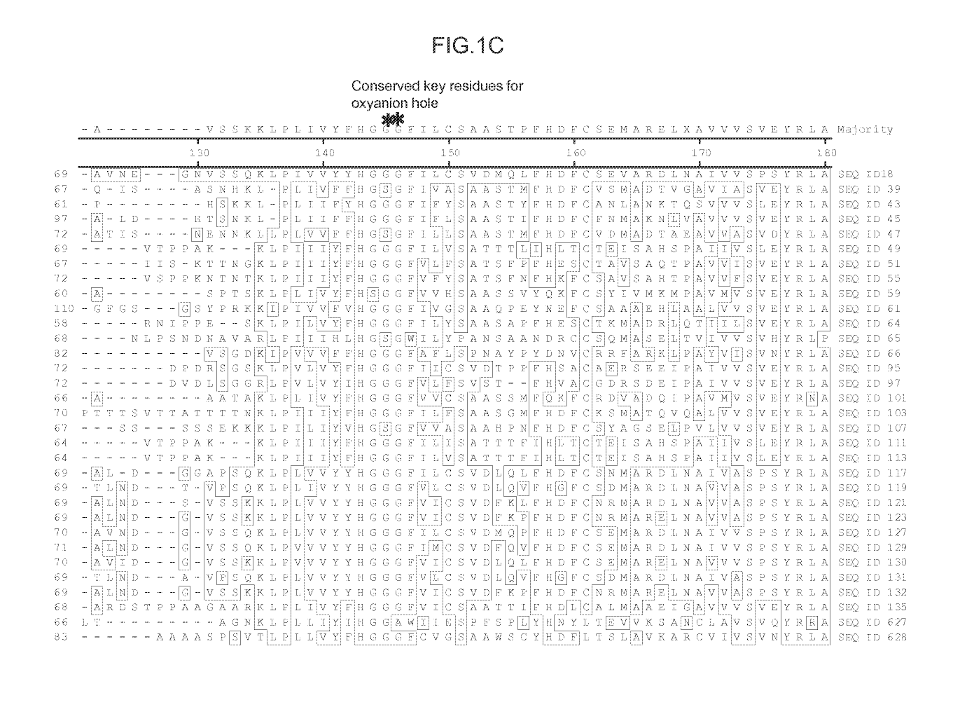

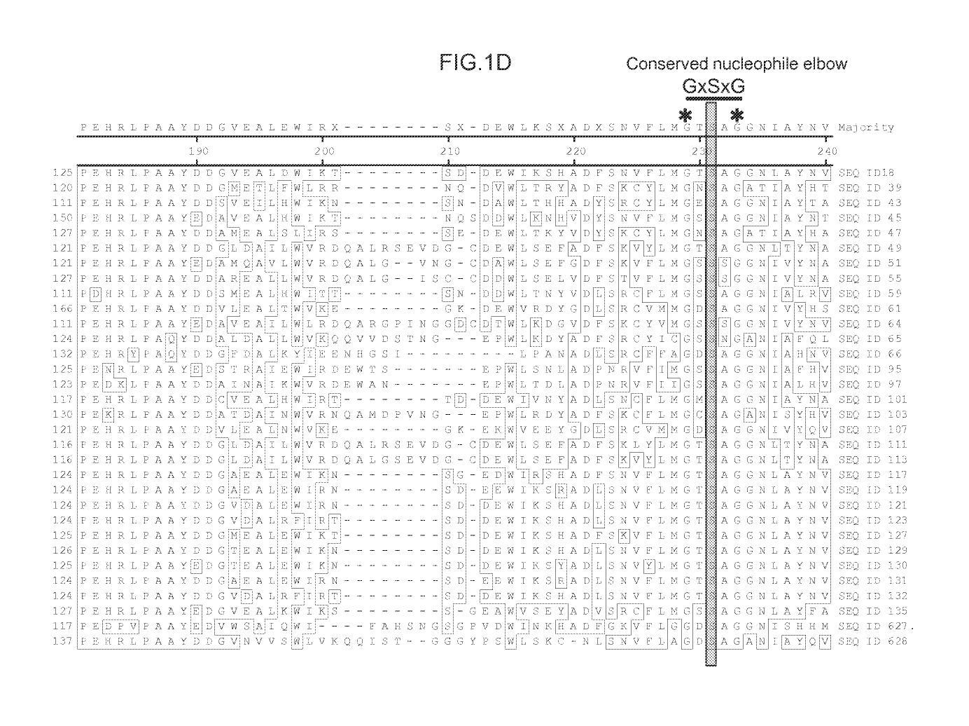

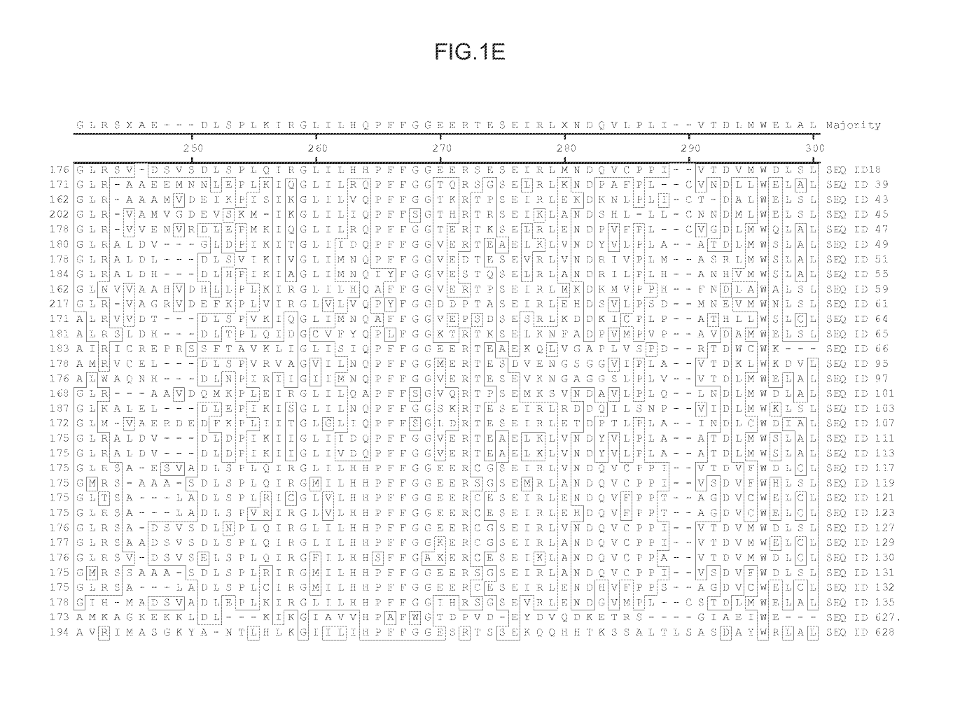

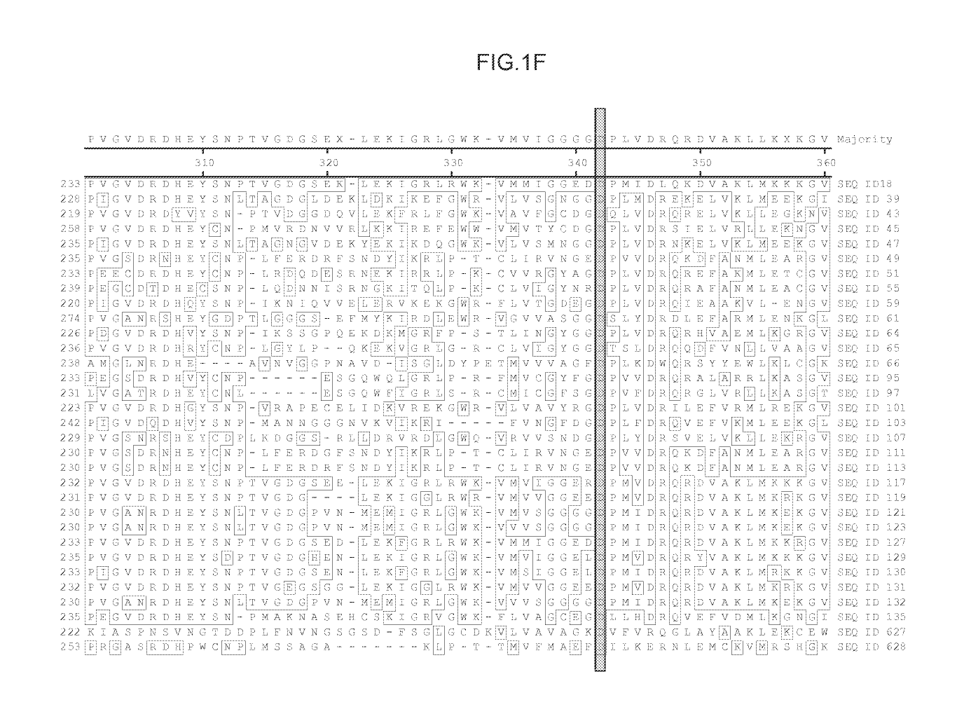

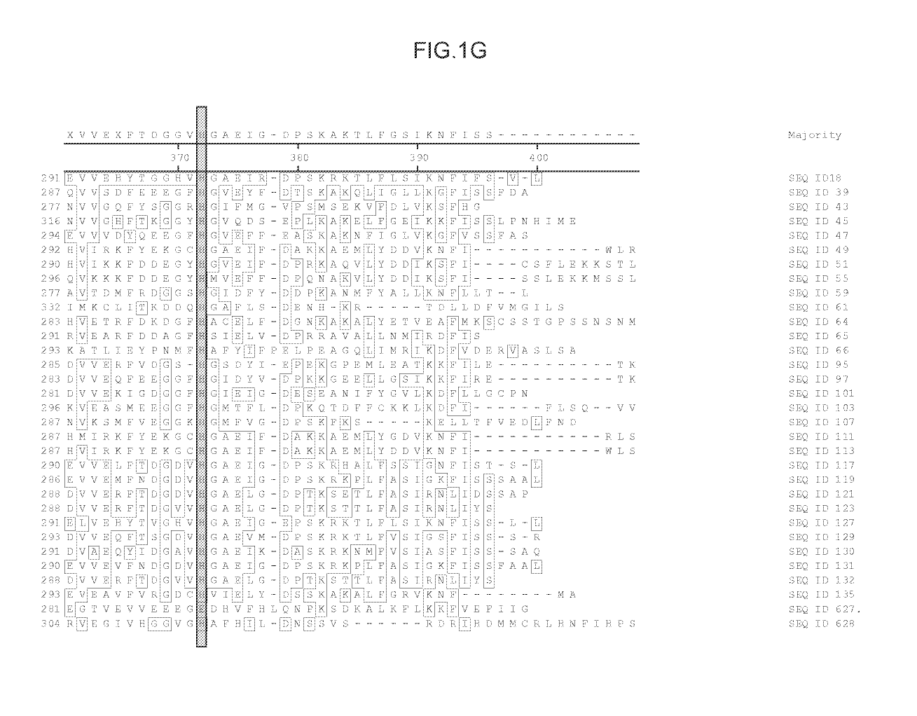

SEQ ID NO:630 is the consensus sequence obtained from the alignment of sequences given in FIG. 1

TABLE 2
DTP4 polypeptides

| | | SEQ ID NO: |
|---|---|---|
| Plant | Clone Designation* | (Amino Acid) |
| Arabidopsis halleri | Araha.2214s0019.1.p | 510 |
| Arabidopsis lyrata | D7LCK9_Al | 511 |
| Arabidopsis thaliana | AT3G05120.1_At_CXE10 | 512 |
| Arabidopsis thaliana | AT3G63010.1_At_CXE14 | 513 |
| Arabidopsis thaliana | AT5G27320.1_At_CXE19 | 514 |
| Arabidopsis thaliana | At5g06570_AtCXE15 | 515 |
| Arabidopsis thaliana | At1g68620_AtCXE6 | 516 |
| Boechera stricta | Bostr.25993s0214.1.p | 517 |
| Boechera stricta | Bostr.26833s0018.1.p | 518 |
| Boechera stricta | Bostr.26675s0205.1.p | 519 |
| Brachypodium distachyon | Bradi1g67930.1_BRADI | 520 |
| Brachypodium distachyon | Bradi3g42207.1_BRADI | 521 |
| Brassica rapa | Brara.E00516.1.p | 522 |
| Brassica rapa | Brara.I00681.1.p | 523 |
| Brassica rapa | Brara.B03796.1.p | 524 |
| Brassica rapa | Brara.B03797.1.p | 525 |
| Brassica rapa | Brara.E01694.1.p | 526 |
| Capsella grandiflora | Cagra.21374s0001.1.p | 527 |
| Capsella grandiflora | Cagra.4003s0009.1.p | 528 |
| Capsella grandiflora | Cagra.2481s0010.1.p | 529 |
| Capsella rubella | R0HTV4_Cr | 530 |
| Capsella rubella | Carubv10011237m | 531 |
| Capsella rubella | Carubv10011932m | 532 |
| Carica papaya | Cpap_18.158_PACid_16411302 | 533 |
| Carica papaya | Cpap_18.159_PACid_16411303 | 534 |
| Eutrema salsugineum | Thhalv10011663m | 535 |
| Glycine max | Glyma07g09030.1 | 536 |
| Glycine max | Glyma02g17010.1 | 537 |
| Glycine max | Glyma03g30460.1 | 538 |
| Glycine max | Glyma09g28580.1 | 539 |
| Glycine max | Glyma09g28590.1 | 540 |
| Glycine max | Glyma10g02790.1 | 541 |
| Glycine max | Glyma10g29910.1 | 542 |
| Glycine max | Glyma16g33320.1 | 543 |
| Glycine max | Glyma16g33330.1 | 544 |
| Glycine max | Glyma16g33340.1 | 545 |
| Glycine max | Glyma20g37430.1 | 546 |
| Glycine max | Glyma02g27090.1 | 547 |
| Glycine max | Glyma03g36380.1 | 548 |
| Glycine max | Glyma06g46520.1 | 549 |
| Glycine max | Glyma06g46520.2 | 550 |
| Glycine max | Glyma10g11060.1 | 551 |
| Glycine max | Glyma12g10250.1 | 552 |
| Glycine max | Glyma19g39030.1 | 553 |
| Glycine max | Glyma08g47930.1 | 554 |
| Glycine max | Glyma10g42260.1 | 555 |
| Glycine max | Glyma17g31740.1 | 556 |
| Glycine max | Glyma18g53580.1 | 557 |
| Glycine max | Glyma20g24780.1 | 558 |
| Gossypium raimondii | Gorai.007G093200.1 | 559 |
| Gossypium raimondii | Gorai.008G282100.1 | 560 |
| Oryza sativa | LOC_Os05g33730.1 | 561 |
| Oryza sativa | LOC_Os06g20200.1 | 562 |
| Oryza sativa | LOC_Os07g41590.1 | 563 |
| Oryza sativa | LOC_Os07g44850.1 | 564 |
| Oryza sativa | LOC_Os07g44900.1 | 565 |
| Oryza sativa | LOC_Os11g13570.1 | 566 |
| Oryza sativa | LOC_Os11g13630.1 | 567 |
| Oryza sativa | LOC_Os11g13670.1 | 568 |
| Oryza sativa | LOC_Os01g06060.1_OsCXE4 | 569 |
| Oryza sativa | LOC_Os01g06210.1_OsCXE2 | 570 |
| Oryza sativa | LOC_Os01g06220.1_OsCXE1 | 571 |
| Oryza sativa | LOC_Os03g57640.1 | 572 |
| Oryza sativa | LOC_Os07g06830.1 | 573 |
| Oryza sativa | LOC_Os07g06840.1 | 574 |
| Oryza sativa | LOC_Os07g06850.1 | 575 |
| Oryza sativa | LOC_Os07g06860.1 | 576 |
| Oryza sativa | LOC_Os07g06880.1 | 577 |
| Oryza sativa | LOC_Os03g15270.1 | 578 |
| Sorghum bicolor | Sb02g038880.1 | 579 |
| Sorghum bicolor | Sb02g041000.1 | 580 |
| Sorghum bicolor | Sb02g041040.1 | 581 |
| Sorghum bicolor | Sb02g041050.1 | 582 |
| Sorghum bicolor | Sb05g007270.1 | 583 |
| Sorghum bicolor | Sb05g007290.1 | 584 |
| Sorghum bicolor | Sb09g020080.1 | 585 |
| Sorghum bicolor | Sb09g020080.2 | 586 |
| Sorghum bicolor | Sb01g005720.1 | 587 |
| Sorghum bicolor | Sb02g003560.1 | 588 |
| Sorghum bicolor | Sb02g003570.1 | 589 |
| Sorghum bicolor | Sb02g003580.1 | 590 |
| Sorghum bicolor | Sb02g003600.1 | 591 |
| Sorghum bicolor | Sb02g003610.1 | 592 |
| Sorghum bicolor | Sb02g003620.1 | 593 |
| Sorghum bicolor | Sb02g003630.1 | 594 |
| Sorghum bicolor | Sb02g020810.1 | 595 |
| Sorghum bicolor | Sb03g005560.1 | 596 |
| Sorghum bicolor | Sb03g005570.1 | 597 |
| Sorghum bicolor | Sb03g005580.1 | 598 |
| Sorghum bicolor | Sb03g005590.1 | 599 |
| Sorghum bicolor | Sb01g040580.1 | 600 |
| Eutrema salsugineum | Thhalv10001557m_PACid_20189097 | 601 |
| Eutrema salsugineum | Thhalv10001767m_PACid_20188989 | 602 |
| Theobroma cacao | Thecc1EG005469t1 | 603 |
| Theobroma cacao | Thecc1EG015038t1_edit | 604 |
| Theobroma cacao | Thecc1EG032452t1 | 605 |
| Vitis vinifera | GSVIVT01027566001 | 606 |
| Vitis vinifera | GSVIVT01027569001 | 607 |
| Zea mays | Maize_DTP4-4 | 608 |
| Zea mays | Maize_DTP4-5 | 609 |
| Zea mays | Maize_DTP4-6 | 610 |
| Zea mays | Maize_DTP4-7 | 611 |
| Zea mays | Maize_DTP4-8 | 612 |
| Zea mays | Maize_DTP4-9 | 613 |
| Zea mays | Maize_DTP4-10 | 614 |
| Zea mays | Maize_DTP4-11 | 615 |
| Zea mays | Maize_DTP4-12 | 616 |
| Zea mays | Maize_DTP4-13 | 617 |
| Zea mays | Maize_DTP4-14 | 618 |
| Zea mays | Maize_DTP4-15 | 619 |
| Zea mays | Maize_DTP4-16 | 620 |
| Zea mays | Maize_DTP4-17 | 621 |
| Zea mays | Maize_DTP4-18 | 622 |
| Zea mays | Maize_DTP4-19 | 623 |
| Zea mays | Maize_DTP4-20 | 624 |
| Zea mays | Maize_DTP4-21 | 625 |
| Zea mays | Maize_DTP4-22 | 626 |

The sequence descriptions and Sequence Listing attached hereto comply with the rules governing nucleotide and/or amino acid sequence disclosures in patent applications as set forth in 37 C.F.R. §1.821-1.825.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

As used herein:

The term “AT-DTP4” generally refers to an Arabidopsis thaliana protein that is encoded by the Arabidopsis thaliana locus At5g62180. The terms “AT-DTP4”, “AT-CXE20”, “AT-carboxyesterase” and “AT-carboxylesterase 20” are used interchangeably herein. “DTP4 polypeptide” refers herein to the AT-DTP4 polypeptide and its homologs or orthologs from other organisms. The terms Zm-DTP4 and Gm-DTP4 refer respectively to Zea mays and Glycine max proteins that are homologous to AT-DTP4.

The term DTP4 polypeptide as described herein refers to any of the DTP4 polypeptides given in Table 1 and Table 2 in the specification.

The term DTP4 polypeptide also encompasses a polypeptide wherein the polypeptide gives an E-value score of 1E-15 or less when queried using a Profile Hidden Markov Model prepared using SEQ ID NOS:18, 29, 33, 45, 47, 53, 55, 61, 64, 65, 77, 78, 101, 103, 105, 107, 111, 115, 131, 132, 135, 137, 139, 141, 144, 433, 559 and 604, the query being carried out using the hmmsearch algorithm wherein the Z parameter is set to 1 billion. The term DTP4 polypeptide also refers herein to a polypeptide wherein the polypeptide gives an E-value score of 1E-15 or less when queried using the Profile Hidden Markov Model given in Table 18.

Nakajima et al (Plant Journal (2006) 46, 880-889) have shown that AT-DTP4 polypeptide sequence has homology to gibberellin receptors, no GA binding capability was detectable in recombinant AT-DTP4 polypeptides.

Based on phylogenetic analysis, Marshall et al have identified the protein encoded by At5g62180 as a carboxylesterase (CXE). By RT-PCR expression analysis, at-cxe20 was shown to be expressed in almost all Arabidopsis tissues (Marshall et al J Mol Evol (2003) 57:487-500).

The main feature of carboxylesterases (or carboxyesterases) is the conserved catalytic triad. The active site is made up of a serine (surrounded by the conserved consensus sequence G-X-S-X-G), a glutamate (or less frequently an aspartate), and a histidine (Marshall et al J Mol Evol (2003) 57:487-500). These residues are dispersed throughout the primary amino acid sequence but come together in the tertiary structure to form a charge relay system, creating a nucleophilic serine that can attack the substrate. Another structural motif of importance is the oxyanion hole, which is involved in stabilizing the substrate-enzyme intermediate during hydrolysis. The oxyanion hole is created by three small amino acids: two glycine residues typically located between b-strand 3 and a-helix 1 and the third located immediately following the catalytic serine residue (Marshall et al J Mol Evol (2003) 57:487-500).

The AT-CXE20 polypeptide has a conserved “nucleophile elbow” (G×S×G) with a unique conformation to activate the nucleophile residue S166, the conserved catalytic triad at S166-H302-D272 and the “oxyanion hole” with the conserved residues G88-G89-G90 for stabilizing the negatively charged transition state.

Some of these conserved sites and residues are shown in the alignment figures (FIG. 1).

Esterases that are part of the alpha/beta hydrolase_3 fold (Pfam domain PF07859) form the group of hydrolases that are expected to provide drought tolerance and/or increased yield for crop plants.

The terms “monocot” and “monocotyledonous plant” are used interchangeably herein. A monocot of the current disclosure includes the Gramineae.

The terms “dicot” and “dicotyledonous plant” are used interchangeably herein. A dicot of the current disclosure includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

An “Expressed Sequence Tag” (“EST”) is a DNA sequence derived from a cDNA library and therefore is a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

A “trait” generally refers to a physiological, morphological, biochemical, or physical characteristic of a plant or a particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, or by agricultural observations such as osmotic stress tolerance or yield. The term “trait” is used interchangeably with the term “phenotype” herein.

“Agronomic characteristic” is a measurable parameter including but not limited to, abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, leaf number, tiller number, growth rate, first pollen shed time, first silk emergence time, anthesis silking interval (ASI), stalk diameter, root architecture, staygreen, relative water content, water use, water use efficiency; dry weight of either main plant, tillers, primary ear, main plant and tillers or cobs; rows of kernels, total plant weight·kernel weight, kernel number, salt tolerance, chlorophyll content, flavonol content, number of yellow leaves, early seedling vigor and seedling emergence under low temperature stress. These agronomic characteristics maybe measured at any stage of the plant development. One or more of these agronomic characteristics may be measured under stress or non-stress conditions, and may show alteration on overexpression of the recombinant constructs disclosed herein.

“Tiller number” herein refers to the average number of tillers on a plant. A tiller is defined as a secondary shoot that has developed and has a tassel capable of shedding pollen (U.S. Pat. No. 7,723,584).

Tillers are grain-bearing branches in monocotyledonous plants. The number of tillers per plant is a key factor that determines yield in the many major cereal crops, such as rice and wheat, therefore by increasing tiller number, there is a potential for increasing the yield of major cereal crops like rice, wheat, and barley.

Abiotic stress may be at least one condition selected from the group consisting of: drought, water deprivation, flood, high light intensity, high temperature, low temperature, salinity, etiolation, defoliation, heavy metal toxicity, anaerobiosis, nutrient deficiency, nutrient excess, UV irradiation, atmospheric pollution (e.g., ozone) and exposure to chemicals (e.g., paraquat) that induce production of reactive oxygen species (ROS).

“Increased stress tolerance” of a plant is measured relative to a reference or control plant, and is a trait of the plant to survive under stress conditions over prolonged periods of time, without exhibiting the same degree of physiological or physical deterioration relative to the reference or control plant grown under similar stress conditions.

A plant with “increased stress tolerance” can exhibit increased tolerance to one or more different stress conditions.

“Stress tolerance activity” of a polypeptide indicates that over-expression of the polypeptide in a transgenic plant confers increased stress tolerance to the transgenic plant relative to a reference or control plant.

A polypeptide with a certain activity, such as a polypeptide with one or more than one activity selected from the group consisting of: increased triple stress tolerance, increased drought stress tolerance, increased nitrogen stress tolerance, increased osmotic stress tolerance, altered ABA response, altered root architecture, increased tiller number; indicates that overexpression of the polypeptide in a plant confers the corresponding phenotype to the plant relative to a reference or control plant. For example, a plant overexpressing a polypeptide with “altered ABA response activity”, would exhibit the phenotype of “altered ABA response”, when compared to a control or reference plant.

Increased biomass can be measured, for example, as an increase in plant height, plant total leaf area, plant fresh weight, plant dry weight or plant seed yield, as compared with control plants.

The ability to increase the biomass or size of a plant would have several important commercial applications. Crop species may be generated that produce larger cultivars, generating higher yield in, for example, plants in which the vegetative portion of the plant is useful as food, biofuel or both.

Increased leaf size may be of particular interest. Increasing leaf biomass can be used to increase production of plant-derived pharmaceutical or industrial products. An increase in total plant photosynthesis is typically achieved by increasing leaf area of the plant. Additional photosynthetic capacity may be used to increase the yield derived from particular plant tissue, including the leaves, roots, fruits or seed, or permit the growth of a plant under decreased light intensity or under high light intensity.

Modification of the biomass of another tissue, such as root tissue, may be useful to improve a plants ability to grow under harsh environmental conditions, including drought or nutrient deprivation, because larger roots may better reach water or nutrients or take up water or nutrients.

For some ornamental plants, the ability to provide larger varieties would be highly desirable. For many plants, including fruit-bearing trees, trees that are used for lumber production, or trees and shrubs that serve as view or wind screens, increased stature provides improved benefits in the forms of greater yield or improved screening.

The growth and emergence of maize silks has a considerable importance in the determination of yield under drought (Fuad-Hassan et al. 2008 Plant Cell Environ. 31:1349-1360). When soil water deficit occurs before flowering, silk emergence out of the husks is delayed while anthesis is largely unaffected, resulting in an increased anthesis-silking interval (ASI) (Edmeades et al. 2000 Physiology and Modeling Kernel set in Maize (eds M. E. Westgate & K. Boote; CSSA (Crop Science Society of America) Special Publication No. 29. Madison, Wis.: CSSA, 43-73). Selection for reduced ASI has been used successfully to increase drought tolerance of maize (Edmeades et al. 1993 Crop Science 33: 1029-1035; Bolanos & Edmeades 1996 Field Crops Research 48:65-80; Bruce et al. 2002 J. Exp. Botany 53:13-25).

Terms used herein to describe thermal time include “growing degree days” (GDD), “growing degree units” (GDU) and “heat units” (HU).

“Transgenic” generally refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, plant propagules, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Propagule” includes all products of meiosis and mitosis able to propagate a new plant, including but not limited to, seeds, spores and parts of a plant that serve as a means of vegetative reproduction, such as corms, tubers, offsets, or runners. Propagule also includes grafts where one portion of a plant is grafted to another portion of a different plant (even one of a different species) to create a living organism. Propagule also includes all plants and seeds produced by cloning or by bringing together meiotic products, or allowing meiotic products to come together to form an embryo or fertilized egg (naturally or with human intervention).

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to co-transformation, retransformation, and crossing lines with different transgenes.

“Transgenic plant” also includes reference to plants which comprise more than one heterologous polynucleotide within their genome. Each heterologous polynucleotide may confer a different trait to the transgenic plant.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” generally refers to the RNA that is without introns and that can be translated into protein by the cell.

“cDNA” generally refers to a DNA that is complementary to and synthesized from a mRNA template using the enzyme reverse transcriptase. The cDNA can be single-stranded or converted into the double-stranded form using the Klenow fragment of DNA polymerase I.

“Coding region” generally refers to the portion of a messenger RNA (or the corresponding portion of another nucleic acid molecule such as a DNA molecule) which encodes a protein or polypeptide. “Non-coding region” generally refers to all portions of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” are used interchangeably herein. The terms “non-coding region” and “non-coding sequence” are used interchangeably herein.

“Mature” protein generally refers to a post-translationally processed polypeptide; i.e., one from which any pre- or pro-peptides present in the primary translation product have been removed.

“Precursor” protein generally refers to the primary product of translation of mRNA; i.e., with pre- and pro-peptides still present. Pre- and pro-peptides may be and are not limited to intracellular localization signals.

“Isolated” generally refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

As used herein the terms non-genomic nucleic acid sequence or non-genomic nucleic acid molecule generally refer to a nucleic acid molecule that has one or more change in the nucleic acid sequence compared to a native or genomic nucleic acid sequence. In some embodiments the change to a native or genomic nucleic acid molecule includes but is not limited to: changes in the nucleic acid sequence due to the degeneracy of the genetic code; codon optimization of the nucleic acid sequence for expression in plants; changes in the nucleic acid sequence to introduce at least one amino acid substitution, insertion, deletion and/or addition compared to the native or genomic sequence; removal of one or more intron associated with a genomic nucleic acid sequence; insertion of one or more heterologous introns; deletion of one or more upstream or downstream regulatory regions associated with a genomic nucleic acid sequence; insertion of one or more heterologous upstream or downstream regulatory regions; deletion of the 5′ and/or 3′ untranslated region associated with a genomic nucleic acid sequence; and insertion of a heterologous 5′ and/or 3′ untranslated region.

“Recombinant” generally refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” generally refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature. The terms “recombinant DNA construct” and “recombinant construct” are used interchangeably herein.

The terms “entry clone” and “entry vector” are used interchangeably herein.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” generally refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably, and refer to a promoter that is expressed predominantly but not necessarily exclusively in one tissue or organ, but that may also be expressed in one specific cell.

“Developmentally regulated promoter” generally refers to a promoter whose activity is determined by developmental events.

“Operably linked” generally refers to the association of nucleic acid fragments in a single fragment so that the function of one is regulated by the other. For example, a promoter is operably linked with a nucleic acid fragment when it is capable of regulating the transcription of that nucleic acid fragment.

“Expression” generally refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein generally refers to both stable transformation and transient transformation.

“Stable transformation” generally refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” generally refers to the introduction of a nucleic acid fragment into the nucleus, or DNA-containing organelle, of a host organism resulting in gene expression without genetically stable inheritance.

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

A “chloroplast transit peptide” is an amino acid sequence which is translated in conjunction with a protein and directs the protein to the chloroplast or other plastid types present in the cell in which the protein is made (Lee et al. (2008) Plant Cell 20:1603-1622). The terms “chloroplast transit peptide” and “plastid transit peptide” are used interchangeably herein. “Chloroplast transit sequence” generally refers to a nucleotide sequence that encodes a chloroplast transit peptide. A “signal peptide” is an amino acid sequence which is translated in conjunction with a protein and directs the protein to the secretory system (Chrispeels (1991) Ann. Rev. Plant Phys. Plant Mol. Biol. 42:21-53). If the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present should be removed and instead a nuclear localization signal included (Raikhel (1992) Plant Phys. 100:1627-1632). A “mitochondrial signal peptide” is an amino acid sequence which directs a precursor protein into the mitochondria (Zhang and Glaser (2002) Trends Plant Sci 7:14-21).

Sequence alignments and percent identity calculations may be determined using a variety of comparison methods designed to detect homologous sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CABIOS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

Complete sequences and figures for vectors described herein (e.g., pHSbarENDs2, pDONRM/Zeo, pDONRM221, pBC-yellow, PHP27840, PHP23236, PHP10523, PHP23235 and PHP28647) are given in PCT Publication No. WO/2012/058528, the contents of which are herein incorporated by reference.

Turning now to the embodiments:

Embodiments include isolated polynucleotides and polypeptides, recombinant DNA constructs useful for conferring drought tolerance, compositions (such as plants or seeds) comprising these recombinant DNA constructs, and methods utilizing these recombinant DNA constructs.

Isolated Polynucleotides and Polypeptides:

The present disclosure includes the following isolated polynucleotides and polypeptides:

An isolated polynucleotide comprising: (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:18, 39, 43, 45, 47, 49, 51, 55, 59, 61, 64, 65, 66, 95, 97, 101, 103, 107, 111, 113, 117, 119, 121, 123, 127, 129, 130, 131, 132, 135, 627 or 628, and combinations thereof; or (ii) a full complement of the nucleic acid sequence of (i), wherein the full complement and the nucleic acid sequence of (i) consist of the same number of nucleotides and are 100% complementary. Any of the foregoing isolated polynucleotides may be utilized in any recombinant DNA constructs (including suppression DNA constructs) of the present disclosure. The polypeptide is preferably a DTP4 polypeptide. The polypeptide preferably has stress tolerance activity, wherein the stress is selected from the group consisting of drought stress, triple stress, osmotic stress and nitrogen stress. The polypeptide may also have at least one activity selected from the group consisting of: carboxylesterase, increased triple stress tolerance, increased drought stress tolerance, increased nitrogen stress tolerance, increased osmotic stress tolerance, altered ABA response, altered root architecture, increased tiller number.

An isolated polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:18, 39, 43, 45, 47, 49, 51,55, 59, 61,64, 65, 66, 95, 97, 101, 103, 107, 111, 113, 117, 119, 121, 123, 127, 129, 130, 131, 132, 135, 627 or 628, and combinations thereof. The polypeptide is preferably a DTP4 polypeptide. The polypeptide preferably has stress tolerance activity, wherein the stress is selected from the group consisting of drought stress, triple stress, nitrogen stress and osmotic stress. The polypeptide may also have at least one activity selected from the group consisting of carboxylesterase, increased triple stress tolerance, increased drought stress tolerance, increased nitrogen stress tolerance, increased osmotic stress tolerance, altered ABA response, altered root architecture, increased tiller number.

An isolated polynucleotide comprising (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:16, 17, 19, 38, 42, 44, 46, 48, 50, 54, 58, 60, 62, 63, 94, 96, 100, 102, 106, 110, 112, 116, 118, 120 or 122, and combinations thereof; or (ii) a full complement of the nucleic acid sequence of (i). Any of the foregoing isolated polynucleotides may be utilized in any recombinant DNA constructs (including suppression DNA constructs) of the present disclosure. The isolated polynucleotide preferably encodes a DTP4 polypeptide. The polypeptide preferably has stress tolerance activity, wherein the stress is selected from the group consisting of drought stress, triple stress, osmotic stress and nitrogen stress. The polypeptide may also have at least one activity selected from the group consisting of: carboxylesterase, increased triple stress tolerance, increased drought stress tolerance, increased nitrogen stress tolerance, increased osmotic stress tolerance, altered ABA response, altered root architecture, increased tiller number.

An isolated polynucleotide comprising a nucleotide sequence, wherein the nucleotide sequence is hybridizable under stringent conditions with a DNA molecule comprising the full complement of SEQ ID NO:16, 17, 19, 38, 42, 44, 46, 48, 50, 54, 58, 60, 62, 63, 94, 96, 100, 102, 106, 110, 112, 116, 118, 120 or 122. The isolated polynucleotide preferably encodes a DTP4 polypeptide. The polypeptide preferably has stress tolerance activity, wherein the stress is selected from the group consisting of drought stress, triple stress, osmotic stress and nitrogen stress.

An isolated polynucleotide comprising a nucleotide sequence, wherein the nucleotide sequence is derived from SEQ ID NO:16, 17, 19, 38, 42, 44, 46, 48, 50, 54, 58, 60, 62, 63, 94, 96, 100, 102, 106, 110, 112, 116, 118, 120 or 122 by alteration of one or more nucleotides by at least one method selected from the group consisting of: deletion, substitution, addition and insertion. The isolated polynucleotide preferably encodes a DTP4 polypeptide. The polypeptide preferably has stress tolerance activity, wherein the stress is selected from the group consisting of drought stress, triple stress, osmotic stress and nitrogen stress. The polypeptide may also have at least one activity selected from the group consisting of: carboxylesterase, increased triple stress tolerance, increased drought stress tolerance, increased nitrogen stress tolerance, increased osmotic stress tolerance, altered ABA response, altered root architecture, increased tiller number.

An isolated polynucleotide comprising a nucleotide sequence, wherein the nucleotide sequence corresponds to an allele of SEQ ID NO:16, 17, 19, 38, 42, 44, 46, 48, 50, 54, 58, 60, 62, 63, 94, 96, 100, 102, 106, 110, 112, 116, 118, 120 or 122.

In any of the preceding embodiments, the DTP4 polypeptide can be any of the DTP4 polypeptide given in Table 1 and Table 2.

In any of the preceding embodiments, the DTP4 polypeptide may be encoded by any of the nucleotide sequences given in Table 1 and Table 2.

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

The protein of the current disclosure may also be a protein which comprises an amino acid sequence comprising deletion, substitution, insertion and/or addition of one or more amino acids in an amino acid sequence presented in SEQ ID NO:18, 39, 43, 45, 47, 49, 51, 55, 59, 61, 64, 65, 66, 95, 97, 101, 103, 107, 111, 113, 117, 119, 121,123, 127, 129, 130, 131,132, 135, 627 or 628. The substitution may be conservative, which means the replacement of a certain amino acid residue by another residue having similar physical and chemical characteristics. Non-limiting examples of conservative substitution include replacement between aliphatic group-containing amino acid residues such as lie, Val, Leu or Ala, and replacement between polar residues such as Lys-Arg, Glu-Asp or Gln-Asn replacement.

Proteins derived by amino acid deletion, substitution, insertion and/or addition can be prepared when DNAs encoding their wild-type proteins are subjected to, for example, well-known site-directed mutagenesis (see, e.g., Nucleic Acid Research, Vol. 10, No. 20, p. 6487-6500, 1982, which is hereby incorporated by reference in its entirety). As used herein, the term “one or more amino acids” is intended to mean a possible number of amino acids which may be deleted, substituted, inserted and/or added by site-directed mutagenesis.

Site-directed mutagenesis may be accomplished, for example, as follows using a synthetic oligonucleotide primer that is complementary to single-stranded phage DNA to be mutated, except for having a specific mismatch (i.e., a desired mutation). Namely, the above synthetic oligonucleotide is used as a primer to cause synthesis of a complementary strand by phages, and the resulting duplex DNA is then used to transform host cells. The transformed bacterial culture is plated on agar, whereby plaques are allowed to form from phage-containing single cells. As a result, in theory, 50% of new colonies contain phages with the mutation as a single strand, while the remaining 50% have the original sequence. At a temperature which allows hybridization with DNA completely identical to one having the above desired mutation, but not with DNA having the original strand, the resulting plaques are allowed to hybridize with a synthetic probe labeled by kinase treatment. Subsequently, plaques hybridized with the probe are picked up and cultured for collection of their DNA.

Techniques for allowing deletion, substitution, insertion and/or addition of one or more amino acids in the amino acid sequences of biologically active peptides such as enzymes while retaining their activity include site-directed mutagenesis mentioned above, as well as other techniques such as those for treating a gene with a mutagen, and those in which a gene is selectively cleaved to remove, substitute, insert or add a selected nucleotide or nucleotides, and then ligated.

The protein of the present disclosure may also be a protein which is encoded by a nucleic acid comprising a nucleotide sequence comprising deletion, substitution, insertion and/or addition of one or more nucleotides in the nucleotide sequence of SEQ ID NO:16, 17, 19, 38, 42, 44, 46, 48, 50, 54, 58, 60, 62, 63, 94, 96, 100, 102, 106, 110, 112, 116, 118, 120 or 122. Nucleotide deletion, substitution, insertion and/or addition may be accomplished by site-directed mutagenesis or other techniques as mentioned above.

The protein of the present disclosure may also be a protein which is encoded by a nucleic acid comprising a nucleotide sequence hybridizable under stringent conditions with the complementary strand of the nucleotide sequence of SEQ ID NO:16, 17, 19, 38, 42, 44, 46, 48, 50, 54, 58, 60, 62, 63, 94, 96, 100, 102, 106, 110, 112, 116, 118, 120 or 122.

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

It is also possible to use a commercially available hybridization kit which uses no radioactive substance as a probe. Specific examples include hybridization with an ECL direct labeling & detection system (Amersham). Stringent conditions include, for example, hybridization at 42° C. for 4 hours using the hybridization buffer included in the kit, which is supplemented with 5% (w/v) Blocking reagent and 0.5 M NaCl, and washing twice in 0.4% SDS, 0.5×SSC at 55° C. for 20 minutes and once in 2×SSC at room temperature for 5 minutes.

DTP4 polypeptides included in the current disclosure are also those that have an E-value score of 1E-15 or less when queried using a Profile Hidden Markov Model (Profile HMM) prepared using SEQ ID NOS:18, 29, 33, 45, 47, 53, 55, 61,64, 65, 77, 78, 101, 103, 105, 107, 111, 115, 131, 132, 135, 137, 139, 141, 144, 433, 559 and 604; the query being carried out using the hmmsearch algorithm wherein the Z parameter is set to 1 billion.

In one embodiment, the E-value score can be 1E-15, 1E-25, 1E-35, 1E-45, 1E-55, 1E-65, 1E-70, 1E-75, 1E-80 or 1E-85.

The terms “Profile HMMs” or “HMM profile” are used interchangeably herein as used herein are statistical models of multiple sequence alignments, or even of single sequences. They capture position-specific information about how conserved each column of the alignment is, and which residues are likely (Krogh et al., 1994, J. Mol. Biol., 235:1501-1531; Eddy, 1998, Curr. Opin. Struct. Biol., 6:361-365.; Durbin et al., Probabilistic Models of Proteins and Nucleic Acids. Cambridge University Press, Cambridge UK. (1998); Eddy, Sean R., March 2010, HMMER User's Guide Version 3.0, Howard Hughes Medical Institute, Janelia Farm Research Campus, Ashburn Va., USA; US patent publication No. US20100293118; U.S. Pat. No. 8,623,623).

The term “E-value” or “Expect value (E)” is a parameter which provides the probability that a match will occur by chance. It provides the statistical significance of the match to a sequence. The lower the E-value, the more significant the hit. It decreases exponentially as the Score (S) of the match increases.

The Z parameter refers to the ability to set the database size, for purposes of E-value calculation (Eddy, Sean R., March 2010, HMMER User's Guide Version 3.0, Howard Hughes Medical Institute, Janelia Farm Research Campus, Ashburn Va., USA).

Recombinant DNA Constructs and Suppression DNA Constructs:

In one embodiment, the present disclosure includes recombinant DNA constructs (including suppression DNA constructs).

In one embodiment, a recombinant DNA construct comprises a polynucleotide operably linked to at least one heterologous regulatory sequence (e.g., a promoter functional in a plant), wherein the polynucleotide comprises (i) a nucleic acid sequence encoding an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:18, 39, 43, 45, 47, 49, 51, 55, 59, 61, 64, 65, 66, 95, 97, 101, 103, 107, 111, 113, 117, 119, 121, 123, 127, 129, 130, 131, 132, 135, 627 or 628, and combinations thereof; or (ii) a full complement of the nucleic acid sequence of (i). The polypeptide may have at least one activity selected from the group consisting of carboxylesterase, increased triple stress tolerance, increased drought stress tolerance, increased nitrogen stress tolerance, increased osmotic stress tolerance, altered ABA response, altered root architecture, increased tiller number,

In another embodiment, a recombinant DNA construct comprises a polynucleotide operably linked to at least one heterologous regulatory sequence (e.g., a promoter functional in a plant), wherein said polynucleotide comprises (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:16, 17, 19, 38, 42, 44, 46, 48, 50, 54, 58, 60, 62, 63, 94, 96, 100, 102, 106, 110, 112, 116, 118, 120 or 122, and combinations thereof; or (ii) a full complement of the nucleic acid sequence of (i).

In another embodiment, a recombinant DNA construct comprises a polynucleotide operably linked to at least one heterologous regulatory sequence (e.g., a promoter functional in a plant), wherein said polynucleotide encodes a DTP4 polypeptide. The DTP4 polypeptide preferably has stress tolerance activity, wherein the stress is selected from the group consisting of drought stress, triple stress, osmotic stress and nitrogen stress. The polypeptide may have at least one activity selected from the group consisting of carboxylesterase, increased triple stress tolerance, increased drought stress tolerance, increased nitrogen stress tolerance, increased osmotic stress tolerance, altered ABA response, altered root architecture, increased tiller number,

In any of the embodiments given herein, the DTP4 polypeptide may be selected from any of the polypeptides listed in Table 1 and Table 2.

The DTP4 polypeptide may be from Arabidopsis thaliana, Zea mays, Glycine max, Glycine tabacina, Glycine soja, Glycine tomentella, Oryza sativa, Brassica napus, Sorghum bicolor, Saccharum officinarum, Triticum aestivum, or any of the plant species disclosed herein.

In one embodiment, a recombinant construct comprises a polynucleotide, wherein the polynucleotide is operably linked to a heterologous promoter, and encodes a polypeptide with at least one activity selected from the group consisting of: carboxylesterase, increased triple stress tolerance, increased drought stress tolerance, increased nitrogen stress tolerance, increased osmotic stress tolerance, altered ABA response, altered root architecture, increased tiller number, wherein the polypeptide gives an E-value score of 1E-15 or less when queried using a Profile Hidden Markov Model prepared using SEQ ID NOS:18, 29, 33, 45, 47, 53, 55, 61, 64, 65, 77, 78, 101, 103, 105, 107, 111, 115, 131, 132, 135, 137, 139, 141, 144, 433, 559 and 604, the query being carried out using the hmmsearch algorithm wherein the Z parameter is set to 1 billion.

In another aspect, the present disclosure includes suppression DNA constructs.

A suppression DNA construct may comprise at least one heterologous regulatory sequence (e.g., a promoter functional in a plant) operably linked to (a) all or part of: (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:18, 39, 43, 45, 47, 49, 51,55, 59, 61, 64, 65, 66, 95, 97, 101, 103, 107, 111, 113, 117, 119, 121, 123, 127, 129, 130, 131, 132, 135, 627 or 628, and combinations thereof, or (ii) a full complement of the nucleic acid sequence of (a)(i); or (b) a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to said all or part of a sense strand or antisense strand from which said region is derived, and wherein said target gene of interest encodes a DTP4 polypeptide; or (c) all or part of: (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:16, 17, 19, 38, 42, 44, 46, 48, 50, 54, 58, 60, 62, 63, 94, 96, 100, 102, 106, 110, 112, 116, 118, 120 or 122, and combinations thereof, or (ii) a full complement of the nucleic acid sequence of (c)(i). The suppression DNA construct may comprise a cosuppression construct, antisense construct, viral-suppression construct, hairpin suppression construct, stem-loop suppression construct, double-stranded RNA-producing construct, RNAi construct, or small RNA construct (e.g., an siRNA construct or an miRNA construct).

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the sense strand (or antisense strand) of the gene of interest.

A suppression DNA construct may comprise 100, 200, 300, 400, 500, 600, 700, 800, 900 or 1000 contiguous nucleotides of the sense strand (or antisense strand) of the gene of interest, and combinations thereof.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as siRNA (short interfering RNA) constructs and miRNA (microRNA) constructs.

Suppression of gene expression may also be achieved by use of artificial miRNA precursors, ribozyme constructs and gene disruption. A modified plant miRNA precursor may be used, wherein the precursor has been modified to replace the miRNA encoding region with a sequence designed to produce a miRNA directed to the nucleotide sequence of interest. Gene disruption may be achieved by use of transposable elements or by use of chemical agents that cause site-specific mutations.

“Antisense inhibition” generally refers to the production of antisense RNA transcripts capable of suppressing the expression of the target gene or gene product. “Antisense RNA” generally refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns, or the coding sequence.

“Cosuppression” generally refers to the production of sense RNA transcripts capable of suppressing the expression of the target gene or gene product. “Sense” RNA generally refers to RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence (see Vaucheret et al., Plant J. 16:651-659 (1998); and Gura, Nature 404:804-808 (2000)).

Another variation describes the use of plant viral sequences to direct the suppression of proximal mRNA encoding sequences (PCT Publication No. WO 98/36083 published on Aug. 20, 1998).

RNA interference generally refers to the process of sequence-specific post-transcriptional gene silencing in animals mediated by short interfering RNAs (siRNAs) (Fire et al., Nature 391:806 (1998)). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 (1999)).

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides (nt) in length that have been identified in both animals and plants (Lagos-Quintana et al., Science 294:853-858 (2001), Lagos-Quintana et al., Curr. Biol. 12:735-739 (2002); Lau et al., Science 294:858-862 (2001); Lee and Ambros, Science 294:862-864 (2001); Llave et al., Plant Cell 14:1605-1619 (2002); Mourelatos et al., Genes Dev. 16:720-728 (2002); Park et al., Curr. Biol. 12:1484-1495 (2002); Reinhart et al., Genes. Dev. 16:1616-1626 (2002)). They are processed from longer precursor transcripts that range in size from approximately 70 to 200 nt, and these precursor transcripts have the ability to form stable hairpin structures.

MicroRNAs (miRNAs) appear to regulate target genes by binding to complementary sequences located in the transcripts produced by these genes. It seems likely that miRNAs can enter at least two pathways of target gene regulation: (1) translational inhibition; and (2) RNA cleavage. MicroRNAs entering the RNA cleavage pathway are analogous to the 21-25 nt short interfering RNAs (siRNAs) generated during RNA interference (RNAi) in animals and posttranscriptional gene silencing (PTGS) in plants, and likely are incorporated into an RNA-induced silencing complex (RISC) that is similar or identical to that seen for RNAi.

The terms “miRNA-star sequence” and “miRNA* sequence” are used interchangeably herein and they refer to a sequence in the miRNA precursor that is highly complementary to the miRNA sequence. The miRNA and miRNA* sequences form part of the stem region of the miRNA precursor hairpin structure.

In one embodiment, there is provided a method for the suppression of a target sequence comprising introducing into a cell a nucleic acid construct encoding a miRNA substantially complementary to the target. In some embodiments the miRNA comprises about 19, 20, 21, 22, 23, 24 or 25 nucleotides. In some embodiments the miRNA comprises 21 nucleotides. In some embodiments the nucleic acid construct encodes the miRNA. In some embodiments the nucleic acid construct encodes a polynucleotide precursor which may form a double-stranded RNA, or hairpin structure comprising the miRNA.

In some embodiments, the nucleic acid construct comprises a modified endogenous plant miRNA precursor, wherein the precursor has been modified to replace the endogenous miRNA encoding region with a sequence designed to produce a miRNA directed to the target sequence. The plant miRNA precursor may be full-length of may comprise a fragment of the full-length precursor. In some embodiments, the endogenous plant miRNA precursor is from a dicot or a monocot. In some embodiments the endogenous miRNA precursor is from Arabidopsis, tomato, maize, soybean, sunflower, sorghum, canola, wheat, alfalfa, cotton, rice, barley, millet, sugar cane or switchgrass.

In some embodiments, the miRNA template, (i.e. the polynucleotide encoding the miRNA), and thereby the miRNA, may comprise some mismatches relative to the target sequence. In some embodiments the miRNA template has >1 nucleotide mismatch as compared to the target sequence, for example, the miRNA template can have 1, 2, 3, 4, 5, or more mismatches as compared to the target sequence. This degree of mismatch may also be described by determining the percent identity of the miRNA template to the complement of the target sequence. For example, the miRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the complement of the target sequence.

In some embodiments, the miRNA template, (i.e. the polynucleotide encoding the miRNA) and thereby the miRNA, may comprise some mismatches relative to the miRNA-star sequence. In some embodiments the miRNA template has >1 nucleotide mismatch as compared to the miRNA-star sequence, for example, the miRNA template can have 1, 2, 3, 4, 5, or more mismatches as compared to the miRNA-star sequence. This degree of mismatch may also be described by determining the percent identity of the miRNA template to the complement of the miRNA-star sequence. For example, the miRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the complement of the miRNA-star sequence.

### Regulatory Sequences:

A recombinant DNA construct (including a suppression DNA construct) of the present disclosure may comprise at least one regulatory sequence.

A regulatory sequence may be a promoter.

A number of promoters can be used in recombinant DNA constructs of the present disclosure. The promoters can be selected based on the desired outcome, and may include constitutive, tissue-specific, inducible, or other promoters for expression in the host organism.

Promoters that cause a gene to be expressed in most cell types at most times are commonly referred to as “constitutive promoters”.

High level, constitutive expression of the candidate gene under control of the 35S or UBI promoter may have pleiotropic effects, although candidate gene efficacy may be estimated when driven by a constitutive promoter. Use of tissue-specific and/or stress-specific promoters may eliminate undesirable effects but retain the ability to enhance stress tolerance. This effect has been observed in Arabidopsis (Kasuga et al. (1999) Nature Biotechnol. 17:287-91).

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)); ALS promoter (U.S. Pat. No. 5,659,026), the constitutive synthetic core promoter SCP1 (International Publication No. 03/033651) and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the disclosure, it may be desirable to use a tissue-specific or developmentally regulated promoter.

A tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter may be used in the methods of the present disclosure which causes the desired temporal and spatial expression.

Promoters which are seed or embryo-specific and may be useful include soybean Kunitz trypsin inhibitor (Kti3, Jofuku and Goldberg, Plant Cell 1:1079-1093 (1989)), patatin (potato tubers) (Rocha-Sosa, M., et al. (1989) EMBO J. 8:23-29), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. U.S.A. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564), and sporamin (sweet potato tuberous root) (Hattori, T., et al. (1990) Plant Mol. Biol. 14:595-604). Promoters of seed-specific genes operably linked to heterologous coding regions in chimeric gene constructions maintain their temporal and spatial expression pattern in transgenic plants. Such examples include Arabidopsis thaliana 2S seed storage protein gene promoter to express enkephalin peptides in Arabidopsis and Brassica napus seeds (Vanderkerckhove et al., Bio/Technology 7:L929-932 (1989)), bean lectin and bean beta-phaseolin promoters to express luciferase (Riggs et al., Plant Sci. 63:47-57 (1989)), and wheat glutenin promoters to express chloramphenicol acetyl transferase (Colot et al., EMBO J 6:3559-3564 (1987)). Endosperm preferred promoters include those described in e.g., U.S. Pat. No. 8,466,342; U.S. Pat. No. 7,897,841; and U.S. Pat. No. 7,847,160.

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Promoters for use include the following: 1) the stress-inducible RD29A promoter (Kasuga et al. (1999) Nature Biotechnol. 17:287-91); 2) the barley promoter, B22E; expression of B22E is specific to the pedicel in developing maize kernels (“Primary Structure of a Novel Barley Gene Differentially Expressed in Immature Aleurone Layers”. Klemsdal, S. S. et al., Mol. Gen. Genet. 228(1/2):9-16 (1991)); and 3) maize promoter, Zag2 (“Identification and molecular characterization of ZAG1, the maize homolog of the Arabidopsis floral homeotic gene AGAMOUS”, Schmidt, R. J. et al., Plant Cell 5(7):729-737 (1993); “Structural characterization, chromosomal localization and phylogenetic evaluation of two pairs of AGAMOUS-like MADS-box genes from maize”, Theissen et al. Gene 156(2):155-166 (1995); NCBI GenBank Accession No. X80206)). Zag2 transcripts can be detected 5 days prior to pollination to 7 to 8 days after pollination (“DAP”), and directs expression in the carpel of developing female inflorescences and Ciml which is specific to the nucleus of developing maize kernels. Ciml transcript is detected 4 to 5 days before pollination to 6 to 8 DAP. Other useful promoters include any promoter which can be derived from a gene whose expression is maternally associated with developing female florets.

Promoters for use also include the following: Zm-GOS2 (maize promoter for “Gene from Oryza sativa”, US publication number US2012/0110700 Sb-RCC (Sorghum promoter for Root Cortical Cell delineating protein, root specific expression), Zm-ADF4 (U.S. Pat. No. 7,902,428; Maize promoter for Actin Depolymerizing Factor), Zm-FTM1 (U.S. Pat. No. 7,842,851; maize promoter for Floral transition MADSs) promoters.

Additional promoters for regulating the expression of the nucleotide sequences in plants are stalk-specific promoters. Such stalk-specific promoters include the alfalfa S2A promoter (GenBank Accession No. EF030816; Abrahams et al., Plant Mol. Biol. 27:513-528 (1995)) and S2B promoter (GenBank Accession No. EF030817) and the like, herein incorporated by reference.

Promoters may be derived in their entirety from a native gene, or be composed of different elements derived from different promoters found in nature, or even comprise synthetic DNA segments.

In one embodiment the at least one regulatory element may be an endogenous promoter operably linked to at least one enhancer element; e.g., a 35S, nos or ocs enhancer element.

Promoters for use may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (Genbank accession number EF030817), and the constitutive promoter GOS2 from Zea mays. Other promoters include root preferred promoters, such as the maize NAS2 promoter, the maize Cyclo promoter (US 2006/0156439, published Jul. 13, 2006), the maize ROOTMET2 promoter (WO05063998, published Jul. 14, 2005), the CR1BIO promoter (WO06055487, published May 26, 2006), the CRWAQ81 (WO05035770, published Apr. 21, 2005) and the maize ZRP2.47 promoter (NCBI accession number: U38790; GI No. 1063664),

Recombinant DNA constructs of the present disclosure may also include other regulatory sequences, including but not limited to, translation leader sequences, introns, and polyadenylation recognition sequences. In another embodiment of the present disclosure, a recombinant DNA construct of the present disclosure further comprises an enhancer or silencer.

The promoters disclosed herein may be used with their own introns, or with any heterologous introns to drive expression of the transgene.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, Mol. Cell Biol. 8:4395-4405 (1988); Callis et al., Genes Dev. 1:1183-1200 (1987).

“Transcription terminator”, “termination sequences”, or “terminator” refer to DNA sequences located downstream of a protein-coding sequence, including polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al., Plant Cell 1:671-680 (1989). A polynucleotide sequence with “terminator activity” generally refers to a polynucleotide sequence that, when operably linked to the 3′ end of a second polynucleotide sequence that is to be expressed, is capable of terminating transcription from the second polynucleotide sequence and facilitating efficient 3′ end processing of the messenger RNA resulting in addition of poly A tail. Transcription termination is the process by which RNA synthesis by RNA polymerase is stopped and both the processed messenger RNA and the enzyme are released from the DNA template.

Improper termination of an RNA transcript can affect the stability of the RNA, and hence can affect protein expression. Variability of transgene expression is sometimes attributed to variability of termination efficiency (Bieri et al (2002) Molecular Breeding 10: 107-117).

Examples of terminators for use include, but are not limited to, PinII terminator, SB-GKAF terminator (U.S. application Ser. No. 14/236,499), Actin terminator, Os-Actin terminator, Ubi terminator, Sb-Ubi terminator, Os-Ubi terminator.

Any plant can be selected for the identification of regulatory sequences and DTP4 polypeptide genes to be used in recombinant DNA constructs and other compositions (e.g. transgenic plants, seeds and cells) and methods of the present disclosure. Examples of suitable plants for the isolation of genes and regulatory sequences and for compositions and methods of the present disclosure would include but are not limited to alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassava, castorbean, cauliflower, celery, cherry, chicory, cilantro, citrus, clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugarbeet, sugarcane, sunflower, sweet potato, sweetgum, switchgrass, tangerine, tea, tobacco, tomato, triticale, turf, turnip, a vine, watermelon, wheat, yarns, and zucchini.

Compositions:

A composition of the present disclosure includes a transgenic microorganism, cell, plant, and seed comprising the recombinant DNA construct. The cell may be eukaryotic, e.g., a yeast, insect or plant cell, or prokaryotic, e.g., a bacterial cell.

A composition of the present disclosure is a plant comprising in its genome any of the recombinant DNA constructs (including any of the suppression DNA constructs) of the present disclosure (such as any of the constructs discussed above). Compositions also include any progeny of the plant, and any seed obtained from the plant or its progeny, wherein the progeny or seed comprises within its genome the recombinant DNA construct (or suppression DNA construct). Progeny includes subsequent generations obtained by self-pollination or out-crossing of a plant. Progeny also includes hybrids and inbreds.

In hybrid seed propagated crops, mature transgenic plants can be self-pollinated to produce a homozygous inbred plant. The inbred plant produces seed containing the newly introduced recombinant DNA construct (or suppression DNA construct). These seeds can be grown to produce plants that would exhibit an altered agronomic characteristic (e.g., an increased agronomic characteristic optionally under stress conditions), or used in a breeding program to produce hybrid seed, which can be grown to produce plants that would exhibit such an altered agronomic characteristic. The seeds may be maize seeds. The stress condition may be selected from the group of drought stress, triple stress and osmotic stress.

The plant may be a monocotyledonous or dicotyledonous plant, for example, a maize or soybean plant. The plant may also be sunflower, sorghum, canola, wheat, alfalfa, cotton, rice, barley, millet, sugar cane or switchgrass. The plant may be a hybrid plant or an inbred plant.

The recombinant DNA construct may be stably integrated into the genome of the plant.

Particular embodiments include but are not limited to the following:

1. A plant (for example, a maize, rice or soybean plant) comprising in its genome a recombinant DNA construct comprising a polynucleotide operably linked to at least one heterologous regulatory sequence, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:18, 39, 43, 45, 47, 49, 51,55, 59, 61,64, 65, 66, 95, 97, 101, 103, 107, 111, 113, 117, 119, 121, 123, 127, 129, 130, 131, 132, 135, 627 or 628, and wherein said plant exhibits at least one phenotype selected from the group consisting of increased triple stress tolerance, increased drought stress tolerance, increased nitrogen stress tolerance, increased osmotic stress tolerance, altered ABA response, altered root architecture, increased tiller number, when compared to a control plant not comprising said recombinant DNA construct. The plant may further exhibit an alteration of at least one agronomic characteristic when compared to the control plant.

The plant may exhibit alteration of at least one agronomic characteristic selected from the group consisting of: abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, leaf number, tiller number, growth rate, first pollen shed time, first silk emergence time, anthesis silking interval (ASI), stalk diameter, root architecture, staygreen, relative water content, water use, water use efficiency, dry weight of either main plant, tillers, primary ear, main plant and tillers or cobs; rows of kernels, total plant weight·kernel weight, kernel number, salt tolerance, chlorophyll content, flavonol content, number of yellow leaves, early seedling vigor and seedling emergence under low temperature stress. These agronomic characteristics maybe measured at any stage of the plant development. One or more of these agronomic characteristics may be measured under stress or non-stress conditions, and may show alteration on overexpression of the recombinant constructs disclosed herein.

2. A plant (for example, a maize, rice or soybean plant) comprising in its genome a recombinant DNA construct comprising a polynucleotide operably linked to at least one regulatory sequence, wherein said polynucleotide encodes a DTP4 polypeptide, and wherein said plant exhibits at least one phenotype selected from the group consisting of increased triple stress tolerance, increased drought stress tolerance, increased nitrogen stress tolerance, increased osmotic stress tolerance, altered ABA response, altered root architecture, increased tiller number, when compared to a control plant not comprising said recombinant DNA construct. The plant may further exhibit an alteration of at least one agronomic characteristic when compared to the control plant.

3. A plant (for example, a maize, rice or soybean plant) comprising in its genome a recombinant DNA construct comprising a polynucleotide operably linked to at least one regulatory sequence, wherein said polynucleotide encodes a DTP4 polypeptide, and wherein said plant exhibits an alteration of at least one agronomic characteristic when compared to a control plant not comprising said recombinant DNA construct.

4. A plant (for example, a maize, rice or soybean plant) comprising in its genome a recombinant DNA construct comprising a polynucleotide operably linked to at least one regulatory element, wherein said polynucleotide comprises a nucleotide sequence, wherein the nucleotide sequence is: (a) hybridizable under stringent conditions with a DNA molecule comprising the full complement of SEQ ID NO:16, 17, 19, 38, 42, 44, 46, 48, 50, 54, 58, 60, 62, 63, 94, 96, 100, 102, 106, 110, 112, 116, 118, 120 or 122; or (b) derived from SEQ ID NO:16, 17, 19, 38, 42, 44, 46, 48, 50, 54, 58, 60, 62, 63, 94, 96, 100, 102, 106, 110, 112, 116, 118, 120 or 122 by alteration of one or more nucleotides by at least one method selected from the group consisting of: deletion, substitution, addition and insertion; and wherein said plant exhibits at least one phenotype selected from the group consisting of increased triple stress tolerance, increased drought stress tolerance, increased nitrogen stress tolerance, increased osmotic stress tolerance, altered ABA response, altered root architecture, increased tiller number, when compared to a control plant not comprising said recombinant DNA construct. The plant may further exhibit an alteration of at least one agronomic characteristic when compared to the control plant.

5. A plant (for example, a maize, rice or soybean plant) comprising in its genome a recombinant DNA construct comprising a polynucleotide operably linked to at least one heterologous regulatory element, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:18, 39, 43, 45, 47, 49, 51,55, 59, 61,64, 65, 66, 95, 97, 101, 103, 107, 111, 113, 117, 119, 121, 123, 127, 129, 130, 131, 132, 135, 627 or 628, and wherein said plant exhibits an alteration of at least one agronomic characteristic when compared to a control plant not comprising said recombinant DNA construct.

6. A plant (for example, a maize, rice or soybean plant) comprising in its genome a recombinant DNA construct comprising a polynucleotide operably linked to at least one heterologous regulatory element, wherein said polynucleotide comprises a nucleotide sequence, wherein the nucleotide sequence is: (a) hybridizable under stringent conditions with a DNA molecule comprising the full complement of SEQ ID NO:16, 17, 19, 38, 42, 44, 46, 48, 50, 54, 58, 60, 62, 63, 94, 96, 100, 102, 106, 110, 112, 116, 118, 120 or 122; or (b) derived from SEQ ID NO:16, 17, 19, 38, 42, 44, 46, 48, 50, 54, 58, 60, 62, 63, 94, 96, 100, 102, 106, 110, 112, 116, 118, 120 or 122 by alteration of one or more nucleotides by at least one method selected from the group consisting of: deletion, substitution, addition and insertion; and wherein said plant exhibits an alteration of at least one agronomic characteristic when compared to a control plant not comprising said recombinant DNA construct.

7. A plant (for example, a maize, rice or soybean plant) comprising in its genome a recombinant DNA construct comprising a polynucleotide operably linked to at least one heterologous regulatory sequence, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:18, 39, 43, 45, 47, 49, 51,55, 59, 61,64, 65, 66, 95, 97, 101, 103, 107, 111, 113, 117, 119, 121, 123, 127, 129, 130, 131, 132, 135, 627 or 628, and wherein said plant exhibits at least one phenotype selected from the group consisting of increased triple stress tolerance, increased drought stress tolerance, increased nitrogen stress tolerance, increased osmotic stress tolerance, altered ABA response, altered root architecture, increased tiller number, when compared to a control plant not comprising said recombinant DNA construct. The plant may further exhibit an increase in yield, biomass, or both when compared to the control plant.

8. A plant (for example, a maize, rice or soybean plant) comprising in its genome a recombinant DNA construct comprising a wherein the polynucleotide is operably linked to a heterologous promoter, and encodes a polypeptide with at least one activity selected from the group consisting of: carboxylesterase, increased triple stress tolerance, increased drought stress tolerance, increased nitrogen stress tolerance, increased osmotic stress tolerance, altered ABA response, altered root architecture, increased tiller number, wherein the polypeptide gives an E-value score of 1E-15 or less when queried using a Profile Hidden Markov Model prepared using SEQ ID NOS:18, 29, 33, 45, 47, 53, 55, 61, 64, 65, 77, 78, 101, 103, 105, 107, 111, 115, 131, 132, 135, 137, 139, 141, 144, 433, 559 and 604, the query being carried out using the hmmsearch algorithm wherein the Z parameter is set to 1 billion, and wherein said plant exhibits at least one phenotype selected from the group consisting of increased triple stress tolerance, increased drought stress tolerance, increased nitrogen stress tolerance, increased osmotic stress tolerance, altered ABA response, altered root architecture, increased tiller number, when compared to a control plant not comprising said recombinant DNA construct. The plant may further exhibit an increase in yield, biomass, or both when compared to the control plant. The polypeptide may give an E-value score of 1E-15, 1E-25, 1E-35, 1E-45, 1E-55, 1E-65, 1E-70, 1E-75, 1E-80 and 1E-85.

9. A plant (for example, a maize, rice or soybean plant) comprising in its genome a suppression DNA construct comprising at least one heterologous regulatory element operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to said all or part of a sense strand or antisense strand from which said region is derived, and wherein said target gene of interest encodes a DTP4 polypeptide, and wherein said plant exhibits an alteration of at least one agronomic characteristic when compared to a control plant not comprising said suppression DNA construct.

10. A plant (for example, a maize, rice or soybean plant) comprising in its genome a suppression DNA construct comprising at least one heterologous regulatory element operably linked to all or part of (a) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:18, 39, 43, 45, 47, 49, 51, 55, 59, 61,64, 65, 66, 95, 97, 101, 103, 107, 111, 113, 117, 119, 121, 123, 127, 129, 130, 131, 132, 135, 627 or 628, or (b) a full complement of the nucleic acid sequence of (a), and wherein said plant exhibits an alteration of at least one agronomic characteristic when compared to a control plant not comprising said suppression DNA construct.

11. A plant (for example, a maize, rice or soybean plant) comprising in its genome a polynucleotide (optionally an endogenous polynucleotide) operably linked to at least one heterologous regulatory element, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:18, 39, 43, 45, 47, 49, 51,55, 59, 61,64, 65, 66, 95, 97, 101, 103, 107, 111, 113, 117, 119, 121, 123, 127, 129, 130, 131, 132, 135, 627 or 628, and wherein said plant exhibits at least one phenotype selected from the group consisting of increased triple stress tolerance, increased drought stress tolerance, increased nitrogen stress tolerance, increased osmotic stress tolerance, altered ABA response, altered root architecture, increased tiller number when compared to a control plant not comprising the recombinant regulatory element. The at least one heterologous regulatory element may comprise an enhancer sequence or a multimer of identical or different enhancer sequences. The at least one heterologous regulatory element may comprise one, two, three or four copies of the CaMV 35S enhancer.

12. Any progeny of the plants in the embodiments described herein, any seeds of the plants in the embodiments described herein, any seeds of progeny of the plants in embodiments described herein, and cells from any of the above plants in embodiments described herein and progeny thereof.

In any of the embodiments described herein, the plant may exhibit alteration of at least one agronomic characteristic selected from the group consisting of: abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, leaf number, tiller number, growth rate, first pollen shed time, first silk emergence time, anthesis silking interval (ASI), stalk diameter, root architecture, staygreen, relative water content, water use, water use efficiency, dry weight of either main plant, tillers, primary ear, main plant and tillers or cobs; rows of kernels, total plant weight·kernel weight, kernel number, salt tolerance, chlorophyll content, flavonol content, number of yellow leaves, early seedling vigor and seedling emergence under low temperature stress. These agronomic characteristics maybe measured at any stage of the plant development. One or more of these agronomic characteristics may be measured under stress or non-stress conditions, and may show alteration on overexpression of the recombinant constructs disclosed herein.

In any of the embodiments described herein, the DTP4 polypeptide may be from Arabidopsis thaliana, Zea mays, Glycine max, Glycine tabacina, Glycine soja, Glycine tomentella, Oryza sativa, Brassica napus, Sorghum bicolor, Saccharum officinarum, Triticum aestivum or any other plant species disclosed herein.

In any of the embodiments described herein, the recombinant DNA construct (or suppression DNA construct) may comprise at least a promoter functional in a plant as a regulatory sequence.

In any of the embodiments described herein or any other embodiments of the present disclosure, the alteration of at least one agronomic characteristic is either an increase or decrease.

In any of the embodiments described herein, the plant may exhibit the alteration of at least one agronomic characteristic when compared, under at least one stress condition, to a control plant not comprising said recombinant DNA construct (or said suppression DNA construct). The at least one stress condition may be selected from the group consisting of drought stress, triple stress, nitrogen stress and osmotic stress.

In one embodiment, “yield” can be measured in many ways, including, for example, test weight, seed weight, seed number per plant, seed number per unit area (i.e. seeds, or weight of seeds, per acre), bushels per acre, tonnes per acre, tons per acre, kilo per hectare.

In any of the embodiments described herein, the plant may exhibit less yield loss relative to the control plants, for example, at least 25%, at least 20%, at least 15%, at least 10% or at least 5% less yield loss, under water limiting conditions, or would have increased yield, for example, at least 5%, at least 10%, at least 15%, at least 20% or at least 25% increased yield, relative to the control plants under water non-limiting conditions.

In any of the embodiments described herein, the plant may exhibit less yield loss relative to the control plants, for example, at least 25%, at least 20%, at least 15%, at least 10% or at least 5% less yield loss, under stress conditions, or would have increased yield, for example, at least 5%, at least 10%, at least 15%, at least 20% or at least 25% increased yield, relative to the control plants under non-stress conditions. The stress may be selected from the group consisting of drought stress, triple stress, nitrogen stress and osmotic stress.

The terms “stress tolerance” or “stress resistance” as used herein generally refers to a measure of a plants ability to grow under stress conditions that would detrimentally affect the growth, vigor, yield, and size, of a “non-tolerant” plant of the same species. Stress tolerant plants grow better under conditions of stress than non-stress tolerant plants of the same species. For example, a plant with increased growth rate, compared to a plant of the same species and/or variety, when subjected to stress conditions that detrimentally affect the growth of another plant of the same species would be said to be stress tolerant. A plant with “increased stress tolerance” can exhibit increased tolerance to one or more different stress conditions.

“Increased stress tolerance” of a plant is measured relative to a reference or control plant, and is a trait of the plant to survive under stress conditions over prolonged periods of time, without exhibiting the same degree of physiological or physical deterioration relative to the reference or control plant grown under similar stress conditions. Typically, when a transgenic plant comprising a recombinant DNA construct or suppression DNA construct in its genome exhibits increased stress tolerance relative to a reference or control plant, the reference or control plant does not comprise in its genome the recombinant DNA construct or suppression DNA construct.

“Drought” generally refers to a decrease in water availability to a plant that, especially when prolonged, can cause damage to the plant or prevent its successful growth (e.g., limiting plant growth or seed yield). “Water limiting conditions” generally refers to a plant growth environment where the amount of water is not sufficient to sustain optimal plant growth and development. The terms “drought” and “water limiting conditions” are used interchangeably herein.

“Drought tolerance” is a trait of a plant to survive under drought conditions over prolonged periods of time without exhibiting substantial physiological or physical deterioration.

“Drought tolerance activity” of a polypeptide indicates that over-expression of the polypeptide in a transgenic plant confers increased drought tolerance to the transgenic plant relative to a reference or control plant.

“Increased drought tolerance” of a plant is measured relative to a reference or control plant, and is a trait of the plant to survive under drought conditions over prolonged periods of time, without exhibiting the same degree of physiological or physical deterioration relative to the reference or control plant grown under similar drought conditions. Typically, when a transgenic plant comprising a recombinant DNA construct or suppression DNA construct in its genome exhibits increased drought tolerance relative to a reference or control plant, the reference or control plant does not comprise in its genome the recombinant DNA construct or suppression DNA construct.

“Triple stress” as used herein generally refers to the abiotic stress exerted on the plant by the combination of drought stress, high temperature stress and high light stress.

The terms “heat stress” and “temperature stress” are used interchangeably herein, and are defined as where ambient temperatures are hot enough for sufficient time that they cause damage to plant function or development, which might be reversible or irreversible in damage. “High temperature” can be either “high air temperature” or “high soil temperature”, “high day temperature” or “high night temperature, or a combination of more than one of these.

In one embodiment of the disclosure, the ambient temperature can be in the range of 30° C. to 36° C. In one embodiment of the disclosure, the duration for the high temperature stress could be in the range of 1-16 hours.

“High light intensity” and “high irradiance” and “light stress” are used interchangeably herein, and refer to the stress exerted by subjecting plants to light intensities that are high enough for sufficient time that they cause photoinhibition damage to the plant.

In one embodiment of the disclosure, the light intensity can be in the range of 250 μE to 450 μE. In one embodiment of the invention, the duration for the high light intensity stress could be in the range of 12-16 hours.

“Triple stress tolerance” is a trait of a plant to survive under the combined stress conditions of drought, high temperature and high light intensity over prolonged periods of time without exhibiting substantial physiological or physical deterioration.

“Paraquat” is an herbicide that exerts oxidative stress on the plants. Paraquat, a bipyridylium herbicide, acts by intercepting electrons from the electron transport chain at PSI. This reaction results in the production of bipyridyl radicals that readily react with dioxygen thereby producing superoxide. Paraquat tolerance in a plant has been associated with the scavenging capacity for oxyradicals (Lannelli, M. A. et al (1999) J Exp Botany, Vol. 50, No. 333, pp. 523-532). Paraquat resistant plants have been reported to have higher tolerance to other oxidative stresses as well.

“Paraquat stress” is defined as stress exerted on the plants by subjecting them to Paraquat concentrations ranging from 0.03 to 0.3 μM.

Many adverse environmental conditions such as drought, salt stress, and use of herbicide promote the overproduction of reactive oxygen species (ROS) in plant cells. ROS such as singlet oxygen, superoxide radicals, hydrogen peroxide (H₂O₂), and hydroxyl radicals are believed to be the major factor responsible for rapid cellular damage due to their high reactivity with membrane lipids, proteins, and DNA (Mittler, R. (2002)Trends Plant Sci Vol. 7 No. 9).

A polypeptide with “triple stress tolerance activity” indicates that over-expression of the polypeptide in a transgenic plant confers increased triple stress tolerance to the transgenic plant relative to a reference or control plant. A polypeptide with “paraquat stress tolerance activity” indicates that over-expression of the polypeptide in a transgenic plant confers increased Paraquat stress tolerance to the transgenic plant relative to a reference or control plant.

Typically, when a transgenic plant comprising a recombinant DNA construct or suppression DNA construct in its genome exhibits increased stress tolerance relative to a reference or control plant, the reference or control plant does not comprise in its genome the recombinant DNA construct or suppression DNA construct.

The terms “percentage germination” and “percentage seedling emergence” are used interchangeably herein, and refer to the percentage of seeds that germinate, when compared to the total number of seeds being tested.

“Germination” as used herein generally refers to the emergence of the radicle.

The term “radicle” as used herein generally refers to the embryonic root of the plant, and is terminal part of embryonic axis. It grows downward in the soil, and is the first part of a seedling to emerge from the seed during the process of germination.

The range of stress and stress response depends on the different plants which are used, i.e., it varies for example between a plant such as wheat and a plant such as Arabidopsis.

Osmosis is defined as the movement of water from low solute concentration to high solute concentration up a concentration gradient.

“Osmotic pressure” of a solution as defined herein is defined as the pressure exerted by the solute in the system. A solution with higher concentration of solutes would have higher osmotic pressure. All solutes exhibit osmotic pressure. Osmotic pressure increases as concentration of the solute increases.

The osmotic pressure exerted by 250 mM NaCl (sodium chloride) is 1.23 MPa (megapascals) (Werner, J. E. et al. (1995) Physiologia Plantarum 93: 659-666).

As used herein, the term “osmotic stress” generally refers to any stress which is associated with or induced by elevated concentrations of osmolytes and which result in a perturbation in the osmotic potential of the intracellular or extracellular environment of a cell. The term “osmotic stress” as used herein generally refers to stress exerted when the osmotic potential of the extracellular environment of the cell, tissue, seed, organ or whole plant is increased and the water potential is lowered and a substance that blocks water absorption (osmolyte) is persistently applied to the cell, tissue, seed, organ or whole plant.

With respect to the osmotic stress assay, the term “quad” as used herein refers to four components that impart osmotic stress. A “quad assay” or “quad media”, as used herein, would therefore comprise four components that impart osmotic stress, e.g., sodium chloride, sorbitol, mannitol and PEG.

An increase in the osmotic pressure of the media solution would result in increase in osmotic potential. Examples of conditions that induce osmotic stress include, but are not limited to, salinity, drought, heat, chilling and freezing.

In one embodiment of the disclosure the osmotic pressure of the media for subjecting the plants to osmotic stress is from 0.4-1.23 MPa. In other embodiments of the disclosure, the osmotic pressure of the media for subjecting the plants to osmotic stress is 0.4 MPa, 0.5 MPa, 0.6 MPa, 0.7 MPa, 0.8 MPa, 0.9 MPa, 1 MPa, 1.1 MPa, 1.2 MPa or 1.23 MPa. In other embodiments of the disclosure, the osmotic pressure of the media for subjecting the plants to osmotic stress is at least 0.4 MPa, 0.5 MPa, 0.6 MPa, 0.7 MPa, 0.8 MPa, 0.9 MPa, 1 MPa, 1.1 MPa, 1.2 MPa or 1.23 MPa. In another embodiment of the disclosure, the osmotic pressure of the media for subjecting the plants to osmotic stress is 1.23 MPa.

“Nitrogen limiting conditions” or “low nitrogen stress” refers to conditions where the amount of total available nitrogen (e.g., from nitrates, ammonia, or other known sources of nitrogen) is not sufficient to sustain optimal plant growth and development. One skilled in the art would recognize conditions where total available nitrogen is sufficient to sustain optimal plant growth and development. One skilled in the art would recognize what constitutes sufficient amounts of total available nitrogen, and what constitutes soils, media and fertilizer inputs for providing nitrogen to plants. Nitrogen limiting conditions will vary depending upon a number of factors, including but not limited to, the particular plant and environmental conditions.

Abscisic acid (ABA), a plant hormone, is known to be involved in important plant physiological functions, such as acquisition of stress response and tolerance to drought and low temperature, as well as seed maturation, dormancy, germination etc. (M. Koomneef et al., Plant Physiol. Biochem. 36:83 (1998); J. Leung & J. Giraudat, Annu. Rev. Plant. Physiol. Plant. Mol. Biol. 49:199 (1998)). Plants subjected to environmental stresses such as drought and low temperature are thought to acquire the ability to adapt to environmental stresses due to the in vivo synthesis of ABA, which causes various changes within the plant cells. A number of genes have been identified that are induced by ABA. This suggests that ABA-induced tolerance to adverse environmental conditions is a complex multigenic event.

The terms “altered ABA response” and “altered ABA sensitivity” are used interchangeably herein, and, as used herein, by these terms it is meant that a plant or plant part exhibits an altered ABA induced response, when compared to a control plant, and includes both hypersensitivity and hyposensitivity to ABA.

“Hypersensitivity” or “enhanced response” of a plant to ABA means that the plant exhibits ABA induced phenotype at lower concentration of ABA than the control plant, or exhibits increased magnitude of response than the control plant when subjected to the same concentration of ABA as the control plant.

“Hyposensitivity” or “decreased response” of a plant to ABA means that the plant exhibits ABA induced phenotype at higher concentration of ABA than the control plant, or exhibits decreased magnitude of response than the control plant when subjected to the same concentration of ABA as the control plant.

Sensitivity to ABA can be assessed at various plant developmental stages. Examples include, but are not limited to, germination, cotyledon expansion, green cotyledons, expansion of the first true leaf, altered root growth rate or developmental arrest in the seedling stage. Moreover, the concentration of ABA at which sensitivity is observed varies in a species dependent manner. For example, transgenic Arabidopsis thaliana will demonstrate sensitivity at a lower concentration than observed in Brassica or soybean.

The term “percentage greenness” or “% greenness” refers herein to the percentage of seedlings that have totally green leaves, wherein the percentage is calculated with respect to the total number of seedlings being tested. “Percentage greenness” as referred to herein is scored as the percentage of seedlings with green leaves compared to seedlings with yellow, brown or purple leaves. “Percentage greenness” can be scored at 1-leaf or 2-leaf stage for seedlings of a monocot plant, wherein the first and second leaves are true leaves. “Percentage greenness” as used herein, can be scored at 3- or 4-leaf stage for seedlings of a dicot plant, wherein two of the leaves are cotyledonary leaves, and the third and fourth leaves are true leaves. To calculate % greenness in the seedlings of a dicot plant, any seedling with any yellow or brown streaks on any of the four leaves is not considered green. To calculate % greenness in the seedlings of a monocot plant, any seedling with any yellow or brown streaks on any of the first or second leaves is not considered green. In one embodiment of the current disclosure, “percentage greenness” is calculated when all the seedlings are subjected to osmotic stress.

“True leaves” as used herein refer to the non-cotyledonary leaves of the plant or the seedling.

The term “percentage leaf emergence” or “% leaf emergence” refers herein to the percentage of seedlings that had fully expanded 1-, 2- or 3-true leaves, wherein the percentage is calculated with respect to the total number of seedlings being tested. “Percentage leaf emergence” can be scored as the appearance of fully expanded first two true leaves for the seedlings of a dicot plant. “Percentage leaf emergence” can be scored as the appearance of fully expanded first 1- or 2-true leaves for the seedlings of a monocot plant. In one embodiment of the current disclosure, the “percentage leaf emergence” is calculated when all the seedlings are subjected to osmotic stress.

One of ordinary skill in the art is familiar with protocols for simulating drought conditions and for evaluating drought tolerance of plants that have been subjected to simulated or naturally-occurring drought conditions. For example, one can simulate drought conditions by giving plants less water than normally required or no water over a period of time, and one can evaluate drought tolerance by looking for differences in physiological and/or physical condition, including (but not limited to) vigor, growth, size, or root length, or in particular, leaf color or leaf area size. Other techniques for evaluating drought tolerance include measuring chlorophyll fluorescence, photosynthetic rates and gas exchange rates.

A drought stress experiment may involve a chronic stress (i.e., slow dry down) and/or may involve two acute stresses (i.e., abrupt removal of water) separated by a day or two of recovery. Chronic stress may last 8-10 days. Acute stress may last 3-5 days. The following variables may be measured during drought stress and well watered treatments of transgenic plants and relevant control plants:

The variable “% area chg_start chronic-acute2” is a measure of the percent change in total area determined by remote visible spectrum imaging between the first day of chronic stress and the day of the second acute stress.

The variable “% area chg_start chronic-end chronic” is a measure of the percent change in total area determined by remote visible spectrum imaging between the first day of chronic stress and the last day of chronic stress.

The variable “% area chg_start chronic-harvest” is a measure of the percent change in total area determined by remote visible spectrum imaging between the first day of chronic stress and the day of harvest.

The variable “% area chg_start chronic-recovery24 hr” is a measure of the percent change in total area determined by remote visible spectrum imaging between the first day of chronic stress and 24 hrs into the recovery (24 hrs after acute stress 2).

The variable “psii_acute1” is a measure of Photosystem II (PSII) efficiency at the end of the first acute stress period. It provides an estimate of the efficiency at which light is absorbed by PSII antennae and is directly related to carbon dioxide assimilation within the leaf.

The variable “psii_acute2” is a measure of Photosystem II (PSII) efficiency at the end of the second acute stress period. It provides an estimate of the efficiency at which light is absorbed by PSII antennae and is directly related to carbon dioxide assimilation within the leaf.

The variable “fv/fm_acute1” is a measure of the optimum quantum yield (Fv/Fm) at the end of the first acute stress−(variable fluorescence difference between the maximum and minimum fluorescence/maximum fluorescence)

The variable “fv/fm_acute2” is a measure of the optimum quantum yield (Fv/Fm) at the end of the second acute stress−(variable fluorescence difference between the maximum and minimum fluorescence/maximum fluorescence).

The variable “leaf rolling_harvest” is a measure of the ratio of top image to side image on the day of harvest.

The variable “leaf rolling_recovery24 hr” is a measure of the ratio of top image to side image 24 hours into the recovery.

The variable “Specific Growth Rate (SGR)” represents the change in total plant surface area (as measured by Lemna Tec Instrument) over a single day (Y(t)=Y0*e^{r*t}). Y(t)=Y0*e^{r*t} is equivalent to % change in Y/Δt where the individual terms are as follows: Y(t)=Total surface area at t; Y0=Initial total surface area (estimated); r=Specific Growth Rate day⁻¹, and t=Days After Planting (“DAP”).

The variable “shoot dry weight” is a measure of the shoot weight 96 hours after being placed into a 104° C. oven.

The variable “shoot fresh weight” is a measure of the shoot weight immediately after being cut from the plant.

The Examples below describe some representative protocols and techniques for simulating drought conditions and/or evaluating drought tolerance.

One can also evaluate drought tolerance by the ability of a plant to maintain sufficient yield (at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% yield) in field testing under simulated or naturally-occurring drought conditions (e.g., by measuring for substantially equivalent yield under drought conditions compared to non-drought conditions, or by measuring for less yield loss under drought conditions compared to a control or reference plant).

One of ordinary skill in the art would readily recognize a suitable control or reference plant to be utilized when assessing or measuring an agronomic characteristic or phenotype of a transgenic plant in any embodiment of the present disclosure in which a control plant is utilized (e.g., compositions or methods as described herein). For example, by way of non-limiting illustrations:

1. Progeny of a transformed plant which is hemizygous with respect to a recombinant DNA construct (or suppression DNA construct), such that the progeny are segregating into plants either comprising or not comprising the recombinant DNA construct (or suppression DNA construct): the progeny comprising the recombinant DNA construct (or suppression DNA construct) would be typically measured relative to the progeny not comprising the recombinant DNA construct (or suppression DNA construct) (i.e., the progeny not comprising the recombinant DNA construct (or the suppression DNA construct) is the control or reference plant).

2. Introgression of a recombinant DNA construct (or suppression DNA construct) into an inbred line, such as in maize, or into a variety, such as in soybean: the introgressed line would typically be measured relative to the parent inbred or variety line (i.e., the parent inbred or variety line is the control or reference plant).

3. Two hybrid lines, where the first hybrid line is produced from two parent inbred lines, and the second hybrid line is produced from the same two parent inbred lines except that one of the parent inbred lines contains a recombinant DNA construct (or suppression DNA construct): the second hybrid line would typically be measured relative to the first hybrid line (i.e., the first hybrid line is the control or reference plant).

4. A plant comprising a recombinant DNA construct (or suppression DNA construct): the plant may be assessed or measured relative to a control plant not comprising the recombinant DNA construct (or suppression DNA construct) but otherwise having a comparable genetic background to the plant (e.g., sharing at least 90%, 91%, 92%, 93%, 94%, 95%, 96%, 97%, 98%, 99%, or 100% sequence identity of nuclear genetic material compared to the plant comprising the recombinant DNA construct (or suppression DNA construct)). There are many laboratory-based techniques available for the analysis, comparison and characterization of plant genetic backgrounds; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLP®s), and Simple Sequence Repeats (SSRs) which are also referred to as Microsatellites.

Furthermore, one of ordinary skill in the art would readily recognize that a suitable control or reference plant to be utilized when assessing or measuring an agronomic characteristic or phenotype of a transgenic plant would not include a plant that had been previously selected, via mutagenesis or transformation, for the desired agronomic characteristic or phenotype.

Methods:

Methods include but are not limited to methods for increasing drought tolerance in a plant, methods for increasing triple stress tolerance in a plant, methods for increasing osmotic stress tolerance in a plant, methods for increasing nitrogen stress tolerance in a plant, methods for evaluating drought tolerance in a plant, methods for evaluating triple stress tolerance in a plant, methods for evaluating osmotic stress tolerance in a plant, methods for evaluating nitrogen stress tolerance in a plant, methods for altering ABA response in a plant, methods for increasing tiller number in a plant, methods for alteration of root architecture in a plant, methods for evaluating altered ABA response in a plant, methods for altering an agronomic characteristic in a plant, methods for determining an alteration of an agronomic characteristic in a plant, and methods for producing seed. The plant may be a monocotyledonous or dicotyledonous plant, for example, a maize or soybean plant. The plant may also be sunflower, sorghum, canola, wheat, alfalfa, cotton, rice, barley, millet, sugar cane or sorghum. The seed may be a maize or soybean seed, for example, a maize hybrid seed or maize inbred seed.

Methods include but are not limited to the following:

A method for transforming a cell (or microorganism) comprising transforming a cell (or microorganism) with any of the isolated polynucleotides or recombinant DNA constructs of the present disclosure. The cell (or microorganism) transformed by this method is also included. In particular embodiments, the cell is eukaryotic cell, e.g., a yeast, insect or plant cell, or prokaryotic, e.g., a bacterial cell. The microorganism may be Agrobacterium, e.g. Agrobacterium tumefaciens or Agrobacterium rhizogenes.

A method for producing a transgenic plant comprising transforming a plant cell with any of the isolated polynucleotides or recombinant DNA constructs (including suppression DNA constructs) of the present disclosure and regenerating a transgenic plant from the transformed plant cell. The disclosure is also directed to the transgenic plant produced by this method, and transgenic seed obtained from this transgenic plant. The transgenic plant obtained by this method may be used in other methods of the present disclosure.

A method for isolating a polypeptide of the disclosure from a cell or culture medium of the cell, wherein the cell comprises a recombinant DNA construct comprising a polynucleotide of the disclosure operably linked to at least one heterologous regulatory sequence, and wherein the transformed host cell is grown under conditions that are suitable for expression of the recombinant DNA construct.

A method of altering the level of expression of a polypeptide of the disclosure in a host cell comprising: (a) transforming a host cell with a recombinant DNA construct of the present disclosure; and (b) growing the transformed host cell under conditions that are suitable for expression of the recombinant DNA construct wherein expression of the recombinant DNA construct results in production of altered levels of the polypeptide of the disclosure in the transformed host cell.

A method of increasing stress tolerance in a plant, wherein the stress is selected from the group consisting of drought stress, triple stress, nitrogen stress and osmotic stress, the method comprising: (a) introducing into a regenerable plant cell a recombinant DNA construct comprising a polynucleotide operably linked to at least one regulatory sequence (for example, a promoter functional in a plant), wherein the polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:18, 39, 43, 45, 47, 49, 51,55, 59, 61, 64, 65, 66, 95, 97, 101, 103, 107, 111, 113, 117, 119, 121, 123, 127, 129, 130, 131, 132, 135, 627 or 628; and (b) regenerating a transgenic plant from the regenerable plant cell after step (a), wherein the transgenic plant comprises in its genome the recombinant DNA construct and exhibits increased stress tolerance, wherein the stress is selected from the group consisting of drought stress, triple stress, nitrogen stress and osmotic stress, when compared to a control plant not comprising the recombinant DNA construct. The method may further comprise (c) obtaining a progeny plant derived from the transgenic plant, wherein said progeny plant comprises in its genome the recombinant DNA construct and exhibits increased stress tolerance, wherein the stress is selected from the group consisting of drought stress, triple stress, nitrogen stress and osmotic stress, when compared to a control plant not comprising the recombinant DNA construct.

A method of increasing stress tolerance, wherein the stress is selected from the group consisting of drought stress, triple stress and osmotic stress the method comprising: (a) introducing into a regenerable plant cell a recombinant DNA construct comprising a polynucleotide operably linked to at least one heterologous regulatory element, wherein said polynucleotide comprises a nucleotide sequence, wherein the nucleotide sequence is: (a) hybridizable under stringent conditions with a DNA molecule comprising the full complement of SEQ ID NO:16, 17, 19, 38, 42, 44, 46, 48, 50, 54, 58, 60, 62, 63, 94, 96, 100, 102, 106, 110, 112, 116, 118, 120 or 122; or (b) derived from SEQ ID NO:16, 17, 19, 38, 42, 44, 46, 48, 50, 54, 58, 60, 62, 63, 94, 96, 100, 102, 106, 110, 112, 116, 118, 120 or 122, by alteration of one or more nucleotides by at least one method selected from the group consisting of: deletion, substitution, addition and insertion; and (b) regenerating a transgenic plant from the regenerable plant cell after step (a), wherein the transgenic plant comprises in its genome the recombinant DNA construct and exhibits increased stress tolerance, wherein the stress is selected from the group consisting of drought stress, triple stress, nitrogen stress and osmotic stress, when compared to a control plant not comprising the recombinant DNA construct. The method may further comprise (c) obtaining a progeny plant derived from the transgenic plant, wherein said progeny plant comprises in its genome the recombinant DNA construct and exhibits increased stress tolerance, wherein the stress is selected from the group consisting of drought stress, triple stress, nitrogen stress and osmotic stress, when compared to a control plant not comprising the recombinant DNA construct.

A method of selecting for (or identifying) increased stress tolerance in a plant, wherein the stress is selected from the group consisting of drought stress, triple stress, nitrogen stress and osmotic stress, the method comprising (a) obtaining a transgenic plant, wherein the transgenic plant comprises in its genome a recombinant DNA construct comprising a polynucleotide operably linked to at least one heterologous regulatory sequence (for example, a promoter functional in a plant), wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:18, 39, 43, 45, 47, 49, 51,55, 59, 61, 64, 65, 66, 95, 97, 101, 103, 107, 111,113, 117, 119, 121, 123, 127, 129, 130, 131, 132, 135, 627 or 628; (b) obtaining a progeny plant derived from said transgenic plant, wherein the progeny plant comprises in its genome the recombinant DNA construct; and (c) selecting (or identifying) the progeny plant with increased stress tolerance, wherein the stress is selected from the group consisting of drought stress, triple stress, nitrogen stress and osmotic stress tolerance, compared to a control plant not comprising the recombinant DNA construct.

In another embodiment, a method of selecting for (or identifying) increased stress tolerance in a plant, wherein the stress is selected from the group consisting of drought stress, triple stress, nitrogen stress and osmotic stress, the method comprising: (a) obtaining a transgenic plant, wherein the transgenic plant comprises in its genome a recombinant DNA construct comprising a polynucleotide operably linked to at least one heterologous regulatory element, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:18, 39, 43, 45, 47, 49, 51,55, 59, 61,64, 65, 66, 95, 97, 101, 103, 107, 111, 113, 117, 119, 121, 123, 127, 129, 130, 131, 132, 135, 627 or 628; (b) growing the transgenic plant of part (a) under conditions wherein the polynucleotide is expressed; and (c) selecting (or identifying) the transgenic plant of part (b) with increased stress tolerance, wherein the stress is selected from the group consisting of drought stress, triple stress, nitrogen stress and osmotic stress, compared to a control plant not comprising the recombinant DNA construct.

A method of selecting for (or identifying) increased stress tolerance in a plant, wherein the stress is selected from the group consisting of drought stress, triple stress, nitrogen stress and osmotic stress the method comprising: (a) obtaining a transgenic plant, wherein the transgenic plant comprises in its genome a recombinant DNA construct comprising a polynucleotide operably linked to at least one heterologous regulatory element, wherein said polynucleotide comprises a nucleotide sequence, wherein the nucleotide sequence is: (i) hybridizable under stringent conditions with a DNA molecule comprising the full complement of SEQ ID NO:16, 17, 19, 38, 42, 44, 46, 48, 50, 54, 58, 60, 62, 63, 94, 96, 100, 102, 106, 110, 112, 116, 118, 120 or 122; or (ii) derived from SEQ ID NO:16, 17, 19, 38, 42, 44, 46, 48, 50, 54, 58, 60, 62, 63, 94, 96, 100, 102, 106, 110, 112, 116, 118, 120 or 122 by alteration of one or more nucleotides by at least one method selected from the group consisting of: deletion, substitution, addition and insertion; (b) obtaining a progeny plant derived from said transgenic plant, wherein the progeny plant comprises in its genome the recombinant DNA construct; and (c) selecting (or identifying) the progeny plant with increased stress tolerance, when compared to a control plant not comprising the recombinant DNA construct.

A method of making a plant that exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, when compared to a control plant, the method comprising the steps of introducing into a plant a recombinant DNA construct comprising a polynucleotide operably linked to at least one heterologous regulatory element, wherein said polynucleotide encodes a polypeptide having an amino acid sequence of at least 80% sequence identity, when compared to SEQ ID NO:18, 39, 43, 45, 47, 49, 51,55, 59, 61,64, 65, 66, 95, 97, 101, 103, 107, 111, 113, 117, 119, 121, 123, 127, 129, 130, 131, 132, 135, 627 or 628.

A method of producing a plant that exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, wherein the method comprises growing a plant from a seed comprising a recombinant DNA construct, wherein the recombinant DNA construct comprises a polynucleotide operably linked to at least one heterologous regulatory element, wherein the polynucleotide encodes a polypeptide having an amino acid sequence of at least 80% sequence identity, when compared to SEQ ID NO:18, 39, 43, 45, 47, 49, 51, 55, 59, 61,64, 65, 66, 95, 97, 101, 103, 107, 111, 113, 117, 119, 121, 123, 127, 129, 130, 131, 132, 135, 627 or 628, wherein the plant exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, when compared to a control plant not comprising the recombinant DNA construct.

A method of making a plant that exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, the method comprising: (a) introducing into a regenerable plant cell a recombinant DNA construct comprising a polynucleotide operably linked to at least one regulatory sequence, wherein the polynucleotide encodes a polypeptide gives an E-value score of 1E-15 or less when queried using a Profile Hidden Markov Model prepared using SEQ ID NOS:18, 29, 33, 45, 47, 53, 55, 61, 64, 65, 77, 78, 101, 103, 105, 107, 111, 115, 131, 132, 135, 137, 139, 141, 144, 433, 559 and 604, the query being carried out using the hmmsearch algorithm wherein the Z parameter is set to 1 billion; (b) regenerating a transgenic plant from the regenerable plant cell of (a), wherein the transgenic plant comprises in its genome the recombinant DNA construct; and (c) obtaining a progeny plant derived from the transgenic plant of (b), wherein said progeny plant comprises in its genome the recombinant DNA construct and exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, when compared to a control plant not comprising the recombinant DNA construct.

A method of increasing in a crop plant at least one phenotype selected from the group consisting of: triple stress tolerance, drought stress tolerance, nitrogen stress tolerance, osmotic stress tolerance, ABA response, tiller number, yield and biomass, the method comprising increasing the expression of a carboxyl esterase in the crop plant. In one embodiment, the crop plant is maize. In one embodiment, the carboxylesterase has at least 80% sequence identity, when compared to SEQ ID NO:18, 39, 43, 45, 47, 49, 51,55, 59, 61,64, 65, 66, 95, 97, 101, 103, 107, 111, 113, 117, 119, 121, 123, 127, 129, 130, 131, 132, 135, 627 or 628. In one embodiment, the carboxylesterase is a DTP4 polypeptide disclosed in Table 1 and Table 2 in the current disclosure. In one embodiment, the carboxylesterase gives an E-value score of 1E-15 or less when queried using a Profile Hidden Markov Model prepared using SEQ ID NOS:18, 29, 33, 45, 47, 53, 55, 61, 64, 65, 77, 78, 101, 103, 105, 107, 111, 115, 131, 132, 135, 137, 139, 141, 144, 433, 559 and 604, the query being carried out using the hmmsearch algorithm wherein the Z parameter is set to 1 billion.

In one embodiment, the carboxylesterase is a polypeptide wherein the polypeptide gives an E-value score of 1E-15 or less when queried using the Profile Hidden Markov Model given in Table 18.

One embodiment encompasses a method of increasing stress tolerance in a plant, wherein the stress is selected from a group consisting of: drought stress, triple stress, nitrogen stress and osmotic stress, the method comprising:

(a) introducing into a regenerable plant cell a recombinant DNA construct comprising a polynucleotide operably linked to at least one regulatory sequence, wherein the polynucleotide encodes a polypeptide gives an E-value score of 1E-15 or less when queried using a Profile Hidden Markov Model prepared using SEQ ID NOS:18, 29, 33, 45, 47, 53, 55, 61,64, 65, 77, 78, 101, 103, 105, 107, 111, 115, 131,132, 135, 137, 139, 141, 144, 433, 559 and 604, the query being carried out using the hmmsearch algorithm wherein the Z parameter is set to 1 billion; (b) regenerating a transgenic plant from the regenerable plant cell of (a), wherein the transgenic plant comprises in its genome the recombinant DNA construct; and (c) obtaining a progeny plant derived from the transgenic plant of (b), wherein said progeny plant comprises in its genome the recombinant DNA construct and exhibits increased tolerance to at least one stress selected from the group consisting of: drought stress, triple stress, nitrogen stress and osmotic stress, when compared to a control plant not comprising the recombinant DNA construct.

A method of selecting for (or identifying) an alteration of an agronomic characteristic in a plant, comprising (a) obtaining a transgenic plant, wherein the transgenic plant comprises in its genome a recombinant DNA construct comprising a polynucleotide operably linked to at least one heterologous regulatory sequence (for example, a promoter functional in a plant), wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:18, 39, 43, 45, 47, 49, 51, 55, 59, 61,64, 65, 66, 95, 97, 101, 103, 107, 111, 113, 117, 119, 121, 123, 127, 129, 130, 131, 132, 135, 627 or 628; (b) obtaining a progeny plant derived from said transgenic plant, wherein the progeny plant comprises in its genome the recombinant DNA construct; and (c) selecting (or identifying) the progeny plant that exhibits an alteration in at least one agronomic characteristic when compared, optionally under at least one stress condition, to a control plant not comprising the recombinant DNA construct. The at least one stress condition may be selected from the group of drought stress, triple stress, nitrogen stress and osmotic stress. The polynucleotide preferably encodes a DTP4 polypeptide. The DTP4 polypeptide preferably has stress tolerance activity, wherein the stress is selected from the group consisting of drought stress, triple stress, nitrogen stress and osmotic stress.

In another embodiment, a method of selecting for (or identifying) an alteration of at least one agronomic characteristic in a plant, comprising: (a) obtaining a transgenic plant, wherein the transgenic plant comprises in its genome a recombinant DNA construct comprising a polynucleotide operably linked to at least one heterologous regulatory element, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:18, 39, 43, 45, 47, 49, 51, 55, 59, 61, 64, 65, 66, 95, 97, 101, 103, 107, 111, 113, 117, 119, 121, 123, 127, 129, 130, 131, 132, 135, 627 or 628, wherein the transgenic plant comprises in its genome the recombinant DNA construct; (b) growing the transgenic plant of part (a) under conditions wherein the polynucleotide is expressed; and (c) selecting (or identifying) the transgenic plant of part (b) that exhibits an alteration of at least one agronomic characteristic when compared to a control plant not comprising the recombinant DNA construct. Optionally, said selecting (or identifying) step (c) comprises determining whether the transgenic plant exhibits an alteration of at least one agronomic characteristic when compared, under at least one condition, to a control plant not comprising the recombinant DNA construct. The at least one agronomic trait may be yield, biomass, or both and the alteration may be an increase. The at least one stress condition may be selected from the group of drought stress, triple stress, nitrogen stress and osmotic stress.

The at least one agronomic characteristic may be abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, leaf number, tiller number, growth rate, first pollen shed time, first silk emergence time, anthesis silking interval (ASI), stalk diameter, root architecture, staygreen, relative water content, water use, water use efficiency, dry weight of either main plant, tillers, primary ear, main plant and tillers or cobs; rows of kernels, total plant weight·kernel weight, kernel number, salt tolerance, chlorophyll content, flavonol content, number of yellow leaves, early seedling vigor and seedling emergence under low temperature stress. These agronomic characteristics maybe measured at any stage of the plant development. One or more of these agronomic characteristics may be measured under stress or non-stress conditions, and may show alteration on overexpression of the recombinant constructs disclosed herein.

A method of selecting for (or identifying) an alteration of an agronomic characteristic in a plant, comprising (a) obtaining a transgenic plant, wherein the transgenic plant comprises in its genome a recombinant DNA construct comprising a polynucleotide operably linked to at least one heterologous regulatory element, wherein said polynucleotide comprises a nucleotide sequence, wherein the nucleotide sequence is: (i) hybridizable under stringent conditions with a DNA molecule comprising the full complement of SEQ ID NO:16, 17, 19, 38, 42, 44, 46, 48, 50, 54, 58, 60, 62, 63, 94, 96, 100, 102, 106, 110, 112, 116, 118, 120 or 122; or (ii) derived from SEQ ID NO:16, 17, 19, 38, 42, 44, 46, 48, 50, 54, 58, 60, 62, 63, 94, 96, 100, 102, 106, 110, 112, 116, 118, 120 or 122 by alteration of one or more nucleotides by at least one method selected from the group consisting of: deletion, substitution, addition and insertion; (b) obtaining a progeny plant derived from said transgenic plant, wherein the progeny plant comprises in its genome the recombinant DNA construct; and (c) selecting (or identifying) the progeny plant that exhibits an alteration in at least one agronomic characteristic when compared, optionally under stress conditions, wherein the stress is selected from the group consisting of drought stress, triple stress, nitrogen stress and osmotic stress, to a control plant not comprising the recombinant DNA construct. The polynucleotide preferably encodes a DTP4 polypeptide. The DTP4 polypeptide preferably has stress tolerance activity, wherein the stress is selected from the group consisting of drought stress, triple stress, nitrogen stress and osmotic stress.

The use of a recombinant DNA construct for producing a plant that exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, when compared to a control plant not comprising said recombinant DNA construct, wherein the recombinant DNA construct comprises a polynucleotide operably linked to at least one heterologous regulatory element, wherein the polynucleotide encodes a polypeptide having an amino acid sequence of at least 70%, 75%, 80%, 85%, 90%, 91%, 92%, 93%, 94%, 95%, 96%, 97%, 98%, 99% or 100% sequence identity, based on the Clustal V or the Clustal W method of alignment, using the respective default parameters, when compared to SEQ ID NO:18, 39, 43, 45, 47, 49, 51, 55, 59, 61, 64, 65, 66, 95, 97, 101, 103, 107, 111, 113, 117, 119, 121, 123, 127, 129, 130, 131, 132, 135, 627 or 628. The polypeptide may be over-expressed in at least one tissue of the plant, or during at least one condition of environmental stress, or both. The plant may be selected from the group consisting of: maize, soybean, sunflower, sorghum, canola, wheat, alfalfa, cotton, rice, barley, millet, sugar cane and switchgrass.

A method of producing seed (for example, seed that can be sold as a drought tolerant product offering) comprising any of the preceding methods, and further comprising obtaining seeds from said progeny plant, wherein said seeds comprise in their genome said recombinant DNA construct (or suppression DNA construct).

A method of producing oil or a seed by-product, or both, from a seed, the method comprising extracting oil or a seed by-product, or both, from a seed that comprises a recombinant DNA construct, wherein the recombinant DNA construct comprises a polynucleotide operably linked to at least one heterologous regulatory element, wherein the polynucleotide encodes a polypeptide having an amino acid sequence of at least 70%, 75%, 80%, 85%, 90%, 91%, 92%, 93%, 94%, 95%, 96%, 97%, 98%, 99% or 100% sequence identity, based on the Clustal V or the Clustal W method of alignment, using the respective default parameters, when compared to SEQ ID NO:18, 39, 43, 45, 47, 49, 51, 55, 59, 61,64, 65, 66, 95, 97, 101, 103, 107, 111, 113, 117, 119, 121, 123, 127, 129, 130, 131, 132, 135, 627 or 628. The seed may be obtained from a plant that comprises the recombinant DNA construct, wherein the plant exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, when compared to a control plant not comprising the recombinant DNA construct. The polypeptide may be over-expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass. The oil or the seed by-product, or both, may comprise the recombinant DNA construct.

Methods of isolating seed oils are well known in the art: (Young et al., Processing of Fats and Oils, In The Lipid Handbook, Gunstone et al., eds., Chapter 5 pp 253 257; Chapman & Hall: London (1994)). Seed by-products include but are not limited to the following: meal, lecithin, gums, free fatty acids, pigments, soap, stearine, tocopherols, sterols and volatiles.

One may evaluate altered root architecture in a controlled environment (e.g., greenhouse) or in field testing. The evaluation may be under simulated or naturally-occurring low or high nitrogen conditions. The altered root architecture may be an increase in root mass. The increase in root mass may be at least 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45% or 50%, when compared to a control plant not comprising the recombinant DNA construct.

In any of the foregoing methods or any other embodiments of methods of the present disclosure, the step of selecting an alteration of an agronomic characteristic in a transgenic plant, if applicable, may comprise selecting a transgenic plant that exhibits an alteration of at least one agronomic characteristic when compared, under varying environmental conditions, to a control plant not comprising the recombinant DNA construct.

In any of the foregoing methods or any other embodiments of methods of the present disclosure, the step of selecting an alteration of an agronomic characteristic in a progeny plant, if applicable, may comprise selecting a progeny plant that exhibits an alteration of at least one agronomic characteristic when compared, under varying environmental conditions, to a control plant not comprising the recombinant DNA construct.

In any of the preceding methods or any other embodiments of methods of the present disclosure, in said introducing step said regenerable plant cell may comprise a callus cell, an embryogenic callus cell, a gametic cell, a meristematic cell, or a cell of an immature embryo. The regenerable plant cells may derive from an inbred maize plant.

In any of the preceding methods or any other embodiments of methods of the present disclosure, said regenerating step may comprise the following: (i) culturing said transformed plant cells in a media comprising an embryogenic promoting hormone until callus organization is observed; (ii) transferring said transformed plant cells of step (i) to a first media which includes a tissue organization promoting hormone; and (iii) subculturing said transformed plant cells after step (ii) onto a second media, to allow for shoot elongation, root development or both.

In any of the preceding methods or any other embodiments of methods of the present disclosure, the at least one agronomic characteristic may be selected from the group consisting of: abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, leaf number, tiller number, growth rate, first pollen shed time, first silk emergence time, anthesis silking interval (ASI), stalk diameter, root architecture, staygreen, relative water content, water use, water use efficiency, dry weight of either main plant, tillers, primary ear, main plant and tillers or cobs; rows of kernels, total plant weight·kernel weight, kernel number, salt tolerance, chlorophyll content, flavonol content, number of yellow leaves, early seedling vigor and seedling emergence under low temperature stress. The alteration of at least one agronomic characteristic may be an increase in yield, greenness or biomass.

In any of the preceding methods or any other embodiments of methods of the present disclosure, the plant may exhibit the alteration of at least one agronomic characteristic when compared, under stress conditions, wherein the stress is selected from the group consisting of drought stress, triple stress, nitrogen stress and osmotic stress, to a control plant not comprising said recombinant DNA construct (or said suppression DNA construct).

In any of the preceding methods or any other embodiments of methods of the present disclosure, alternatives exist for introducing into a regenerable plant cell a recombinant DNA construct comprising a polynucleotide operably linked to at least one regulatory sequence. For example, one may introduce into a regenerable plant cell a regulatory sequence (such as one or more enhancers, optionally as part of a transposable element), and then screen for an event in which the regulatory sequence is operably linked to an endogenous gene encoding a polypeptide of the instant disclosure.

The introduction of recombinant DNA constructs of the present disclosure into plants may be carried out by any suitable technique, including but not limited to direct DNA uptake, chemical treatment, electroporation, microinjection, cell fusion, infection, vector-mediated DNA transfer, bombardment, or Agrobacterium-mediated transformation. Techniques for plant transformation and regeneration have been described in International Patent Publication WO 2009/006276, the contents of which are herein incorporated by reference.

The development or regeneration of plants containing the foreign, exogenous isolated nucleic acid fragment that encodes a protein of interest is well known in the art. The regenerated plants may be self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present disclosure containing a desired polypeptide is cultivated using methods well known to one skilled in the art.

### EMBODIMENTS

1. A plant comprising in its genome a recombinant DNA construct comprising a polynucleotide operably linked to at least one heterologous regulatory element, wherein said polynucleotide encodes a polypeptide having an amino acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO:18, 39, 43, 45, 47, 49, 51, 55, 59, 61,64, 65, 66, 95, 97, 101, 103, 107, 111, 113, 117, 119, 121, 123, 127, 129, 130, 131, 132, 135, 627 or 628, and wherein said plant exhibits at least one phenotype selected from the group consisting of: increased triple stress tolerance, increased drought stress tolerance, increased nitrogen stress tolerance, increased osmotic stress tolerance, altered ABA response, altered root architecture, and increased tiller number, when compared to a control plant not comprising said recombinant DNA construct.

2. A plant comprising in its genome a recombinant DNA construct comprising a polynucleotide operably linked to at least one heterologous regulatory element, wherein said polynucleotide encodes a polypeptide having an amino acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO:18, 39, 43, 45, 47, 49, 51, 55, 59, 61,64, 65, 66, 95, 97, 101, 103, 107, 111, 113, 117, 119, 121, 123, 127, 129, 130, 131, 132, 135, 627 or 628, and wherein said plant exhibits an increase in yield, biomass, or both, when compared to a control plant not comprising said recombinant DNA construct.

3. The plant of embodiment 2, wherein said plant exhibits said increase in yield, biomass, or both when compared, under water limiting conditions, to said control plant not comprising said recombinant DNA construct.

4. The plant of any one of embodiments 1 to 3, wherein said plant is selected from the group consisting of: Arabidopsis, maize, soybean, sunflower, sorghum, canola, wheat, alfalfa, cotton, rice, barley, millet, sugar cane and switchgrass.

5. Seed of the plant of any one of embodiments 1 to 4, wherein said seed comprises in its genome a recombinant DNA construct comprising a polynucleotide operably linked to at least one heterologous regulatory element, wherein said polynucleotide encodes a polypeptide having an amino acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO:18, 39, 43, 45, 47, 49, 51, 55, 59, 61,64, 65, 66, 95, 97, 101, 103, 107, 111, 113, 117, 119, 121, 123, 127, 129, 130, 131, 132, 135, 627 or 628, and wherein a plant produced from said seed exhibits an increase in at least one phenotype selected from the group consisting of: drought stress tolerance, triple stress tolerance, osmotic stress tolerance, nitrogen stress tolerance, tiller number, yield and biomass, when compared to a control plant not comprising said recombinant DNA construct.

6. A method of increasing stress tolerance in a plant, wherein the stress is selected from a group consisting of: drought stress, triple stress, nitrogen stress and osmotic stress, the method comprising:
(a) introducing into a regenerable plant cell a recombinant DNA construct comprising a polynucleotide operably linked to at least one heterologous regulatory sequence, wherein the polynucleotide encodes a polypeptide having an amino acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO:18, 39, 43, 45, 47, 49, 51, 55, 59, 61, 64, 65, 66, 95, 97, 101, 103, 107, 111, 113, 117, 119, 121, 123, 127, 129, 130, 131, 132, 135, 627 or 628;
(b) regenerating a transgenic plant from the regenerable plant cell of (a), wherein the transgenic plant comprises in its genome the recombinant DNA construct; and
(c) obtaining a progeny plant derived from the transgenic plant of (b), wherein said progeny plant comprises in its genome the recombinant DNA construct and exhibits increased tolerance to at least one stress selected from the group consisting of drought stress, triple stress, nitrogen stress and osmotic stress, when compared to a control plant not comprising the recombinant DNA construct.

7. A method of selecting for increased stress tolerance in a plant, wherein the stress is selected from a group consisting of: drought stress, triple stress, nitrogen stress and osmotic stress, the method comprising:
(a) obtaining a transgenic plant, wherein the transgenic plant comprises in its genome a recombinant DNA construct comprising a polynucleotide operably linked to at least one heterologous regulatory element, wherein said polynucleotide encodes a polypeptide having an amino acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO:18, 39, 43, 45, 47, 49, 51,55, 59, 61,64, 65, 66, 95, 97, 101, 103, 107, 111, 113, 117, 119, 121, 123, 127, 129, 130, 131, 132, 135, 627 or 628;
(b) growing the transgenic plant of part (a) under conditions wherein the polynucleotide is expressed; and
(c) selecting the transgenic plant of part (b) with increased stress tolerance, wherein the stress is selected from the group consisting of: drought stress, triple stress, nitrogen stress and osmotic stress, when compared to a control plant not comprising the recombinant DNA construct.

8. A method of selecting for an alteration of yield, biomass, or both in a plant, comprising:
(a) obtaining a transgenic plant, wherein the transgenic plant comprises in its genome a recombinant DNA construct comprising a polynucleotide operably linked to at least one regulatory element, wherein said polynucleotide encodes a polypeptide having an amino acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO:18, 39, 43, 45, 47, 49, 51,55, 59, 61,64, 65, 66, 95, 97, 101, 103, 107, 111, 113, 117, 119, 121, 123, 127, 129, 130, 131, 132, 135, 627 or 628;
(b) growing the transgenic plant of part (a) under conditions wherein the polynucleotide is expressed; and
(c) selecting the transgenic plant of part (b) that exhibits an alteration of yield, biomass or both when compared to a control plant not comprising the recombinant DNA construct.

9. The method of embodiment 8, wherein said selecting step (c) comprises determining whether the transgenic plant of (b) exhibits an alteration of yield, biomass or both when compared, under water limiting conditions, to a control plant not comprising the recombinant DNA construct.

10. The method of embodiment 8 or embodiment 9, wherein said alteration is an increase.

11. The method of any one of embodiments 6 to 10, wherein said plant is selected from the group consisting of: Arabidopsis, maize, soybean, sunflower, sorghum, canola, wheat, alfalfa, cotton, rice, barley, millet, sugar cane and switchgrass.

12. An isolated polynucleotide comprising:
(a) a nucleotide sequence encoding a polypeptide with stress tolerance activity, wherein the stress is selected from a group consisting of drought stress, triple stress, nitrogen stress and osmotic stress, and wherein the polypeptide has an amino acid sequence of at least 95%, 96%, 97%, 98%, 99% or 100% sequence identity when compared to SEQ ID NO:18, 39, 43, 45, 47, 49, 51, 55, 59, 61, 64, 65, 66, 95, 97, 101, 103, 107, 111, 113, 117, 119, 121, 123, 127, 129, 130, 131, 132, 135, 627 or 628; or
(b) the full complement of the nucleotide sequence of (a).

13. The polynucleotide of embodiment 12, wherein the amino acid sequence of the polypeptide comprises less than 100% sequence identity to SEQ ID NO:18, 39, 43, 45, 47, 49, 51, 55, 59, 61, 64, 65, 66, 95, 97, 101, 103, 107, 111, 113, 117, 119, 121, 123, 127, 129, 130, 131, 132, 135, 627 or 628.

14. The polynucleotide of embodiment 12 wherein the nucleotide sequence comprises SEQ ID NO:16, 17, 19, 38, 42, 44, 46, 48, 50, 54, 58, 60, 62, 63, 94, 96, 100, 102, 106, 110, 112, 116, 118, 120 or 122.

15. A plant or seed comprising a recombinant DNA construct, wherein the recombinant DNA construct comprises the polynucleotide of any one of embodiments 12 to 14 operably linked to at least one heterologous regulatory sequence.

16. A plant comprising in its genome an endogenous polynucleotide operably linked to at least one heterologous regulatory element, wherein said endogenous polynucleotide encodes a polypeptide having an amino acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO:18, 39, 43, 45, 47, 49, 51, 55, 59, 61,64, 65, 66, 95, 97, 101, 103, 107, 111, 113, 117, 119, 121, 123, 127, 129, 130, 131, 132, 135, 627 or 628, and wherein said plant exhibits at least one phenotype selected from the group consisting of increased triple stress tolerance, increased drought stress tolerance, increased nitrogen stress tolerance, increased osmotic stress tolerance, altered ABA response, altered root architecture, increased tiller number, when compared to a control plant not comprising the heterologous regulatory element.

17. A method of increasing in a crop plant at least one phenotype selected from the group consisting of: triple stress tolerance, drought stress tolerance, nitrogen stress tolerance, osmotic stress tolerance, ABA response, tiller number, yield and biomass, the method comprising increasing the expression of a carboxyl esterase in the crop plant.

18. The method of embodiment 17, wherein the crop plant is maize.

19. The method of embodiment 17 or embodiment 18, wherein the carboxyl esterase has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO:18, 39, 43, 45, 47, 49, 51, 55, 59, 61, 64, 65, 66, 95, 97, 101, 103, 107, 111, 113, 117, 119, 121, 123, 127, 129, 130, 131, 132, 135, 627 or 628. The carboxyl esterase may comprise at least one of the elements present in consensus SEQ ID NO:630 selected from the group consisting of: a conserved “nucleophile elbow” (G×S×G), a conserved catalytic triad of S-H-D and a “oxyanion hole” with the conserved residues G-G-G.

20. The method of embodiment 17 or embodiment 18, wherein the carboxylesterase gives an E-value score of 1E-15 or less when queried using a Profile Hidden Markov Model prepared using SEQ ID NOS:18, 29, 33, 45, 47, 53, 55, 61, 64, 65, 77, 78, 101, 103, 105, 107, 111, 115, 131, 132, 135, 137, 139, 141, 144, 433, 559 and 604, the query being carried out using the hmmsearch algorithm wherein the Z parameter is set to 1 billion.

21. A recombinant DNA construct comprising a polynucleotide, wherein the polynucleotide is operably linked to a heterologous promoter, and encodes a polypeptide with at least one activity selected from the group consisting of: carboxylesterase, increased triple stress tolerance, increased drought stress tolerance, increased nitrogen stress tolerance, increased osmotic stress tolerance, altered ABA response, altered root architecture, increased tiller number, wherein the polypeptide gives an E-value score of 1E-15 or less when queried using a Profile Hidden Markov Model prepared using SEQ ID NOS:18, 29, 33, 45, 47, 53, 55, 61, 64, 65, 77, 78, 101, 103, 105, 107, 111, 115, 131, 132, 135, 137, 139, 141, 144, 433, 559 and 604, the query being carried out using the hmmsearch algorithm wherein the Z parameter is set to 1 billion.

22. A plant comprising the recombinant construct of embodiment 21, wherein the plant exhibits increased yield, biomass, or both, when compared to a plant not comprising the recombinant construct.

23. A method of making a plant, that exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, the method comprising:
(a) introducing into a regenerable plant cell the recombinant DNA construct of embodiment 21;
(b) regenerating a transgenic plant from the regenerable plant cell of (a), wherein the transgenic plant comprises in its genome the recombinant DNA construct; and
(c) obtaining a progeny plant derived from the transgenic plant of (b), wherein said progeny plant comprises in its genome the recombinant DNA construct of embodiment 21 and exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, when compared to a control plant not comprising the recombinant DNA construct.

24. A method of increasing stress tolerance in a plant, wherein the stress is selected from a group consisting of: drought stress, triple stress, nitrogen stress and osmotic stress, the method comprising:
(a) introducing into a regenerable plant cell the recombinant DNA construct of embodiment 21;
(b) regenerating a transgenic plant from the regenerable plant cell of (a), wherein the transgenic plant comprises in its genome the recombinant DNA construct; and
(c) obtaining a progeny plant derived from the transgenic plant of (b), wherein said progeny plant comprises in its genome the recombinant DNA construct of embodiment 21 and exhibits increased tolerance to at least one stress selected from the group consisting of: drought stress, triple stress, nitrogen stress and osmotic stress, when compared to a control plant not comprising the recombinant DNA construct.

25. A method of making a plant that exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, when compared to a control plant, the method comprising the steps of introducing into a plant a recombinant DNA construct comprising a polynucleotide operably linked to at least one heterologous regulatory element, wherein said polynucleotide encodes a polypeptide having an amino acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO:18, 39, 43, 45, 47, 49, 51, 55, 59, 61, 64, 65, 66, 95, 97, 101, 103, 107, 111, 113, 117, 119, 121, 123, 127, 129, 130, 131, 132, 135, 627 or 628.

26. A method of producing a plant that exhibits at least one trait selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, wherein the method comprises growing a plant from a seed comprising a recombinant DNA construct, wherein the recombinant DNA construct comprises a polynucleotide operably linked to at least one heterologous regulatory element, wherein the polynucleotide encodes a polypeptide having an amino acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO:18, 39, 43, 45, 47, 49, 51, 55, 59, 61, 64, 65, 66, 95, 97, 101, 103, 107, 111, 113, 117, 119, 121, 123, 127, 129, 130, 131, 132, 135, 627 or 628, wherein the plant exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, when compared to a control plant not comprising the recombinant DNA construct.

27. A method of producing a seed, the method comprising the following:
(a) crossing a first plant with a second plant, wherein at least one of the first plant and the second plant comprises a recombinant DNA construct, wherein the recombinant DNA construct comprises a polynucleotide operably linked to at least one heterologous regulatory element, wherein the polynucleotide encodes a polypeptide having an amino acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO:18, 39, 43, 45, 47, 49, 51,55, 59, 61,64, 65, 66, 95, 97, 101, 103, 107, 111, 113, 117, 119, 121, 123, 127, 129, 130, 131, 132, 135, 627 or 628; and
(b) selecting a seed of the crossing of step (a), wherein the seed comprises the recombinant DNA construct.

28. The method of embodiment 27, wherein a plant grown from the seed of part (b) exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, when compared to a control plant not comprising the recombinant DNA construct.

29. A method of producing oil or a seed by-product, or both, from a seed, the method comprising extracting oil or a seed by-product, or both, from a seed that comprises a recombinant DNA construct, wherein the recombinant DNA construct comprises a polynucleotide operably linked to at least one heterologous regulatory element, wherein the polynucleotide encodes a polypeptide having an amino acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO:18, 39, 43, 45, 47, 49, 51, 55, 59, 61,64, 65, 66, 95, 97, 101, 103, 107, 111, 113, 117, 119, 121, 123, 127, 129, 130, 131, 132, 135, 627 or 628.

30. The method of embodiment 29, wherein the seed is obtained from a plant that comprises the recombinant DNA construct and exhibits at least one trait selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, when compared to a control plant not comprising the recombinant DNA construct.

31. The method of embodiment 29 or embodiment 30, wherein the oil or the seed by-product, or both, comprises the recombinant DNA construct.

32. A plant comprising in its genome a recombinant DNA construct comprising a polynucleotide operably linked to at least one heterologous regulatory element, wherein said polynucleotide encodes a polypeptide having an amino acid sequence of at least 95% sequence identity, when compared to SEQ ID NO:18, and wherein said plant exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, when compared to a control plant not comprising said recombinant DNA construct. The amino acid sequence of the polypeptide may have less than 100% sequence identity to SEQ ID NO:18.

33. A method of making a plant that exhibits at least one phenotype selected from the group consisting of: increased triple stress tolerance, increased drought stress tolerance, increased nitrogen stress tolerance, increased osmotic stress tolerance, altered ABA response, altered root architecture, increased tiller number, increased yield and increased biomass, when compared to a control plant, the method comprising the steps of introducing into a plant a recombinant DNA construct comprising a polynucleotide operably linked to at least one heterologous regulatory element, wherein said polynucleotide encodes a polypeptide having an amino acid sequence of at least 95% sequence identity, when compared to SEQ ID NO:18. The amino acid sequence of the polypeptide may have less than 100% sequence identity to SEQ ID NO:18.

In any of the above embodiments 1-33, the polypeptide may comprise at least one of the elements present in consensus SEQ ID NO:630 selected from the group consisting of: a conserved “nucleophile elbow” (G×S×G), a conserved catalytic triad of S-H-D and a “oxyanion hole” with the conserved residues G-G-G.

### EXAMPLES

The present disclosure is further illustrated in the following Examples, in which parts and percentages are by weight and degrees are Celsius, unless otherwise stated. It should be understood that these Examples, while indicating embodiments of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various usages and conditions. Thus, various modifications of the disclosure in addition to those shown and described herein will be apparent to those skilled in the art from the foregoing description. Such modifications are also intended to fall within the scope of the appended claims.

### Example 1

### Creation of an Arabidopsis Population with Activation-Tagged Genes

Arabidopsis activation-tagged populations were created using known methods. The resulting T1 seed were sown on soil, and transgenic seedlings were selected by spraying with glufosinate (Finale®; AgrEvo; Bayer Environmental Science). A total of 100,000 glufosinate resistant T1 seedlings were selected. T2 seed from each line was kept separate.

### Example 2

### Screens to Identify Lines with Enhanced Drought Tolerance

Activation-tagged lines can be subjected to a quantitative drought stress screen (PCT Publication No. WO/2012/058528). Lines with a significant delay in yellow color accumulation and/or with significant maintenance of rosette leaf area, when compared to the average of the whole flat, are designated as Phase 1 hits. Phase 1 hits are re-screened in duplicate under the same assay conditions. When either or both of the Phase 2 replicates show a significant difference (score of greater than 0.9) from the whole flat mean, the line is then considered a validated drought tolerant line.

### Example 3

### Screen to Identify Lines with Enhanced ABA Hypersensitivity

The activation tagged lines described in Example 1 can be subjected to independent ABA sensitivity screens. The screen is done as described in International Patent Application No. PCT/US12/62374.

Screening of transgenic plant lines is done on medium supplemented with low concentration of ABA.

Wild-type and most of transgenic seeds display consistent germination profiles with 0.6 μM ABA. Therefore 0.6 μM ABA is used for phase 1 mutant screen.

Germination is scored as the emergence of radicle over a period of 3 days. Seeds are counted manually using a magnifying lens. The data is analyzed as percentage germination to the total number of seeds that were inoculated. The germination curves are plotted. Like wild-type, most of the transgenic lines have >90% of germination rate at Day 3. Therefore for a line to qualify as outlier, it has to show a significantly lower germination rate (<75%) at Day 3. Usually the cutoff value (75% germination rate) is at least four SD away from the average value of the 96 lines. Data for germination count of all lines and their graphs at 48 hrs, 72 hrs is documented.

### Example 4

### Identification of Activation-Tagged AT-DTP4 Polypeptide Gene from the Drought Tolerant Activation-Tagged Line

An activation-tagged line (No. 121463) showing drought tolerance was further analyzed. DNA from the line was extracted, and genes flanking the insert in the mutant line were identified using SAIFF PCR (Siebert et al., Nucleic Acids Res. 23:1087-1088 (1995)). A PCR amplified fragment was identified that contained T-DNA border sequence and Arabidopsis genomic sequence. Genomic sequence flanking the insert was obtained, and the candidate gene was identified by alignment to the completed Arabidopsis genome. For a given integration event, the annotated gene nearest the 35S enhancer elements/insert was the candidate for gene that is activated in the line. In the case of line 121463, the gene nearest the 35S enhancers at the integration site was At5g62180 (SEQ ID NO:16; NCBI GI No. 30697645), encoding a DTP4 polypeptide (SEQ ID NO:18; NCBI GI No. 75180635).

### Example 5

### Identification of Activation-Tagged AT-DTP4 Polypeptide Gene from the Activation-Tagged Line Showing ABA-Hypersensitivity

An activation-tagged line (No. 990013; 35S0059G11) showing ABA-hypersensitivity was further analyzed. DNA from the line was extracted, and genes flanking the insert in the mutant line were identified using SAIFF PCR (Siebert et al., Nucleic Acids Res. 23:1087-1088 (1995)). A PCR amplified fragment was identified that contained T-DNA border sequence and Arabidopsis genomic sequence. Genomic sequence flanking the insert was obtained, and the candidate gene was identified by alignment to the completed Arabidopsis genome. For a given integration event, the annotated gene nearest the 35S enhancer elements/junction was the candidate for gene that is activated in the line. In the case of line 990013, the gene nearest the 35S enhancers at the integration site was At5g62180 (SEQ ID NO:16; NCBI GI No. 30697645), encoding a DTP4 polypeptide (SEQ ID NO:18; NCBI GI No. 75180635).

### Example 6

### Validation of Arabidopsis Candidate Gene At5g62180 (AT-DTP4 Polypeptide) for Drought Tolerance

Candidate genes can be transformed into Arabidopsis and overexpressed under the 35S promoter (PCT Publication No. WO/2012/058528). If the same or similar phenotype is observed in the transgenic line as in the parent activation-tagged line, then the candidate gene is considered to be a validated “lead gene” in Arabidopsis.

The candidate Arabidopsis DTP4 polypeptide gene (At5g62180; SEQ ID NO:16; NCBI GI No. 30697645) was tested for its ability to confer drought tolerance.

The candidate gene was cloned behind the 35S promoter in pBC-yellow to create the 35S promoter::At5g62180 expression construct, pBC-Yellow-At5g62180.

Transgenic T1 seeds were selected by yellow fluorescence, and T1 seeds were plated next to wild-type seeds and grown under water limiting conditions. Growth conditions and imaging analysis were as described in Example 2. It was found that the original drought tolerance phenotype from activation tagging could be recapitulated in wild-type Arabidopsis plants that were transformed with a construct where At5g62180 was directly expressed by the 35S promoter. The drought tolerance score, as determined by the method of PCT Publication No. WO/2012/058528, was 1.35.

### Example 7

### Validation of Arabidopsis Candidate Gene At5g62180 (AT-DTP4 Polypeptide) for ABA-Hypersensitivity Via Transformation into Arabidopsis

The candidate Arabidopsis DTP4 polypeptide gene (At5g62180; SEQ ID NO:16; NCBI GI No. 30697645) was tested for its ability to confer ABA-hypersensitivity in the following manner.

The At5g62180 cDNA protein-coding region was synthesized and cloned into the transformation vector.

Transgenic T1 seeds were selected, and used for the germination assay as described below. It was found that the original ABA hypersensitivity phenotype could be recapitulated in wild-type Arabidopsis plants that were transformed with a construct where At5g62180 was directly expressed by the 35S promoter.

### Assay Conditions:

Seeds were surface sterilized and stratified for 96 hrs. About 100 seeds were inoculated in one plate and stratified for 96 hrs, then cultured in a growth chamber programmed for 16 h of light at 22° C. temperature and 50% relative humidity. Germination was scored as the emergence of radicle.

### Observations and Results:

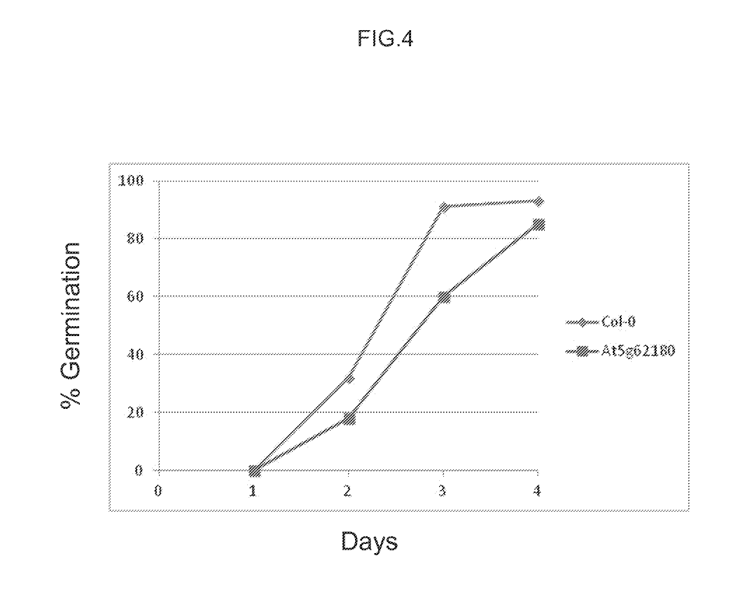

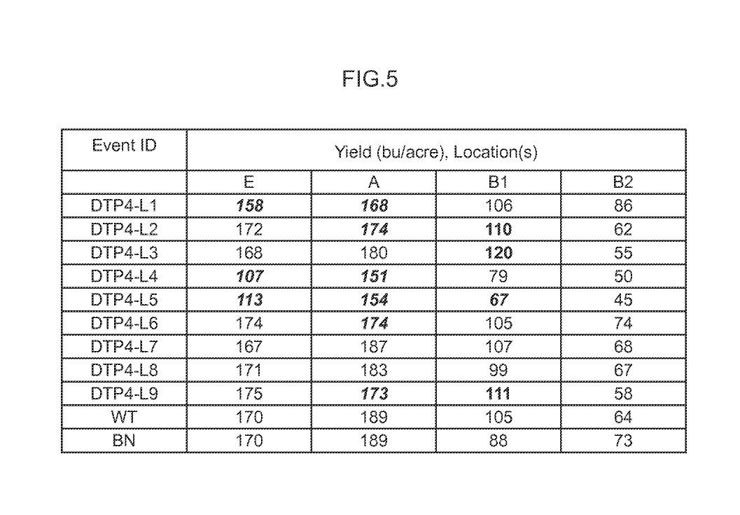

Germination was scored as the emergence of radicle in ½ MS media and 1 μM ABA over a period of 4 days. Seeds were counted manually using a magnifying lens. The data was analyzed as percentage germination to the total number of seeds that were inoculated. The cut-off value was at least 2 StandDev below control. The germination curves were plotted. Wild-type col-0 plants had >90% of germination rate at Day 3. The line with pBC-yellow-At5g62180 showed <75% germination on Day 3, as shown in FIG. 4.

### Example 8

### Characterization of cDNA Clones Encoding DTP4 Polypeptides

cDNA libraries representing mRNAs from various tissues of Zea mays, Dennstaedtia punctilobula, Sesbania bispinosa, Artemisia tridentata, Lamium amplexicaule, Delosperma nubigenum, Peperomia caperata, and other plant species were prepared and cDNA clones encoding DTP4 polypeptides were identified.

Table 3 gives additional information about some of the other DTP4 polypeptides disclosed herein.

TABLE 3
Description of Some DTP4 Polypeptides

| SEQ ID NO | | |
|---|---|---|
| (aa | | |
| sequence) | Contig | Description |
| 119 | Bn_Bo | assembled contig from |
| | | Brassica napus and Brassica |
| | | oleracea ESTs |
| 121 | Bole_someBnap_prot | assembled contig from |
| | | Brassica napus and Brassica |
| | | oleracea ESTs |
| 123 | B-napus_2-1 | assembled contig from more |
| | | than one Brassica napus ESTs |
| 125 | Csinensis plus | assembled contig from Citrus |
| | | sinensis and Citrus clementina |
| 137 | GSVIVT01027568001; | Vitis vinifera |
| 139 | GSVIVT01027566001 | Vitis vinifera |
| 141 | GSVIVT01027569001 | Vitis vinifera |

The BLAST search using the AT-DTP4 polypeptide and maize sequences from clones listed in Table 1 revealed similarity of the polypeptides encoded by the cDNAs to the DTP4 polypeptides from various organisms. As shown in Table 1, Table 2 and FIG. 1, certain cDNAs encoded polypeptides similar to DTP4 polypeptide from Arabidopsis (GI No. 75180635; SEQ ID NO:18). Shown in Table 4 and Table 5 (patent literature) are the BLAST results for some of the DTP4 polypeptides disclosed herein, that are one or more of the following: individual Expressed Sequence Tag (“EST”), the sequences of the entire cDNA inserts comprising the indicated cDNA clones (“Full-Insert Sequence” or “FIS”), the sequences of contigs assembled from two or more EST, FIS or PCR sequences (“Contig”), or sequences encoding an entire or functional protein derived from an FIS or a contig (“Complete Gene Sequence” or “CGS”). Also shown in Table 4 and 5 are the percent sequence identity values for each pair of amino acid sequences using the Clustal V method of alignment with default parameters.

TABLE 4
BLASTP Results for DTP4 polypeptides

| | | | BLASTP | Percent |
|---|---|---|---|---|
| Sequence | | NCBI GI No. | pLog of | Sequence |
| (SEQ ID NO) | Status | (SEQ ID NO) | E-value | Identity |
| cfp2n.pk010.p21 | FIS | 194704970 | >180 | 100 |
| (SEQ ID NO: 21) | | (SEQ ID NO: 82) | | |
| cfp2n.pk070.m7 | FIS | 195636334 | >180 | 100 |
| (SEQ ID NO: 23) | | (SEQ ID NO: 84) | | |
| cfp3n.pk007.i9 | FIS | 194707422 | >180 | 99.7 |
| (SEQ ID NO: 25) | | (SEQ ID NO: 86) | | |
| pco524093 | CGS | 223948401 | >180 | 100 |
| (SEQ ID NO: 27) | | (SEQ ID NO: 88) | | |
| Maize_DTP4-1 | CGS | 194704970 | >180 | 100 |
| (SEQ ID NO: 29) | | (SEQ ID NO: 82) | | |
| Maize_DTP4-2 | CGS | 23495723 | >180 | 68.2 |
| (SEQ ID NO: 31) | | (SEQ ID NO: 90) | | |
| Maize_DTP4-3 | CGS | 215768720 | >180 | 73.6 |
| (SEQ ID NO: 33) | | (SEQ ID NO: 92) | | |

TABLE 5
BLASTP Results for DTP4 polypeptides

| | | | BLASTP | Percent |
|---|---|---|---|---|
| Sequence | | Reference | pLog of | Sequence |
| (SEQ ID NO) | Status | (SEQ ID NO) | E-value | Identity |
| At5g62180 | CGS | SEQ ID NO: 12 of | >180 | 100 |
| | | U.S. Pat. No. | | |
| | | 7,915,050 | | |
| | | (SEQ ID NO: 81) | | |
| cfp2n.pk010.p21 | FIS | SEQ ID NO: | >180 | 97.6 |
| (SEQ ID NO: 21) | | 260345 | | |
| | | of US20120216318 | | |
| | | (SEQ ID NO: 83) | | |
| cfp2n.pk070.m7 | FIS | SEQ ID NO: | >180 | 100 |
| (SEQ ID NO: 23) | | 331675 | | |
| | | of US20120216318 | | |
| | | (SEQ ID NO: 85) | | |
| cfp3n.pk007.i9 | FIS | SEQ ID NO: 7332 | >180 | 97.6 |
| (SEQ ID NO: 25) | | of U.S. Pat. No. | | |
| | | 8,343,764 | | |
| | | (SEQ ID NO: 87) | | |
| pco524093 | CGS | SEQ ID NO: 16159 | >180 | 100 |
| (SEQ ID NO: 27) | | of U.S. Pat. No. | | |
| | | 7,569,389 | | |
| | | (SEQ ID NO: 89) | | |
| Maize_DTP4-1 | CGS | SEQ ID NO: | >180 | 97.6 |
| (SEQ ID NO: 29) | | 260345 | | |
| | | of US20120216318 | | |
| | | (SEQ ID NO: 83) | | |
| Maize_DTP4-2 | CGS | SEQ ID NO: 50819 | >180 | 100 |
| (SEQ ID NO: 31) | | of US20120017292 | | |
| | | (SEQ ID NO: 91) | | |
| Maize_DTP4-3 | CGS | SEQ ID NO: 10044 | >180 | 90 |
| (SEQ ID NO: 33) | | of U.S. Pat. No. | | |
| | | 8,362,325 | | |
| | | (SEQ ID NO: 93) | | |

FIG. 1A-FIG. 1G show the alignment of the DTP4 polypeptides which were tested in ABA sensitivity assays (SEQ ID NOS:18, 39, 43, 45, 47, 49, 51, 55, 59, 61, 64, 65, 66, 95, 97, 99, 101, 103, 107, 111, 113, 117, 119, 121,123, 127, 129, 130, 131, 132, 135, 627 and 628). Residues that are identical to the residue of consensus sequence (SEQ ID NO:630) at a given position are enclosed in a box. A consensus sequence is presented where a residue is shown if identical in all sequences, otherwise, a period is shown.

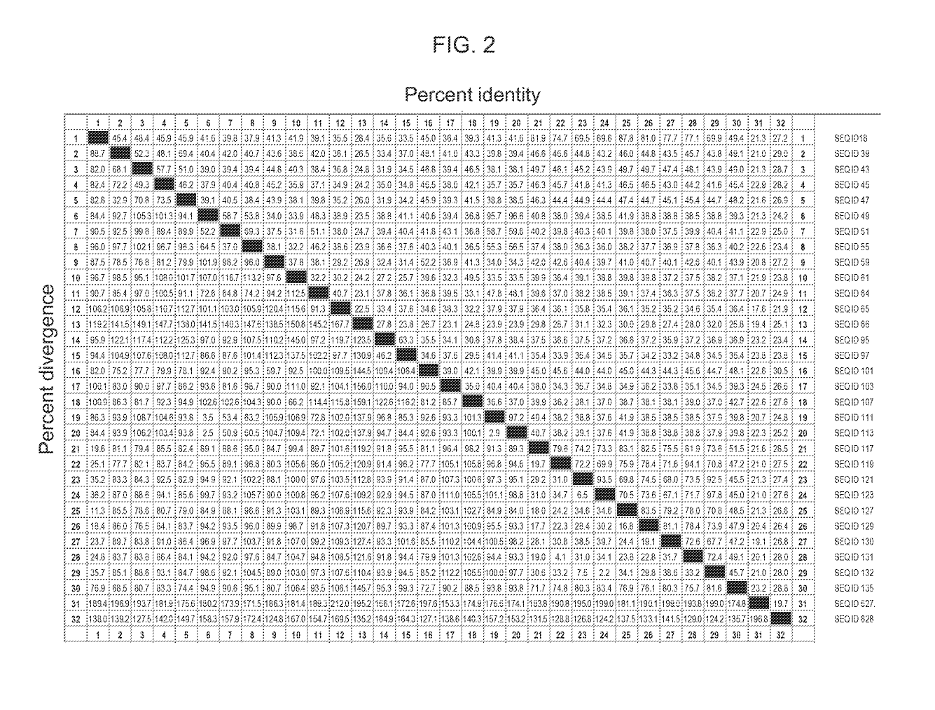

FIG. 2 shows the percent sequence identity and the divergence values for each pair of amino acids sequences of DTP4 polypeptides displayed in FIG. 1A-1G.

Sequence alignments and percent identity calculations were performed using the Megalign® program of the LASERGENE® bioinformatics computing suite (DNASTAR® Inc., Madison, Wis.). Multiple alignment of the sequences was performed using the Clustal V method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE=1, GAP PENALTY=3, WINDOW=5 and DIAGONALS SAVED=5.

Sequence alignments and BLAST scores and probabilities indicate that the nucleic acid fragments comprising the instant cDNA clones encode DTP4 polypeptides.

### Example 9

### Preparation of a Plant Expression Vector Containing a Homolog to the Arabidopsis Lead Gene

Sequences homologous to the Arabidopsis AT-DTP4 polypeptide can be identified using sequence comparison algorithms such as BLAST (Basic Local Alignment Search Tool; Altschul et al., J. Mol. Biol. 215:403-410 (1993); see also the explanation of the BLAST algorithm on the world wide web site for the National Center for Biotechnology Information at the National Library of Medicine of the National Institutes of Health). Sequences encoding homologous DTP4 polypeptides can be PCR-amplified by any of the following methods.

Method 1 (RNA-based): If the 5′ and 3′ sequence information for the protein-coding region, or the 5′ and 3′ UTR, of a gene encoding a DTP4 polypeptide homolog is available, gene-specific primers can be designed as outlined in Example 5. RT-PCR can be used with plant RNA to obtain a nucleic acid fragment containing the protein-coding region flanked by attB1 (SEQ ID NO:10) and attB2 (SEQ ID NO:11) sequences. The primer may contain a consensus Kozak sequence (CAACA) upstream of the start codon.

Method 2 (DNA-based): Alternatively, if a cDNA clone is available for a gene encoding a DTP4 polypeptide homolog, the entire cDNA insert (containing 5′ and 3′ non-coding regions) can be PCR amplified. Forward and reverse primers can be designed that contain either the attB1 sequence and vector-specific sequence that precedes the cDNA insert or the attB2 sequence and vector-specific sequence that follows the cDNA insert, respectively. For a cDNA insert cloned into the vector pBulescript SK+, the forward primer VC062 (SEQ ID NO:14) and the reverse primer VC063 (SEQ ID NO:15) can be used.

Method 3 (genomic DNA): Genomic sequences can be obtained using long range genomic PCR capture. Primers can be designed based on the sequence of the genomic locus and the resulting PCR product can be sequenced. The sequence can be analyzed using the FGENESH (Salamov, A. and Solovyev, V. (2000) Genome Res., 10: 516-522) program, and optionally, can be aligned with homologous sequences from other species to assist in identification of putative introns.

The above methods can be modified according to procedures known by one skilled in the art. For example, the primers of Method 1 may contain restriction sites instead of attB1 and attB2 sites, for subsequent cloning of the PCR product into a vector containing attB1 and attB2 sites. Additionally, Method 2 can involve amplification from a cDNA clone, a lambda clone, a BAC clone or genomic DNA.

A PCR product obtained by either method above can be combined with the GATEWAY® donor vector, such as pDONR™/Zeo (INVITROGEN™) or pDONR™ 221 (INVITROGEN™), using a BP Recombination Reaction. This process removes the bacteria lethal ccdB gene, as well as the chloramphenicol resistance gene (CAM) from pDONR™ 221 and directionally clones the PCR product with flanking attB1 and attB2 sites to create an entry clone. Using the INVITROGEN™ GATEWAY® CLONASE™ technology, the sequence encoding the homologous DTP4 polypeptide from the entry clone can then be transferred to a suitable destination vector, such as pBC-Yellow, PHP27840 or PHP23236 (PCT Publication No. WO/2012/058528; herein incorporated by reference), to obtain a plant expression vector for use with Arabidopsis, soybean and corn, respectively.

Sequences of the attP1 and attP2 sites of donor vectors pDONR™/Zeo or pDONR™ 221 are given in SEQ ID NOs:2 and 3, respectively. The sequences of the attR1 and attR2 sites of destination vectors pBC-Yellow, PHP27840 and PHP23236 are given in SEQ ID NOs:8 and 9, respectively. A BP Reaction is a recombination reaction between an Expression Clone (or an attB-flanked PCR product) and a Donor (e.g., pDONR™) Vector to create an Entry Clone. A LR Reaction is a recombination between an Entry Clone and a Destination Vector to create an Expression Clone. A Donor Vector contains attP1 and attP2 sites. An Entry Clone contains attL1 and attL2 sites (SEQ ID NOs:4 and 5, respectively). A Destination Vector contains attR1 and attR2 site. An Expression Clone contains attB1 and attB2 sites. The attB1 site is composed of parts of the attL1 and attR1 sites. The attB2 site is composed of parts of the attL2 and attR2 sites.

Alternatively a MultiSite GATEWAY® LR recombination reaction between multiple entry clones and a suitable destination vector can be performed to create an expression vector.

### Example 10

### Preparation of Soybean Expression Vectors and Transformation of Soybean with Validated Arabidopsis Lead Genes

Soybean plants can be transformed to overexpress a validated Arabidopsis lead gene or the corresponding homologs from various species in order to examine the resulting phenotype.

The same GATEWAY® entry clone described in Example 5 can be used to directionally clone each gene into the PHP27840 vector (PCT Publication No. WO/2012/058528) such that expression of the gene is under control of the SCP1 promoter (International Publication No. 03/033651).

Soybean embryos may then be transformed with the expression vector comprising sequences encoding the instant polypeptides. Techniques for soybean transformation and regeneration have been described in International Patent Publication WO 2009/006276, the contents of which are herein incorporated by reference.

T1 plants can be subjected to a soil-based drought stress. Using image analysis, plant area, volume, growth rate and color analysis can be taken at multiple times before and during drought stress. Overexpression constructs that result in a significant delay in wilting or leaf area reduction, yellow color accumulation and/or increased growth rate during drought stress will be considered evidence that the Arabidopsis gene functions in soybean to enhance drought tolerance.

Soybean plants transformed with validated genes can then be assayed under more vigorous field-based studies to study yield enhancement and/or stability under well-watered and water-limiting conditions.

### Example 11

### Transformation of Maize with Validated Arabidopsis Lead Genes Using Particle Bombardment

Maize plants can be transformed to overexpress a validated Arabidopsis lead gene or the corresponding homologs from various species in order to examine the resulting phenotype.

The same GATEWAY® entry clone described in Example 5 can be used to directionally clone each gene into a maize transformation vector. Expression of the gene in the maize transformation vector can be under control of a constitutive promoter such as the maize ubiquitin promoter (Christensen et al., (1989) Plant Mol. Biol. 12:619-632 and Christensen et al., (1992) Plant Mol. Biol. 18:675-689)

The recombinant DNA construct described above can then be introduced into corn cells by particle bombardment. Techniques for corn transformation by particle bombardment have been described in International Patent Publication WO 2009/006276, the contents of which are herein incorporated by reference.

T1 plants can be subjected to a soil-based drought stress. Using image analysis, plant area, volume, growth rate and color analysis can be taken at multiple times before and during drought stress. Overexpression constructs that result in a significant delay in wilting or leaf area reduction, yellow color accumulation and/or increased growth rate during drought stress will be considered evidence that the Arabidopsis gene functions in maize to enhance drought tolerance.

### Example 12

### Electroporation of Agrobacterium tumefaciens LBA4404

Electroporation competent cells (40 μL), such as Agrobacterium tumefaciens LBA4404 containing PHP10523 (PCT Publication No. WO/2012/058528), are thawed on ice (20-30 min). PHP10523 contains VIR genes for T-DNA transfer, an Agrobacterium low copy number plasmid origin of replication, a tetracycline resistance gene, and a Cos site for in vivo DNA bimolecular recombination. Meanwhile the electroporation cuvette is chilled on ice. The electroporator settings are adjusted to 2.1 kV. A DNA aliquot (0.5 μL parental DNA at a concentration of 0.2 μg-1.0 μg in low salt buffer or twice distilled H₂O) is mixed with the thawed Agrobacterium tumefaciens LBA4404 cells while still on ice. The mixture is transferred to the bottom of electroporation cuvette and kept at rest on ice for 1-2 min. The cells are electroporated (Eppendorf electroporator 2510) by pushing the “pulse” button twice (ideally achieving a 4.0 millisecond pulse). Subsequently, 0.5 mL of room temperature 2×YT medium (or SOC medium) are added to the cuvette and transferred to a 15 mL snap-cap tube (e.g., FALCON™ tube). The cells are incubated at 28-30° C., 200-250 rpm for 3 h.

Aliquots of 250 μL are spread onto plates containing YM medium and 50 μg/mL spectinomycin and incubated three days at 28-30° C. To increase the number of transformants one of two optional steps can be performed:

Option 1: Overlay plates with 30 μL of 15 mg/mL rifampicin. LBA4404 has a chromosomal resistance gene for rifampicin. This additional selection eliminates some contaminating colonies observed when using poorer preparations of LBA4404 competent cells.

Option 2: Perform two replicates of the electroporation to compensate for poorer electrocompetent cells.

Identification of Transformants:

Four independent colonies are picked and streaked on plates containing AB minimal medium and 50 μg/mL spectinomycin for isolation of single colonies. The plates are incubated at 28° C. for two to three days. A single colony for each putative co-integrate is picked and inoculated with 4 mL of 10 g/L bactopeptone, 10 g/L yeast extract, 5 g/L sodium chloride and 50 mg/L spectinomycin. The mixture is incubated for 24 h at 28° C. with shaking. Plasmid DNA from 4 mL of culture is isolated using Qiagen® Miniprep and an optional Buffer PB wash. The DNA is eluted in 30 μL. Aliquots of 2 L are used to electroporate 20 L of DH10b+20 L of twice distilled H₂O as per above. Optionally a 15 L aliquot can be used to transform 75-100 μL of INVITROGEN™ Library Efficiency DH5α. The cells are spread on plates containing LB medium and 50 μg/mL spectinomycin and incubated at 37° C. overnight.

Three to four independent colonies are picked for each putative co-integrate and inoculated 4 mL of 2×YT medium (10 g/L bactopeptone, 10 g/L yeast extract, 5 g/L sodium chloride) with 50 μg/mL spectinomycin. The cells are incubated at 37° C. overnight with shaking. Next, isolate the plasmid DNA from 4 mL of culture using QIAprep® Miniprep with optional Buffer PB wash (elute in 50 μL). Use 8 L for digestion with Sail (using parental DNA and PHP10523 as controls). Three more digestions using restriction enzymes BamHI, EcoRI, and HindIII are performed for 4 plasmids that represent 2 putative co-integrates with correct Sail digestion pattern (using parental DNA and PHP10523 as controls). Electronic gels are recommended for comparison.

### Example 13

### Transformation of Maize Using Agrobacterium

Maize plants can be transformed to overexpress a validated Arabidopsis lead gene or the corresponding homologs from various species in order to examine the resulting phenotype.

Agrobacterium-mediated transformation of maize is performed essentially as described by Zhao et al. in Meth. Mol. Biol. 318:315-323 (2006) (see also Zhao et al., Mol. Breed. 8:323-333 (2001) and U.S. Pat. No. 5,981,840 issued Nov. 9, 1999, incorporated herein by reference). The transformation process involves bacterium innoculation, co-cultivation, resting, selection and plant regeneration.

1. Immature Embryo Preparation:

Immature maize embryos are dissected from caryopses and placed in a 2 mL microtube containing 2 mL PHI-A medium.

2. Agrobacterium Infection and Co-Cultivation of Immature Embryos:

2.1 Infection Step:

PHI-A medium of (1) is removed with 1 mL micropipettor, and 1 mL of Agrobacterium suspension is added. The tube is gently inverted to mix. The mixture is incubated for 5 min at room temperature.

2.2 Co-Culture Step:

The Agrobacterium suspension is removed from the infection step with a 1 mL micropipettor. Using a sterile spatula the embryos are scraped from the tube and transferred to a plate of PHI-B medium in a 100×15 mm Petri dish. The embryos are oriented with the embryonic axis down on the surface of the medium. Plates with the embryos are cultured at 20° C., in darkness, for three days. L-Cysteine can be used in the co-cultivation phase. With the standard binary vector, the co-cultivation medium supplied with 100-400 mg/L L-cysteine is critical for recovering stable transgenic events.

3. Selection of Putative Transgenic Events:

To each plate of PHI-D medium in a 100×15 mm Petri dish, 10 embryos are transferred, maintaining orientation and the dishes are sealed with parafilm. The plates are incubated in darkness at 28° C. Actively growing putative events, as pale yellow embryonic tissue, are expected to be visible in six to eight weeks. Embryos that produce no events may be brown and necrotic, and little friable tissue growth is evident. Putative transgenic embryonic tissue is subcultured to fresh PHI-D plates at two-three week intervals, depending on growth rate. The events are recorded.

4. Regeneration of T0 Plants:

Embryonic tissue propagated on PHI-D medium is subcultured to PHI-E medium (somatic embryo maturation medium), in 100×25 mm Petri dishes and incubated at 28° C., in darkness, until somatic embryos mature, for about ten to eighteen days. Individual, matured somatic embryos with well-defined scutellum and coleoptile are transferred to PHI-F embryo germination medium and incubated at 28° C. in the light (about 80 μE from cool white or equivalent fluorescent lamps). In seven to ten days, regenerated plants, about 10 cm tall, are potted in horticultural mix and hardened-off using standard horticultural methods.

Media for Plant Transformation:
1. PHI-A: 4 g/L CHU basal salts, 1.0 mL/L 1000× Eriksson's vitamin mix, 0.5 mg/L thiamin HCl, 1.5 mg/L 2,4-D, 0.69 g/L L-proline, 68.5 g/L sucrose, 36 g/L glucose, pH 5.2. Add 100 μM acetosyringone (filter-sterilized).
2. PHI-B: PHI-A without glucose, increase 2,4-D to 2 mg/L, reduce sucrose to 30 g/L and supplemented with 0.85 mg/L silver nitrate (filter-sterilized), 3.0 g/L Gelrite®, 100 μM acetosyringone (filter-sterilized), pH 5.8.
3. PHI-C: PHI-B without Gelrite® and acetosyringonee, reduce 2,4-D to 1.5 mg/L and supplemented with 8.0 g/L agar, 0.5 g/L 2-[N-morpholino]ethane-sulfonic acid (MES) buffer, 100 mg/L carbenicillin (filter-sterilized).
4. PHI-D: PHI-C supplemented with 3 mg/L bialaphos (filter-sterilized).
5. PHI-E: 4.3 g/L of Murashige and Skoog (MS) salts, (Gibco, BRL 11117-074), 0.5 mg/L nicotinic acid, 0.1 mg/L thiamine HCl, 0.5 mg/L pyridoxine HCl, 2.0 mg/L glycine, 0.1 g/L myo-inositol, 0.5 mg/L zeatin (Sigma, Cat. No. Z-0164), 1 mg/L indole acetic acid (IAA), 26.4 μg/L abscisic acid (ABA), 60 g/L sucrose, 3 mg/L bialaphos (filter-sterilized), 100 mg/L carbenicillin (filter-sterilized), 8 g/L agar, pH 5.6.
6. PHI-F: PHI-E without zeatin, IAA, ABA; reduce sucrose to 40 g/L; replacing agar with 1.5 g/L Gelrite®; pH 5.6.

Plants can be regenerated from the transgenic callus by first transferring clusters of tissue to N6 medium supplemented with 0.2 mg per liter of 2,4-D. After two weeks the tissue can be transferred to regeneration medium (Fromm et al., Bio/Technology 8:833-839 (1990)).

Transgenic T0 plants can be regenerated and their phenotype determined. T1 seed can be collected.

Furthermore, a recombinant DNA construct containing a validated Arabidopsis gene can be introduced into an elite maize inbred line either by direct transformation or introgression from a separately transformed line.

Transgenic plants, either inbred or hybrid, can undergo more vigorous field-based experiments to study yield enhancement and/or stability under water limiting and water non-limiting conditions.

Subsequent yield analysis can be done to determine whether plants that contain the validated Arabidopsis lead gene have an improvement in yield performance (under water limiting or non-limiting conditions), when compared to the control (or reference) plants that do not contain the validated Arabidopsis lead gene. Specifically, water limiting conditions can be imposed during the flowering and/or grain fill period for plants that contain the validated Arabidopsis lead gene and the control plants. Plants containing the validated Arabidopsis lead gene would have less yield loss relative to the control plants, for example, at least 25%, at least 20%, at least 15%, at least 10% or at least 5% less yield loss, under water limiting conditions, or would have increased yield, for example, at least 5%, at least 10%, at least 15%, at least 20% or at least 25% increased yield, relative to the control plants under water non-limiting conditions.

### Example 14A

### Preparation of Arabidopsis Lead Gene (At5g62180) Expression Vector for Transformation of Maize

Using INVITROGEN™ GATEWAY® technology, an LR Recombination Reaction was performed to create the precursor plasmid pEV-DTP4. The vector pEV-DTP4 contains the following expression cassette:

Ubiquitin promoter::At5g62180(SEQ ID NO:17)::PinII terminator; cassette overexpressing the gene of interest, Arabidopsis DTP4 polypeptide.

The At5g62180 sequence with alternative codons, SEQ ID NO:19, was also cloned to create the precursor plasmid pEV-DTP4ac, which contains the following expression cassette: Ubiquitin promoter::At5g62180 (SEQ ID NO:19)::SB-GKAF terminator; cassette overexpressing the gene of interest, Arabidopsis DTP4 polypeptide.

The SB-GKAF terminator is described in U.S. application Ser. No. 14/236,499, herein incorporated by reference.

### Example 14B

### Transformation of Maize with the Arabidopsis Lead Gene (At5q62180) Using Agrobacterium

The DTP4 polypeptide expression cassette present in vector pEV-DTP4, and the DTP4 polypeptide expression cassette present in vector pEV-DTP4ac can be introduced into a maize inbred line, or a transformable maize line derived from an elite maize inbred line, using Agrobacterium-mediated transformation as described in Examples 12 and 13.

Vector pEV-DTP4 can be electroporated into the LBA4404 Agrobacterium strain containing vector PHP10523 (PCT Publication No. WO/2012/058528) to create the co-integrate vector pCV-DTP4. The co-integrate vector is formed by recombination of the 2 plasmids, pEV-DTP4 and PHP10523, through the COS recombination sites contained on each vector. The co-integrate vector pCV-DTP4 contains the same expression cassette as above (Example 14A) in addition to other genes (TET, TET, TRFA, ORI terminator, CTL, ORI V, VIR C1, VIR C2, VIR G, VIR B) needed for the Agrobacterium strain and the Agrobacterium-mediated transformation.

Similarly, the vector pEV-DTP4ac and PHP10523 were recombined to give the co-integrate vector pCV-DTP4ac. The co-integrate vector pCV-DTP4ac contains the same expression cassette as pEV-DTP4ac (Example 14A) in addition to other genes (TET, TET, TRFA, ORI terminator, CTL, ORI V, VIR C1, VIR C2, VIR G, VIR B) needed for the Agrobacterium strain and the Agrobacterium-mediated transformation

### Example 15

### Preparation of the Destination Vector PHP23236 for Transformation into Gaspe Flint Derived Maize Lines

Destination vector PHP23236 was obtained by transformation of Agrobacterium strain LBA4404 containing plasmid PHP10523 with plasmid PHP23235 and isolation of the resulting co-integration product. Plasmids PHP23236, PHP10523 and PHP23235 are described in PCT Publication No. WO/2012/058528, herein incorporated by reference. Destination vector PHP23236, can be used in a recombination reaction with an entry clone as described in Example 16 to create a maize expression vector for transformation of Gaspe Flint-derived maize lines.

### Example 16

### Preparation of Plasmids for Transformation into Gaspe Flint Derived Maize Lines

Using the INVITROGEN™ GATEWAY® LR Recombination technology, the protein-coding region of the At5g62180 candidate gene, was directionally cloned into the destination vector PHP23236 (PCT Publication No. WO/2012/058528) to create an expression vector, pGF-DTP4. This expression vector contains the protein-coding region of interest, encoding the DTP4 polypeptide, under control of the UBI promoter and is a T-DNA binary vector for Agrobacterium-mediated transformation into corn as described, but not limited to, the examples described herein.

### Example 17

### Transformation of Gaspe Flint Derived Maize Lines with a Validated Arabidopsis Lead Gene

Maize plants can be transformed to overexpress the Arabidopsis lead gene or the corresponding homologs from other species in order to examine the resulting phenotype. Gaspe Flint derived maize lines can be transformed and analyzed as previously described in PCT Publication No. WO/2012/058528, the contents of which are herein incorporated by reference.

### Example 18A

### Evaluation of Gaspe Flint Derived Maize Lines for Drought Tolerance

Transgenic Gaspe Flint derived maize lines containing the candidate gene can be screened for tolerance to drought stress in the following manner.

Transgenic maize plants are subjected to well-watered conditions (control) and to drought-stressed conditions. Transgenic maize plants are screened at the T1 stage or later.

For plant growth, the soil mixture consists of ⅓ TURFACE®, ⅓ SB300 and ⅓ sand. All pots are filled with the same amount of soil±10 grams. Pots are brought up to 100% field capacity (“FC”) by hand watering. All plants are maintained at 60% FC using a 20-10-20 (N-P-K) 125 ppm N nutrient solution. Throughout the experiment pH is monitored at least three times weekly for each table. Starting at 13 days after planting (DAP), the experiment can be divided into two treatment groups, well watered and reduce watered. All plants comprising the reduced watered treatment are maintained at 40% FC while plants in the well watered treatment are maintained at 80% FC. Reduced watered plants are grown for 10 days under chronic drought stress conditions (40% FC). All plants are imaged daily throughout chronic stress period. Plants are sampled for metabolic profiling analyses at the end of chronic drought period, 22 DAP. At the conclusion of the chronic stress period all plants are imaged and measured for chlorophyll fluorescence. Reduced watered plants are subjected to a severe drought stress period followed by a recovery period, 23-31 DAP and 32-34 DAP respectively. During the severe drought stress, water and nutrients are withheld until the plants reached 8% FC. At the conclusion of severe stress and recovery periods all plants are again imaged and measured for chlorophyll fluorescence. The probability of a greater Student's t Test is calculated for each transgenic mean compared to the appropriate null mean (either segregant null or construct null). A minimum (P<t) of 0.1 is used as a cut off for a statistically significant result.

### Example 18B

### Evaluation of Maize Lines for Drought Tolerance

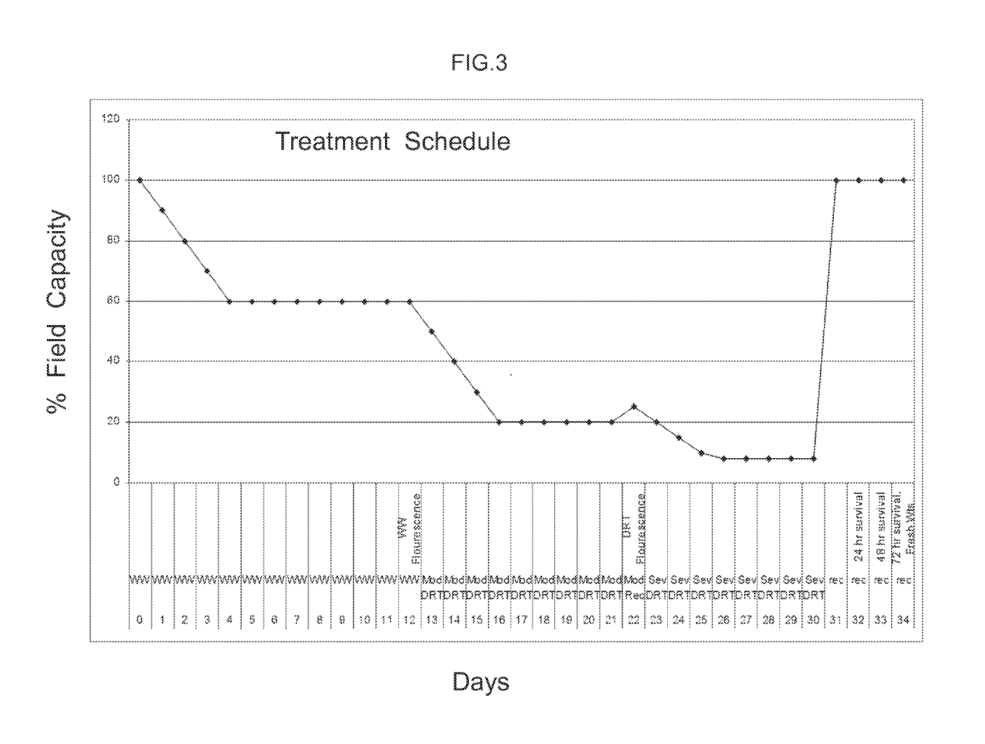

Lines with Enhanced Drought Tolerance can also be screened using the following method (see also FIG. 3 for treatment schedule):

Transgenic maize seedlings are screened for drought tolerance by measuring chlorophyll fluorescence performance, biomass accumulation, and drought survival. Transgenic plants are compared against the null plant (i.e., not containing the transgene). Experimental design is a Randomized Complete Block and Replication consist of 13 positive plants from each event and a construct null (2 negatives each event).

Plant are grown at well watered (WW) conditions=60% Field Capacity (% FC) to a three leaf stage. At the three leaf stage and under WW conditions the first fluorescence measurement is taken on the uppermost fully extended leaf at the inflection point, in the leaf margin and avoiding the mid rib.

This is followed by imposing a moderate drought stress (FIG. 3, day 13, MOD DRT) by maintaining 20% FC for duration of 9 to 10 days. During this stress treatment leaves may appear gray and rolling may occur. At the end of MOD DRT period, plants are recovered (MOD rec) by increasing to 25% FC. During this time, leaves will begin to unroll. This is a time sensitive step that may take up to 1 hour to occur and can be dependent upon the construct and events being tested. When plants appear to have recovered completed (leaves unrolled), the second fluorescence measurement is taken.

This is followed by imposing a severe drought stress (SEV DRT) by withholding all water until the plants collapse. Duration of severe drought stress is 8-10 days and/or when plants have collapse. Thereafter, a recovery (REC) is imposed by watering all plants to 100% FC. Maintain 100% FC 72 hours. Survival score (yes/no) is recorded after 24, 48 and 72 hour recovery.

The entire shoot (Fresh) is sampled and weights are recorded (Fresh shoot weights). Fresh shoot material is then dried for 120 hrs at 70 degrees at which time a Dry Shoot weight is recorded.

Measured variables are defined as follows:

The variable “Fv′/Fm′ no stress” is a measure of the optimum quantum yield (Fv′/Fm′) under optimal water conditions on the uppermost fully extended leaf (most often the third leaf) at the inflection point, in the leaf margin and avoiding the mid rib. Fv′/Fm′ provides an estimate of the maximum efficiency of PSII photochemistry at a given PPFD, which is the PSII operating efficiency if all the PSII centers were open (Q_{A} oxidized).

The variable “Fv′/Fm′ stress” is a measure of the optimum quantum yield (Fv′/Fm′) under water stressed conditions (25% field capacity). The measure is preceded by a moderate drought period where field capacity drops from 60% to 20%. At which time the field capacity is brought to 25% and the measure collected.

The variable “phiPSII_no stress” is a measure of Photosystem II (PSII) efficiency under optimal water conditions on the uppermost fully extended leaf (most often the third leaf) at the inflection point, in the leaf margin and avoiding the mid rib. The phiPSII value provides an estimate of the PSII operating efficiency, which estimates the efficiency at which light absorbed by PSII is used for Q_{A} reduction.

The variable “phiPSII_stress” is a measure of Photosystem II (PSII) efficiency under water stressed conditions (25% field capacity). The measure is preceded by a moderate drought period where field capacity drops from 60% to 20%. At which time the field capacity is brought to 25% and the measure collected.

### Example 19A

### Yield Analysis of Maize Lines with the Arabidopsis Lead Gene

A recombinant DNA construct containing a validated Arabidopsis gene can be introduced into an elite maize inbred line either by direct transformation or introgression from a separately transformed line.

Transgenic plants either inbred or hybrid, can undergo more vigorous field-based experiments to study yield enhancement and/or stability under well-watered and water-limiting conditions.

Subsequent yield analysis can be done to determine whether plants that contain the validated Arabidopsis lead gene have an improvement in yield performance under water-limiting conditions, when compared to the control plants that do not contain the validated Arabidopsis lead gene. Specifically, drought conditions can be imposed during the flowering and/or grain fill period for plants that contain the validated Arabidopsis lead gene and the control plants. Reduction in yield can be measured for both. Plants containing the validated Arabidopsis lead gene have less yield loss relative to the control plants, for example, at least 25%, at least 20%, at least 15%, at least 10% or at least 5% less yield loss.

The above method may be used to select transgenic plants with increased yield, under water-limiting conditions and/or well-watered conditions, when compared to a control plant not comprising said recombinant DNA construct. Plants containing the validated Arabidopsis lead gene may have increased yield, under water-limiting conditions and/or well-watered conditions, relative to the control plants, for example, at least 5%, at least 10%, at least 15%, at least 20% or at least 25% increased yield.

### Example 19B

### Yield Analysis of Maize Lines Transformed with pCV-DTP4 Encoding the Arabidopsis Lead Gene At5g62180

Nine transgenic events were field tested at 3 locations, Locations “A”, “E”, and “B”. At the “B” location, drought conditions were imposed during flowering (“B1”; flowering stress) and during the grain fill period (“B2”; grain fill stress). The “A” location was well-watered, and the “E” location experienced mild drought during the grain-filling period. Yield data (bushel/acre; bu/ac) of the 9 transgenic events is shown in FIG. 5 together with the wt and bulk null control (BN). Statistical significance is reported at P<0.1 for a two-tailed test.

The significant values (with p-value less than or equal to 0.1 with a 2-tailed test) are shown in bold when the value is greater than the null comparator and in bold and italics when that value is less than the null.

In the most severe “B2” location it was neutral. In an intermediate “B1” location three events were positive but the experiment was unreliable because of the unexpected divergence between null and wild type performance.

### Example 19C

### Yield Analysis of Maize Lines Transformed with pCV-DTP4ac Encoding the Arabidopsis Lead Gene At5q62180

First Year Testing:

The AT-DTP4 polypeptide (SEQ ID NO:18) encoded by the nucleotide sequence (SEQ ID NO:19) present in the vector pCV-DTP4ac was introduced into a transformable maize line derived from an elite maize inbred line as described in Examples 14A and 14B.

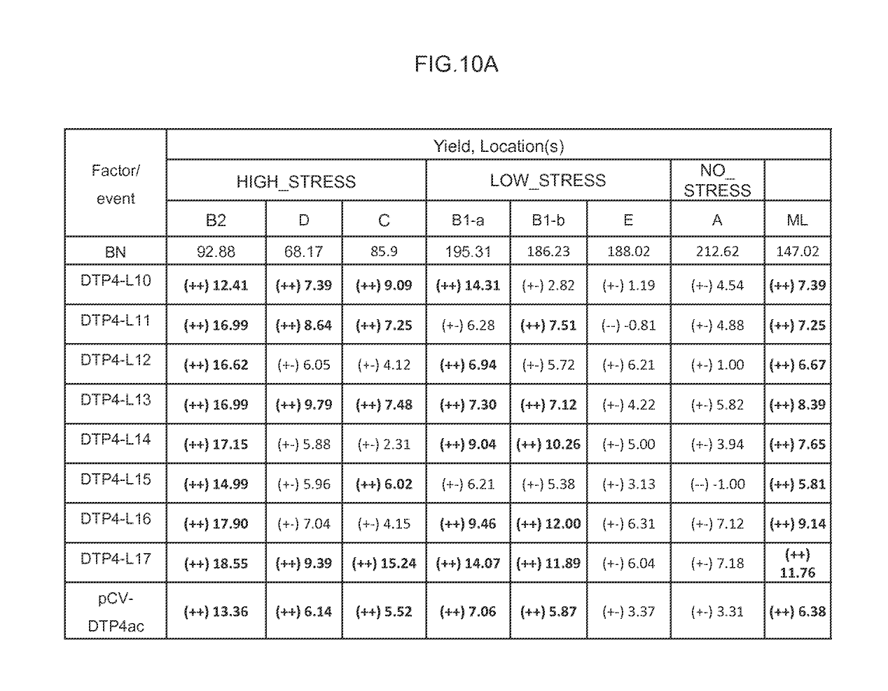

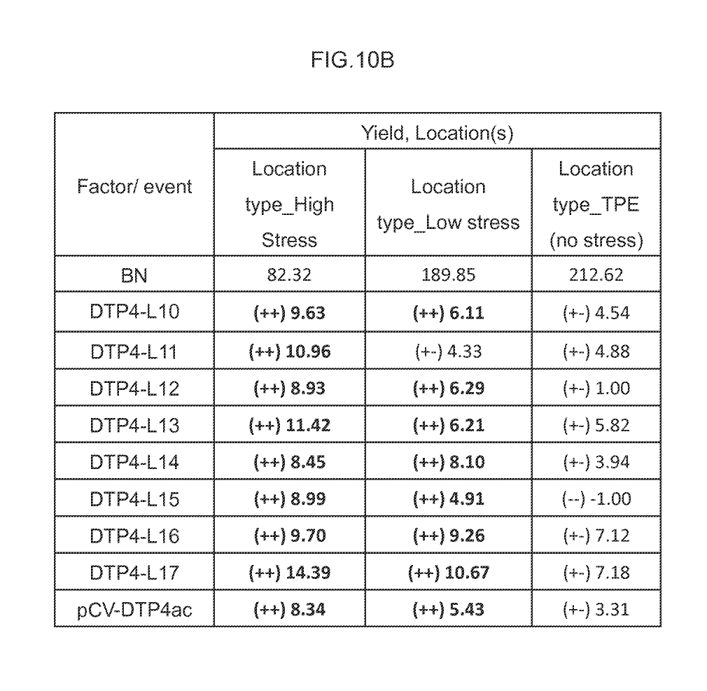

Eight transgenic events were field tested at 5 locations A, E, C, D, and B. At the location B, mild drought conditions were imposed during flowering (this treatment was divided into 2 areas B1-a and B1-b) and severe drought conditions were imposed during the grain fill period (“grain fill stress; B2). The “A” location was well-watered, and the “E” location experienced mild drought during the grain-filling period. Both “C” and “D” locations experienced severe stress (FIG. 10).

Yield data were collected in all locations, with 3-6 replicates per location.

Yield data (bushel/acre; bu/ac) for the 8 transgenic events is shown in FIGS. 10A and 10B together with the bulk null control (BN). Yield analysis was by ASREML (VSN International Ltd), and the values are BLUPs (Best Linear Unbiased Prediction) (Cullis, B. R et al (1998) Biometrics 54: 1-18, Gilmour, A. R. et al (2009). ASReml User Guide 3.0, Gilmour, A. R., et al (1995) Biometrics 51: 1440-50).

As shown in FIG. 10A, consistent effect of the transgene on yield was seen in at all the locations that resulted in a significant positive effect in 3-8 events., with the positive event magnitude ranging from 4 to 16 bu/ac.

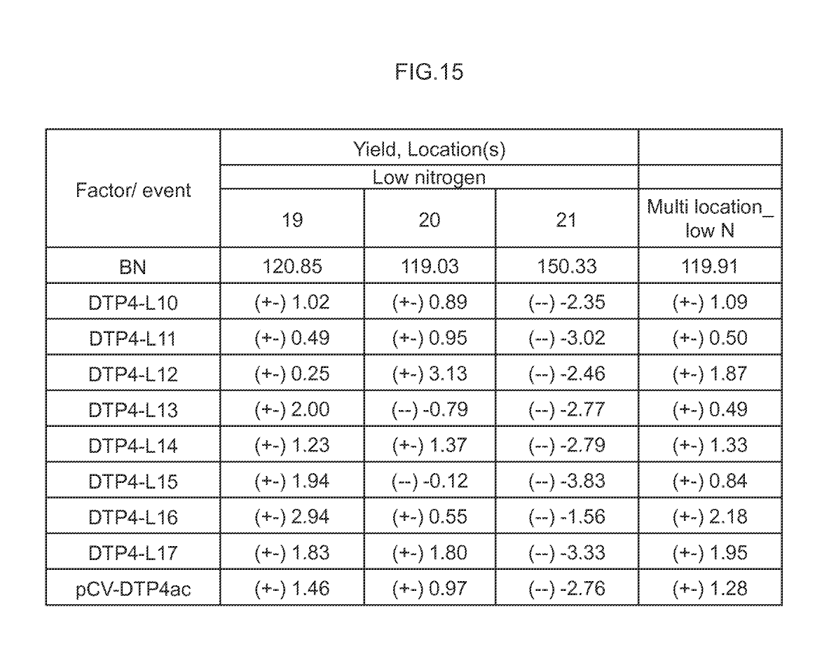

FIG. 10B shows the yield analysis by grouping locations into “high stress”, “low stress” and “no stress (TPE)” category. As can be seen from FIG. 15B, positive effect of the transgene on yield was seen for all 8 transgenic events in high stress and low stress locations, and in 2 events in the “no stress category”.

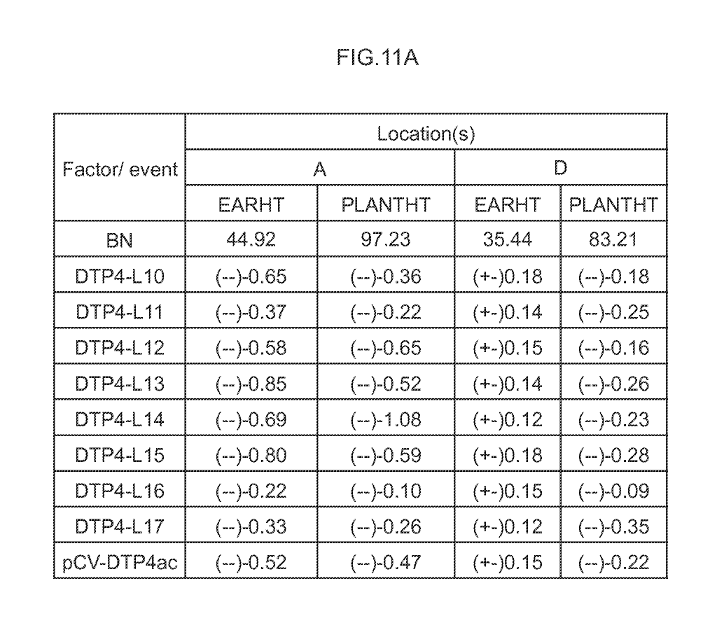

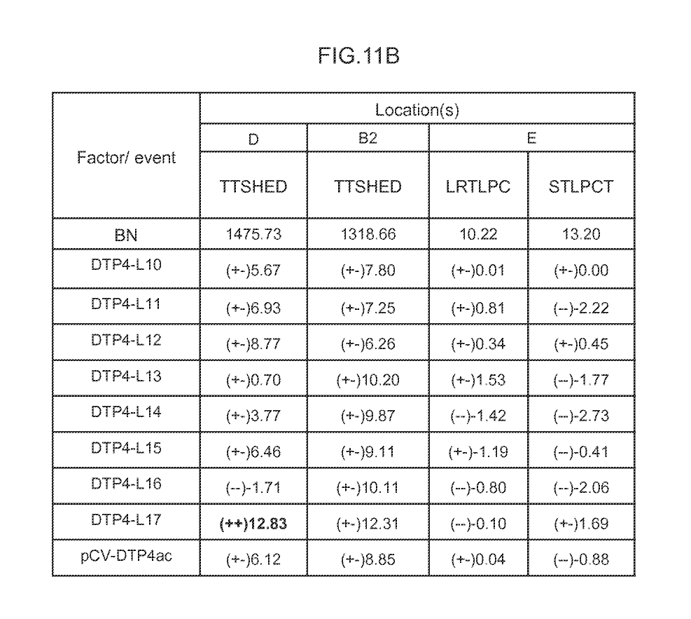

Effect of the transgene on other agronomic characteristics were also evaluated; such as plant and ear height (EARHT, PLTHT; at location “A” (no-stress) and location “D” (high-stress) locations), thermal time to shed (TTSHED: locations “D” and B2-b (location B at grain filling stress); both high-stress locations), percent root lodging or stalk lodging (LRTLPC, STLPCT; at the location “E” (low stress location). As shown in FIG. 11A and FIG. 11B, no effect of the transgene on these characteristics was observed.

Second Year Testing:

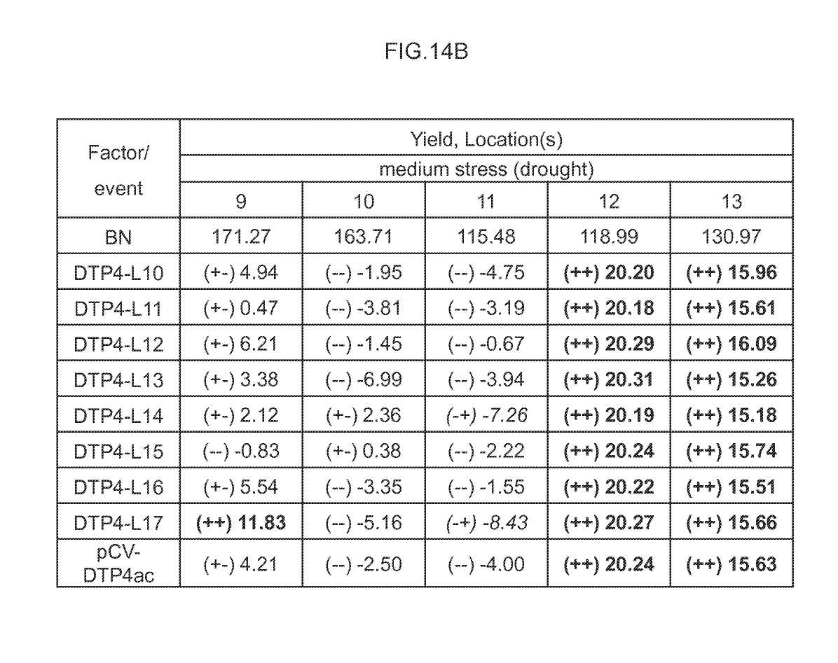

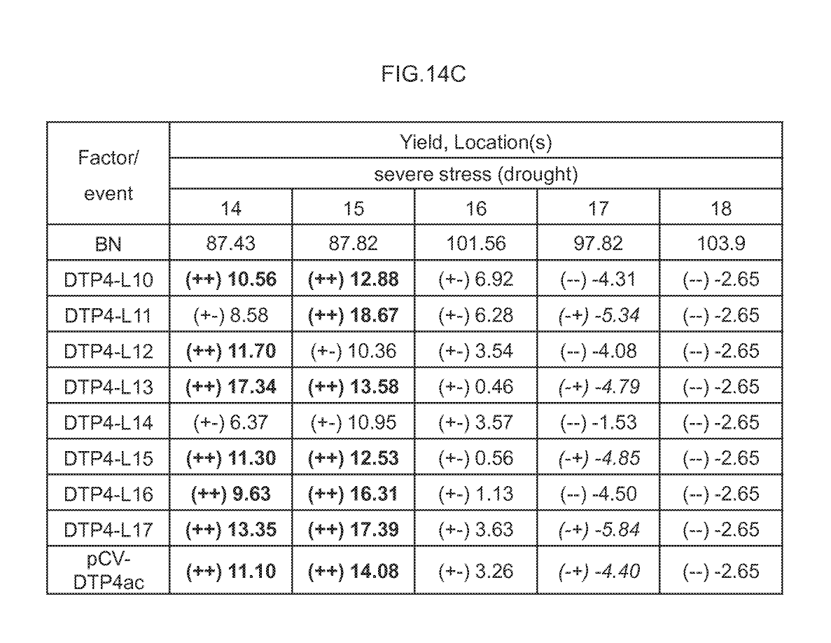

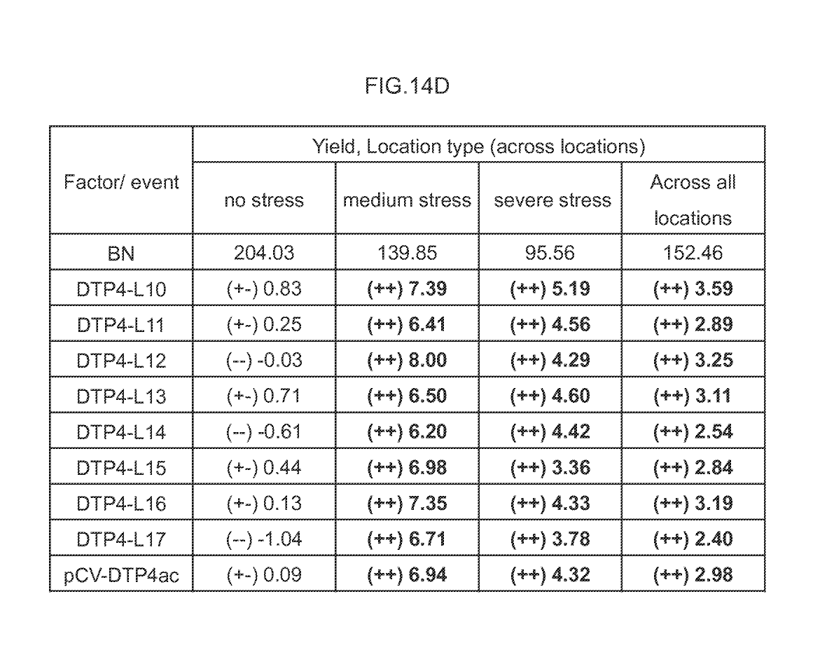

The eight transgenic events field tested for the first year, were field tested for a second year multiple locations with different levels of drought stress: no stress (8 locations; 1-8 in FIG. 14A); medium stress (5 locations; 9-13 in FIG. 14A); and severe stress (5 locations; 14-18 in FIG. 14A).

The eight transgenic events were also tested in three low nitrogen locations (locations 19-21 in FIG. 14A)

Yield data were collected in all locations, with 3-6 replicates per location.

Yield data (bushel/acre; bu/ac) for the 8 transgenic events is shown in FIG. 14A-14C for the drought stress, and in FIG. 15 the yield data in response to low nitrogen is shown; all the data are shown with the bulk null control (BN). Yield analysis was by ASREML (VSN International Ltd), and the values are BLUPs (Best Linear Unbiased Prediction) (Cullis, B. R et al (1998) Biometrics 54: 1-18, Gilmour, A. R. et al (2009). ASReml User Guide 3.0, Gilmour, A. R., et al (1995) Biometrics 51: 1440-50). FIG. 14D shows the multi-location anlaysis for the “no stress”, “medium stress” and “severe stress” locations, along with the multi-location analysis for all the drought stress locations.

As shown in FIG. 14A-FIG. 14D, effect of the transgene on yield was seen in at least one location with no stress, at least 2 locations in medium and severe stress; the multi-location analysis in FIG. 14D shows consistent positive effect of the transgene on yield., with the positive event magnitude ranging from 15 to 20 bu/ac, under medium stress.

FIG. 14D shows the yield analysis by grouping locations into “high stress”, “low stress” and “no stress” category. As can be seen from FIG. 14B, positive effect of the transgene on yield was seen for all 8 transgenic events in medium stress and severe stress locations, and in 2 events in the “no stress category”.

As shown in FIG. 15, no positive effect of the transgene on yield was observed under low nitrogen conditions.

### Example 19D

### Yield Analysis of Maize Lines Transformed with pCV-AT-CXE8ac Encoding the Arabidopsis DTP4 Homolog AT-CXE8

The AT-CXE8 polypeptide (SEQ ID NO:64) encoded by the nucleotide sequence (SEQ ID NO:63), with alternative codons, was cloned as described in Example 14A and Example 14B; using the Invitrogen Gateway technology.

The At2g45600 sequence with alternative codons, SEQ ID NO:63 was also cloned to create the precursor plasmid pEV-CXE8ac, which contains the following expression cassette: Zm Ubiquitin promoter::At2g45600 (SEQ ID NO:63)::Sb-Ubi terminator; cassette overexpressing the gene of interest, the AT-DTP4 homolog, Arabidopsis CXE8 polypeptide.

The AT-CXE8 polypeptide (SEQ ID NO:64) encoded by the nucleotide sequence (SEQ ID NO:63) present in the vector pCV-AT-CXE8ac was introduced into a transformable maize line derived from an elite maize inbred line as described in Examples 14A and 14B.

Seven transgenic events were field tested at 7 locations.

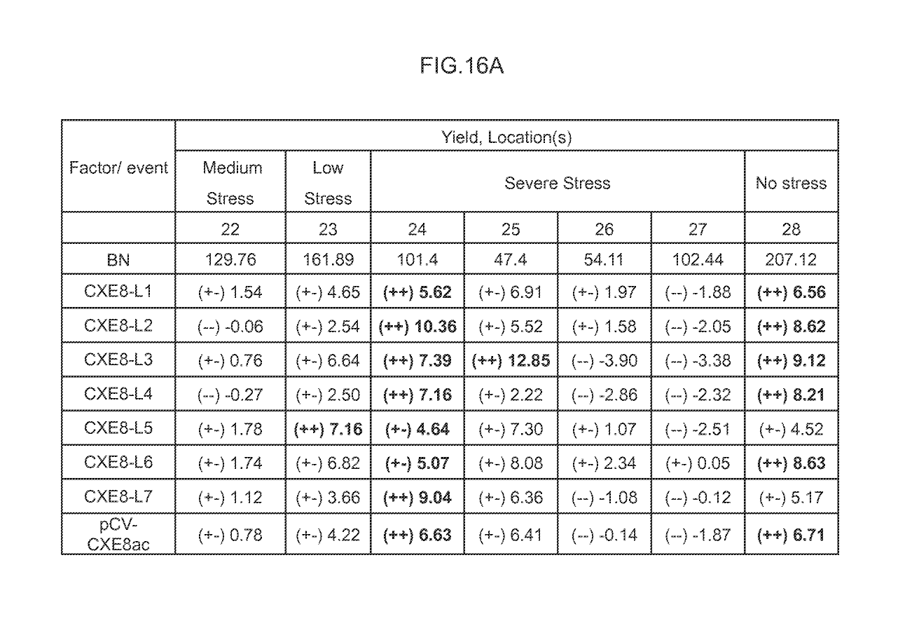

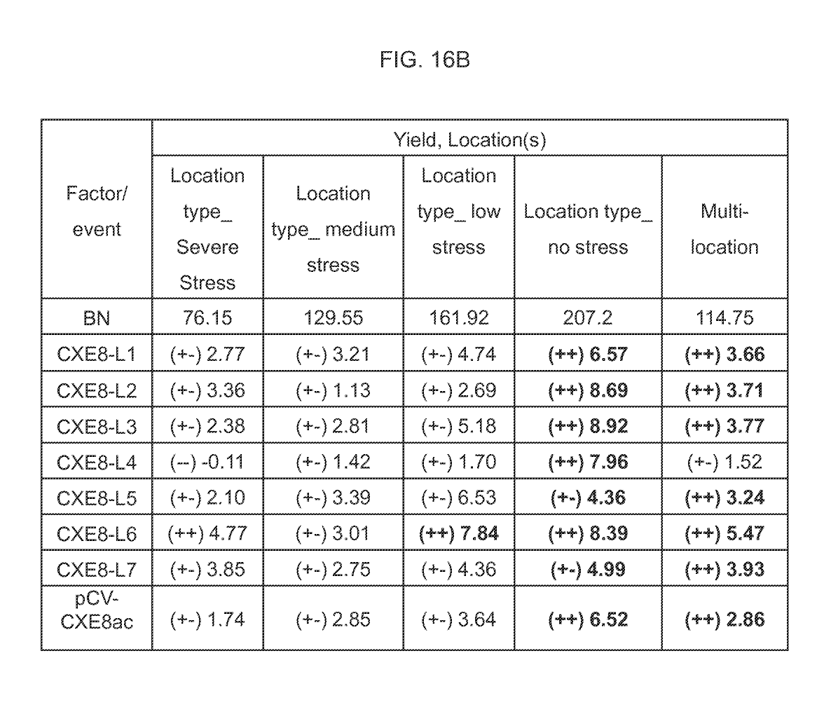

The seven transgenic events were field tested at multiple locations with different levels of drought stress: no stress (1 location; location 28 in FIG. 16A); medium stress (1 location; location 22 in FIG. 16A); and severe stress (4 locations; locations 24-27 in FIG. 16A).

Yield data were collected in all locations, with 3-6 replicates per location.

Yield data (bushel/acre; bu/ac) for the seven transgenic events is shown in FIGS. 16A and 16B together with the bulk null control (BN). Yield analysis was by ASREML (VSN International Ltd), and the values are BLUPs (Best Linear Unbiased Prediction) (Cullis, B. R et al (1998) Biometrics 54: 1-18, Gilmour, A. R. et al (2009). ASReml User Guide 3.0, Gilmour, A. R., et al (1995) Biometrics 51: 1440-50).

As shown in FIG. 16A, consistent effect of the transgene on yield was seen at no stress and severe stress locations, that resulted in a significant positive effect in 3-8 events, with the positive event magnitude ranging from 5 to 10 bu/ac.

FIG. 16B shows the yield analysis across locations, grouped by drought stress levels. As can be seen from FIG. 16B, positive effect of the transgene on yield was seen for 6 transgenic events in across location analysis, after taking all stress level locations together.

### Example 20A

### Preparation of Maize DTP4 Polypeptide Lead Gene Expression Vector for Transformation of Maize

The protein-coding region of the maize DTP4 homologs disclosed in the application can be introduced into the INVITROGEN™ vector pENTR/D-TOPO® to create entry clones.

Using INVITROGEN™ GATEWAY® technology, LR Recombination Reaction can be performed with the entry clones and a destination vector to create precursor plasmids. These vectors contain the following expression cassette:

Ubiquitin promoter::Zm-DTP4-Polypeptide::Pin II terminator; cassette overexpressing the gene of interest.

### Example 20B

### Transformation of Maize with Maize DTP4 Polypeptide Lead Gene Using Agrobacterium

The maize DTP4 polypeptide expression cassette present in the vectors from the above example can be introduced into a maize inbred line, or a transformable maize line derived from an elite maize inbred line, using Agrobacterium-mediated transformation as described in Examples 12 and 13.

Any or of these vectors can be electroporated into the LBA4404 Agrobacterium strain containing vector PHP10523 (PCT Publication No. WO/2012/058528) to create a co-integrate vector. The co-integrate vector is formed by recombination of the 2 plasmids, the precursor plasmid and PHP10523, through the COS recombination sites contained on each vector. The co-integrate vector contains the same 3 expression cassettes as above (Example 20A) in addition to other genes (TET, TET, TRFA, ORI terminator, CTL, ORI V, VIR C1, VIR C2, VIR G, VIR B) needed for the Agrobacterium strain and the Agrobacterium-mediated transformation.

### Example 21

### Preparation of Maize Expression Plasmids for Transformation into Gaspe Flint Derived Maize Lines

Using the INVITROGEN™ GATEWAY® Recombination technology described in Example 9, the clones encoding maize DTP4 polypeptide homologs disclosed herein can be directionally cloned into the destination vector PHP23236 (PCT Publication No. WO/2012/058528) to create expression vectors. Each expression vector contains the cDNA of interest under control of the UBI promoter and is a T-DNA binary vector for Agrobacterium-mediated transformation into corn as described, but not limited to, the examples described herein.

### Example 22

### Transformation and Evaluation of Soybean with Soybean Homologs of Validated Lead Genes

Based on homology searches, one or several candidate soybean homologs of validated Arabidopsis lead genes can be identified and also be assessed for their ability to enhance drought tolerance in soybean. Vector construction, plant transformation and phenotypic analysis will be similar to that in previously described Examples.

### Example 23

### Transformation of Arabidopsis with Maize and Soybean Homologs of Validated Lead Genes

Soybean and maize homologs to validated Arabidopsis lead genes can be transformed into Arabidopsis under control of the 35S promoter and assessed for their ability to enhance drought tolerance in Arabidopsis. Vector construction, plant transformation and phenotypic analysis will be similar to that in previously described Examples.

### Example 24

### Transformation of Arabidopsis with DTP4 Polypeptides from Other Species

Any of the DTP4 polypeptides disclosed herein, including the ones given in Table 1 or Table 2, can be transformed into Arabidopsis under control of the 35S promoter and assessed for their ability to enhance drought tolerance, or in any of the other assays described herein, in Arabidopsis. Vector construction, plant transformation and phenotypic analysis will be similar to that in previously described Examples.

### Example 25A

### Osmotic Stress Assay

To assay the osmotic stress tolerance of a transgenic line, a combination of osmolytes in the media, such as water soluble inorganic salts, sugar alcohols and high molecular weight non-penetrating osmolytes can be used to select for osmotically-tolerant plant lines.

The osmotic stress agents used in this quad stress assay are:
1) NaCl (sodium chloride)
2) Sorbitol
3) Mannitol
4) Polyethylene Glycol (PEG)
   By providing these agents in the media, we aim to mimic multiple stress conditions in the in vitro environment thereby giving the plant the opportunity to respond to four stress agents.

### Methods and Materials:

As there are four stress agents being used together, a quarter of each together in a solution will denote 100% stress or an osmotic pressure of 1.23 MPa. Therefore the following concentrations of each component are used in 100% quad media.

| Stress agents | Concentrations |
|---|---|
| NaCl— | 62.5 mM |
| Sorbitol- | 125 mM |
| Mannitol- | 125 mM |
| PEG- | 10% |

### Assay Conditions:

Seeds are surface sterilized and stratified for 48 hrs. About 100 seeds are inoculated in one plate and cultured in a growth chamber programmed for 16 h of light at 22′C temperature and 50% relative humidity. Germination is scored as the emergence of radicle.

### Assay Plan:

A 6-day assay and an extended 10-day assay are done to test the seeds transgenic Arabidopsis line for osmotic stress tolerance.

Day 0—Surface sterilized seeds of different drought leads and stratify
Day 2—Inoculated onto quad media
Day 4—Counted for germination (48 hrs)
Day 5—Counted for germination (72 hrs) I Take pictures or Scan plates from 48 hrs to 96 hrs.
Day 6—Counted for germination (96 hrs)
For the extended 10-day assay, germination is scored from 48 hrs to 96 hrs. On day 7, 8, 9 and 10, the emerged seedlings were checked for greenness and four leaf stage.

### Preparation of Media:

Germination medium (GM or 0% quad media) for 1 liter:

| | | |
|---|---|---|
| MS salt | 4.3 | g |
| Sucrose | 10 | g |
| 1000x Vitamin mix | 1 | ml |
| MES (pH 5.7 with KOH) | 10 | ml |
| Phytagel (0.3%) | 3 | g |

To this the quad agents (the four osmolytes) are added by individually weighing the specific amounts in grams for their respective concentrations. Quad media preparation chart for all concentrations of osmolytes is given in Table 6.

TABLE 6
Quad Media Preparation Chart

| | 10% | 20% | 30% | 40% | 50% | 60% | 70% | 80% | 90% | 100% |
|---|---|---|---|---|---|---|---|---|---|---|
| NaCl | 0.36 | 0.731 | 1.09 | 1.46 | 1.82 | 2.19 | 2.55 | 2.9 | 3.29 | 3.656 |
| Mannitol | 2.27 | 4.55 | 8.83 | 9.1 | 11.38 | 13.66 | 15.93 | 18.2 | 20.49 | 22.77 |
| Sorbitol | 2.27 | 4.55 | 6.83 | 9.1 | 11.38 | 13.66 | 15.93 | 18.2 | 20.49 | 22.77 |
| PEG | 10 | 20 | 30 | 40 | 50 | 60 | 70 | 80 | 90 | 100 |

### Sterilization of Seeds:

Approximately 100 μl of Arabidopsis Columbia wild type seeds (col wt) and the seeds of the transgenic line to be tested are taken in 1.75 ml microfuge tubes and sterilized in ethanol for 1 min 30 sec followed by one wash with sterile water. Then they are subjected to bleach treatment (4% bleach with Tween 20) for 2 min 30 sec. This is followed by 4 to 5 washes in sterile water. Seeds are stratified at 4° C. for 48 hrs before inoculation.

### Inoculation of Seeds:

Stratified seeds are plated onto a single plate of each quad stress concentration as given in Table 6. Plates are cultured in the chambers set at 16 h of light at 22° C. temperature and 50% relative humidity. Germination is scored as the emergence of radicle over a period of 48 to 96 hrs. Seeds are counted manually using a magnifying lens. Plates are scanned at 800 dpi using Epson scanner 10,000 XL and photographed. In case of the extended assay, leaf greenness (manual) and true leaf emergence i.e, 4Leaf stage (manual scoring) are also scored over a period of 10 days to account for the growth rate and health of the germinated seedlings.

The data is analyzed as percentage germination to the total number of seeds that are inoculated. Analyzed data is represented in the form of bar graphs and sigmoid curves by plotting quad concentrations against percent germination.

### Example 25B

### Seedling Emergence Under Osmotic Stress of Transgenic Arabidopsis Seeds with AT-DTP4 Proteins

T1 seeds from transgenic Arabidopsis line with AT-DTP4 protein, containing the 35S promoter::At5g62180 expression construct pBC-Yellow-At5g62180, generated as described above, were tested for seedling emergence under osmotic stress as described in Example 25A.

Arabidopsis Columbia seeds were used as wild-type control and at 60% there was a dip in germination and thereafter a decline and zero germination at 100%, as shown in Table 7.

Table 7 presents the percentage germination data at 48 hours for seedling emergence under osmotic stress.

TABLE 7
Percentage Germination Data in Arabidopsis

| Quad % in the | % Germination | % Germination for |
|---|---|---|
| Media | for WT | At5g62180 |
| 0 | 96 | 90 |
| 10 | 80 | 87 |
| 20 | 76 | 90 |
| 30 | 69 | 92 |
| 40 | 52 | 90 |
| 50 | 29 | 82 |
| 60 | 20 | 66 |
| 70 | 10 | 54 |
| 80 | 2 | 9 |
| 90 | 6 | 65 |
| 100 | 0 | 2 |

### Seedling Emergence Under Osmotic Stress—10 Day Assay:

The results in Table 7 demonstrate that the transgenic Arabidopsis line (Line ID 64) containing the 35S promoter::At5g62180 expression construct, pBC-Yellow-At5g62180, which was previously selected as having a drought tolerance and ABA-hypersensitivity phenotype, also demonstrates increased seedling emergence under osmotic stress.

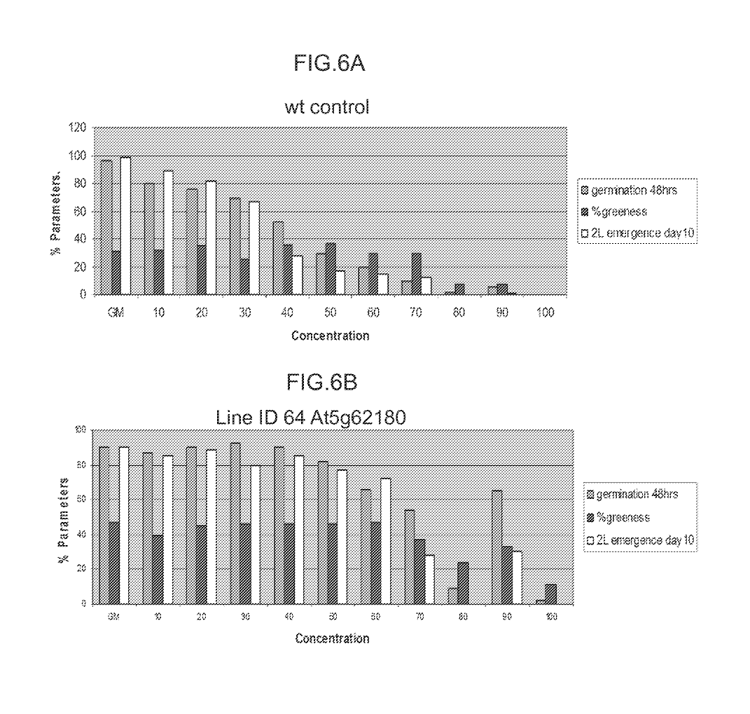

The osmotic stress assay for Line ID 64 was repeated, and scored for percentage greenness and percentage leaf emergence in an extended 10 day assay as well. The line was scored at 0% (GM or growth media), 10%, 20%, 30%, 40%, 50%, 60%, 70%, 80%, 90% and 100% quad, for germination at 48 hours, and for percentage greenness and percentage leaf emergence in an extended 10 day assay. The results are shown in FIG. 6A and FIG. 6B.

Percentage greenness and percentage leaf emergence were assayed. Percentage greenness was scored as the percentage of seedlings with green leaves (cotyledonary or true leaves) compared to yellow, brown or purple leaves. Greenness was scored manually and if there was any yellow or brown streaks on any of the 4 leaves, it was not considered green. Greenness was counted for seedlings with total green leaves only.

The leaf emergence was scored as the appearance of fully expanded leaves 1 and 2, after the two cotyledonary leaves had fully expanded. Therefore, the percentage leaf emergence is the number of seedlings with 2 true leaves or 4 leaves in total (2 cotyledonary and 2 true leaves).

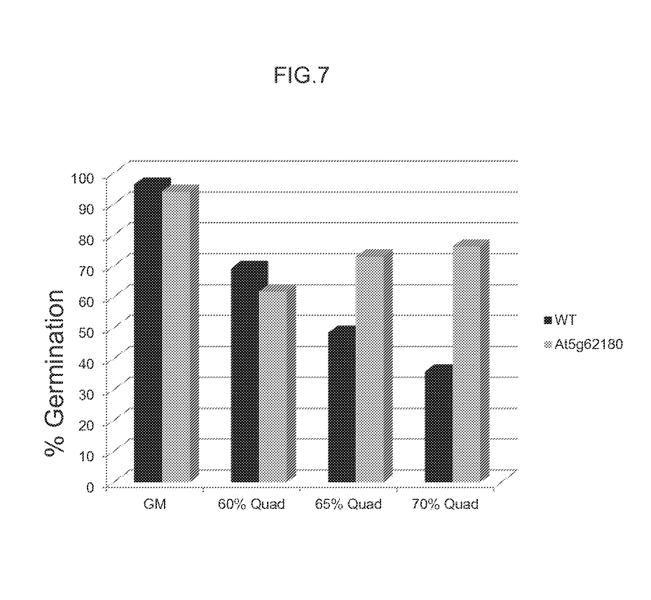

The percentage germination experiment at 48 hours was repeated once more with bulked seeds, in triplicates, and the results are shown in FIG. 7. Seeds were plated on MSO plate containing MS media+methionine sulphoximine and selected plants transplanted to the soil, seeds harvested and assayed.

### Example 26A

### ABA/Root Growth Assay

Plants being sessile have evolved a higher adaptability to overcome adverse environmental challenges. The phytohormone abscisic acid (ABA) is a key endogenous messenger in plants' responses to such stresses and therefore understanding ABA signaling is essential for improving plant performance especially under drought stress. Drought is a very complicated phenomenon involving several key regulators and in order to capture wide spectrum of such players a multi-assay approach is imperative. A root growth assay has been developed keeping this objective in mind.

In the ABA/Root assay, the sensitivity of root growth on media containing ABA post germination on MS media is used as the assay criterion. MS media comprises of MS basal salts, MS vitamins, sucrose and phytagel as a gelling agent. ABA/Root assay will enable us to potentially capture both hypersensitive and hyposensitive outliers/leads making it a powerful tool for screening of new genes and as a cross validation assay.

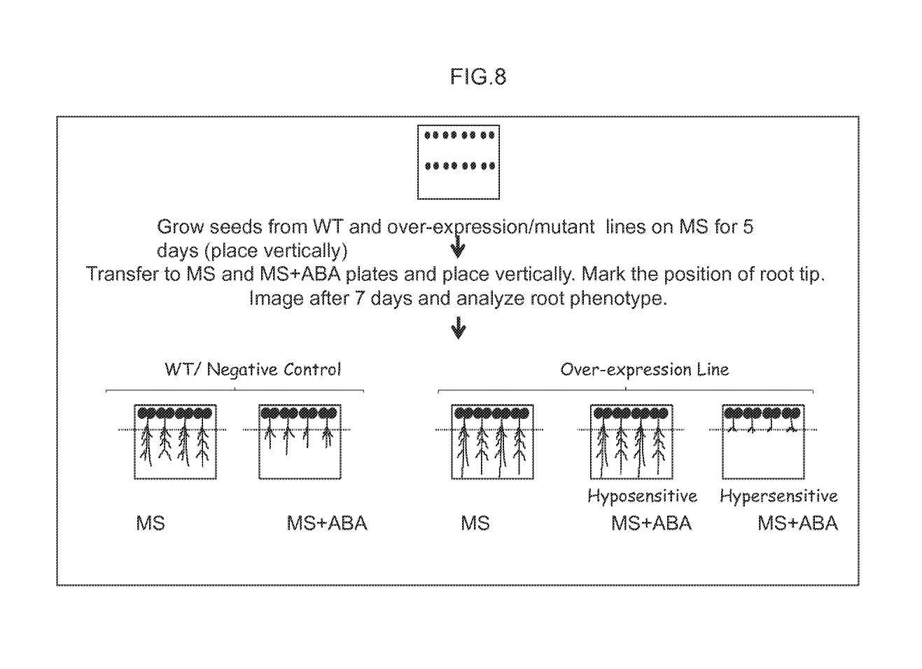

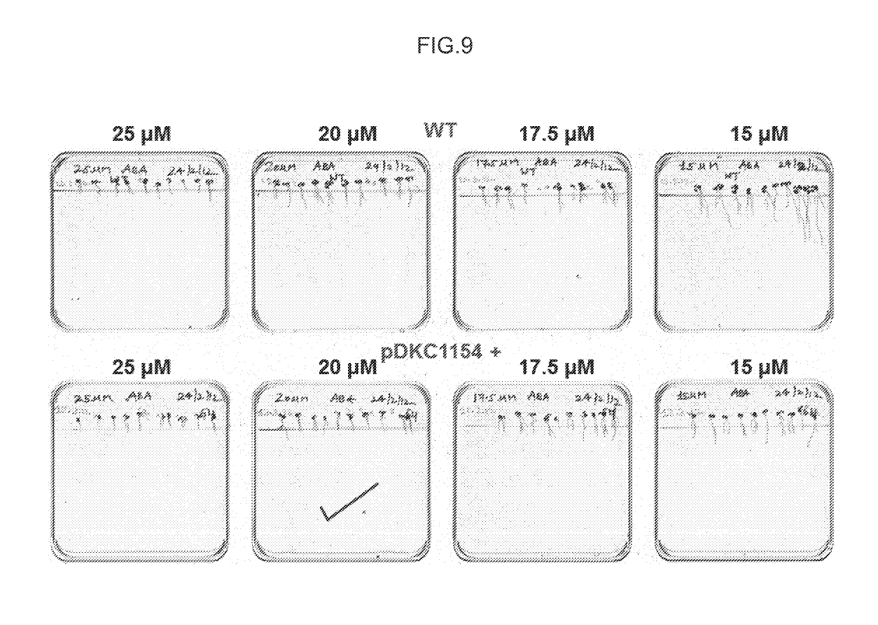

The ABA/Root assay is a two phase assay. Phase I includes growing seeds on plain germination/MS media vertically under 230 μMol light intensity. After 5 days of germination, seedlings are picked and transferred to media comprising ABA. The position of the root tip at the time of transfer is marked. The seedlings are allowed to grow vertically for 7 days on media containing ABA with daily rotation of plates such that each plate receives uniform light. On the seventh day, the plates are imaged and root phenotypes are analyzed. The overall schematic of the assay is presented in FIG. 8.

### Example 26B

### ABA/Root Growth Assay with Transgenic Arabidopsis Seeds with AT-DTP4 Polypeptide

In this assay, an ABA hypersensitive outlier would be expected to have seedlings arrested at the point of transfer whereas in an ABA hyposensitive outlier the roots would continue to grow because of their inability to sense ABA in the media. For lines that are insensitive, would be expected to behave similar to WT, which would be the negative control.

### Assay Conditions:

WT seeds and transgenic seeds containing the pBC-yellow-At5g62180 construct described in Example 5A were used for this assay. Seeds were surface sterilized first with 100% ethanol followed with bleach+Tween 20 solution followed by 4 washes of sterile water and stratified for 48 hrs. Two rows of around 30 stratified seeds each were sown on germination media and the plates were kept vertically in the growth chamber for 5 days. The growth chamber settings were 16 h of 230 μMol light at 22° C. temperature and 50% relative humidity. After 5 days, the seedlings were picked one by one and transferred to media containing different concentrations of ABA, 0, 2.5, 5, 10, 15, 17.5, 20, 25 and 30 μM ABA. The seedlings were grown vertically for 7 days. After 7 days, root phenotypes were analyzed and recorded. The representative results for the concentrations in the range 15-25 μM are shown in FIG. 9.

### Example 27

### ABA Sensitivity Assay

### Percentage Germination Assay with DTP4 Polypeptides in Arabidopsis

DTP4 polypeptides homologous to AT-DTP4 (SEQ ID NO:18) were tested for their ability to confer ABA-hypersensitivity by a percentage germination assay as described in Example 7.

The cDNA protein-coding region for each of these homologs was synthesized and cloned into the transformation vector. The homologs were tested for ABA hypersensitivity on 2 ABA concentrations, 1 μM and 2 μM.

Transgenic T2 seeds were selected, and used for the germination assay as described in Example 7. Two Sesbania bispinosa homologs sesgr1n.pk107.c11 and sesgr1n.pk079.h12 and (SEQ ID NOS:44 and 46, respectively), showed ABA hypersensitivity when they were directly expressed by the 35S promoter.

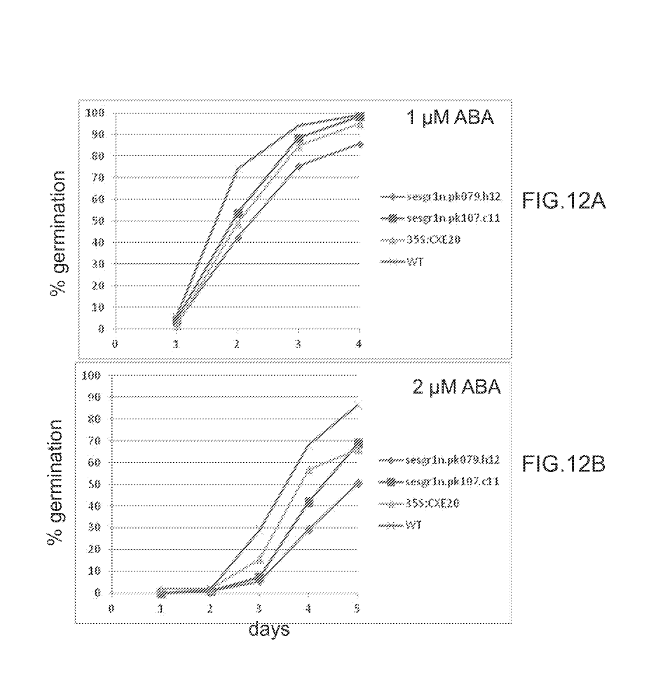

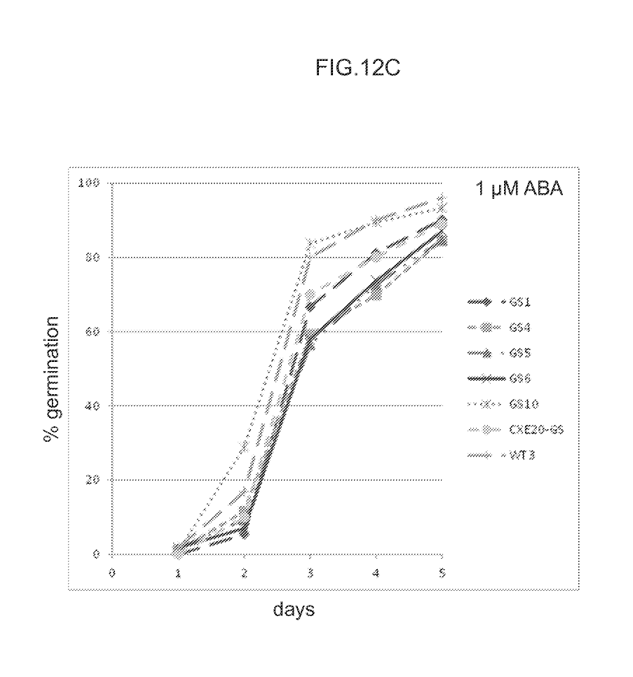

At 1 μM ABA, wild-type col-0 plants had >90% of germination rate at Day 5. The transgenic line with AtDTP4 construct showed <90% germination on Day 5, as shown in FIG. 12A. The line with a construct expressing the DTP4 homologs sesgr1n.pk079.h12 (SEQ ID NO:47) showed about 70% germination, and that expressing the DTP4 homolog sesgr1n.pk107.c11 (SEQ ID NO:45) showed about 80% germination on day 3.

At 2 μM ABA, wild-type col-0 plants had >90% of germination rate at Day 5. The transgenic line with AtDTP4 construct showed <70% germination on Day 5, as shown in FIG. 12B. The line with a construct expressing the DTP4 homolog sesgr1n.pk079.h12 (SEQ ID NO:47) showed <50% germination, and that expressing the DTP4 homolog sesgr1n.pk107.c11 (SEQ ID NO:45) showed <70% germination on day 5.

FIG. 12C shows the percentage germination assay for transgenic Arabidopsis plants expressing some of the other DTP4 homologs that were tested, given in Table 9 and Table 10, respectively.

### Example 28

### ABA Sensitivity Assay

### Green Cotyledon Assay with DTP4 Polypeptides in Arabidopsis

The DTP4 polypeptides given in Table 8 and Table 9 were tested for their ability to confer ABA hypersensitivity by a percentage green cotyledon assay as described below.

The cDNA protein-coding region for each of these homologs was synthesized and cloned into the transformation vector. The homologs were tested for ABA hypersensitivity on 2 μM ABA containing medium.

### Assay Conditions:

Seeds were surface sterilized and stratified for 96 hrs. About 100 seeds were inoculated in one plate and stratified for 96 hrs, then cultured in a growth chamber programmed for 16 h of light at 22° C. temperature and 50% relative humidity. Seedlings with green cotyledons were scored.

### Observations and Results:

Seedlings with green and expanded cotyledons ware scored in ½ MS media and 2 μM ABA on Day 5-7. Seeds were counted manually using a magnifying lens. The data was analyzed as percentage seedlings with green cotyledons to the total number of seeds that were inoculated. Wild-type col-0 plants normally have ˜60-70% of seedlings with green cotyledons. The line with pBC-yellow-At5g62180 (AtDTP4 expression construct described and some homologs had scores<45% in this assay.

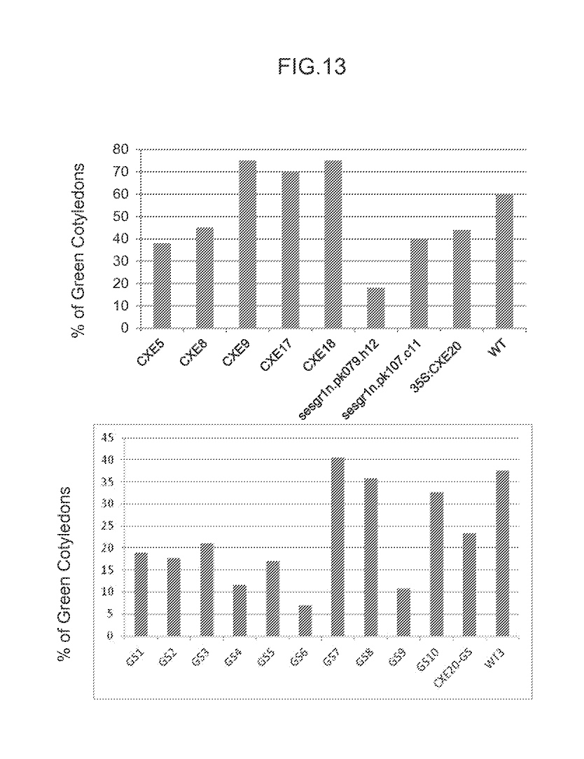

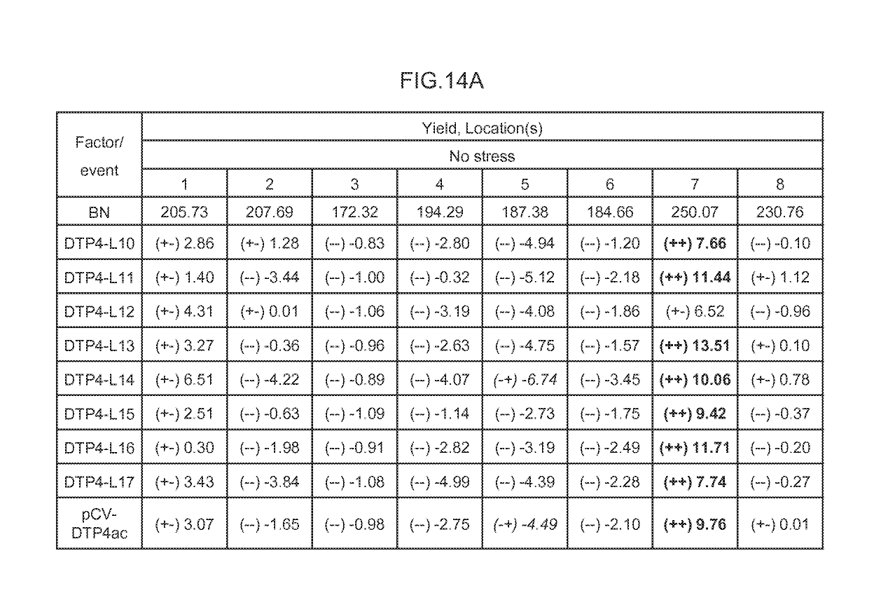

FIG. 13 and FIG. 12C show the green cotyledon assay and percentage germination assay results respectively (Example 27) for transgenic Arabidopsis plants expressing some of the other DTP4 polypeptides that were tested, given in Table 8 and Table 9, respectively.

TABLE 8
ABA Sensitivity Assay with DTP4 Polypeptides

| | | | Percentage | Cotyledon |
|---|---|---|---|---|
| | SEQ | | germination | greening |
| Clone ID | ID NO | Type | assay | assay |
| ATDTP4 | 18 | Type II | Positive | Positive |
| sesgr1n.pk117.j17 | 39 | Type II | Neutral | Neutral |
| sesgr1n.pk062.h8 | 43 | Type II | Neutral | Neutral |
| sesgr1n.pk107.c11 | 45 | Type II | Positive | Positive |
| sesgr1n.pk079.h12 | 47 | Type II | Positive | Positive |
| arttr1n.pk125.i16 | 49 | Type II | neutral | neutral |
| arttr1n.pk029.e11 | 51 | Type II | neutral | neutral |
| arttr1n.pk120.m9 | 55 | Type II | neutral | neutral |
| hengr1n.pk028.m4 | 59 | Type II | neutral | neutral |
| icegr1n.pk156.e13 | 61 | Type II | neutral | neutral |
| pepgr1n.pk190.l24 | 95 | Type II | neutral | neutral |
| pepgr1n.pk082.c4 | 97 | Type II | neutral | neutral |
| hengr1n.pk014.d12 | 101 | Type II | neutral | neutral |
| ecalgr1n.pk137.m22 | 103 | Type II | neutral | neutral |
| ahgr1c.pk108.k16 | 107 | Type II | neutral | neutral |
| arttr1n.pk193.a17 | 111 | Type II | neutral | neutral |
| arttr1n.pk090.l10 | 113 | Type II | neutral | neutral |
| At-cxe5 | 627 | Type III | neutral | Positive |
| At-cxe8 | 64 | Type II | neutral | Positive |
| At-cxe9 | 65 | Type II | neutral | Negative |
| At-cxe17 | 628 | Type VI | neutral | negative |
| At-cxe18 | 66 | Type IV | neutral | negative |

TABLE 9
ABA Sensitivity Assay with DTP4 Polypeptides

| | | ID in | | | |
|---|---|---|---|---|---|
| | | graph | | | |
| | | in | | | |
| | | FIG. | | | |
| | | 12C | | | |
| | SEQ | and | | Percentage | Cotyledon |
| | ID | FIG. | | germination | greening |
| Clone ID | NO | 13 | Type | assay | assay |
| Thhalv10005595m | 117 | GS3 | Type II | Neutral | Positive |
| Bn-Bo | 119 | GS6 | Type II | Positive | Positive |
| B-ole-someBnap | 121 | GS8 | Type II | Neutral | Neutral |
| B-napus2-1 | 123 | GS9 | Type II | Neutral | Positive |
| D7MLB3_Al | 127 | GS1 | Type II | Positive | Positive |
| R0I9H0_Cr | 129 | GS2 | Type II | Neutral | Positive |
| R0EXR3_Cr | 130 | GS4 | Type II | Positive | Positive |
| M4F4A4_Bp | 131 | GS5 | Type II | Positive | Positive |
| M4EKG1_Bp | 132 | GS7 | Type II | Neutral | Neutral |
| GSVIVT01010672001 | 135 | GS10 | Type II | Neutral | Neutral |

### Example 29A

### ABA Sensitivity Assay

### Root Architecture Assay in Arabidopsis

To test transgenic plants for alteration in root architecture in response to ABA, the root architecture assay is done as described in this example.

Seeds are sterilized using 50% household bleach 0.01% Triton X-100 solution and on petri plates containing the following medium: 0.5×N-Free Hoagland's, 8 mM KNO₃, 1% sucrose, 1 mM MES and 1% PHYTAGEL™, supplemented with 0.1 μM ABA, at a density of 4 seeds/plate. Typically 10 plates are placed in a rack. Plates are kept for three days at 4° C. to stratify seeds and then held vertically for 12 days at 22° C. light and 20° C. dark. Photoperiod is 16 h; 8 h dark, average light intensity is ˜180 μmol/m²/s. Racks (typically holding 10 plates each) are rotated every alternate day within each shelf. At day 12, plates are evaluated for seedling status, whole plate scan are taken, and analyzed for root area.

These seedlings grown on vertical plates are analyzed for root growth with the software WINRHIZO® (Regent Instruments Inc), an image analysis system specifically designed for root measurement. WINRHIZO® uses the contrast in pixels to distinguish the light root from the darker background. To identify the maximum amount of roots without picking up background, the pixel classification is kept at 150-170 and the filter feature is used to remove objects that have a length/width ratio less than 10.0. The area on the plates analyzed is from the edge of the plant's leaves to about 1 cm from the bottom of the plate. The exact same WINRHIZO® settings and area of analysis is used to analyze all plates within a batch. The total root length score given by WINRHIZO® for a plate is divided by the number of plants that have germinated and have grown halfway down the plate. Eight plates for every line are grown and their scores are averaged. This average is then compared to the average of eight plates containing wild type seeds that have been grown at the same time.

Thirty seedlings from transgenic are compared to same number in control and probability value was generated. Transgenics with probability value (p-value) equal to and or more than E-03 is considered is validated in RA assay.

### Example 29B

### Root Architecture Assay for Transgenic AT-DTP4 Arabidopsis Plants

The Arabidopsis DTP4 polypeptide gene (At5g62180; SEQ ID NO:16; NCBI GI No. 30697645) was tested for its ability to confer altered ABA sensitivity or in the following manner.

T3 seeds from seven single insertion events (named E3, E4, E5, E6, E7, E8 and E9) from transgenic Arabidopsis line with AT-DTP4 protein, containing the 35S promoter::At expression construct pBC-yellow-At5g62180, generated as described in Example 6, were tested for alteration of root architecture due to presence of ABA in the media, as described in Example 27A.

Non-transformed Columbia seeds grown in the same conditions and at the same time of the single insertion events served as a control. Single line event and control seeds were subjected to the Root Architecture Assay, to test ABA sensitivity, following the procedure described in Example 29A.

Eight plates having 32 seedlings were scanned, and the pixel values obtained for each of the 32 roots of each event was compared with the pixel values obtained for the control.

T-test analysis was performed to show that the AT-DTP4 transgenic plants have better root growth under 0.1 μM ABA, indicating altered ABA sensitivity as compared to the wt plants.

The p-value for different events, done as 2 different experiments on 2 different days, is given in Table 10. The ones with probability value (p-value) equal to and or more than E-03 are shown in bold.

TABLE 10
P-values for RA Assay with AT-DTP4 Transgenic Plants

| Events | ttest (experiment 1) | ttest (experiment 2) |
|---|---|---|
| E3 | 5.77E−01 | 1.29E−01 |
| E4 | 5.14E−04 | 4.69E−02 |
| E5 | 6.36E−01 | 8.3E−01 |
| E6 | 3.43E−07 | 1.11E−07 |
| E7 | 2.08E−02 | 1.21E−01 |
| E8 | 3.92E−04 | 3.12E−03 |
| E9 | 8.22E−07 | 6.27E−05 |

### Example 30

### Detection of DTP4 Protein in Transgenic Maize Leaves by Mass Spectrometry

The transgenic maize events from the two constructs used in the field yield trials described in Example 19 were regrown in a growth chamber until stage V5 to provide leaf samples for detection of DTP4 protein by mass spectrometry. Leaves were excised and ground in liquid nitrogen, and then the frozen powder was lyophilized. The protein from 10 mg of lyophilized leaf powder per sample was extracted and subjected to analysis by mass spectrometry. AT-DTP4 protein was detected in all 8 events of the pCV-DTP4ac construct.

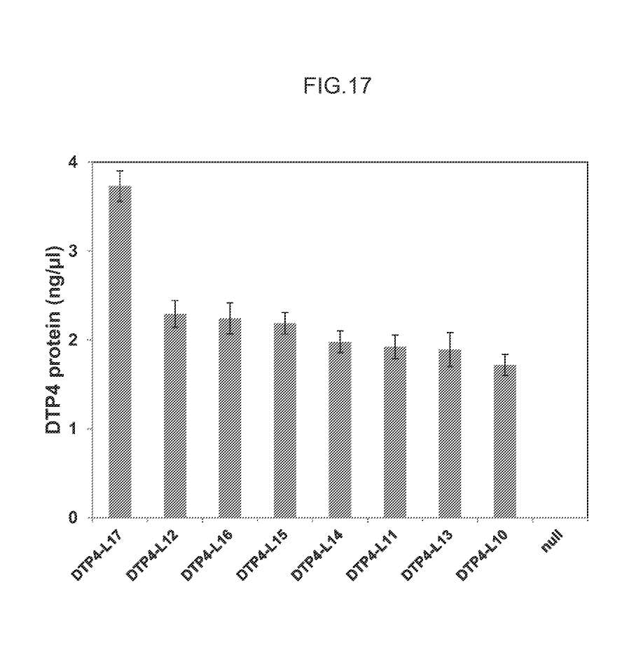

Field grown transgenic events for construct pCV-DTP4ac were also used for DTP4 protein detection by the same mass spec method (FIG. 17). The DTP4 protein was detected in V9 leaves of all transgenic events, but not in leaves of null plants. The greatest amount of DTP4 protein in the field grown plants was detected in event DTP4-L17, as was observed with the data for growth chamber grow plants.

### Example 31

### Tiller Number Assay with Transgenic Maize Plants Overexpressing AT-DTP4ac Tiller Production Under Field Conditions

The AT-DTP4 (pCV-DTP4ac) was introduced into a transformable maize line derived from an elite maize inbred line.

Six transgenic events were field tested at 2 locations A (Flowering stress) and B (Well-watered) in 2014. The trials were field physiological frame work. At the location A, mild drought conditions were imposed during flowering. The “B” location was well-watered. Tiller number data were collected in all locations, with 4 replicates per location. Tiller number per plant was counted for 20 plants in the middle of plot.

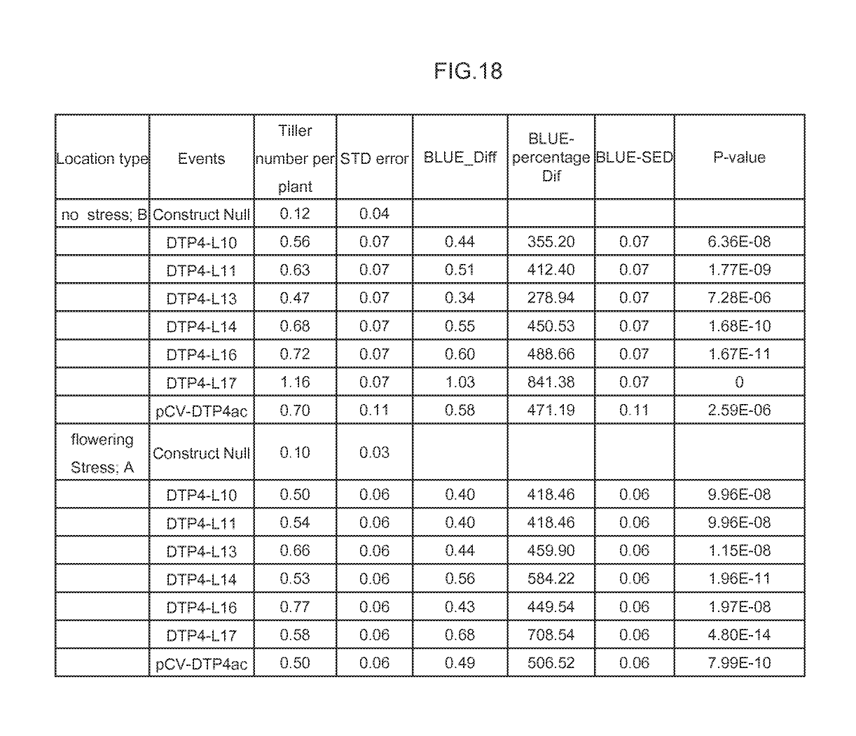

Tiller number (tiller number per plant) for the 6 transgenic events is shown in FIG. 18. Tiller number per plant of transgenic plants was significantly greater than construct null (CN).

### Example 32

### ABA Sensitivity Assay

### Root and Shoot Growth Assay with AT-DTP4ac Polypeptide in Maize

As described in Examples 5, 7, and 25, overexpressing DTP4 in Arabidopsis resulted in increased sensitivity to ABA. To determine whether transgenic maize plants overexpressing AT-DTP4 (SEQ ID NO:18) were also ABA hypersensitive, a maize ABA assay was performed with transgenic events and corresponding event nulls of construct pCV-DTP4ac. Maize seeds were germinated in paper towel rolls for 4 days in water, and then either no ABA or 10 μM ABA treatments were applied for 7 additional days. Root and shoot growth was measured before and after the ABA treatment, and differences were recorded. A positive control event from another construct known to give ABA hypersensitivity was included. Six replications were done, with 5 seeds per germination roll.

### Materials and Methods

An experiment with the current protocol was completed in 11 days, starting with germination of seeds in water (0 DAP). After four days germination, five seeds of an entry have initial root and shoot measurements were recorded and were then transferred to an individual germination roll that has been ascribed with a 10 μM or 0 μM ABA treatment (0 DAT). Following an additional 7 days in the growth chamber, final root and shoot measurements were recorded for each roll (7 DAT).

Traits were averaged over the five plants in a germination roll. Root growth and shoot growth traits were calculated as the difference of the final and initial measurements. Initial measurements were also analyzed to determine if differences were present prior to treatment. Comparisons were conducted between treatments and entries, on the event and construct level using a spatial adjustment. The experimental design was a multi-time split plot with replications sometimes conducted over several days.

Results:

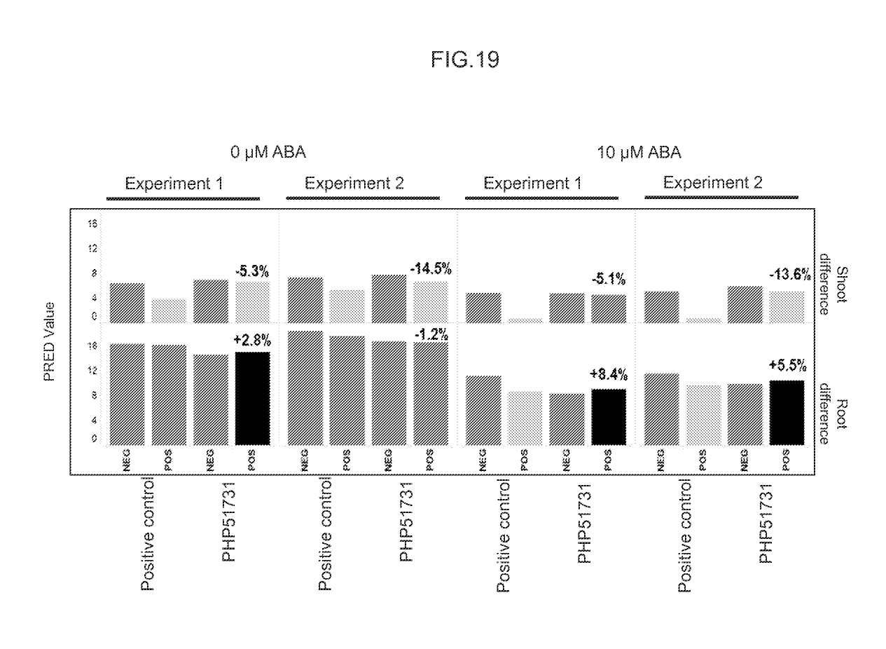

Construct level results from 2 different experiments was done on two different days, results are shown in FIG. 19.

The positive control showed significant decreases in shoot and root growth in the 10 μM ABA treatment, as expected for an ABA hypersensitive control. In contrast, four AT-DTP4ac transgenic events had significantly increased root growth, and no events had significantly decreased shoot growth, suggesting decreased sensitivity to ABA. Thus, overexpressing AT-DTP4 in both Arabidopsis and maize altered ABA sensitivity.

### Example 33

### Triple Stress Assay with Transgenic Maize Plants Overexpressing AT-DTP4ac

The triple stress assay was used to test AT-DTP4ac and other AT-DTP4 homologs for their ability to confer stress resistance following a drought, light and heat stress combination.

### Material and Methods

Maize plants were grown to the V4 stage in a growth chamber under conditions of 27° C. daytime/15° C. nighttime temperatures, 15 hour photoperiod, 60% relative humidity and 800 μmol m⁻¹ sec⁻¹ light intensity (Table 11). During this period plants were fertigated to maintain well-watered conditions. After this 21 day period, initial plant measurements (0 days after treatment, or DAT) were recorded prior to “triple stress”, including volumetric soil water content, hyperspectral imaging, and chlorophyll fluorescence. The triple stress was initiated by increasing temperatures to 38° C. daytime/27° C. nighttime, increasing the light intensity 1300 μmol m⁻¹ sec⁻¹, and water was withheld. Measurements were again collected at 3 and 6 days after treatment. At the 6 DAT measurements, plant biomass was destructively harvested for fresh and dry weights. Significant differences were determined for traits at the event and construct level for 12 replicates.

TABLE 11
Experimental Procedure for the Triple Stress Assay

| Date | Event |
|---|---|
| 1 week or less prior to | Pot filling |
| planting | |
| 0 DAP | Planting, Start “Normal” Conditions |
| 7 DAP | Thinning to 1 plant pot⁻¹ |
| 0 DAT or 20 DAP | Initial measurements, Occurs as plants are |
| | under “Normal” Conditions |
| 21 DAP | Growth Chamber Program switches to |
| | “Triple Stress” Program |
| 3 DAT or 24 DAP | Second set of measurements. |
| 6 DAT or 27 DAP | Final set of measurements including destructive |
| | harvest. |

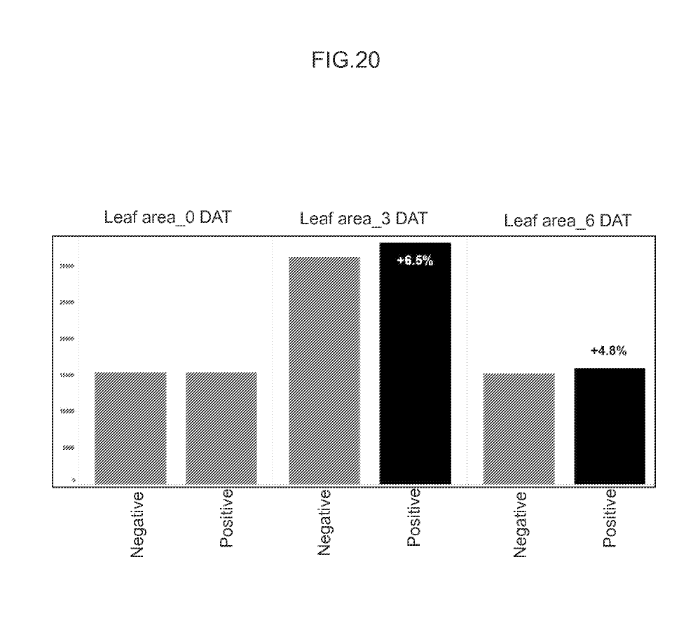

Results: During triple stress, plants with pCV-DTP4ac had greater leaf area compared to null as measured in pixel area with a hyperspectral camera (FIG. 20). Significant differences were not observed in biomass measurements, soil water content or chlorophyll fluorescence parameters.

FIG. 20 shows construct level response of plants with pCV-DTP4ac (UBI:AT-DTP4) for leaf area during triple stress. Significant differences are presented at a P<0.1, with black bars indicating significantly positive construct level response, dark grey bars indicate a comparison that is not significantly different. Numbers indicate the percent difference relative to construct null.

### Example 34

### Osmotic Stress Assay with Transgenic Maize Plants Overexpressing AT-DTP4ac

An osmotic stress assay was used to test the ability of DTP4 polypeptides to confer osmotic stress resistance in transgenic maize plants overexpressing DTP4 polypeptides.

These experiments are a variation of the osmotic stress assay described in Example 25.

Material and Methods:

All experiments were conducted in one Percival growth chamber that is maintained under completely darkened conditions at 25 degrees C., with a relative humidity of 95%. For each experiment, one construct with all available events (transgenics and event nulls) were tested in Nunc Bioassay Plates (245×245×25 mm, approximately 225 ml volume).

Two treatments were done: control and quad osmotic stress (70% concentration; ψ_{w}=−1.0 MPa)

Each event (transgenic, event null) per treatment contained six replicates.

### Media Preparation:

Quad Stress (70%) media:
MS Salt—1.1 g/L
MES Hydrate-0.3905 g/L
PEG 8000—70 g/L
Mannitol—15.94 g/L
Sorbitol—15.94 g/L
NaCl—2.557 g/L
Adjust media to 5.70 with 1 M KOH
Phytagel—8 g/L
Control Media:
MS Salt—1.1 g/L
MES Hydrate—0.3905 g/L
Adjust media to 5.70 with 1 M KOH
Phytagel—8 g/L

Results: Seed germination data were collected at 24, 32, 48, 56, 72, and 96 hours after plating. The water potentials of the control and quad stress (70% concentration) media were measured via a vapor pressure osmometer at the end of each experiment

Significant inhibition was found in seed germination in response to quad stress, relative to control at 48-96 h. All available events (total of eight) of PHP51731 were tested twice with reproducible results. AT-DTP4ac transgenic events consistently demonstrated significantly reduced sensitivity to quad stress, relative to null.

During two experiments, seven of eight transgenic events exhibited significantly reduced germination sensitivity to quad stress, relative to comparable nulls.

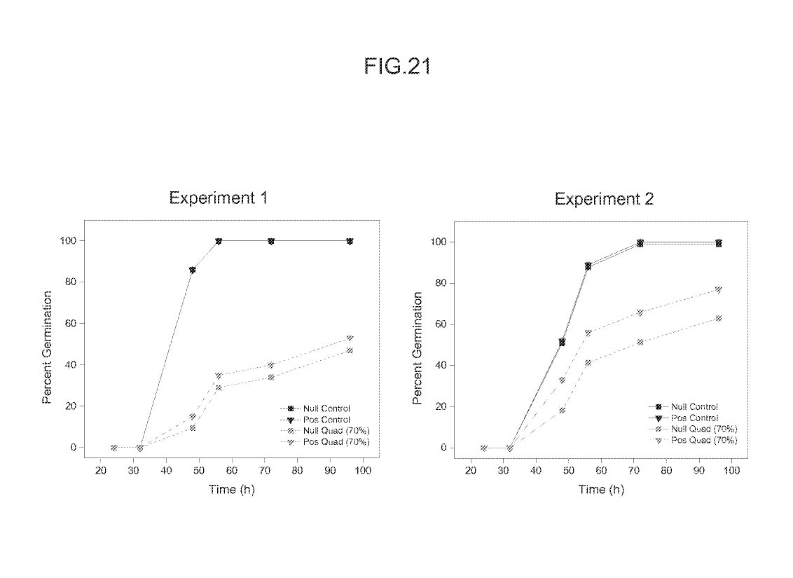

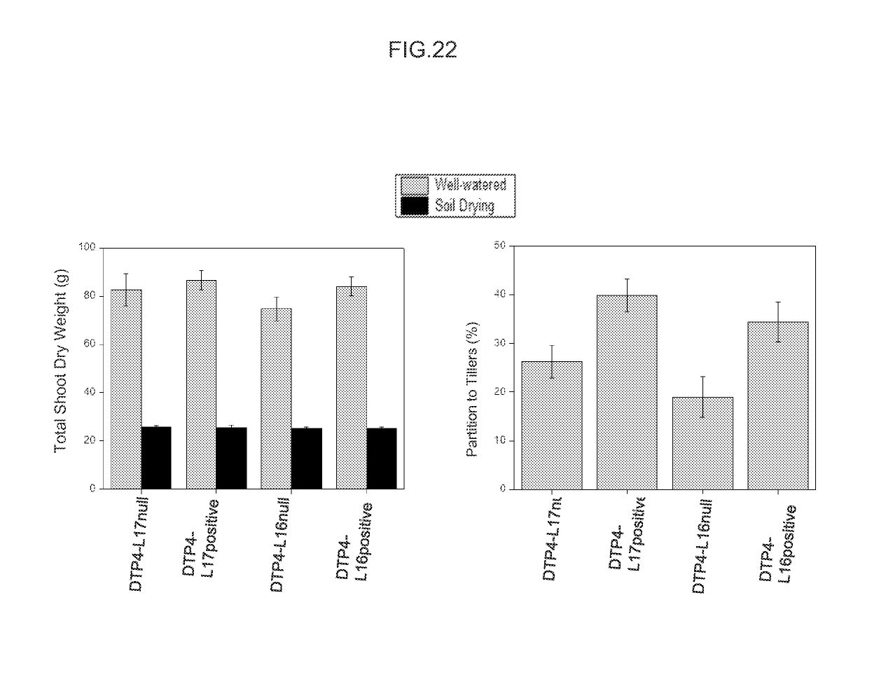

Results are shown in Table 12 and FIG. 21.

TABLE 12
Osmotic Stress Assay With AT-DTP4 Overexpressing Maize Plants

| Construct | Control | Quad Stress |
|---|---|---|
| pCV-DTP4ac | Neutral | Positive (p < 0.05) |

### Example 35

### Tall Clear Tube Root Growth Assay with Transgenic Maize Plants Overexpressing AT-DTP4ac to Evaluate Root and Shoot Development

This assay was developed and used to evaluate root growth developmental plasticity in transgenic maize plants overexpressing DTP4 polypeptides in response to well-watered and soil drying conditions.

### Material and Methods:

The experiments were performed in greenhouse. Maize seeds were imbibed on germination paper that was pre-soaked in water for a 48 h period. Uniform maize seedlings (with root lengths between 10-22 mm) were transplanted into clear acrylic tubes (1.5 meters in length, approximately 38 L volume) containing a 3:1 Dynamix to sand media. The soil media was supplemented with Scott's Osmocote Plus (15-9-12) to provide a slow release of nutrients throughout the course of each experiment. For each experiment, one construct with two selected events (transgenic and event null) were tested. Two treatments were done: well watered and drought. The drought cycle was induced between V3-V4 growth stages, for three weeks. Each event (transgenic, event null) per treatment contained 6 replicates.

Measurements were done to monitor lateral growth development with depth and time, a total of 40 root windows were permanently installed by a custom fabrication vendor, according to design specifications. To delineate the differing depths, each root window has been systematically assigned a number designation. Lateral root growth is monitored on a weekly basis following water withholding by taking a series of photographs of each root window at the different depth increments with a digital camera with an attached polarizing filter. To ensure that standardized photographs were taken, the camera is installed on a customized designed and fabricated acrylic jig. All images were sent for automated quantitative analysis.

Soil water content measurements: The apparent dielectric constant of the uppermost 100 cm of soil was quantified bi-weekly using a soil moisture probe in all plants during the drought period to better interpret as well as compare the timing and pattern of root development both within as well as between genotypes. Plant growth quantification: plant height and leaf number data were collected bi-weekly, during the drought period. The harvest measurements done were for shoot fresh weight, shoot dry weight, total leaf area, primary root length; data were collected for all plants.

TABLE 13
Tall Clear Tube Root Assay With AT-DTP4 Overexpressing Maize Plants

| Construct | Promoter | Events | Treatment | Major Difference |
|---|---|---|---|---|
| pCV- | ZM-UBI | DTP4-L17 (TG, | WW, Soil | TG positive |
| DTP4ac | | event null) | drying | produced more |
| | | | | tillers, under WW |
| | | | | conditions, |
| | | | | relative to null |
| | | DTP4-L16 (TG, | WW, Soil | TG positive |
| | | event null) | drying | produced more |
| | | | | tillers, under WW |
| | | | | conditions, |
| | | | | relative to null |

### Example 36

### Expression of AT-DTP4 Fusion Protein in E. coli

### Protein Purification and Esterase Activity Assays

The pET28a expression vector was used to express AT-DTP4 fusion protein containing 20 additional N-terminal amino acids, including a 6 histidine tag. The amino acid sequence of the fusion protein is presented as SEQ ID NO:629. E. coli cultures were grown at 37° C. in 2×YT media to an OD₆₀₀ₙₘ of 0.6. Transgene expression was then induced with 0.5 mM IPTG and the culture was grown an additional 20 hours at 20° C. The fusion protein was purified from E. coli extracts using cobalt affinity chromatography, and a high degree of purity was achieved. Aliquots of the purified protein were stored frozen at −80° C. in 10% glycerol. Aliquots were then thawed and dialyzed against 50 mM Tris-HCl pH 8, prior to performing esterase activity assays with p-nitrophenyl acetate as substrate.

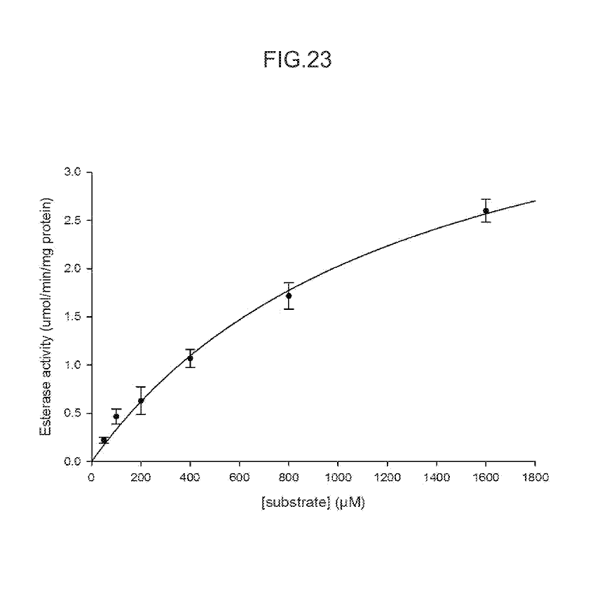

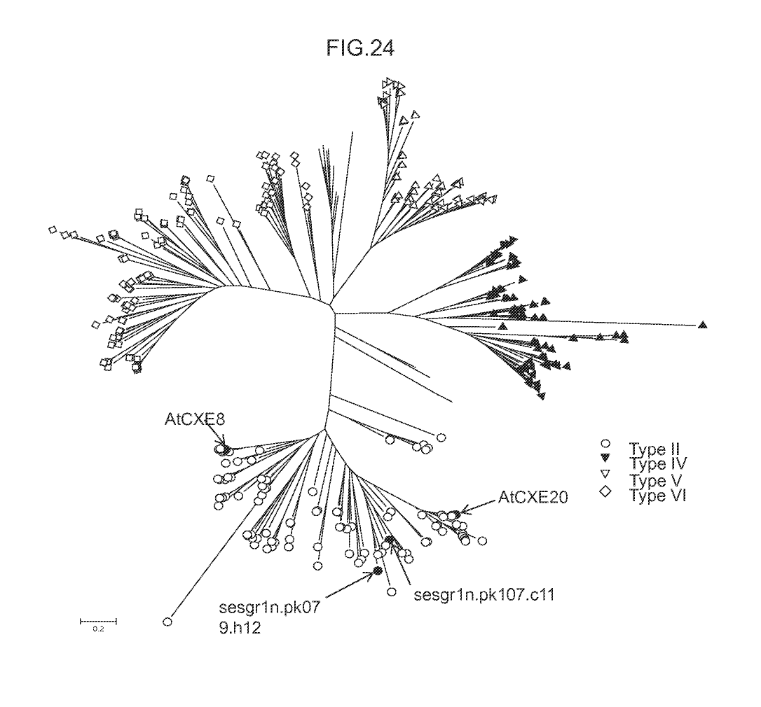

Esterase activity with this substrate was monitored by observing an increase in absorbance at a wavelength of 405 nm, because the p-nitrophenol product absorbs at 405 nm. The activity assays were done with 1 μg of protein in 50 mM Tris-HCl, pH 8, with an assay volume of 200 μl, using 96 well flat bottom microtiter plates. Control reactions without enzyme were done and rates were subtracted from the plus enzyme reaction rates to correct for autohydrolysis of substrate. The purified AT-DTP4 protein had obvious esterase activity with p-nitrophenyl acetate as substrate (FIG. 23). Dialyzed protein was quantitated by absorbance at 280 nm, using a value of 1 OD (280 nm)=0.92 mg/ml.

### Example 37

### Traits Observed in Field Plots in Transgenic Maize Plants Overexpressing AT-DTP4 Polypeptide

Field plots were observed in well watered conditions with transgenic maize plants transformed with pCV-DTP4ac. A randomized complete block design was used with 2 row plots and 4 field replications. Five consecutive evenly spaced plants in each row were tagged for observation, for a total of 10 plants per plot. In some plots, fewer than 10 plants were used for observations. For one trait, tiller number at V12, all the plants of a plot were used, except for the end plant on each side of each row. For another trait, stalk diameter, only 3 events were measured. Descriptions of the traits measured, a summary of the results are presented in Table 14, and detailed results are presented in Table 15. At the construct level, small but statistically significant differences from nulls were observed for several traits, including decreases in plant height at V12, leaf number at V9, and growth rate from V9 to V12. Increased tiller number was observed at V12. Pollen shed was about half a day later, and because silks emerged before pollen shed in these well watered conditions, the ASI was negative and larger due to the delayed shed.

TABLE 14
Trait Description and Result Summary in Field Plots

| | | Significant |
|---|---|---|
| | | difference |
| | | from null |
| | | (construct |
| Trait | Description | level) |
| PLTHT.V9 | Plant height (cm) at V9, to top leaf collar | No |
| LFN.V9 | Leaf number with visible collar at V9 | Yes; |
| | | decreased |
| TILN.V12 | Tiller number at V12 | Yes; |
| | | increased |
| PLTHT.V12 | Plant height (cm) at V12, to top leaf | Yes; |
| | collar | decreased |
| LFN.V12 | Leaf number with visible collar at V12 | No |
| PLTHT.V17 | Plant height (cm) at V17 to V18, to top | No |
| | leaf collar | |
| GR.V9V12 | Growth rate (cm/day) from June 23 to | Yes; |
| | July 3 (V9 to V12). | decreased |
| GR.V12V17 | Growth rate (cm/day) from June 23 to | No |
| | July 3 (V9 to V12). | |
| SHED | First pollen shed for 50% of plants in | Yes; later |
| | plot, days after planting | |
| SILK | First silk emergence for 50% of plants in | No |
| | plot, days after planting | |
| ASI | Anthesis to silk interval (silk date - shed | Yes; larger |
| | date) | |
| PLTHT.R3 | Plant height (cm), final, to bottom of | No |
| | tassel | |
| EARHT | Ear height (cm) | No |
| LFN.R3 | Leaf number, final | No |
| EARLP | Ear leaf position | No |
| STKD | Stalk diameter (cm), perpendicular to | No |
| | groove, mid internode below ear | |
| STAGRN.ER4 | Stay green, lowest leaf that is >50% | No |
| | green, ear leaf position = 0, Very early | |
| | R4. | |
| STAGRN.R4 | Stay green, lowest leaf that is >50% | No |
| | green, ear leaf position = 0. R4. | |

TABLE 15
Traits Observed In Field Plots

| | | | | | significantly |
|---|---|---|---|---|---|
| | | | | | different |
| | | | | | from |
| | | | | | null (** p |
| | | | | | value <0.05; |
| | | mean | Difference | | * p Value |
| Trait | Event | value | from null | p Value | <0.1) |
| PLTHT.V9 | DTP4-L10 | 50.07 | −1.51 | 0.182 | |
| PLTHT.V9 | DTP4-L11 | 50.41 | −1.17 | 0.308 | |
| PLTHT.V9 | DTP4-L12 | 50.69 | −0.90 | 0.449 | |
| PLTHT.V9 | DTP4-L13 | 51.69 | 0.10 | 0.928 | |
| PLTHT.V9 | DTP4-L14 | 50.27 | −1.32 | 0.234 | |
| PLTHT.V9 | DTP4-L15 | 50.91 | −0.68 | 0.567 | |
| PLTHT.V9 | DTP4-L16 | 51.94 | 0.36 | 0.747 | |
| PLTHT.V9 | DTP4-L17 | 50.18 | −1.40 | 0.226 | |
| PLTHT.V9 | Construct | 50.77 | −0.82 | 0.402 | |
| PLTHT.V9 | null | 51.59 | 0.00 | | |
| LFN.V9 | DTP4-L10 | 8.89 | −0.11 | 0.012 | ** |
| LFN.V9 | DTP4-L11 | 8.82 | −0.19 | 0.000 | ** |
| LFN.V9 | DTP4-L12 | 8.94 | −0.06 | 0.163 | |
| LFN.V9 | DTP4-L13 | 8.97 | −0.04 | 0.393 | |
| LFN.V9 | DTP4-L14 | 8.90 | −0.10 | 0.016 | ** |
| LFN.V9 | DTP4-L15 | 8.92 | −0.08 | 0.083 | * |
| LFN.V9 | DTP4-L16 | 8.97 | −0.04 | 0.409 | |
| LFN.V9 | DTP4-L17 | 8.88 | −0.12 | 0.007 | ** |
| LFN.V9 | Construct | 8.91 | −0.09 | 0.018 | ** |
| LFN.V9 | null | 9.00 | 0.00 | | |
| TILN.V12 | DTP4-L10 | 0.07 | 0.06 | 0.035 | ** |
| TILN.V12 | DTP4-L11 | 0.06 | 0.06 | 0.063 | * |
| TILN.V12 | DTP4-L12 | 0.07 | 0.06 | 0.051 | * |
| TILN.V12 | DTP4-L13 | 0.06 | 0.05 | 0.115 | |
| TILN.V12 | DTP4-L14 | 0.09 | 0.08 | 0.013 | ** |
| TILN.V12 | DTP4-L15 | 0.07 | 0.06 | 0.039 | ** |
| TILN.V12 | DTP4-L16 | 0.09 | 0.08 | 0.009 | ** |
| TILN.V12 | DTP4-L17 | 0.14 | 0.13 | 0.000 | ** |
| TILN.V12 | Construct | 0.08 | 0.07 | 0.006 | ** |
| TILN.V12 | null | 0.01 | 0.00 | | |
| PLTHT.V12 | DTP4-L10 | 98.99 | −3.28 | 0.033 | ** |
| PLTHT.V12 | DTP4-L11 | 99.11 | −3.16 | 0.042 | ** |
| PLTHT.V12 | DTP4-L12 | 98.68 | −3.59 | 0.024 | ** |
| PLTHT.V12 | DTP4-L13 | 99.60 | −2.67 | 0.080 | * |
| PLTHT.V12 | DTP4-L14 | 98.72 | −3.55 | 0.020 | ** |
| PLTHT.V12 | DTP4-L15 | 99.19 | −3.08 | 0.050 | * |
| PLTHT.V12 | DTP4-L16 | 99.86 | −2.41 | 0.112 | |
| PLTHT.V12 | DTP4-L17 | 99.01 | −3.25 | 0.036 | ** |
| PLTHT.V12 | Construct | 99.14 | −3.12 | 0.026 | ** |
| PLTHT.V12 | null | 102.27 | 0.00 | | |
| LFN.V12 | DTP4-L10 | 11.75 | −0.13 | 0.162 | |
| LFN.V12 | DTP4-L11 | 11.73 | −0.15 | 0.111 | |
| LFN.V12 | DTP4-L12 | 11.73 | −0.14 | 0.120 | |
| LFN.V12 | DTP4-L13 | 11.77 | −0.11 | 0.232 | |
| LFN.V12 | DTP4-L14 | 11.76 | −0.11 | 0.214 | |
| LFN.V12 | DTP4-L15 | 11.74 | −0.13 | 0.147 | |
| LFN.V12 | DTP4-L16 | 11.78 | −0.10 | 0.268 | |
| LFN.V12 | DTP4-L17 | 11.76 | −0.12 | 0.198 | |
| LFN.V12 | Construct | 11.75 | −0.12 | 0.145 | |
| LFN.V12 | null | 11.88 | 0.00 | | |
| PLTHT.V17 | DTP4-L10 | 195.38 | −1.80 | 0.400 | |
| PLTHT.V17 | DTP4-L11 | 195.26 | −1.92 | 0.376 | |
| PLTHT.V17 | DTP4-L12 | 194.76 | −2.42 | 0.275 | |
| PLTHT.V17 | DTP4-L13 | 196.13 | −1.05 | 0.621 | |
| PLTHT.V17 | DTP4-L14 | 194.78 | −2.40 | 0.257 | |
| PLTHT.V17 | DTP4-L15 | 195.97 | −1.21 | 0.582 | |
| PLTHT.V17 | DTP4-L16 | 196.36 | −0.82 | 0.699 | |
| PLTHT.V17 | DTP4-L17 | 194.82 | −2.36 | 0.278 | |
| PLTHT.V17 | Construct | 195.43 | −1.75 | 0.367 | |
| PLTHT.V17 | null | 197.18 | 0.00 | | |
| GR.V9V12 | DTP4-L10 | 4.84 | −0.22 | 0.006 | ** |
| GR.V9V12 | DTP4-L11 | 4.84 | −0.22 | 0.006 | ** |
| GR.V9V12 | DTP4-L12 | 4.84 | −0.22 | 0.006 | ** |
| GR.V9V12 | DTP4-L13 | 4.84 | −0.22 | 0.006 | ** |
| GR.V9V12 | DTP4-L14 | 4.84 | −0.22 | 0.006 | ** |
| GR.V9V12 | DTP4-L15 | 4.84 | −0.22 | 0.006 | ** |
| GR.V9V12 | DTP4-L16 | 4.84 | −0.22 | 0.006 | ** |
| GR.V9V12 | DTP4-L17 | 4.84 | −0.22 | 0.006 | ** |
| GR.V9V12 | Construct | 4.84 | −0.22 | 0.006 | ** |
| GR.V9V12 | null | 5.06 | 0.00 | | |
| GR.V12V17 | DTP4-L10 | 8.76 | 0.12 | 0.170 | |
| GR.V12V17 | DTP4-L11 | 8.75 | 0.12 | 0.183 | |
| GR.V12V17 | DTP4-L12 | 8.75 | 0.12 | 0.188 | |
| GR.V12V17 | DTP4-L13 | 8.76 | 0.12 | 0.167 | |
| GR.V12V17 | DTP4-L14 | 8.75 | 0.12 | 0.184 | |
| GR.V12V17 | DTP4-L15 | 8.76 | 0.13 | 0.155 | |
| GR.V12V17 | DTP4-L16 | 8.76 | 0.12 | 0.170 | |
| GR.V12V17 | DTP4-L17 | 8.75 | 0.11 | 0.202 | |
| GR.V12V17 | Construct | 8.76 | 0.12 | 0.168 | |
| GR.V12V17 | null | 8.64 | 0.00 | | |
| PLTHT.R3 | DTP4-L10 | 264.02 | −0.66 | 0.703 | |
| PLTHT.R3 | DTP4-L11 | 264.02 | −0.66 | 0.703 | |
| PLTHT.R3 | DTP4-L12 | 264.02 | −0.66 | 0.703 | |
| PLTHT.R3 | DTP4-L13 | 264.02 | −0.66 | 0.703 | |
| PLTHT.R3 | DTP4-L14 | 264.02 | −0.66 | 0.703 | |
| PLTHT.R3 | DTP4-L15 | 264.02 | −0.66 | 0.703 | |
| PLTHT.R3 | DTP4-L16 | 264.02 | −0.66 | 0.703 | |
| PLTHT.R3 | DTP4-L17 | 264.02 | −0.66 | 0.703 | |
| PLTHT.R3 | Construct | 264.02 | −0.66 | 0.703 | |
| PLTHT.R3 | null | 264.68 | 0.00 | | |
| EARHT | DTP4-L10 | 105.34 | 1.98 | 0.243 | |
| EARHT | DTP4-L11 | 105.34 | 1.98 | 0.243 | |
| EARHT | DTP4-L12 | 105.34 | 1.98 | 0.243 | |
| EARHT | DTP4-L13 | 105.34 | 1.98 | 0.243 | |
| EARHT | DTP4-L14 | 105.34 | 1.98 | 0.243 | |
| EARHT | DTP4-L15 | 105.34 | 1.98 | 0.243 | |
| EARHT | DTP4-L16 | 105.34 | 1.98 | 0.243 | |
| EARHT | DTP4-L17 | 105.34 | 1.98 | 0.243 | |
| EARHT | Construct | 105.34 | 1.98 | 0.243 | |
| EARHT | null | 103.36 | 0.00 | | |
| LFN.R3 | DTP4-L10 | 18.63 | −0.24 | 0.024 | ** |
| LFN.R3 | DTP4-L11 | 18.67 | −0.20 | 0.060 | |
| LFN.R3 | DTP4-L12 | 18.79 | −0.08 | 0.460 | |
| LFN.R3 | DTP4-L13 | 18.84 | −0.03 | 0.793 | |
| LFN.R3 | DTP4-L14 | 18.83 | −0.04 | 0.655 | |
| LFN.R3 | DTP4-L15 | 18.85 | −0.02 | 0.870 | |
| LFN.R3 | DTP4-L16 | 18.83 | −0.04 | 0.722 | |
| LFN.R3 | DTP4-L17 | 18.84 | −0.03 | 0.789 | |
| LFN.R3 | Construct | 18.79 | −0.08 | 0.344 | |
| LFN.R3 | null | 18.87 | 0.00 | | |
| EARLP | DTP4-L10 | 11.89 | −0.01 | 0.927 | |
| EARLP | DTP4-L11 | 11.92 | 0.02 | 0.793 | |
| EARLP | DTP4-L12 | 11.95 | 0.06 | 0.527 | |
| EARLP | DTP4-L13 | 11.95 | 0.06 | 0.521 | |
| EARLP | DTP4-L14 | 12.01 | 0.12 | 0.192 | |
| EARLP | DTP4-L15 | 11.93 | 0.04 | 0.672 | |
| EARLP | DTP4-L16 | 11.96 | 0.07 | 0.455 | |
| EARLP | DTP4-L17 | 11.95 | 0.05 | 0.564 | |
| EARLP | Construct | 11.95 | 0.05 | 0.523 | |
| EARLP | null | 11.89 | 0.00 | | |
| Shed | DTP4-L10 | 70.37 | 0.40 | 0.215 | |
| Shed | DTP4-L11 | 70.37 | 0.40 | 0.215 | |
| Shed | DTP4-L12 | 70.46 | 0.49 | 0.126 | |
| Shed | DTP4-L13 | 70.27 | 0.30 | 0.342 | |
| Shed | DTP4-L14 | 70.46 | 0.49 | 0.126 | |
| Shed | DTP4-L15 | 70.65 | 0.68 | 0.037 | ** |
| Shed | DTP4-L16 | 70.27 | 0.30 | 0.342 | |
| Shed | DTP4-L17 | 70.65 | 0.68 | 0.037 | ** |
| Shed | Construct | 70.44 | 0.47 | 0.095 | * |
| Shed | null | 69.97 | 0.00 | | |
| Silk | DTP4-L10 | 69.55 | 0.05 | 0.877 | |
| Silk | DTP4-L11 | 69.58 | 0.08 | 0.799 | |
| Silk | DTP4-L12 | 69.61 | 0.11 | 0.723 | |
| Silk | DTP4-L13 | 69.55 | 0.05 | 0.877 | |
| Silk | DTP4-L14 | 69.61 | 0.11 | 0.723 | |
| Silk | DTP4-L15 | 69.61 | 0.11 | 0.723 | |
| Silk | DTP4-L16 | 69.55 | 0.05 | 0.877 | |
| Silk | DTP4-L17 | 69.67 | 0.17 | 0.579 | |
| Silk | Construct | 69.59 | 0.09 | 0.757 | |
| Silk | null | 69.51 | 0.00 | | |
| ASI | DTP4-L10 | −0.84 | −0.42 | 0.063 | * |
| ASI | DTP4-L11 | −0.84 | −0.42 | 0.063 | * |
| ASI | DTP4-L12 | −0.84 | −0.42 | 0.063 | * |
| ASI | DTP4-L13 | −0.84 | −0.42 | 0.063 | * |
| ASI | DTP4-L14 | −0.84 | −0.42 | 0.063 | * |
| ASI | DTP4-L15 | −0.84 | −0.42 | 0.063 | * |
| ASI | DTP4-L16 | −0.84 | −0.42 | 0.063 | * |
| ASI | DTP4-L17 | −0.84 | −0.42 | 0.063 | * |
| ASI | Construct | −0.84 | −0.42 | 0.063 | * |
| ASI | null | −0.43 | 0.00 | | |
| STKD | DTP4-L13 | 17.18 | −0.13 | 0.275 | |
| STKD | DTP4-L16 | 17.18 | −0.13 | 0.275 | |
| STKD | DTP4-L17 | 17.18 | −0.13 | 0.275 | |
| STKD | Construct | 17.18 | −0.13 | 0.275 | |
| STKD | null | 17.31 | 0.00 | | |
| STAGRN.ER4 | DTP4-L10 | −3.41 | −0.08 | 0.476 | |
| STAGRN.ER4 | DTP4-L11 | −3.41 | −0.08 | 0.476 | |
| STAGRN.ER4 | DTP4-L12 | −3.41 | −0.08 | 0.476 | |
| STAGRN.ER4 | DTP4-L13 | −3.41 | −0.08 | 0.476 | |
| STAGRN.ER4 | DTP4-L14 | −3.41 | −0.08 | 0.476 | |
| STAGRN.ER4 | DTP4-L15 | −3.41 | −0.08 | 0.476 | |
| STAGRN.ER4 | DTP4-L16 | −3.41 | −0.08 | 0.476 | |
| STAGRN.ER4 | DTP4-L17 | −3.41 | −0.08 | 0.476 | |
| STAGRN.ER4 | Construct | −3.41 | −0.08 | 0.476 | |
| STAGRN.ER4 | null | −3.32 | 0.00 | | |
| STAGRN.R4 | DTP4-L10 | −2.26 | −0.23 | 0.129 | |
| STAGRN.R4 | DTP4-L11 | −2.15 | −0.12 | 0.447 | |
| STAGRN.R4 | DTP4-L12 | −2.20 | −0.17 | 0.296 | |
| STAGRN.R4 | DTP4-L13 | −2.10 | −0.07 | 0.664 | |
| STAGRN.R4 | DTP4-L14 | −2.14 | −0.11 | 0.476 | |
| STAGRN.R4 | DTP4-L15 | −2.25 | −0.22 | 0.165 | |
| STAGRN.R4 | DTP4-L16 | −2.04 | −0.01 | 0.950 | |
| STAGRN.R4 | DTP4-L17 | −2.27 | −0.24 | 0.124 | |
| STAGRN.R4 | Construct | −2.18 | −0.15 | 0.272 | |
| STAGRN.R4 | null | −2.03 | 0.00 | | |

### Example 38

### Traits Observed in Field Pots

In addition to the field plots described in Example 37, a field pot study was also performed at a well-watered location. Growing maize plants in pots allowed the option of imposing drought stress in a well-watered location by irrigating less, because plants in pots received more water from irrigation than from rainfall, due to the small neck size of the pots and the fact that water drained quickly from pots. The pots were 10 liter volume, 7.75″×18″ square treepots. A split plot design was used, with treatment being the whole plot, event the split plot, and transgenic event and event null the split plot. So throughout the experiment, each event was adjacent to its corresponding event null. There were six pots per replication, comprising three transgenic events and the three corresponding event nulls. 30 replications in the well watered treatment and 30 replications in the drought stressed treatment were done. In each treatment, 15 of the 30 reps were harvested at R1, and the other 15 reps were harvested at R6. Descriptions of the traits measured, and a summary of the results for the pot study are presented in Table 16, and results are presented in Table 17. At the construct level in the well watered treatment, significant differences from nulls were observed for the following traits: increased tiller number at V4 and V6, reduced plant height at V10, V13, V16, and R1, reduced leaf number at V10, decreased growth rate from V6 to V10, decreased flavonols, decreased water use efficiency, decreased dry weight of the main shoot at R1, increased dry weight of tillers at R1, delayed shed and silk time, and increased vegetative dry weight at R6. At the construct level in the drought stressed treatment, significant differences from nulls were observed for the following traits: increased tillers at V4 and V6, decreased plant height at V6, V10, and V13, decreased leaf number at V10, V13, and at maturity, decreased flavonols, decreased dry weight of the main shoot at R1, increased dry weight of tillers and ear at R1, earlier silking time, decreased ASI, decreased yellow leaves (increased stay green) at 3 dates, decreased vegetative dry weight at R6, and increased dry weight of kernels (yield), ear, kernel number, and harvest index at R6. A summary is given in Table 16, and the numbers for different events are given in Table 17.

Significance of many of these traits in determining plant health, yield and biomass are well known in the art. For example, chlorophyll and flavonol measurement using Dualex instrument, measurement of other traits such as harvest index, water use efficiency, plant height, dry weight, kernel weight etc is well known in the art (Cerovic et al Physiologia Plantarum 146: 251-260. 2012; Sinclair, T. R.; Crop Sci. 38:638-643(1998), Edmeades et al (1999) Crop Sci. 39:1306-1315, Andrade et al Crop Sci. 42:1173-1179 (2002), Berke et al (1995) Crop Sci. 39:1542-1549, Garwood et al Crop Science, Vol. 10, January-February 1970).

TABLE 16
Trait Descriptions for Field Pot Study

| | | Significant | Significant |
|---|---|---|---|
| | | difference from | difference from |
| | | null in ww | null in drought |
| Trait | Trait description | (construct level) | (construct level) |
| TILN.V4 | Tiller number at V4 | Yes; increased | Yes; increased |
| PLTHT.V6 | Plant height (cm) at V6 | No | Yes; decreased |
| LFN.V6 | Leaf number with visible collar | No | No |
| | at V6 | | |
| TILN.V6 | Tiller number at V6 | Yes; increased | Yes; increased |
| PLTHT.V10 | Plant height (cm) at V10 | Yes; decreased | Yes; decreased |
| LFN.V10 | Leaf number with visible collar | Yes; decreased | Yes; decreased |
| | at V10 | | |
| PLTHT.V13 | Plant height (cm) at V13 | Yes; decreased | Yes; decreased |
| LFN.V13 | Leaf number with visible collar | Yes; decreased | No |
| | at V13 | | |
| PLTHT.V16 | Plant height (cm), about V16 | Yes; decreased | No |
| | and V15 for WW and DRT. | | |
| DUALEX.CHL | Chlorophyll by Dualex | No | No |
| | instrument, near middle of | | |
| | 11th leaf. | | |
| DUALEX.FLV | Flavonols by Dualex | Yes; decreased | Yes; decreased |
| | instrument, near middle of | | |
| | 11th leaf. | | |
| DUALEX.NBI | Nitrogen band index by Dualex | No | No |
| | instrument, near middle of | | |
| | 11th leaf. | | |
| RWC | Relative water content (%), | ND | No |
| | 10th leaf, R1 DRT pots | | |
| WU | Water use (initial pot weight − | No | ND |
| | 24 hr pot weight, WW R1) | | |
| WUE | Water use/total biomass | Yes; decreased | ND |
| PLTHT.R1 | Plant height (cm) at R1 | Yes; decreased | No |
| PLTHT.R6 | Plant height (cm) at R6 | ND | ND |
| EARHT | Ear height (cm), final | No | No |
| LFN | Leaf number, final | No | Yes; decreased |
| EARLP | Position of ear leaf (done for | No | Yes; negative |
| | R6 plants only) | | |
| DWMAIN.R1 | Dry weight of main plant at R1 | Yes; decreased | Yes; decreased |
| DWTIL.R1 | Dry weight of tillers at R1 | Yes; increased | Yes; increased |
| DWVEG.R1 | Dry weight of mainplant and | Yes; decreased | No |
| | tillers, minus ear, R1 | | |
| DWEAR.R1 | Dry weight of primary ear at | Yes; decreased | Yes, increased |
| | R1 | | |
| DWTOT.R1 | Dry weight of total plant at R1 | Yes; decreased | No |
| SHED | First pollen shed | Yes; delayed | No |
| SILK | First silk emergence | Yes, delayed | Yes; earlier |
| ASI | Silk date − shed date | No | Yes; decreased |
| GR.V6V10 | Growth rate (cm/day) from | Yes; decreased | Yes; decreased |
| | June 18 to July 1 (V6 to V10) | | |
| GR.V10V13 | Growth rate (cm/day) from | No | No |
| | July 1 to July 9 (V10 to V13) | | |
| YL.date 1 | Number of leaves >50% | ND | Yes; decreased |
| | yellow on date 1 | | |
| YL.date 2 | Number of leaves >50% | ND | Yes; decreased |
| | yellow on date 2 | | |
| YL.date 3 | Number of leaves >50% | ND | Yes; decreased |
| | yellow on date 3 | | |
| DWVEG.R6 | Dry weight (g) at R6 of all | Yes; increased | Yes; decreased |
| | plant parts except for ear | | |
| ROW | Rows of kernels (rows are the | No | No |
| | long way) | | |
| DWK | Dry weight of kernels (g). | No | Yes; increased |
| | (yield) | | |
| DWCOB | Dry weight of cob (g) (by math, | No | No |
| | DWEAR − DWK) | | |
| DWEAR | Dry weight of ear (g) | No | Yes; increased |
| KN | Kernel number | No | Yes; increased |
| X100KW | 100 kernel dry weight (g) | No | No |
| DWTOT | Dry weight of total plant (by | No | No |
| | math, DWVEG.R6 + EARW) | | |
| HI | Harvest index, DWK/DWTOT | No | Yes; increased at |
| | | | R6 |

ND: “not determined”

TABLE 17
Traits Observed in Field Pots.

| | | | | Event | | | |
|---|---|---|---|---|---|---|---|
| | | | Event or | null or | | | significantly |
| TREAT- | | Event or | construct | construct | Difference | | different |
| MENT | TRAIT | construct | mean | null mean | from null | p Value | from null |
| WW | TILN.V4 | DTP4-L13 | 1.26 | 0.97 | 0.29 | 0.10061240 | |
| WW | TILN.V4 | DTP4-L16 | 1.37 | 0.97 | 0.40 | 0.02068587 | ** |
| WW | TILN.V4 | DTP4-L17 | 1.36 | 0.94 | 0.42 | 0.01560281 | ** |
| WW | TILN.V4 | Construct | 1.33 | 0.96 | 0.37 | 0.00027952 | ** |
| WW | PLTHT.V6 | DTP4-L13 | 22.37 | 22.21 | 0.16 | 0.57160892 | |
| WW | PLTHT.V6 | DTP4-L16 | 21.98 | 22.05 | −0.06 | 0.82730554 | |
| WW | PLTHT.V6 | DTP4-L17 | 21.50 | 22.13 | −0.83 | 0.02710131 | ** |
| WW | PLTHT.V6 | Construct | 21.95 | 22.13 | −0.18 | 0.28166786 | |
| WW | LFN.V6 | DTP4-L13 | 5.93 | 5.83 | 0.10 | 0.24382458 | |
| WW | LFN.V6 | DTP4-L16 | 5.83 | 5.83 | 0.00 | 1.00000000 | |
| WW | LFN.V6 | DTP4-L17 | 5.86 | 5.93 | −0.07 | 0.42198554 | |
| WW | LFN.V6 | Construct | 5.88 | 5.87 | 0.01 | 0.83305644 | |
| WW | TILN.V6 | DTP4-L13 | 2.69 | 2.37 | 0.33 | 0.01376576 | ** |
| WW | TILN.V6 | DTP4-L16 | 2.67 | 2.27 | 0.40 | 0.00242878 | ** |
| WW | TILN.V6 | DTP4-L17 | 2.66 | 2.40 | 0.26 | 0.05289162 | * |
| WW | TILN.V6 | Construct | 2.67 | 2.34 | 0.33 | 0.00002468 | ** |
| WW | PLTHT.V10 | DTP4-L13 | 88.91 | 90.98 | −2.07 | 0.05629611 | * |
| WW | PLTHT.V10 | DTP4-L16 | 87.84 | 91.09 | −3.25 | 0.00269623 | ** |
| WW | PLTHT.V10 | DTP4-L17 | 82.85 | 89.11 | −6.46 | 0.00000001 | ** |
| WW | PLTHT.V10 | Construct | 86.47 | 90.39 | −3.93 | 0.00000000 | ** |
| WW | LFN.V10 | DTP4-L13 | 10.04 | 10.07 | −0.03 | 0.61109281 | |
| WW | LFN.V10 | DTP4-L16 | 9.93 | 9.97 | −0.03 | 0.58640533 | |
| WW | LFN.V10 | DTP4-L17 | 9.83 | 10.00 | −0.17 | 0.00599107 | ** |
| WW | LFN.V10 | Construct | 9.93 | 10.01 | −0.08 | 0.02779416 | ** |
| WW | PLTHT.V13 | DTP4-L13 | 137.88 | 139.92 | −2.05 | 0.10694161 | |
| WW | PLTHT.V13 | DTP4-L16 | 138.94 | 139.32 | −2.37 | 0.05908537 | * |
| WW | PLTHT.V13 | DTP4-L17 | 130.10 | 137.89 | −7.79 | 0.00000001 | ** |
| WW | PLTHT.V13 | Construct | 134.97 | 139.04 | −4.07 | 0.00000012 | ** |
| WW | LFN.V13 | DTP4-L13 | 13.04 | 13.07 | −0.03 | 0.66799636 | |
| WW | LFN.V13 | DTP4-L16 | 12.97 | 13.00 | −0.03 | 0.64259662 | |
| WW | LFN.V13 | DTP4-L17 | 12.90 | 13.03 | −0.14 | 0.06236167 | * |
| WW | LFN.V13 | Construct | 12.97 | 13.03 | −0.07 | 0.11105689 | |
| WW | PLTHT.V16 | DTP4-L13 | 190.40 | 193.60 | −3.20 | 0.02670512 | ** |
| WW | PLTHT.V16 | DTP4-L16 | 187.88 | 192.08 | −4.20 | 0.00336881 | ** |
| WW | PLTHT.V16 | DTP4-L17 | 179.91 | 188.77 | −8.86 | 0.00000001 | ** |
| WW | PLTHT.V16 | Construct | 186.06 | 191.48 | −5.42 | 0.00000000 | ** |
| WW | DUALEX.CHL | DTP4-L13 | 45.09 | 44.69 | 0.40 | 0.78788150 | |
| WW | DUALEX.CHL | DTP4-L16 | 43.87 | 44.62 | −0.75 | 0.62014378 | |
| WW | DUALEX.CHL | DTP4-L17 | 43.18 | 44.79 | −1.63 | 0.27233009 | |
| WW | DUALEX.CHL | Construct | 44.04 | 44.70 | −0.66 | 0.44885445 | |
| WW | DUALEX.FLV | DTP4-L13 | 0.83 | 0.88 | −0.06 | 0.34590013 | |
| WW | DUALEX.FLV | DTP4-L16 | 0.84 | 0.88 | −0.03 | 0.58952091 | |
| WW | DUALEX.FLV | DTP4-L17 | 0.80 | 0.89 | −0.09 | 0.11031364 | |
| WW | DUALEX.FLV | Construct | 0.82 | 0.88 | −0.06 | 0.08113570 | * |
| WW | DUALEX.NBI | DTP4-L13 | 59.53 | 54.80 | 4.73 | 0.23817731 | |
| WW | DUALEX.NBI | DTP4-L16 | 55.40 | 53.47 | 1.93 | 0.62848327 | |
| WW | DUALEX.NBI | DTP4-L17 | 57.27 | 53.35 | 3.92 | 0.33121059 | |
| WW | DUALEX.NBI | Construct | 57.40 | 53.87 | 3.53 | 0.13539637 | |
| WW | WU | DTP4-L13 | 1168.57 | 1104.31 | 64.26 | 0.06497045 | * |
| WW | WU | DTP4-L16 | 1127.24 | 1009.67 | 117.57 | 0.00144349 | ** |
| WW | WU | DTP4-L17 | 1015.46 | 1112.83 | −97.36 | 0.00628317 | ** |
| WW | WU | Construct | 1103.76 | 1075.60 | 28.16 | 0.17655244 | |
| WW | WUE | DTP4-L13 | 0.13 | 0.14 | −0.01 | 0.04438817 | ** |
| WW | WUE | DTP4-L16 | 0.13 | 0.14 | −0.01 | 0.00774256 | ** |
| WW | WUE | DTP4-L17 | 0.13 | 0.13 | −0.01 | 0.13121996 | |
| WW | WUE | Construct | 0.13 | 0.14 | −0.01 | 0.00079553 | ** |
| WW | PLTHT.R1 | DTP4-L13 | 261.68 | 259.12 | 2.56 | 0.24461804 | |
| WW | PLTHT.R1 | DTP4-L16 | 256.35 | 252.12 | 4.22 | 0.03844392 | ** |
| WW | PLTHT.R1 | DTP4-L17 | 246.04 | 260.39 | −14.35 | 0.00000000 | ** |
| WW | PLTHT.R1 | Construct | 254.69 | 257.21 | −2.52 | 0.01432462 | ** |
| WW | EARHT | DTP4-L13 | 104.02 | 108.16 | −4.14 | 0.18688931 | |
| WW | EARHT | DTP4-L16 | 113.19 | 109.85 | 3.34 | 0.26579653 | |
| WW | EARHT | DTP4-L17 | 108.27 | 109.87 | −1.60 | 0.58734218 | |
| WW | EARHT | Construct | 108.50 | 109.30 | −0.80 | 0.63293108 | |
| WW | LFN | DTP4-L13 | 18.90 | 18.87 | 0.03 | 0.80990694 | |
| WW | LFN | DTP4-L16 | 18.93 | 18.87 | 0.06 | 0.60450049 | |
| WW | LFN | DTP4-L17 | 18.86 | 18.73 | 0.13 | 0.30082730 | |
| WW | LFN | Construct | 18.90 | 18.82 | 0.07 | 0.30080168 | |
| WW | EARLP | DTP4-L13 | 11.01 | 11.34 | −0.33 | 0.02957145 | ** |
| WW | EARLP | DTP4-L16 | 11.14 | 11.27 | −0.13 | 0.37839219 | |
| WW | EARLP | DTP4-L17 | 11.51 | 11.47 | 0.03 | 0.82043803 | |
| WW | EARLP | Construct | 11.22 | 11.36 | −0.14 | 0.10366448 | |
| WW | DWMAIN.R1 | DTP4-L13 | 141.28 | 154.43 | −13.14 | 0.00329335 | ** |
| WW | DWMAIN.R1 | DTP4-L16 | 135.89 | 141.91 | −6.02 | 0.14381188 | |
| WW | DWMAIN.R1 | DTP4-L17 | 126.35 | 145.84 | −19.49 | 0.00000722 | ** |
| WW | DWMAIN.R1 | Construct | 134.51 | 147.39 | −12.89 | 0.00000011 | ** |
| WW | DWTIL.R1 | DTP4-L13 | 10.70 | 6.90 | 3.80 | 0.01819417 | ** |
| WW | DWTIL.R1 | DTP4-L16 | 11.45 | 5.30 | 6.14 | 0.00035140 | * |
| WW | DWTIL.R1 | DTP4-L17 | 10.53 | 8.63 | 1.91 | 0.29347209 | |
| WW | DWTIL.R1 | Construct | 10.89 | 6.94 | 3.95 | 0.00000977 | ** |
| WW | DWVEG.R1 | DTP4-L13 | 154.58 | 159.77 | −5.19 | 0.32753594 | |
| WW | DWVEG.R1 | DTP4-L16 | 149.77 | 145.31 | 4.47 | 0.39342235 | |
| WW | DWVEG.R1 | DTP4-L17 | 137.68 | 154.33 | −16.65 | 0.00206288 | ** |
| WW | DWVEG.R1 | Construct | 147.34 | 153.14 | −5.79 | 0.06184574 | * |
| WW | DWEAR.R1 | DTP4-L13 | 2.40 | 2.27 | 0.13 | 0.46947926 | |
| WW | DWEAR.R1 | DTP4-L16 | 2.23 | 2.01 | 0.23 | 0.19670390 | |
| WW | DWEAR.R1 | DTP4-L17 | 1.27 | 2.18 | −0.92 | 0.00000063 | ** |
| WW | DWEAR.R1 | Construct | 1.97 | 2.15 | −0.18 | 0.07229087 | * |
| WW | DWTOT.R1 | DTP4-L13 | 158.68 | 162.14 | −5.46 | 0.30802330 | |
| WW | DWTOT.R1 | DTP4-L16 | 151.81 | 147.25 | 4.56 | 0.38844940 | |
| WW | DWTOT.R1 | DTP4-L17 | 139.30 | 156.50 | −17.20 | 0.00164783 | ** |
| WW | DWTOT.R1 | Construct | 149.28 | 155.30 | −6.03 | 0.05436711 | ** |
| WW | SHED | DTP4-L13 | 61.47 | 61.44 | 0.03 | 0.83820281 | |
| WW | SHED | DTP4-L16 | 81.59 | 61.46 | 0.14 | 0.36890286 | |
| WW | SHED | DTP4-L17 | 61.99 | 61.45 | 0.54 | 0.00066393 | ** |
| WW | SHED | Construct | 61.69 | 61.45 | 0.24 | 0.00828503 | ** |
| WW | SILK | DTP4-L13 | 62.48 | 62.42 | 0.06 | 0.74458746 | |
| WW | SILK | DTP4-L16 | 62.73 | 62.48 | 0.26 | 0.12897308 | |
| WW | SILK | DTP4-L17 | 62.90 | 62.50 | 0.40 | 0.02421494 | ** |
| WW | SILK | Construct | 62.70 | 62.47 | 0.24 | 0.01767604 | ** |
| WW | ASI | DTP4-L13 | 1.00 | 0.97 | 0.03 | 0.88029003 | |
| WW | ASI | DTP4-L16 | 1.13 | 1.00 | 0.13 | 0.52025914 | |
| WW | ASI | DTP4-L17 | 0.89 | 1.04 | −0.15 | 0.48299805 | |
| WW | ASI | Construct | 1.01 | 1.00 | 0.00 | 0.96659995 | |
| WW | GR.V6V10 | DTP4-L13 | 5.12 | 5.29 | −0.16 | 0.02138426 | ** |
| WW | GR.V6V10 | DTP4-L16 | 5.05 | 5.30 | −0.25 | 0.00051092 | ** |
| WW | GR.V6V10 | DTP4-L17 | 4.72 | 5.16 | −0.44 | 0.00000000 | ** |
| WW | GR.V6V10 | Construct | 4.96 | 5.25 | −0.28 | 0.00000000 | ** |
| WW | GR.V10V13 | DTP4-L13 | 6.05 | 6.06 | 0.00 | 0.97370623 | |
| WW | GR.V10V13 | DTP4-L16 | 6.05 | 5.99 | 0.05 | 0.57289713 | |
| WW | GR.V10V13 | DTP4-L17 | 5.90 | 5.99 | −0.09 | 0.34364716 | |
| WW | GR.V10V13 | Construct | 6.00 | 6.01 | −0.01 | 0.80674055 | |
| WW | STKD | DTP4-L13 | 19.66 | 19.57 | 0.09 | 0.81915673 | |
| WW | STKD | DTP4-L16 | 19.41 | 19.14 | 0.26 | 0.50007008 | |
| WW | STKD | DTP4-L17 | 18.47 | 19.44 | −0.97 | 0.01514156 | ** |
| WW | STKD | Construct | 19.18 | 19.38 | −0.21 | 0.36986927 | |
| WW | DWVEG.R6 | DTP4-L13 | 185.19 | 177.14 | 8.05 | 0.472471015 | |
| WW | DWVEG.R6 | DTP4-L16 | 186.95 | 160.32 | 26.63 | 0.020291776 | ** |
| WW | DWVEG.R6 | DTP4-L17 | 179.65 | 173.34 | 6.30 | 0.559822469 | |
| WW | DWVEG.R6 | Construct | 183.93 | 170.27 | 13.66 | 0.035936981 | ** |
| WW | ROW | DTP4-L13 | 15.59 | 15.91 | −0.32 | 0.479182267 | |
| WW | ROW | DTP4-L16 | 15.46 | 15.23 | 0.23 | 0.617709691 | |
| WW | ROW | DTP4-L17 | 15.67 | 15.46 | 0.21 | 0.637947442 | |
| WW | ROW | Construct | 15.57 | 15.53 | 0.04 | 0.874855288 | |
| WW | DWK | DTP4-L13 | 191.37 | 198.81 | −7.44 | 0.438648208 | |
| WW | DWK | DTP4-L16 | 185.09 | 182.01 | 3.08 | 0.752787745 | |
| WW | DWK | DTP4-L17 | 201.76 | 190.13 | 11.63 | 0.234351744 | |
| WW | DWK | Construct | 192.74 | 190.32 | 2.42 | 0.666015774 | |
| WW | DWCOB | DTP4-L13 | 29.01 | 30.61 | −1.60 | 0.30686635 | |
| WW | DWCOB | DTP4-L16 | 28.21 | 27.05 | 1.16 | 0.46548495 | |
| WW | DWCOB | DTP4-L17 | 29.98 | 29.34 | 0.64 | 0.688236714 | |
| WW | DWCOB | Construct | 29.07 | 29.00 | 0.07 | 0.940788565 | |
| WW | DWEAR | DTP4-L13 | 220.38 | 229.44 | −9.06 | 0.408924123 | |
| WW | DWEAR | DTP4-L16 | 213.27 | 209.02 | 4.25 | 0.703637408 | |
| WW | DWEAR | DTP4-L17 | 231.74 | 219.47 | 12.28 | 0.271297248 | |
| WW | DWEAR | Construct | 221.80 | 219.31 | 2.49 | 0.697630835 | |
| WW | KN | DTP4-L13 | 622.83 | 672.22 | −49.39 | 0.094181834 | |
| WW | KN | DTP4-L16 | 596.25 | 605.66 | −9.41 | 0.75222573 | |
| WW | KN | DTP4-L17 | 650.05 | 614.89 | 35.16 | 0.238313504 | |
| WW | KN | Construct | 623.04 | 630.92 | −7.88 | 0.645325941 | |
| WW | X100KW | DTP4-L13 | 30.64 | 29.59 | 1.05 | 0.253513862 | |
| WW | X100KW | DTP4-L16 | 31.21 | 30.16 | 1.05 | 0.262791041 | |
| WW | X100KW | DTP4-L17 | 31.00 | 30.80 | 0.20 | 0.833547395 | |
| WW | X100KW | Construct | 30.95 | 30.18 | 0.77 | 0.15555604 | |
| WW | DWTOT | DTP4-L13 | 411.37 | 413.61 | −2.24 | 0.916063317 | |
| WW | DWTOT | DTP4-L16 | 404.92 | 374.02 | 30.90 | 0.15154254 | |
| WW | DWTOT | DTP4-L17 | 420.11 | 393.88 | 26.23 | 0.203301266 | |
| WW | DWTOT | Construct | 412.14 | 393.84 | 18.30 | 0.136197041 | |
| WW | HI | DTP4-L13 | 0.48 | 0.49 | −0.02 | 0.257690608 | |
| WW | HI | DTP4-L16 | 0.47 | 0.50 | −0.03 | 0.035508997 | ** |
| WW | HI | DTP4-L17 | 0.50 | 0.48 | 0.02 | 0.200998462 | |
| WW | HI | Construct | 0.48 | 0.49 | −0.01 | 0.236486772 | |
| DRT | TILN.V4 | DTP4-L13 | 1.18 | 0.81 | 0.37 | 0.03737189 | ** |
| DRT | TILN.V4 | DTP4-L16 | 1.34 | 0.61 | 0.73 | 0.00004332 | ** |
| DRT | TILN.V4 | DTP4-L17 | 1.28 | 0.74 | 0.53 | 0.00263586 | ** |
| DRT | TILN.V4 | Construct | 1.27 | 0.72 | 0.54 | 0.00000022 | ** |
| DRT | PLTHT.V6 | DTP4-L13 | 22.11 | 22.20 | −0.09 | 0.71441900 | |
| DRT | PLTHT.V6 | DTP4-L16 | 21.77 | 21.84 | −0.07 | 0.78976699 | |
| DRT | PLTHT.V6 | DTP4-L17 | 21.57 | 22.18 | −0.61 | 0.01802070 | ** |
| DRT | PLTHT.V6 | Construct | 21.81 | 22.07 | −0.26 | 0.08476641 | ** |
| DRT | LFN.V6 | DTP4-L13 | 5.98 | 5.95 | 0.03 | 0.45412771 | |
| DRT | LFN.V6 | DTP4-L16 | 5.95 | 5.95 | 0.00 | 1.00000000 | |
| DRT | LFN.V6 | DTP4-L17 | 5.88 | 5.98 | −0.10 | 0.02565660 | ** |
| DRT | LFN.V6 | Construct | 5.94 | 5.96 | −0.02 | 0.38752173 | |
| DRT | TILN.V6 | DTP4-L13 | 2.60 | 2.17 | 0.43 | 0.00206163 | ** |
| DRT | TILN.V6 | DTP4-L16 | 2.77 | 2.10 | 0.67 | 0.00000321 | ** |
| DRT | TILN.V6 | DTP4-L17 | 2.80 | 2.37 | 0.43 | 0.00206163 | ** |
| DRT | TILN.V6 | Construct | 2.72 | 2.21 | 0.51 | 0.00000000 | ** |
| DRT | PLTHT.V10 | DTP4-L13 | 92.20 | 93.42 | −1.21 | 0.25004790 | |
| DRT | PLTHT.V10 | DTP4-L16 | 91.37 | 92.32 | −0.95 | 0.37703904 | |
| DRT | PLTHT.V10 | DTP4-L17 | 88.49 | 92.79 | −4.30 | 0.00007419 | ** |
| DRT | PLTHT.V10 | Construct | 90.69 | 92.84 | −2.15 | 0.00066349 | ** |
| DRT | LFN.V10 | DTP4-L13 | 10.00 | 10.07 | −0.07 | 0.30588796 | |
| DRT | LFN.V10 | DTP4-L16 | 10.07 | 10.03 | 0.03 | 0.60828418 | |
| DRT | LFN.V10 | DTP4-L17 | 9.93 | 10.10 | −0.17 | 0.01109122 | ** |
| DRT | LFN.V10 | Construct | 10.00 | 10.07 | −0.07 | 0.07704135 | |
| DRT | PLTHT.V13 | DTP4-L13 | 135.66 | 138.16 | −2.50 | 0.05873323 | * |
| DRT | PLTHT.V13 | DTP4-L16 | 135.10 | 134.55 | 0.56 | 0.67020840 | |
| DRT | PLTHT.V13 | DTP4-L17 | 134.27 | 137.87 | −3.60 | 0.00667808 | ** |
| DRT | PLTHT.V13 | Construct | 135.01 | 136.86 | −1.85 | 0.01485240 | ** |
| DRT | LFN.V13 | DTP4-L13 | 12.90 | 12.97 | −0.07 | 0.39420034 | |
| DRT | LFN.V13 | DTP4-L16 | 12.87 | 12.80 | 0.07 | 0.39420034 | |
| DRT | LFN.V13 | DTP4-L17 | 12.80 | 12.97 | −0.17 | 0.03413080 | ** |
| DRT | LFN.V13 | Construct | 12.86 | 12.91 | −0.06 | 0.21929525 | |
| DRT | PLTHT.V16 | DTP4-L13 | 173.44 | 174.19 | −0.75 | 0.60570222 | |
| DRT | PLTHT.V16 | DTP4-L16 | 172.78 | 172.69 | 0.07 | 0.96121412 | |
| DRT | PLTHT.V16 | DTP4-L17 | 172.46 | 174.43 | −1.97 | 0.16863300 | |
| DRT | PLTHT.V16 | Construct | 172.89 | 173.77 | −0.88 | 0.28458043 | |
| DRT | DUALEX.CHL | DTP4-L13 | 41.39 | 42.09 | −0.70 | 0.55913823 | |
| DRT | DUALEX.CHL | DTP4-L16 | 41.68 | 41.04 | 0.65 | 0.58371725 | |
| DRT | DUALEX.CHL | DTP4-L17 | 41.81 | 41.80 | 0.01 | 0.99403591 | |
| DRT | DUALEX.CHL | Construct | 41.63 | 41.64 | −0.01 | 0.98483459 | |
| DRT | DUALEX.FLV | DTP4-L13 | 0.73 | 0.81 | −0.08 | 0.10497460 | |
| DRT | DUALEX.FLV | DTP4-L16 | 0.72 | 0.86 | −0.14 | 0.00384718 | ** |
| DRT | DUALEX.FLV | DTP4-L17 | 0.78 | 0.72 | 0.06 | 0.21800652 | |
| DRT | DUALEX.FLV | Construct | 0.74 | 0.80 | −0.05 | 0.05661318 | * |
| DRT | DUALEX.NBI | DTP4-L13 | 59.84 | 54.78 | 5.07 | 0.15566030 | |
| DRT | DUALEX.NBI | DTP4-L16 | 62.23 | 53.77 | 8.47 | 0.01832608 | ** |
| DRT | DUALEX.NBI | DTP4-L17 | 57.05 | 63.58 | −6.54 | 0.07037471 | * |
| DRT | DUALEX.NBI | Construct | 59.71 | 57.38 | 2.33 | 0.25803990 | |
| DRT | RWC | DTP4-L13 | 63.07 | 60.05 | 3.02 | 0.00001012 | ** |
| DRT | RWC | DTP4-L16 | 63.26 | 60.92 | 2.34 | 0.00167083 | ** |
| DRT | RWC | DTP4-L17 | 60.35 | 63.71 | −3.37 | 0.00005237 | ** |
| DRT | RWC | Construct | 62.22 | 61.56 | 0.66 | 0.11241576 | |
| DRT | PLTHT.R1 | DTP4-L13 | 240.17 | 238.29 | 1.88 | 0.26645568 | |
| DRT | PLTHT.R1 | DTP4-L16 | 238.63 | 237.80 | −1.18 | 0.46424610 | |
| DRT | PLTHT.R1 | DTP4-L17 | 236.32 | 241.45 | −5.13 | 0.00738054 | ** |
| DRT | PLTHT.R1 | Construct | 237.70 | 239.18 | −1.48 | 0.24612187 | |
| DRT | EARHT | DTP4-L13 | 108.91 | 108.11 | 0.79 | 0.82357635 | |
| DRT | EARHT | DTP4-L16 | 112.05 | 115.87 | −3.82 | 0.25470767 | |
| DRT | EARHT | DTP4-L17 | 110.96 | 111.45 | −0.49 | 0.89285465 | |
| DRT | EARHT | Construct | 110.64 | 111.81 | −1.17 | 0.56248804 | |
| DRT | LFN | DTP4-L13 | 18.77 | 18.90 | −0.13 | 0.36030764 | |
| DRT | LFN | DTP4-L16 | 18.77 | 18.97 | −0.20 | 0.15503518 | |
| DRT | LFN | DTP4-L17 | 18.77 | 18.87 | −0.10 | 0.47612562 | |
| DRT | LFN | Construct | 18.77 | 18.91 | −0.14 | 0.07918268 | * |
| DRT | EARLP | DTP4-L13 | 11.44 | 11.66 | −0.21 | 0.25184046 | |
| DRT | EARLP | DTP4-L16 | 11.30 | 11.62 | −0.31 | 0.08839970 | * |
| DRT | EARLP | DTP4-L17 | 11.45 | 11.49 | −0.04 | 0.83236924 | |
| DRT | EARLP | Construct | 11.40 | 11.59 | −0.19 | 0.07795055 | * |
| DRT | DWMAIN.R1 | DTP4-L13 | 128.67 | 135.77 | −7.11 | 0.00067236 | ** |
| DRT | DWMAIN.R1 | DTP4-L16 | 127.87 | 130.60 | −2.73 | 0.22100772 | |
| DRT | DWMAIN.R1 | DTP4-L17 | 124.70 | 128.09 | −3.38 | 0.17081765 | |
| DRT | DWMAIN.R1 | Construct | 127.08 | 131.49 | −4.41 | 0.00111012 | ** |
| DRT | DWTIL.R1 | DTP4-L13 | 6.43 | 3.90 | 2.53 | 0.09173020 | ** |
| DRT | DWTIL.R1 | DTP4-L16 | 6.31 | 3.04 | 3.27 | 0.02219856 | ** |
| DRT | DWTIL.R1 | DTP4-L17 | 9.25 | 2.41 | 6.84 | 0.00001569 | ** |
| DRT | DWTIL.R1 | Construct | 7.33 | 3.12 | 4.21 | 0.00000292 | ** |
| DRT | DWVEG.R1 | DTP4-L13 | 135.77 | 138.70 | −2.92 | 0.44895425 | |
| DRT | DWVEG.R1 | DTP4-L16 | 135.94 | 131.22 | 4.72 | 0.22226946 | |
| DRT | DWVEG.R1 | DTP4-L17 | 133.02 | 130.33 | 2.68 | 0.50819162 | |
| DRT | DWVEG.R1 | Construct | 134.91 | 133.42 | 1.49 | 0.52385836 | |
| DRT | DWEAR.R1 | DTP4-L13 | 1.28 | 1.30 | −0.02 | 0.94930818 | |
| DRT | DWEAR.R1 | DTP4-L16 | 1.44 | 1.02 | 0.42 | 0.14805885 | |
| DRT | DWEAR.R1 | DTP4-L17 | 1.70 | 0.92 | 0.78 | 0.01358393 | ** |
| DRT | DWEAR.R1 | Construct | 1.48 | 1.08 | 0.40 | 0.03074121 | ** |
| DRT | DWTOT.R1 | DTP4-L13 | 136.64 | 139.84 | −3.19 | 0.41581367 | |
| DRT | DWTOT.R1 | DTP4-L16 | 137.62 | 132.58 | 5.04 | 0.20337115 | |
| DRT | DWTOT.R1 | DTP4-L17 | 134.83 | 131.47 | 3.35 | 0.41814064 | |
| DRT | DWTOT.R1 | Construct | 136.36 | 134.63 | 1.73 | 0.46821874 | |
| DRT | SHED | DTP4-L13 | 61.80 | 61.97 | −0.17 | 0.57903541 | |
| DRT | SHED | DTP4-L16 | 61.97 | 82.43 | −0.47 | 0.12144252 | |
| DRT | SHED | DTP4-L17 | 62.63 | 61.83 | 0.80 | 0.00835084 | ** |
| DRT | SHED | Construct | 62.13 | 62.08 | 0.06 | 0.74866113 | |
| DRT | SILK | DTP4-L13 | 65.11 | 65.66 | −0.55 | 0.23958634 | |
| DRT | SILK | DTP4-L16 | 65.20 | 65.29 | −0.10 | 0.83421664 | |
| DRT | SILK | DTP4-L17 | 64.98 | 66.26 | −1.28 | 0.01041071 | ** |
| DRT | SILK | Construct | 65.09 | 65.73 | −0.64 | 0.01991307 | ** |
| DRT | ASI | DTP4-L13 | 3.38 | 3.88 | −0.50 | 0.34118899 | |
| DRT | ASI | DTP4-L16 | 3.51 | 2.89 | 0.62 | 0.22779519 | |
| DRT | ASI | DTP4-L17 | 2.48 | 4.64 | −2.17 | 0.00014762 | ** |
| DRT | ASI | Construct | 3.12 | 3.80 | −0.68 | 0.02729734 | ** |
| DRT | GR.V6V10 | DTP4-L13 | 5.39 | 5.50 | −0.11 | 0.13336177 | |
| DRT | GR.V6V10 | DTP4-L16 | 5.34 | 5.40 | −0.06 | 0.41283559 | |
| DRT | GR.V6V10 | DTP4-L17 | 5.14 | 5.42 | −0.28 | 0.00013367 | ** |
| DRT | GR.V6V10 | Construct | 5.29 | 5.44 | −0.15 | 0.00056568 | ** |
| DRT | GR.V10V13 | DTP4-L13 | 5.35 | 5.55 | −0.19 | 0.14897867 | |
| DRT | GR.V10V13 | DTP4-L16 | 5.43 | 5.27 | 0.15 | 0.26051959 | |
| DRT | GR.V10V13 | DTP4-L17 | 5.59 | 5.44 | 0.15 | 0.25486733 | |
| DRT | GR.V10V13 | Construct | 5.46 | 5.42 | 0.04 | 0.62904773 | |
| DRT | YL.7.15 | DTP4-L13 | 6.98 | 7.11 | −0.13 | 0.19340561 | |
| DRT | YL.7.15 | DTP4-L16 | 6.74 | 6.94 | −0.20 | 0.04179433 | ** |
| DRT | YL.7.15 | DTP4-L17 | 6.75 | 6.98 | −0.23 | 0.01922373 | ** |
| DRT | YL.7.15 | Construct | 6.82 | 7.01 | −0.19 | 0.00123685 | ** |
| DRT | YL.8.1 | DTP4-L13 | 9.88 | 10.41 | −0.53 | 0.05275571 | * |
| DRT | YL.8.1 | DTP4-L16 | 9.96 | 9.98 | −0.02 | 0.94170108 | |
| DRT | YL.8.1 | DTP4-L17 | 9.70 | 9.97 | −0.27 | 0.33026794 | |
| DRT | YL.8.1 | Construct | 9.85 | 10.12 | −0.27 | 0.09054654 | * |
| DRT | YL.8.11 | DTP4-L13 | 10.99 | 11.76 | −0.77 | 0.00606165 | ** |
| DRT | YL.8.11 | DTP4-L16 | 10.84 | 10.87 | −0.04 | 0.89082227 | |
| DRT | YL.8.11 | DTP4-L17 | 10.44 | 10.95 | −0.51 | 0.07267146 | * |
| DRT | YL.8.11 | Construct | 10.75 | 11.19 | −0.44 | 0.00749806 | ** |
| DRT | DWVEG.R6 | DTP4-L13 | 114.68 | 118.93 | −4.26 | 0.610440457 | |
| DRT | DWVEG.R6 | DTP4-L16 | 119.38 | 120.98 | −1.60 | 0.848086748 | |
| DRT | DWVEG.R6 | DTP4-L17 | 120.64 | 146.04 | −25.40 | 0.005617906 | ** |
| DRT | DWVEG.R6 | Construct | 118.23 | 128.65 | −10.42 | 0.037370754 | ** |
| DRT | ROW.1 | DTP4-L13 | 15.56 | 15.07 | 0.49 | 0.392964042 | |
| DRT | ROW.1 | DTP4-L16 | 15.33 | 16.12 | −0.78 | 0.158014994 | |
| DRT | ROW.1 | DTP4-L17 | 15.12 | 15.00 | 0.12 | 0.843163311 | |
| DRT | ROW.1 | Construct | 15.34 | 15.40 | −0.06 | 0.858801963 | |
| DRT | DWK | DTP4-L13 | 95.65 | 79.98 | 15.67 | 0.151305864 | |
| DRT | DWK | DTP4-L16 | 93.74 | 84.95 | 8.78 | 0.393267032 | |
| DRT | DWK | DTP4-L17 | 84.86 | 62.85 | 22.00 | 0.049180594 | ** |
| DRT | DWK | Construct | 91.42 | 75.93 | 15.49 | 0.013697301 | ** |
| DRT | DWCOB | DTP4-L13 | 15.50 | 14.52 | 0.98 | 0.406520641 | |
| DRT | DWCOB | DTP4-L16 | 15.83 | 15.23 | 0.61 | 0.585311369 | |
| DRT | DWCOB | DTP4-L17 | 15.67 | 15.00 | 0.66 | 0.580121718 | |
| DRT | DWCOB | Construct | 15.67 | 14.92 | 0.75 | 0.265159919 | |
| DRT | DWEAR | DTP4-L13 | 110.98 | 94.32 | 16.66 | 0.149473434 | |
| DRT | DWEAR | DTP4-L16 | 109.53 | 100.18 | 9.36 | 0.390106055 | |
| DRT | DWEAR | DTP4-L17 | 100.57 | 77.88 | 22.69 | 0.055285154 | |
| DRT | DWEAR | Construct | 107.03 | 90.79 | 16.24 | 0.014608231 | ** |
| DRT | KN | DTP4-L13 | 373.56 | 282.57 | 90.99 | 0.042767336 | ** |
| DRT | KN | DTP4-L16 | 341.47 | 324.97 | 16.50 | 0.696092766 | |
| DRT | KN | DTP4-L17 | 315.12 | 228.80 | 86.32 | 0.058673186 | |
| DRT | KN | Construct | 343.38 | 278.78 | 64.60 | 0.012066189 | ** |
| DRT | X100KW | DTP4-L13 | 26.08 | 27.58 | −1.50 | 0.156126321 | |
| DRT | X100KW | DTP4-L16 | 28.07 | 26.87 | 1.19 | 0.228474518 | |
| DRT | X100KW | DTP4-L17 | 27.11 | 26.80 | 0.32 | 0.768899771 | |
| DRT | X100KW | Construct | 27.09 | 27.08 | 0.00 | 0.994088329 | |
| DRT | DWTOT | DTP4-L13 | 218.34 | 204.63 | 13.70 | 0.25823758 | |
| DRT | DWTOT | DTP4-L16 | 219.85 | 216.85 | 3.00 | 0.803698091 | |
| DRT | DWTOT | DTP4-L17 | 212.97 | 209.09 | 3.88 | 0.767715805 | |
| DRT | DWTOT | Construct | 217.05 | 210.19 | 6.86 | 0.339825972 | |
| DRT | HI | DTP4-L13 | 0.39 | 0.31 | 0.08 | 0.147886079 | |
| DRT | HI | DTP4-L16 | 0.36 | 0.37 | 0.00 | 0.937885549 | |
| DRT | HI | DTP4-L17 | 0.34 | 0.21 | 0.13 | 0.025392684 | ** |
| DRT | HI | Construct | 0.36 | 0.30 | 0.07 | 0.033449426 | ** |

(WW = well watered; DRT = drought stressed; ** p value < 0.05; * p value < 0.1))

### Example 39

### Profile HMM Specific to DTP4

Profile HMMs are statistical models of multiple sequence alignments, or even of single sequences. They capture position-specific information about how conserved each column of the alignment is, and which residues are likely.

### Description:

HMMER® (biosequence analysis using profile hidden Markov models) is used to search sequence databases for homologs of protein sequences, and to make protein sequence alignments. HMMER® can be used to search sequence databases with single query sequences, but it becomes particularly powerful when the query is a multiple sequence alignment of a sequence family. HMMER® makes a profile of the query that assigns a position-specific scoring system for substitutions, insertions, and deletions. HMMER® profiles are probabilistic models called “profile hidden Markov models” (profile HMMs) (Krogh et al., 1994, J. Mol. Biol., 235:1501-1531; Eddy, 1998, Curr. Opin. Struct. Biol., 6:361-365.; Durbin et al., Probabilistic Models of Proteins and Nucleic Acids. Cambridge University Press, Cambridge UK. 1998, Eddy, Sean R., March 2010, HMMER User's Guide Version 3.0, Howard Hughes Medical Institute, Janelia Farm Research Campus, Ashburn Va., USA; US patent publication No. US20100293118). Compared to BLAST, FASTA, and other sequence alignment and database search tools based on older scoring methodology, HMMER® aims to be significantly more accurate and more able to detect remote homologs, because of the strength of its underlying probability models.

### Method for Creating Profile HMMs Specific to DTP4 Gene Family

### Step1: Identification of Homologs of AT-DTP4:

Homologs for AT-CXE20 were identified by querying protein sequence of AT-DTP4 using BLAST and Jackhammer within an in house database of protein sequences generated by compilation of protein sequences from UniProt and translated ORFs from various plant genomes that were retrieved from NCBI and internal sequencing cDNA sequencing data. Homologs thus identified were aligned using the software MUSCLE (Edgar, Robert C. (2004), Nucleic Acids Research 19; 32(5):1792-7) using the MEGA6 program (Phylogenetic and molecular evolutionary analyses were conducted using MEGA version 6 (Tamura K., et al (2013) Mol. Biol. Evol. 30 (12): 2725-2729). Phylogenetic analysis was done with the MEGA6 program, and the Maximum Likelihood method (Jones D. T., et al (1992). Comp Appl Biosci 8: 275-282; Tamura K., et al (2013) Mol. Biol. Evol. 30 (12): 2725-2729).

Branches of the resulting tree were annotated according to Marshall et al J Mol Evol (2003) 57:487-500. Utilizing the Marshall nomenclature, a subset of genes from CXE tree, Type II, Type IV, Type V, and Type VI were isolated and realigned. A new Maximum Likelihood tree was built using just these proteins.

### Step 2: Identify and Realign Type II Carboxylesterases

Proteins specific to the Type II lead branch were realigned and a new tree was built with the same process as step 1. Proteins from the new Type II specific tree were then picked based on the branching pattern in order to get one protein per sub branch. These proteins, SEQ ID NOS:18, 29, 33, 45, 47, 53, 55, 61,64, 65, 77, 78, 101, 103, 105, 107, 111, 115, 131, 132, 135, 137, 139, 141, 144, 433, 559 and 604, were realigned and used for the HMM build in step 3.

Step 3: Creating profile HMM for DTP4

HMMbuild module of HMMER® 3.0 was used to create a profile HMM for DTP4 based on Multiple Sequence Alignment (MSA) of homologs of AT-CXE20.

### Step 4: Using Profile to Search Protein Database

Profile HMM created was queried in a database of protein sequences described in Step 1. Hits retrieved were further examined as described in Step 5.

### Step5: Determining Specificity of Profile to Identify DTP4 Related Protein Sequences

All protein sequences that matched the profile HMM of CXE20 with an E-value of less than 0.001 over at least 80% length of the HMM profile were regarded as statistically significant and corresponding to gene family. Since all statistically significant protein hits obtained are members of CXE20 gene family, it is suggested that profile HMM for CXE20 described here is specific to prioritize ranking of the Type II carboxylesterases, and identify other members of the carboxylesterase family. The HMM profile for CXE20 family is shown in the appended Table 18.

### Example 40

### Targeted Regulation or Mutagenesis of an Endogenous DTP4 Gene

The skilled artisan will further appreciate that changes can be introduced by mutation of the nucleic acid sequences, thereby leading to changes in either the expression of encoded mRNAs or the amino acid sequence of the encoded polypeptide e.g., DTP4, resulting in alteration of the biological activity of the mRNA or protein, respectively, or both. See for example methods described in U.S. patent application Ser. No. 14/463,687 filed on Aug. 20, 2014, incorporated by reference in its entirety herein. Thus, variant nucleic acid molecules can be created by introducing one or more nucleotide substitutions, additions and/or deletions into the corresponding nucleic acid sequence or surrounding sequences disclosed herein. Such variant nucleic acid sequences are also encompassed by the present disclosure.

Variant nucleic acid sequences can be made by introducing sequence changes randomly along all or part of the genic region, including, but not limited to, chemical or irradiation mutagenesis and oligonucleotide-mediated mutagenesis (OMM) (Beetham et al. 1999; Okuzaki and Toriyama 2004). Alternatively or additionally, sequence changes can be introduced at specific selected sites using double-strand-break technologies such as ZNFs, custom designed homing endonucleases, TALENs, CRISPR/CAS (also referred to as guide RNA/Cas endonuclease systems (U.S. patent application Ser. No. 14/463,687 filed on Aug. 20, 2014), or other protein and/or nucleic acid based mutagenesis technologies. The resultant variants can be screened for altered activity. It will be appreciated that the techniques are often not mutually exclusive. Indeed, the various methods can be used singly or in combination, in parallel or in series, to create or access diverse sequence variants.

TABLE 18
HMM profile

| | | | HMMER3/b [3.0 \| March 2010] | | | | | | | | |
|---|---|---|---|---|---|---|---|---|---|---|---|
| | | | NAME CXE20_TypeIIBranch_limit_one_perSubBranch | | | | | | | | |
| | | | LENG 326 | | | | | | | | |
| | | | ALPH amino | | | | | | | | |
| | | | RF no | | | | | | | | |
| | | | CS no | | | | | | | | |
| | | | MAP yes | | | | | | | | |
| | | | DATE Thu Nov 13 16:23:57 2014 | | | | | | | | |
| | | | NSEQ 28 | | | | | | | | |
| | | | EFFN 1.124512 | | | | | | | | |
| | | | CKSUM 701189305 | | | | | | | | |
| | | | STATS LOCAL MSV −11.1717 0.70062 | | | | | | | | |
| | | | STATS LOCAL VITERBI −12.0197 0.70062 | | | | | | | | |
| | | | STATS LOCAL FORWARD −5.8925 0.70062 | | | | | | | | |
| HMM | A | C | D | E | F | G | H | I | K | L | M |
| | m−>m | m−>i | m−>d | i−>m | i−>i | d−>m | d−>d | | | | |
| COMPO | 2.50926 | 4.25552 | 2.8369 | 2.71139 | 3.33164 | 2.91186 | 3.58549 | 2.84504 | 2.73821 | 2.42491 | 3.61156 |
| | 2.68593 | 4.42274 | 2.77541 | 2.73137 | 3.46258 | 2.40556 | 3.72518 | 3.29342 | 2.67758 | 2.69305 | 4.24363 |
| | 0.84551 | 1.24233 | 1.26602 | 2.01075 | 0.14374 | 0 | * | | | | |
| 1 | 2.51668 | 4.86384 | 2.87354 | 2.09458 | 4.13606 | 2.98579 | 3.57833 | 3.56272 | 2.2325 | 3.13434 | 3.60294 |
| | 2.68618 | 4.42225 | 2.77519 | 2.73123 | 3.46354 | 2.40513 | 3.72494 | 3.29354 | 2.67741 | 2.69355 | 4.2469 |
| | 0.0295 | 3.93385 | 4.65619 | 0.61958 | 0.77255 | 0.70739 | 0.67911 | | | | |
| 2 | 2.39992 | 4.84776 | 2.4917 | 2.27086 | 4.10996 | 3.22751 | 3.58926 | 3.53813 | 2.37511 | 3.00262 | 3.41645 |
| | 2.68618 | 4.42225 | 2.77519 | 2.73123 | 3.46354 | 2.40513 | 3.72494 | 3.29354 | 2.67741 | 2.69355 | 4.2469 |
| | 0.02778 | 3.99313 | 4.71547 | 0.61958 | 0.77255 | 0.67547 | 0.71114 | | | | |
| 3 | 2.49757 | 4.94613 | 2.67419 | 2.21463 | 4.23287 | 3.19058 | 3.57886 | 3.67816 | 2.16759 | 3.1002 | 3.53885 |
| | 2.68618 | 4.42225 | 2.77519 | 2.73123 | 3.46354 | 2.40513 | 3.72494 | 3.29354 | 2.67741 | 2.69355 | 4.2469 |
| | 0.02645 | 4.04144 | 4.76379 | 0.61958 | 0.77255 | 0.52031 | 0.90223 | | | | |
| 4 | 2.51011 | 4.75462 | 2.90058 | 2.39419 | 3.98528 | 3.15565 | 3.65758 | 3.39418 | 2.47537 | 3.01332 | 3.00755 |
| | 2.68618 | 4.42225 | 2.77519 | 2.73123 | 3.46354 | 2.40513 | 3.72494 | 3.29354 | 2.67741 | 2.69355 | 4.2469 |
| | 0.02353 | 4.15705 | 4.8794 | 0.61958 | 0.77255 | 0.60407 | 0.79094 | | | | |
| 5 | 2.49208 | 4.73514 | 3.04507 | 2.37102 | 3.95612 | 3.44744 | 3.50593 | 3.18094 | 2.41281 | 2.85253 | 3.81156 |
| | 2.68618 | 4.42225 | 2.77519 | 2.73123 | 3.46354 | 2.40513 | 3.72494 | 3.29354 | 2.67741 | 2.69355 | 4.2469 |
| | 0.02353 | 4.15705 | 4.8794 | 0.61958 | 0.77255 | 0.56577 | 0.83915 | | | | |
| 6 | 2.54242 | 4.91918 | 2.53966 | 2.21759 | 4.19078 | 3.41485 | 3.61804 | 3.62671 | 2.394 | 3.04389 | 3.75267 |
| | 2.68618 | 4.42225 | 2.77519 | 2.73123 | 3.46354 | 2.40513 | 3.72494 | 3.29354 | 2.67741 | 2.69355 | 4.2469 |
| | 0.02307 | 4.17665 | 4.899 | 0.61958 | 0.77255 | 0.5825 | 0.81758 | | | | |
| 7 | 2.54402 | 4.87337 | 2.78891 | 2.41637 | 4.13149 | 3.19872 | 3.45235 | 3.55859 | 2.26532 | 2.7798 | 3.71016 |
| | 2.68618 | 4.42225 | 2.77519 | 2.73123 | 3.46354 | 2.40513 | 3.72494 | 3.29354 | 2.67741 | 2.69355 | 4.2469 |
| | 0.02307 | 4.17665 | 4.899 | 0.61958 | 0.77255 | 0.53702 | 0.87824 | | | | |
| 8 | 2.37704 | 4.51025 | 3.25209 | 2.70389 | 3.76346 | 3.45872 | 3.78012 | 2.80156 | 2.67734 | 2.81709 | 3.43996 |
| | 2.68618 | 4.42225 | 2.77519 | 2.73123 | 3.46354 | 2.40513 | 3.72494 | 3.29354 | 2.67741 | 2.69355 | 4.2469 |
| | 0.03938 | 4.19951 | 3.74604 | 0.61958 | 0.77255 | 0.55617 | 0.85191 | | | | |
| 9 | 2.51441 | 3.9357 | 3.19229 | 2.63786 | 3.54278 | 3.46497 | 3.73718 | 3.17037 | 2.51696 | 2.74557 | 3.69024 |
| | 2.68618 | 4.42225 | 2.77519 | 2.73123 | 3.46354 | 2.40513 | 3.72494 | 3.29354 | 2.67741 | 2.69355 | 4.2469 |
| | 0.02292 | 4.18305 | 4.9054 | 0.61958 | 0.77255 | 0.57526 | 0.82681 | | | | |
| 10 | 2.45991 | 4.53639 | 3.21275 | 2.65303 | 3.71899 | 3.5 | 3.73377 | 2.55299 | 2.43896 | 2.77209 | 3.38373 |
| | 2.68618 | 4.42225 | 2.77519 | 2.73123 | 3.46354 | 2.40513 | 3.72494 | 3.29354 | 2.67741 | 2.69355 | 4.2469 |
| | 0.02292 | 4.18305 | 4.9054 | 0.61958 | 0.77255 | 0.46321 | 0.99226 | | | | |
| 11 | 2.36766 | 4.34189 | 3.22428 | 2.8129 | 3.14532 | 3.58698 | 3.84004 | 2.60558 | 2.86376 | 2.50783 | 2.86048 |
| | 2.6862 | 4.42232 | 2.77526 | 2.73107 | 3.4636 | 2.40519 | 3.72501 | 3.29341 | 2.6774 | 2.69361 | 4.24673 |
| | 0.16912 | 1.90651 | 4.96231 | 0.84355 | 0.56244 | 0.47318 | 0.97557 | | | | |
| 12 | 2.74238 | 4.13315 | 1.60717 | 2.15785 | 4.48655 | 3.39894 | 3.68695 | 3.9551 | 2.42318 | 3.47378 | 4.25285 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 13 | 2.47298 | 4.43247 | 3.40454 | 2.87901 | 3.47482 | 3.46218 | 3.89595 | 3.06143 | 2.84119 | 2.41019 | 3.66713 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 14 | 2.73996 | 4.49232 | 3.23879 | 2.94928 | 2.69271 | 3.65463 | 3.79189 | 3.01588 | 2.93588 | 2.69147 | 3.60827 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 15 | 2.48769 | 5.06628 | 2.55476 | 2.17819 | 3.95365 | 3.41774 | 3.61498 | 3.83898 | 1.85574 | 3.17541 | 4.11754 |
| | 2.68618 | 4.42225 | 2.77519 | 2.73123 | 3.46354 | 2.40513 | 3.72494 | 3.29354 | 2.67741 | 2.69355 | 4.2469 |
| | 0.04727 | 4.25552 | 3.44244 | 0.61958 | 0.77255 | 0.48576 | 0.9551 | | | | |
| 16 | 2.48405 | 4.22073 | 3.7005 | 3.12776 | 2.55502 | 3.64194 | 3.27538 | 2.48512 | 3.05421 | 2.2444 | 3.32947 |
| | 2.68618 | 4.42225 | 2.77519 | 2.73123 | 3.46354 | 2.40513 | 3.72494 | 3.29354 | 2.67741 | 2.69355 | 4.2469 |
| | 0.02185 | 4.2301 | 4.95245 | 0.61958 | 0.77255 | 0.51883 | 0.90441 | | | | |
| 17 | 3.13366 | 3.92313 | 4.83811 | 4.25221 | 2.78383 | 4.35472 | 4.66344 | 2.16434 | 4.11395 | 0.96672 | 2.5582 |
| | 2.68618 | 4.42225 | 2.77519 | 2.73123 | 3.46354 | 2.40513 | 3.72494 | 3.29354 | 2.67741 | 2.69355 | 4.2469 |
| | 0.02185 | 4.2301 | 4.95245 | 0.61958 | 0.77255 | 0.51883 | 0.90441 | | | | |
| 18 | 2.71352 | 5.1651 | 2.71849 | 2.3519 | 4.49724 | 2.7822 | 3.63847 | 3.96317 | 1.91694 | 3.45632 | 4.22248 |
| | 2.68618 | 4.42225 | 2.77519 | 2.73123 | 3.46354 | 2.40513 | 3.72494 | 3.29354 | 2.67741 | 2.69355 | 4.2469 |
| | 0.02185 | 4.2301 | 4.95245 | 0.61958 | 0.77255 | 0.46563 | 0.98816 | | | | |
| 19 | 3.29145 | 4.5849 | 5.09144 | 4.5213 | 2.86124 | 4.60569 | 4.95187 | 1.22139 | 4.39349 | 1.40019 | 2.67523 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 20 | 2.5071 | 4.38003 | 3.4532 | 2.88775 | 3.52986 | 3.58488 | 3.61016 | 2.669 | 2.73495 | 2.5982 | 3.48719 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 21 | 2.62803 | 4.75161 | 3.07898 | 2.46141 | 3.97002 | 3.47631 | 3.30131 | 3.08914 | 2.40303 | 2.40822 | 3.82851 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 22 | 2.90861 | 5.22831 | 2.27588 | 2.37203 | 4.69297 | 3.33461 | 3.92914 | 4.19276 | 2.88341 | 3.76602 | 4.61584 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 23 | 2.20575 | 4.38127 | 3.4243 | 3.21249 | 4.49761 | 3.11767 | 4.35898 | 3.90077 | 3.31371 | 3.60277 | 4.45076 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 24 | 3.09358 | 5.68885 | 1.00725 | 2.21734 | 4.97028 | 3.34539 | 3.9055 | 4.52687 | 2.93739 | 4.0077 | 4.8622 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 25 | 2.33146 | 4.60859 | 2.75755 | 2.86722 | 4.63275 | 0.94201 | 4.25777 | 4.06508 | 3.27582 | 3.7369 | 4.58297 |
| | 2.68619 | 4.42226 | 2.7751 | 2.73124 | 3.46355 | 2.40514 | 3.72495 | 3.29355 | 2.67742 | 2.69356 | 4.24691 |
| | 0.07416 | 2.73969 | 4.97786 | 0.42151 | 1.06727 | 0.48576 | 0.9551 | | | | |
| 26 | 2.38454 | 4.33004 | 3.59565 | 3.28357 | 4.42041 | 3.11148 | 4.34501 | 3.7987 | 3.29928 | 3.51775 | 4.35848 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 27 | 3.03147 | 3.82107 | 4.83556 | 4.26047 | 2.92881 | 4.29441 | 4.66038 | 1.84664 | 4.11892 | 1.29519 | 3.17251 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 28 | 2.55623 | 3.34433 | 4.00897 | 3.51787 | 3.75796 | 3.44628 | 4.31812 | 2.50251 | 3.39914 | 2.70649 | 3.70826 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 29 | 2.93208 | 3.75576 | 3.62881 | 2.97162 | 4.3204 | 3.65869 | 3.81008 | 3.6711 | 2.13704 | 3.26094 | 4.14624 |
| | 2.68618 | 4.42225 | 2.77519 | 2.73123 | 3.46354 | 2.40513 | 3.72494 | 3.29354 | 2.67741 | 2.69355 | 4.2469 |
| | 0.04839 | 4.25552 | 3.40981 | 0.61958 | 0.77255 | 0.48576 | 0.9551 | | | | |
| 30 | 2.54708 | 4.90962 | 2.76214 | 2.41345 | 3.89732 | 3.43334 | 3.29521 | 3.6061 | 2.40981 | 2.90911 | 3.97281 |
| | 2.68618 | 4.42225 | 2.77519 | 2.73123 | 3.46354 | 2.40513 | 3.72494 | 3.29354 | 2.67741 | 2.69355 | 4.2469 |
| | 0.02188 | 4.22901 | 4.95136 | 0.61958 | 0.77255 | 0.5202 | 0.90239 | | | | |
| 31 | 2.60652 | 4.28625 | 3.55578 | 2.98681 | 3.1365 | 3.22339 | 3.86541 | 2.63665 | 2.93303 | 2.10958 | 3.39293 |
| | 2.68618 | 4.42225 | 2.77519 | 2.73123 | 3.46354 | 2.40513 | 3.72494 | 3.29354 | 2.67741 | 2.69355 | 4.2469 |
| | 0.02188 | 4.22901 | 4.95136 | 0.61958 | 0.77255 | 0.5202 | 0.90239 | | | | |
| 32 | 2.57655 | 4.18133 | 2.6128 | 2.24332 | 4.194 | 3.27508 | 3.62824 | 3.62945 | 2.21816 | 3.07846 | 3.68912 |
| | 2.68618 | 4.42225 | 2.77519 | 2.73123 | 3.46354 | 2.40513 | 3.72494 | 3.29354 | 2.67741 | 2.69355 | 4.2469 |
| | 0.05407 | 4.22901 | 3.26833 | 0.61958 | 0.77255 | 0.5202 | 0.90239 | | | | |
| 33 | 2.43549 | 4.1946 | 3.49837 | 3.13013 | 2.6266 | 3.62993 | 3.34687 | 2.40398 | 2.96131 | 2.1902 | 3.30392 |
| | 2.6862 | 4.42227 | 2.77521 | 2.73125 | 3.46318 | 2.40515 | 3.72496 | 3.29356 | 2.67743 | 2.69357 | 4.24692 |
| | 0.14273 | 2.07379 | 4.91987 | 0.31478 | 1.30915 | 0.44282 | 1.02785 | | | | |
| 34 | 2.57139 | 4.50456 | 3.38791 | 3.15943 | 4.24021 | 3.24832 | 4.26813 | 3.7144 | 3.16197 | 3.19789 | 4.34698 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 35 | 2.62406 | 4.0722 | 3.00641 | 2.46344 | 3.90714 | 3.48517 | 3.51516 | 3.1024 | 2.4237 | 2.603 | 3.78153 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 36 | 2.63863 | 4.21241 | 4.10815 | 3.53968 | 3.37574 | 3.82852 | 4.18783 | 2.15385 | 3.43691 | 1.94731 | 3.33898 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 37 | 2.44402 | 4.92605 | 2.82999 | 2.26477 | 4.19092 | 3.43704 | 3.65541 | 3.62256 | 2.44347 | 3.19924 | 3.99706 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 38 | 2.06142 | 4.68315 | 2.99137 | 2.57347 | 3.92558 | 3.46724 | 3.72229 | 3.17286 | 2.49658 | 2.75494 | 3.80199 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 39 | 2.43112 | 4.94294 | 2.73453 | 2.33796 | 4.24985 | 3.41808 | 3.65886 | 3.68913 | 2.44198 | 3.25309 | 4.04181 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 40 | 2.61022 | 5.0076 | 2.54438 | 2.20551 | 4.31413 | 2.92472 | 3.69421 | 3.45771 | 2.50376 | 3.31933 | 4.11535 |
| | 2.68613 | 4.42227 | 2.77512 | 2.73118 | 3.46356 | 2.40515 | 3.72497 | 3.29356 | 2.67743 | 2.69357 | 4.24692 |
| | 0.1859 | 1.98299 | 3.44244 | 0.28909 | 1.38209 | 0.48576 | 0.9551 | | | | |
| 41 | 2.50594 | 5.12907 | 2.16441 | 2.17619 | 4.44596 | 3.24587 | 3.63132 | 3.91774 | 2.40334 | 3.26819 | 4.18518 |
| | 2.68618 | 4.42225 | 2.77519 | 2.73123 | 3.46354 | 2.40513 | 3.72494 | 3.29354 | 2.67741 | 2.69355 | 4.2469 |
| | 0.04271 | 4.2301 | 3.6025 | 0.61958 | 0.77255 | 0.46563 | 0.98816 | | | | |
| 42 | 2.43273 | 4.45224 | 3.32672 | 2.67828 | 3.46154 | 3.19626 | 3.57269 | 2.88541 | 2.73136 | 2.37107 | 3.55001 |
| | 2.68618 | 4.42225 | 2.77519 | 2.73123 | 3.46354 | 2.40513 | 3.72494 | 3.29354 | 2.67741 | 2.69355 | 4.2469 |
| | 0.02173 | 4.23564 | 4.95798 | 0.61958 | 0.77255 | 0.51178 | 0.91485 | | | | |
| 43 | 2.53076 | 4.2533 | 2.78041 | 2.303 | 3.98666 | 3.46117 | 3.678 | 2.95489 | 2.49702 | 3.01573 | 3.84078 |
| | 2.68618 | 4.42225 | 2.77519 | 2.73123 | 3.46354 | 2.40513 | 3.72494 | 3.29354 | 2.67741 | 2.69355 | 4.2469 |
| | 0.14374 | 4.23564 | 2.12511 | 0.61958 | 0.77255 | 0.51178 | 0.91485 | | | | |
| 44 | 2.41809 | 4.60677 | 3.10443 | 2.57679 | 3.89136 | 3.18884 | 3.2356 | 3.2878 | 2.56095 | 2.69666 | 3.77607 |
| | 2.68618 | 4.42225 | 2.77519 | 2.73123 | 3.46354 | 2.40513 | 3.72494 | 3.29354 | 2.67741 | 2.69355 | 4.2469 |
| | 0.02452 | 4.11641 | 4.83876 | 0.61958 | 0.77255 | 0.64612 | 0.74249 | | | | |
| 45 | 2.46812 | 4.8969 | 2.63698 | 2.27415 | 4.17091 | 2.73607 | 3.61436 | 3.60494 | 2.39812 | 3.08692 | 3.97031 |
| | 2.68618 | 4.42225 | 2.77519 | 2.73123 | 3.46354 | 2.40513 | 3.72494 | 3.29354 | 2.67741 | 2.69355 | 4.2469 |
| | 0.04517 | 4.11641 | 3.58037 | 0.61958 | 0.77255 | 0.64612 | 0.74249 | | | | |
| 46 | 2.3075 | 4.66654 | 3.05527 | 2.40601 | 3.89215 | 3.21979 | 3.6661 | 3.03209 | 2.42765 | 2.7747 | 3.76439 |
| | 2.68602 | 4.42237 | 2.77515 | 2.73105 | 3.46366 | 2.40516 | 3.72507 | 3.29344 | 2.67748 | 2.69367 | 4.24702 |
| | 0.22448 | 1.64512 | 4.81861 | 1.22198 | 0.34906 | 0.51688 | 0.90728 | | | | |
| 47 | 2.49806 | 4.46973 | 3.28676 | 2.73345 | 3.68782 | 3.24592 | 3.7795 | 3.05723 | 2.69223 | 2.37261 | 3.61507 |
| | 2.68618 | 4.42225 | 2.77519 | 2.73123 | 3.46354 | 2.40513 | 3.72494 | 3.29354 | 2.67741 | 2.69355 | 4.2469 |
| | 0.02301 | 4.17918 | 4.90153 | 0.61958 | 0.77255 | 0.57965 | 0.8212 | | | | |
| 48 | 1.98571 | 4.23793 | 3.80646 | 3.27496 | 3.55955 | 3.53029 | 4.10193 | 2.42909 | 3.20483 | 2.56808 | 3.51392 |
| | 2.68618 | 4.42225 | 2.77519 | 2.73123 | 3.46354 | 2.40513 | 3.72494 | 3.29354 | 2.67741 | 2.69355 | 4.2469 |
| | 0.02301 | 4.17918 | 4.90153 | 0.61958 | 0.77255 | 0.53544 | 0.88047 | | | | |
| 49 | 2.52321 | 4.15057 | 3.86583 | 3.29091 | 2.86312 | 3.67168 | 3.42234 | 2.58852 | 3.19417 | 1.74174 | 3.2549 |
| | 2.68618 | 4.42225 | 2.77519 | 2.73123 | 3.46354 | 2.40513 | 3.72494 | 3.29354 | 2.67741 | 2.69355 | 4.2469 |
| | 0.0225 | 4.20135 | 4.92369 | 0.61958 | 0.77255 | 0.44534 | 1.02334 | | | | |
| 50 | 2.57415 | 3.70327 | 3.54281 | 2.99502 | 2.88557 | 3.50393 | 3.67525 | 2.93372 | 2.94353 | 2.64768 | 3.53896 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 51 | 3.03784 | 5.20192 | 3.34441 | 2.79178 | 4.67364 | 3.6687 | 3.73953 | 4.05042 | 1.01628 | 3.51995 | 4.37009 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 52 | 3.45489 | 5.58801 | 0.49459 | 2.66181 | 5.02904 | 3.55014 | 4.33669 | 4.71613 | 3.50291 | 4.26044 | 5.2638 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 53 | 3.2969 | 4.54806 | 5.17406 | 4.66171 | 3.64699 | 4.76175 | 5.30335 | 1.05901 | 4.56121 | 1.86412 | 3.42387 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 54 | 2.51523 | 4.42651 | 3.44965 | 2.99116 | 3.99447 | 3.31959 | 4.03957 | 2.9669 | 2.94654 | 3.03715 | 3.92065 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 55 | 3.09752 | 4.45738 | 4.74599 | 4.17112 | 3.20323 | 4.31658 | 4.61812 | 1.7523 | 4.02848 | 1.12071 | 3.09044 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 56 | 2.95093 | 5.36942 | 2.06163 | 2.30169 | 4.78524 | 3.33332 | 3.90261 | 4.31652 | 2.88264 | 3.84293 | 4.68099 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 57 | 2.49505 | 5.01318 | 2.73197 | 2.37818 | 4.30747 | 3.42483 | 3.31799 | 3.4409 | 2.18505 | 3.29665 | 4.07007 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 58 | 2.32785 | 5.06295 | 2.5553 | 2.14521 | 4.36867 | 3.41263 | 3.63946 | 3.82674 | 2.41158 | 3.24953 | 4.12734 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 59 | 2.58408 | 5.04757 | 2.91658 | 2.41742 | 4.36735 | 3.44485 | 3.28417 | 3.81115 | 1.88888 | 3.34431 | 4.1289 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 60 | 2.67551 | 5.11786 | 2.86099 | 2.2947 | 4.44275 | 2.77213 | 3.62687 | 3.90806 | 2.05061 | 3.41107 | 4.17233 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 61 | 2.6752 | 4.38485 | 3.94962 | 3.47431 | 3.78519 | 3.58218 | 4.36024 | 2.42683 | 3.36171 | 2.60602 | 3.69799 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 62 | 2.74023 | 4.27007 | 3.95316 | 3.39508 | 2.21757 | 3.24876 | 3.88109 | 2.74526 | 3.28785 | 2.45056 | 3.38906 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 63 | 2.47751 | 4.32335 | 4.57047 | 3.99043 | 2.93453 | 4.13362 | 4.46921 | 2.15166 | 3.86231 | 1.36854 | 2.88249 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 64 | 3.49583 | 5.27252 | 4.0307 | 3.48176 | 4.72839 | 3.88626 | 4.181 | 4.31306 | 2.49598 | 3.75907 | 4.76361 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 65 | 3.39897 | 4.66895 | 5.18289 | 4.6399 | 3.35134 | 4.77256 | 5.17956 | 1.40889 | 4.51103 | 1.06135 | 3.12718 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 66 | 4.03171 | 5.15176 | 4.9864 | 4.76441 | 1.03859 | 4.69029 | 3.58318 | 3.66248 | 4.58458 | 2.92483 | 4.23313 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 67 | 2.60001 | 4.01559 | 3.52525 | 2.94265 | 3.57277 | 3.62175 | 3.85913 | 2.87602 | 2.66258 | 1.95829 | 3.51841 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 68 | 3.34403 | 5.01806 | 3.99464 | 3.90067 | 4.83531 | 3.68485 | 4.93048 | 4.50058 | 3.99508 | 4.07601 | 5.15317 |
| | 2.68624 | 4.42231 | 2.77526 | 2.7313 | 3.4636 | 2.40519 | 3.72501 | 3.29343 | 2.67747 | 2.69361 | 4.24696 |
| | 0.10652 | 3.05267 | 2.92226 | 1.44843 | 0.2678 | 0.48576 | 0.9551 | | | | |
| 69 | 2.55117 | 4.7618 | 3.06314 | 2.50467 | 3.99427 | 3.46137 | 3.48497 | 3.40061 | 2.40879 | 2.92192 | 3.84451 |
| | 2.68618 | 4.42225 | 2.77519 | 2.73123 | 3.46354 | 2.40513 | 3.72494 | 3.29354 | 2.67741 | 2.69355 | 4.2469 |
| | 0.02236 | 4.20746 | 4.9298 | 0.61958 | 0.77255 | 0.49133 | 0.94627 | | | | |
| 70 | 2.56411 | 5.04965 | 2.70387 | 2.30316 | 4.35826 | 3.12173 | 3.6087 | 3.81895 | 2.16106 | 3.10089 | 4.10159 |
| | 2.68595 | 4.42197 | 2.77513 | 2.73135 | 3.46366 | 2.40524 | 3.72506 | 3.29366 | 2.67752 | 2.69325 | 4.24701 |
| | 0.30874 | 1.43126 | 3.62615 | 0.92349 | 0.50605 | 0.5132 | 0.91275 | | | | |
| 71 | 2.50555 | 4.72414 | 2.86941 | 2.47129 | 3.94827 | 3.46074 | 3.68229 | 3.08718 | 2.50999 | 2.68833 | 3.81165 |
| | 2.68618 | 4.42225 | 2.77519 | 2.73123 | 3.46354 | 2.40513 | 3.72494 | 3.29354 | 2.67741 | 2.69355 | 4.2469 |
| | 0.0222 | 4.21476 | 4.9371 | 0.61958 | 0.77255 | 0.49367 | 0.9426 | | | | |
| 72 | 2.47978 | 4.79346 | 2.92515 | 2.49358 | 4.11532 | 2.99681 | 3.69883 | 3.53503 | 2.50706 | 2.94697 | 3.94872 |
| | 2.68618 | 4.42225 | 2.77519 | 2.73123 | 3.46354 | 2.40513 | 3.72494 | 3.29354 | 2.67741 | 2.69355 | 4.2469 |
| | 0.02172 | 4.23615 | 4.9585 | 0.61958 | 0.77255 | 0.51112 | 0.91584 | | | | |
| 73 | 2.51652 | 5.04278 | 2.35508 | 2.35664 | 4.07333 | 3.01732 | 3.62311 | 3.81118 | 2.33308 | 3.33843 | 4.10612 |
| | 2.68618 | 4.42225 | 2.77519 | 2.73123 | 3.46354 | 2.40513 | 3.72494 | 3.29354 | 2.67741 | 2.69355 | 4.2469 |
| | 0.02172 | 4.23615 | 4.9585 | 0.61958 | 0.77255 | 0.51112 | 0.91584 | | | | |
| 74 | 2.4036 | 4.97197 | 2.94459 | 2.28813 | 4.25551 | 3.42854 | 3.08243 | 3.69814 | 2.33949 | 3.13083 | 4.03101 |
| | 2.68618 | 4.42225 | 2.7752 | 2.73123 | 3.46354 | 2.40509 | 3.72495 | 3.29354 | 2.67741 | 2.69355 | 4.2469 |
| | 0.07519 | 3.39218 | 3.24944 | 0.52674 | 0.89288 | 0.47022 | 0.98046 | | | | |
| 75 | 2.36557 | 5.00022 | 2.5047 | 2.29532 | 4.32719 | 3.39386 | 3.66439 | 3.77666 | 2.45668 | 3.32577 | 4.10981 |
| | 2.68618 | 4.42225 | 2.77519 | 2.73123 | 3.46354 | 2.40513 | 3.72494 | 3.29354 | 2.67741 | 2.69355 | 4.2469 |
| | 0.10324 | 4.22386 | 2.48352 | 0.61958 | 0.77255 | 0.52665 | 0.89301 | | | | |
| 76 | 2.37005 | 4.78587 | 2.89583 | 2.45451 | 4.07558 | 2.86288 | 3.65932 | 3.49383 | 2.30297 | 3.09654 | 3.90856 |
| | 2.68618 | 4.42225 | 2.77519 | 2.73123 | 3.46354 | 2.40513 | 3.72494 | 3.29354 | 2.67741 | 2.69355 | 4.2469 |
| | 0.0439 | 4.14445 | 3.60841 | 0.61958 | 0.77255 | 0.61749 | 0.775 | | | | |
| 77 | 2.2382 | 4.92392 | 2.92666 | 2.38657 | 4.20323 | 2.98808 | 3.42033 | 3.6382 | 2.19866 | 3.04102 | 3.99042 |
| | 2.68618 | 4.42225 | 2.77519 | 2.73123 | 3.46354 | 2.40513 | 3.72494 | 3.29354 | 2.67741 | 2.69355 | 4.2469 |
| | 0.02431 | 4.12486 | 4.84721 | 0.61958 | 0.77255 | 0.4013 | 1.107 | | | | |
| 78 | 3.19412 | 5.32193 | 3.68555 | 2.93841 | 4.78873 | 3.82072 | 3.70741 | 4.11892 | 1.10927 | 3.26775 | 4.40561 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 79 | 3.53779 | 4.88359 | 4.9722 | 4.50399 | 3.18088 | 4.67427 | 5.01813 | 2.25476 | 4.25136 | 0.62548 | 3.04227 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 80 | 3.34403 | 5.01806 | 3.99464 | 3.90067 | 4.83531 | 3.68485 | 4.93048 | 4.50058 | 3.99508 | 4.07601 | 5.15317 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 81 | 3.33414 | 4.60031 | 5.16347 | 4.62514 | 3.43761 | 4.73363 | 5.17762 | 1.37077 | 4.50893 | 1.24191 | 3.21741 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 82 | 3.292 | 4.54513 | 5.17125 | 4.65208 | 3.60993 | 4.74672 | 5.26682 | 1.17401 | 4.55049 | 1.71895 | 3.38924 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 83 | 3.28325 | 4.53586 | 5.16709 | 4.64982 | 3.63354 | 4.74272 | 5.27171 | 1.25881 | 4.55018 | 1.76836 | 3.41367 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 84 | 3.91195 | 5.12186 | 4.76206 | 4.46281 | 1.66798 | 4.58974 | 2.86357 | 3.73412 | 4.30503 | 3.04323 | 4.28664 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 85 | 3.24584 | 4.59746 | 4.64092 | 4.15596 | 1.14932 | 4.26725 | 3.8925 | 2.65965 | 4.00611 | 2.16645 | 3.48035 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 86 | 3.5141 | 5.23055 | 3.59545 | 3.41016 | 3.63357 | 3.83874 | 0.54184 | 4.27958 | 3.24679 | 3.71577 | 4.8026 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 87 | 2.48654 | 4.47887 | 3.1466 | 3.03623 | 4.63948 | 0.91669 | 4.34061 | 4.15339 | 3.33413 | 3.79889 | 4.62401 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 88 | 2.43732 | 4.38898 | 3.43238 | 3.3142 | 4.67367 | 0.85033 | 4.50634 | 4.18462 | 3.54341 | 3.85592 | 4.68173 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 89 | 2.48302 | 4.42572 | 3.42777 | 3.33446 | 4.68012 | 0.75761 | 4.53494 | 4.21053 | 3.57682 | 3.8896 | 4.73139 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 90 | 3.45123 | 4.74457 | 4.76377 | 4.34485 | 0.9128 | 4.38 | 3.72076 | 3.13876 | 4.17526 | 2.53937 | 3.15414 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 91 | 3.27809 | 4.52947 | 5.16227 | 4.66292 | 3.73986 | 4.75469 | 5.34698 | 1.28533 | 4.56683 | 2.11511 | 3.51654 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 92 | 3.31181 | 4.61513 | 5.09693 | 4.51309 | 2.42796 | 4.58582 | 4.89103 | 1.93601 | 4.37544 | 0.99721 | 2.6995 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 93 | 2.42489 | 2.89568 | 4.26787 | 3.67504 | 2.35307 | 3.75528 | 4.05709 | 2.42 | 3.52393 | 1.91889 | 3.16797 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 94 | 2.6689 | 4.76953 | 3.08531 | 2.65838 | 4.22485 | 3.39832 | 3.53907 | 3.67922 | 2.56446 | 3.27754 | 4.10936 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 95 | 1.24467 | 4.2906 | 3.79855 | 3.36088 | 3.92698 | 3.31343 | 4.27409 | 2.99526 | 3.31074 | 2.92265 | 3.86557 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 96 | 1.81174 | 4.97888 | 2.27992 | 2.35233 | 4.35005 | 3.38339 | 3.72643 | 3.79388 | 2.54816 | 3.35863 | 4.1546 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 97 | 2.50149 | 4.6684 | 3.00833 | 2.57649 | 3.68742 | 3.48823 | 3.33848 | 3.26916 | 2.56256 | 2.83978 | 3.38488 |
| | 2.68618 | 4.42225 | 2.77519 | 2.73123 | 3.46354 | 2.40513 | 3.72494 | 3.29354 | 2.67741 | 2.69355 | 4.2469 |
| | 0.04041 | 4.25552 | 3.67217 | 0.61958 | 0.77255 | 0.48576 | 0.9551 | | | | |
| 98 | 2.28347 | 4.59251 | 3.18671 | 2.62694 | 3.57625 | 3.07578 | 3.73009 | 3.16659 | 2.54968 | 2.58303 | 3.68484 |
| | 2.68618 | 4.42225 | 2.77519 | 2.73123 | 3.46354 | 2.40513 | 3.72494 | 3.29354 | 2.67741 | 2.69355 | 4.2469 |
| | 0.02171 | 4.23681 | 4.95916 | 0.61958 | 0.77255 | 0.51028 | 0.91711 | | | | |
| 99 | 2.46026 | 4.19767 | 3.73923 | 3.16479 | 3.01682 | 3.42545 | 3.92465 | 2.28309 | 3.09077 | 2.25911 | 2.84623 |
| | 2.68618 | 4.42225 | 2.77519 | 2.73123 | 3.46354 | 2.40513 | 3.72494 | 3.29354 | 2.67741 | 2.69355 | 4.2469 |
| | 0.02171 | 4.23681 | 4.95916 | 0.61958 | 0.77255 | 0.47073 | 0.97962 | | | | |
| 100 | 2.64539 | 3.49328 | 3.50742 | 3.34387 | 1.72824 | 3.7061 | 3.95929 | 2.55477 | 3.25032 | 2.36467 | 3.29097 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 101 | 2.92034 | 5.26854 | 2.37287 | 2.36847 | 4.40694 | 3.42588 | 1.40936 | 4.02235 | 2.63506 | 3.55809 | 4.39678 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 102 | 2.66189 | 5.14316 | 2.13318 | 2.13532 | 4.472 | 3.10674 | 3.61773 | 3.94939 | 2.17841 | 3.31547 | 4.19091 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 103 | 2.33684 | 3.91895 | 3.56873 | 3.00135 | 2.29386 | 3.60441 | 3.55768 | 2.7882 | 2.83109 | 2.44158 | 3.40552 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 104 | 3.10601 | 0.41162 | 4.68435 | 4.52332 | 4.45138 | 3.63217 | 5.08906 | 3.68031 | 4.36155 | 3.56769 | 4.70691 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 105 | 2.19908 | 4.81464 | 3.03062 | 2.39845 | 3.6726 | 3.30857 | 3.67017 | 3.47442 | 2.37197 | 3.0811 | 3.89602 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 106 | 2.31515 | 5.06582 | 2.54861 | 2.23118 | 4.37887 | 3.41569 | 3.60955 | 3.84293 | 2.30627 | 3.22652 | 4.11559 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 107 | 2.54452 | 4.1425 | 4.05777 | 3.47337 | 2.99581 | 3.75268 | 4.05102 | 2.30514 | 3.18757 | 2.02639 | 2.03451 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 108 | 0.94419 | 4.26429 | 3.70371 | 3.42811 | 4.51236 | 2.74569 | 4.46111 | 3.92542 | 3.4878 | 3.62397 | 4.43848 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 109 | 2.13381 | 4.996 | 2.60313 | 2.33449 | 4.29109 | 3.4252 | 3.63143 | 3.73807 | 2.24504 | 3.28346 | 4.06117 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 110 | 2.34793 | 5.13779 | 2.57319 | 1.98404 | 4.46817 | 3.40727 | 3.06308 | 3.94571 | 2.21015 | 3.43577 | 4.18445 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 111 | 2.4266 | 4.14415 | 4.24471 | 3.66152 | 2.94245 | 3.83706 | 4.16521 | 2.04202 | 3.53512 | 1.63873 | 3.22367 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 112 | 2.6543 | 4.86037 | 2.99217 | 2.41084 | 4.11197 | 3.3 | 3.51798 | 3.52965 | 2.46142 | 2.9452 | 3.94192 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 113 | 1.16524 | 4.29772 | 3.74505 | 3.27702 | 3.94663 | 3.29199 | 4.21077 | 3.09091 | 3.21612 | 2.83092 | 3.88199 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 114 | 3.29166 | 4.54773 | 5.16554 | 4.6417 | 3.57716 | 4.73506 | 5.2389 | 1.24662 | 4.53759 | 1.61105 | 3.35831 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 115 | 3.22756 | 4.52084 | 5.00047 | 4.48885 | 3.67836 | 4.61741 | 5.17302 | 1.67203 | 4.37376 | 2.17264 | 3.20592 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 116 | 2.14283 | 4.32426 | 4.43999 | 3.87926 | 3.22525 | 4.1091 | 4.49504 | 2.0212 | 3.77574 | 1.90406 | 3.34717 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 117 | 2.55096 | 4.46721 | 3.59442 | 3.44696 | 4.49053 | 3.19268 | 4.53558 | 4.07531 | 3.54843 | 3.77096 | 4.67466 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 118 | 2.66485 | 4.31652 | 4.17925 | 3.64964 | 3.49325 | 3.8828 | 4.35877 | 2.25778 | 3.53449 | 1.91036 | 3.41621 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 119 | 2.92591 | 5.47948 | 1.92947 | 1.30203 | 4.7768 | 3.36561 | 3.44454 | 4.2859 | 2.70549 | 3.77178 | 4.5739 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 120 | 3.84785 | 5.17916 | 4.42837 | 4.24429 | 2.41835 | 4.29566 | 3.86875 | 3.81079 | 4.093 | 3.1612 | 4.4472 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 121 | 3.49583 | 5.27252 | 4.0307 | 3.48176 | 4.72839 | 3.88626 | 4.181 | 4.31306 | 2.49598 | 3.75907 | 4.76361 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 122 | 3.05693 | 4.63825 | 4.01003 | 3.65419 | 3.34838 | 3.91996 | 4.36755 | 2.68232 | 3.43279 | 0.82632 | 3.47935 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 123 | 0.78484 | 3.63503 | 4.02275 | 3.66567 | 4.30301 | 3.09883 | 4.51946 | 3.5561 | 3.5848 | 3.3719 | 4.25341 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 124 | 3.34403 | 5.01806 | 3.99464 | 3.90067 | 4.83531 | 3.68485 | 4.93048 | 4.50058 | 3.99508 | 4.07601 | 5.15317 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 125 | 3.01119 | 5.30786 | 2.5308 | 0.89517 | 4.7118 | 3.40936 | 3.95419 | 4.09498 | 2.83177 | 3.71332 | 4.62081 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 126 | 2.87112 | 5.22503 | 2.55133 | 2.38702 | 4.46183 | 3.44072 | 1.60056 | 3.97854 | 2.36584 | 3.50913 | 4.32976 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 127 | 3.3081 | 5.33152 | 3.78728 | 3.07699 | 4.83307 | 3.86274 | 3.77499 | 4.17432 | 1.79447 | 3.59147 | 4.49111 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 128 | 3.6167 | 4.975 | 4.80486 | 4.46322 | 3.33052 | 4.46513 | 5.00174 | 2.57039 | 4.22888 | 0.51488 | 3.26288 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 129 | 3.34403 | 5.01806 | 3.99464 | 3.90067 | 4.83531 | 3.68485 | 4.93048 | 4.50058 | 3.99508 | 4.07601 | 5.15317 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 130 | 0.57778 | 4.43246 | 3.90078 | 3.73115 | 4.4826 | 3.24313 | 4.70044 | 3.66968 | 3.75883 | 3.5317 | 4.534 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 131 | 1.09664 | 4.46203 | 3.48594 | 3.0602 | 3.91557 | 3.39018 | 4.07905 | 3.17677 | 2.95982 | 2.89608 | 3.4621 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 132 | 3.94642 | 5.14309 | 4.81231 | 4.55796 | 1.84154 | 4.60234 | 3.60923 | 3.63287 | 4.39292 | 2.92259 | 4.22715 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 133 | 3.00215 | 5.60916 | 1.37851 | 1.56534 | 4.89385 | 3.35789 | 3.49917 | 4.41969 | 2.78481 | 3.8899 | 4.70611 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 134 | 3.45489 | 5.58801 | 0.49459 | 2.66181 | 5.02904 | 3.55014 | 4.33669 | 4.71613 | 3.50291 | 4.26044 | 5.2638 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 135 | 1.15958 | 3.63692 | 3.8944 | 3.46324 | 4.20666 | 1.97236 | 4.36614 | 3.56413 | 3.41022 | 3.29846 | 4.14315 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 136 | 2.28315 | 4.21242 | 3.75361 | 2.9764 | 3.23681 | 3.66541 | 3.94331 | 2.56676 | 3.10337 | 2.10265 | 2.95782 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 137 | 2.72163 | 5.47373 | 1.79923 | 1.35158 | 4.7792 | 3.36708 | 3.78704 | 4.28314 | 2.68891 | 3.76711 | 4.56605 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 138 | 0.79209 | 4.25149 | 3.78593 | 3.53079 | 4.46614 | 3.06674 | 4.51555 | 3.75432 | 3.54784 | 3.55222 | 4.40622 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 139 | 3.33894 | 4.60488 | 5.16628 | 4.62801 | 3.43382 | 4.73808 | 5.18085 | 1.34876 | 4.51121 | 1.24049 | 3.21303 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 140 | 2.53982 | 4.86299 | 2.93834 | 2.35311 | 4.11108 | 3.45389 | 2.91627 | 3.53257 | 2.23139 | 2.51832 | 3.36704 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 141 | 3.14158 | 3.9883 | 4.43671 | 3.94113 | 2.43765 | 4.10081 | 3.88801 | 2.89559 | 3.74377 | 2.29885 | 3.57298 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 142 | 2.6268 | 4.42605 | 4.89134 | 4.36111 | 3.66987 | 4.48221 | 4.98904 | 1.32971 | 4.25959 | 2.08651 | 3.49478 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 143 | 3.25241 | 5.39101 | 3.71565 | 2.95953 | 4.91102 | 3.84901 | 3.70354 | 4.22048 | 1.39736 | 3.60124 | 4.46615 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 144 | 2.609 | 5.15173 | 2.2255 | 2.1938 | 4.4729 | 3.40238 | 3.63574 | 3.94845 | 2.31029 | 3.44779 | 4.2053 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 145 | 2.65663 | 5.02967 | 2.89993 | 2.27601 | 4.32765 | 3.06402 | 3.26627 | 3.60889 | 2.38934 | 3.31486 | 4.09139 |
| | 2.68618 | 4.42225 | 2.77519 | 2.73123 | 3.46354 | 2.40513 | 3.72494 | 3.29354 | 2.67741 | 2.69355 | 4.2469 |
| | 0.20749 | 4.25552 | 1.75337 | 0.61958 | 0.77255 | 0.48576 | 0.9551 | | | | |
| 146 | 1.81814 | 4.2716 | 3.53266 | 2.9675 | 3.38925 | 3.56426 | 3.84427 | 2.73772 | 2.88407 | 2.44525 | 3.18321 |
| | 2.68618 | 4.42225 | 2.77519 | 2.73123 | 3.46354 | 2.40513 | 3.72494 | 3.29354 | 2.67741 | 2.69355 | 4.2469 |
| | 0.06346 | 4.07363 | 3.11296 | 0.61958 | 0.77255 | 0.68685 | 0.69948 | | | | |
| 147 | 2.25203 | 4.55727 | 2.93689 | 2.5659 | 3.74719 | 3.45084 | 3.67538 | 3.13232 | 2.54399 | 2.49491 | 3.18094 |
| | 2.68601 | 4.4222 | 2.7748 | 2.73117 | 3.46379 | 2.40501 | 3.72519 | 3.2933 | 2.6776 | 2.6938 | 4.24686 |
| | 0.53954 | 0.89548 | 4.7591 | 1.2896 | 0.32211 | 0.36328 | 1.18872 | | | | |
| 148 | 2.65415 | 3.18473 | 2.60629 | 2.32833 | 4.19207 | 2.21188 | 3.68897 | 3.62047 | 2.40697 | 3.20657 | 4.01202 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 149 | 2.95227 | 5.5143 | 1.51524 | 1.48816 | 4.81612 | 3.37033 | 3.80443 | 4.32435 | 2.7102 | 3.80498 | 4.61201 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 150 | 2.64631 | 5.20955 | 2.41236 | 1.91823 | 4.52272 | 3.39858 | 3.67522 | 3.99815 | 2.37496 | 3.50144 | 4.2719 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 151 | 3.2118 | 4.72114 | 4.38042 | 3.9584 | 2.73383 | 4.02996 | 4.03427 | 3.0267 | 3.65069 | 2.50896 | 3.74225 |
| | 2.68618 | 4.42225 | 2.77519 | 2.73123 | 3.46354 | 2.40513 | 3.72494 | 3.29354 | 2.67741 | 2.69355 | 4.2469 |
| | 0.06553 | 4.25552 | 3.01102 | 0.61958 | 0.77255 | 0.48576 | 0.9551 | | | | |
| 152 | 3.35631 | 4.65203 | 5.06637 | 4.50452 | 3.00735 | 4.66836 | 4.99974 | 1.87686 | 4.38618 | 0.91426 | 3.01592 |
| | 2.68618 | 4.42225 | 2.77519 | 2.73123 | 3.46354 | 2.40513 | 3.72494 | 3.29354 | 2.67741 | 2.69355 | 4.2469 |
| | 0.02225 | 4.21224 | 4.93459 | 0.61958 | 0.77255 | 0.54094 | 0.87276 | | | | |
| 153 | 2.57213 | 5.02638 | 2.98342 | 2.34543 | 4.3417 | 3.45414 | 3.62375 | 3.77932 | 1.801 | 3.30831 | 4.09133 |
| | 2.68618 | 4.42225 | 2.77519 | 2.73123 | 3.46354 | 2.40513 | 3.72494 | 3.29354 | 2.67741 | 2.69355 | 4.2469 |
| | 0.02225 | 4.21224 | 4.93459 | 0.61958 | 0.77255 | 0.54094 | 0.87276 | | | | |
| 154 | 2.62432 | 5.28573 | 2.00127 | 1.76343 | 4.60388 | 3.37014 | 3.6823 | 4.09412 | 2.37067 | 3.57989 | 4.34911 |
| | 2.68618 | 4.42225 | 2.77519 | 2.73123 | 3.46354 | 2.40513 | 3.72494 | 3.29354 | 2.67741 | 2.69355 | 4.2469 |
| | 0.02225 | 4.21224 | 4.93459 | 0.61958 | 0.77255 | 0.54094 | 0.87276 | | | | |
| 155 | 2.81824 | 4.46049 | 3.7236 | 3.17906 | 2.60538 | 3.46919 | 2.83604 | 2.9695 | 3.09007 | 2.34936 | 3.57361 |
| | 2.68618 | 4.42225 | 2.77519 | 2.73123 | 3.46354 | 2.40513 | 3.72494 | 3.29354 | 2.67741 | 2.69355 | 4.2469 |
| | 0.02225 | 4.21224 | 4.93459 | 0.61958 | 0.77255 | 0.54094 | 0.87276 | | | | |
| 156 | 1.47609 | 4.28772 | 3.67705 | 3.2009 | 3.82281 | 2.50801 | 4.13442 | 2.97689 | 3.15373 | 2.71017 | 3.76755 |
| | 2.68618 | 4.42225 | 2.77519 | 2.73123 | 3.46354 | 2.40513 | 3.72494 | 3.29354 | 2.67741 | 2.69355 | 4.2469 |
| | 0.02225 | 4.21224 | 4.93459 | 0.61958 | 0.77255 | 0.54094 | 0.87276 | | | | |
| 157 | 3.39607 | 5.53382 | 0.53169 | 2.61585 | 4.96615 | 3.50591 | 4.28354 | 4.64066 | 3.43962 | 4.19216 | 5.19108 |
| | 2.68618 | 4.42225 | 2.77519 | 2.73123 | 3.46354 | 2.40513 | 3.72494 | 3.29354 | 2.67741 | 2.69355 | 4.2469 |
| | 0.02225 | 4.21224 | 4.93459 | 0.61958 | 0.77255 | 0.54094 | 0.87276 | | | | |
| 158 | 2.59342 | 4.18619 | 4.09052 | 3.51858 | 1.90243 | 3.78729 | 4.00775 | 2.55852 | 3.40225 | 1.86364 | 3.22649 |
| | 2.68618 | 4.42225 | 2.77519 | 2.73123 | 3.46354 | 2.40513 | 3.72494 | 3.29354 | 2.67741 | 2.69355 | 4.2469 |
| | 0.02225 | 4.21224 | 4.93459 | 0.61958 | 0.77255 | 0.54094 | 0.87276 | | | | |
| 159 | 2.49675 | 4.49787 | 3.13901 | 2.95607 | 4.43779 | 3.14537 | 4.20364 | 3.98887 | 3.13126 | 3.63856 | 4.47861 |
| | 2.68618 | 4.42225 | 2.77519 | 2.73123 | 3.46354 | 2.40513 | 3.72494 | 3.29354 | 2.67741 | 2.69355 | 4.2469 |
| | 0.02225 | 4.21224 | 4.93459 | 0.61958 | 0.77255 | 0.54094 | 0.87276 | | | | |
| 160 | 2.8841 | 5.20295 | 3.14784 | 2.51492 | 4.59649 | 3.57921 | 3.64956 | 4.00008 | 1.65277 | 3.46379 | 4.2695 |
| | 2.68618 | 4.42225 | 2.77519 | 2.73123 | 3.46354 | 2.40513 | 3.72494 | 3.29354 | 2.67741 | 2.69355 | 4.2469 |
| | 0.02225 | 4.21224 | 4.93459 | 0.61958 | 0.77255 | 0.54094 | 0.87276 | | | | |
| 161 | 2.6219 | 1.93898 | 4.27871 | 3.76034 | 3.51947 | 3.62944 | 4.35392 | 2.36237 | 3.62209 | 2.39602 | 3.48141 |
| | 2.68618 | 4.42225 | 2.77519 | 2.73123 | 3.46354 | 2.40513 | 3.72494 | 3.29354 | 2.67741 | 2.69355 | 4.2469 |
| | 0.02225 | 4.21224 | 4.93459 | 0.61958 | 0.77255 | 0.45274 | 1.01028 | | | | |
| 162 | 3.62928 | 4.89339 | 4.80118 | 4.44183 | 0.97228 | 4.47872 | 3.68242 | 3.26598 | 4.27962 | 2.65017 | 3.8916 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 163 | 3.38998 | 4.66903 | 5.16045 | 4.59769 | 3.27725 | 4.73483 | 5.09818 | 1.68059 | 4.47497 | 0.96503 | 2.80972 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 164 | 2.87451 | 3.82956 | 4.3442 | 3.76954 | 3.20791 | 3.99597 | 4.30617 | 2.22984 | 3.62126 | 1.98337 | 1.40906 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 165 | 3.21325 | 4.93276 | 3.96373 | 3.91957 | 5.02096 | 0.32494 | 4.99846 | 4.73153 | 4.15183 | 4.33001 | 5.32112 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 166 | 2.599 | 3.61046 | 3.07338 | 2.69714 | 3.77099 | 3.18004 | 3.77854 | 3.05056 | 2.67549 | 2.82596 | 3.3173 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 167 | 2.73773 | 4.90439 | 2.63189 | 2.52675 | 4.27273 | 3.36026 | 3.2046 | 3.83506 | 2.74112 | 3.42567 | 4.26347 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 168 | 1.1864 | 4.42374 | 3.36272 | 3.00088 | 4.34072 | 3.17173 | 4.14428 | 3.74875 | 3.06264 | 3.41264 | 4.23486 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 169 | 3.21325 | 4.93276 | 3.96373 | 3.91957 | 5.02096 | 0.32494 | 4.99846 | 4.73153 | 4.15183 | 4.33001 | 5.32112 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 170 | 1.42474 | 4.31808 | 3.64137 | 3.4459 | 4.60884 | 1.14769 | 4.5307 | 4.00013 | 3.57302 | 3.7257 | 4.55933 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 171 | 2.67644 | 4.75042 | 2.92595 | 2.72735 | 4.45964 | 3.28645 | 4.05372 | 3.89194 | 2.9131 | 3.56299 | 4.42685 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 172 | 3.36706 | 4.64594 | 5.13029 | 4.57809 | 3.33733 | 4.72482 | 5.10688 | 1.07176 | 4.44057 | 1.46644 | 2.60671 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 173 | 1.21161 | 4.2581 | 3.83859 | 3.39579 | 3.97834 | 3.25475 | 4.29246 | 3.10257 | 3.34173 | 3.01087 | 3.91839 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 174 | 3.7362 | 4.96075 | 4.86903 | 4.53057 | 1.47683 | 4.55341 | 3.65638 | 3.34755 | 4.35966 | 2.21297 | 3.94457 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 175 | 2.6582 | 4.88376 | 3.02439 | 2.47396 | 3.34774 | 3.47113 | 2.5466 | 3.55648 | 2.43588 | 3.14092 | 3.95352 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 176 | 1.68939 | 4.30246 | 3.97245 | 3.4616 | 3.68491 | 3.6115 | 4.28948 | 2.46435 | 3.38214 | 2.48409 | 3.61002 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 177 | 1.47593 | 4.35612 | 3.42796 | 3.17287 | 4.52111 | 1.38586 | 4.32882 | 3.95721 | 3.31242 | 3.62019 | 4.43073 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 178 | 3.51212 | 4.80626 | 5.15544 | 4.58484 | 3.17208 | 4.80452 | 5.10508 | 2.16226 | 4.44142 | 0.73151 | 2.49265 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 179 | 2.53136 | 4.85113 | 3.15726 | 2.57206 | 4.09761 | 3.5279 | 3.47496 | 3.49952 | 2.26118 | 2.98281 | 3.59713 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 180 | 1.55978 | 4.30143 | 3.68081 | 3.15647 | 3.69725 | 3.43196 | 4.06403 | 2.89411 | 3.09857 | 2.73744 | 3.38369 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 181 | 1.92279 | 3.87083 | 3.73426 | 3.17374 | 3.41056 | 3.31117 | 3.96755 | 2.72845 | 3.09618 | 1.82663 | 3.39405 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 182 | 2.60049 | 5.08818 | 2.06666 | 2.0011 | 4.39685 | 3.21005 | 3.63204 | 3.85978 | 2.39913 | 3.25905 | 3.71619 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 183 | 2.22137 | 4.44866 | 3.19998 | 2.7811 | 3.605 | 3.55543 | 3.24533 | 2.78385 | 2.74656 | 2.14455 | 3.54717 |
| | 2.68614 | 4.42231 | 2.77504 | 2.73122 | 3.4636 | 2.40512 | 3.72481 | 3.2936 | 2.67747 | 2.69361 | 4.24696 |
| | 0.2552 | 1.7373 | 3.01102 | 0.66958 | 0.71729 | 0.48576 | 0.9551 | | | | |
| 184 | 3.07385 | 5.68653 | 1.03444 | 1.94309 | 4.9656 | 3.32234 | 3.87645 | 4.49781 | 2.91082 | 3.98007 | 4.83283 |
| | 2.68618 | 4.42225 | 2.77519 | 2.73123 | 3.46354 | 2.40513 | 3.72494 | 3.29354 | 2.67741 | 2.69355 | 4.2469 |
| | 0.02225 | 4.21224 | 4.93459 | 0.61958 | 0.77255 | 0.54094 | 0.87276 | | | | |
| 185 | 3.04101 | 4.43494 | 4.58213 | 4.00585 | 2.96286 | 4.23225 | 4.55171 | 2.17464 | 3.88822 | 1.07497 | 2.89473 |
| | 2.68618 | 4.42225 | 2.77519 | 2.73123 | 3.46354 | 2.40513 | 3.72494 | 3.29354 | 2.67741 | 2.69355 | 4.2469 |
| | 0.02225 | 4.21224 | 4.93459 | 0.61958 | 0.77255 | 0.54094 | 0.87276 | | | | |
| 186 | 2.69084 | 5.16486 | 2.48287 | 1.97237 | 4.48364 | 2.92995 | 3.43767 | 3.9601 | 2.21919 | 3.45956 | 4.22139 |
| | 2.68618 | 4.42225 | 2.77519 | 2.73123 | 3.46354 | 2.40513 | 3.72494 | 3.29354 | 2.67741 | 2.69355 | 4.2469 |
| | 0.02225 | 4.21224 | 4.93459 | 0.61958 | 0.77255 | 0.45274 | 1.01028 | | | | |
| 187 | 2.37009 | 4.63835 | 3.06685 | 2.7739 | 4.37155 | 2.81692 | 4.01156 | 3.78019 | 2.60484 | 3.41563 | 4.2528 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 188 | 2.8598 | 4.42464 | 4.84922 | 4.27933 | 3.33456 | 4.37237 | 4.74656 | 1.70815 | 4.14979 | 1.40021 | 2.51879 |
| | 2.68618 | 4.42225 | 2.77519 | 2.73123 | 3.46354 | 2.40513 | 3.72494 | 3.29354 | 2.67741 | 2.69355 | 4.2469 |
| | 0.05262 | 4.25552 | 3.29484 | 0.61958 | 0.77255 | 0.48576 | 0.9551 | | | | |
| 189 | 2.679 | 4.4764 | 3.02533 | 2.26362 | 4.27448 | 3.47031 | 3.63103 | 3.44239 | 1.70468 | 3.10517 | 4.04335 |
| | 2.68618 | 4.42225 | 2.77519 | 2.73123 | 3.46354 | 2.40513 | 3.72494 | 3.29354 | 2.67741 | 2.69355 | 4.2469 |
| | 0.02197 | 4.22487 | 4.94721 | 0.61958 | 0.77255 | 0.46175 | 0.99473 | | | | |
| 190 | 3.3305 | 4.59281 | 5.1498 | 4.63453 | 3.55994 | 4.75878 | 5.26513 | 0.946 | 4.51485 | 1.73726 | 3.34238 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 191 | 2.50701 | 4.89444 | 2.89753 | 2.44443 | 4.15319 | 3.45239 | 3.64741 | 3.05723 | 2.2654 | 3.05665 | 3.96092 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 192 | 3.21325 | 4.93276 | 3.96373 | 3.91957 | 5.02096 | 0.32494 | 4.99846 | 4.73153 | 4.15183 | 4.33001 | 5.32112 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 193 | 3.12638 | 3.86135 | 4.81627 | 4.2433 | 3.27324 | 4.37152 | 4.72982 | 2.0895 | 4.11109 | 1.0071 | 2.82613 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 194 | 3.0038 | 4.36972 | 4.66061 | 4.12512 | 3.58144 | 3.67296 | 4.73573 | 1.13957 | 4.00155 | 2.18188 | 3.45746 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 195 | 3.52178 | 4.80496 | 5.21863 | 4.63827 | 2.89178 | 4.81139 | 5.07413 | 2.12954 | 4.51017 | 0.79194 | 2.14929 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 196 | 2.65124 | 4.97391 | 2.76308 | 2.40569 | 4.23984 | 3.43825 | 2.85334 | 3.21521 | 2.40605 | 3.24722 | 4.03573 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 197 | 2.63916 | 4.89772 | 3.08076 | 2.56258 | 4.09886 | 3.51087 | 3.07763 | 3.37711 | 2.40665 | 3.14796 | 3.9932 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 198 | 2.42207 | 4.46056 | 3.52606 | 3.21767 | 4.11878 | 3.29998 | 4.27039 | 3.06106 | 3.17028 | 3.10701 | 4.11363 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 199 | 3.02795 | 4.47618 | 4.25522 | 3.73944 | 1.39929 | 4.01864 | 3.12192 | 2.96004 | 3.61816 | 2.457 | 3.26936 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 200 | 3.58253 | 4.88367 | 4.91148 | 4.50196 | 0.97239 | 4.57554 | 4.18077 | 2.69128 | 4.33385 | 1.60163 | 3.3383 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 201 | 2.36072 | 4.3236 | 3.51036 | 3.28723 | 4.60346 | 1.11963 | 4.42077 | 4.04762 | 3.41991 | 3.71756 | 4.52365 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 202 | 2.39294 | 4.48907 | 3.5425 | 3.45586 | 4.72578 | 0.65686 | 4.62656 | 4.18515 | 3.67885 | 3.9002 | 4.78688 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 203 | 2.46696 | 4.3893 | 3.43591 | 2.57615 | 3.54102 | 3.58601 | 3.83757 | 2.77171 | 2.72898 | 2.50837 | 3.3286 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 204 | 2.51844 | 5.16458 | 2.57081 | 1.77211 | 4.49008 | 3.40716 | 3.08068 | 3.9664 | 2.14849 | 3.45936 | 4.2153 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 205 | 2.96185 | 5.12312 | 3.2279 | 2.53337 | 4.55724 | 3.61344 | 3.74682 | 3.94072 | 2.1849 | 3.4487 | 4.2943 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 206 | 2.45604 | 3.85306 | 3.70865 | 3.21226 | 3.90372 | 3.28954 | 4.14552 | 2.91497 | 3.14302 | 2.96121 | 3.85001 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 207 | 2.49946 | 5.15602 | 2.56064 | 1.83876 | 4.48321 | 3.04861 | 3.62648 | 3.96107 | 2.26932 | 3.45302 | 4.20576 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 208 | 1.90602 | 4.30156 | 3.63579 | 3.40197 | 4.54129 | 3.06744 | 4.46933 | 3.93437 | 3.47177 | 3.663 | 4.49961 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 209 | 3.14356 | 5.5952 | 2.25011 | 0.81135 | 4.9211 | 3.40191 | 3.97367 | 4.43712 | 2.96883 | 3.9694 | 4.85339 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 210 | 2.37718 | 4.16524 | 3.96337 | 3.38462 | 3.28408 | 3.7344 | 4.03432 | 2.07666 | 3.00165 | 1.80389 | 3.12199 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 211 | 2.66507 | 5.21127 | 3.24607 | 2.55853 | 4.61169 | 3.63561 | 3.68164 | 3.99649 | 1.81126 | 3.46672 | 4.28808 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 212 | 2.63955 | 4.34791 | 3.5221 | 2.96571 | 3.25704 | 3.61127 | 3.85879 | 2.84249 | 2.89256 | 1.65315 | 3.4542 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 213 | 2.22286 | 4.37073 | 3.44616 | 2.54278 | 3.33023 | 3.25665 | 3.83303 | 2.568 | 2.74422 | 2.58376 | 3.06763 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 214 | 2.58034 | 5.27089 | 1.92951 | 2.25116 | 4.61496 | 3.37067 | 3.75818 | 4.0935 | 2.62474 | 3.60964 | 4.40214 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 215 | 3.14939 | 5.49467 | 0.76444 | 2.4005 | 5.02233 | 2.66996 | 4.09716 | 4.6021 | 3.203 | 4.12792 | 5.0223 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 216 | 2.4882 | 5.01531 | 2.94405 | 2.39158 | 4.31308 | 3.05376 | 3.31424 | 3.76108 | 2.30985 | 3.29748 | 4.07233 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 217 | 2.62023 | 4.17052 | 3.85283 | 3.27554 | 3.28797 | 3.67909 | 3.56574 | 2.16353 | 3.17431 | 2.36821 | 3.08873 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 218 | 3.01014 | 3.37642 | 4.75985 | 4.17116 | 2.87029 | 4.22161 | 4.54719 | 2.1197 | 4.02208 | 1.08314 | 2.73819 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 219 | 2.56239 | 4.49247 | 3.38583 | 3.07732 | 4.11907 | 3.28084 | 4.16133 | 3.54676 | 3.04325 | 2.87578 | 4.17089 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 220 | 2.6177 | 4.361 | 3.51255 | 3.00735 | 3.56971 | 3.53493 | 3.93577 | 2.90704 | 2.93536 | 1.51918 | 3.54787 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 221 | 2.352 | 3.4778 | 3.32874 | 2.65448 | 3.62786 | 3.54475 | 3.57969 | 2.66373 | 2.73498 | 2.69126 | 3.56621 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 222 | 1.88353 | 4.27599 | 3.5753 | 3.01584 | 3.44578 | 3.58952 | 3.88616 | 2.6495 | 2.95645 | 2.44986 | 3.41792 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 223 | 2.60199 | 4.1717 | 3.2724 | 2.7169 | 3.74804 | 3.20409 | 3.78685 | 2.75611 | 2.69063 | 2.70202 | 3.6666 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 224 | 3.13751 | 5.60334 | 0.85777 | 2.30642 | 4.69686 | 3.38916 | 3.15717 | 4.47109 | 3.00456 | 3.96566 | 4.85469 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 225 | 2.57839 | 4.19752 | 3.9564 | 3.37554 | 3.10269 | 3.74858 | 4.03631 | 2.54713 | 3.11039 | 1.54297 | 3.01766 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 226 | 2.83801 | 3.502 | 4.42801 | 3.8439 | 3.05406 | 3.98561 | 4.31581 | 2.36567 | 3.70484 | 1.56202 | 1.78511 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 227 | 3.26391 | 4.77044 | 4.39688 | 3.99452 | 2.70417 | 4.04528 | 4.02134 | 3.11204 | 3.6702 | 2.56627 | 3.80967 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 228 | 2.49314 | 5.24256 | 2.22698 | 1.83454 | 4.5628 | 3.39193 | 3.67321 | 4.04724 | 2.28451 | 3.53789 | 4.3018 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 229 | 3.04748 | 4.5113 | 3.8273 | 3.78287 | 2.98645 | 4.15599 | 4.4144 | 2.27082 | 3.68251 | 0.95084 | 3.15372 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 230 | 1.28917 | 3.34957 | 3.88699 | 3.43097 | 4.10644 | 3.14885 | 4.32107 | 3.43592 | 3.37015 | 3.18956 | 4.04758 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 231 | 3.6167 | 4.975 | 4.80486 | 4.46322 | 3.33052 | 4.46513 | 5.00174 | 2.57039 | 4.22888 | 0.51488 | 3.26288 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 232 | 3.34403 | 5.01806 | 3.99464 | 3.90067 | 4.83531 | 3.68485 | 4.93048 | 4.50058 | 3.99508 | 4.07601 | 5.15317 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 233 | 2.63713 | 4.55028 | 2.95478 | 2.30553 | 3.72673 | 3.53787 | 3.77189 | 2.67286 | 2.58802 | 2.57738 | 3.6445 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 234 | 2.61909 | 4.65694 | 2.69579 | 2.81048 | 4.65735 | 0.91836 | 4.24118 | 4.15384 | 3.2676 | 3.79429 | 4.6376 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 235 | 1.7832 | 3.74573 | 3.89601 | 3.3887 | 3.71468 | 3.35467 | 4.19431 | 2.91873 | 3.31614 | 2.77255 | 3.67982 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 236 | 3.05465 | 5.54253 | 0.92516 | 2.25842 | 4.93194 | 3.32878 | 3.94719 | 4.46136 | 3.00238 | 3.99095 | 4.85699 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 237 | 3.12369 | 5.18896 | 3.56345 | 2.94224 | 4.64329 | 3.74495 | 3.7627 | 4.00967 | 2.05338 | 3.47496 | 4.36413 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 238 | 3.0673 | 5.53935 | 0.86896 | 2.26452 | 4.94767 | 3.3187 | 3.97556 | 4.52216 | 3.06249 | 4.04571 | 4.92102 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 239 | 3.5141 | 5.23055 | 3.59545 | 3.41016 | 3.63357 | 3.83874 | 0.54184 | 4.27958 | 3.24679 | 3.71577 | 4.8026 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 240 | 2.72756 | 4.97101 | 2.89474 | 1.46299 | 4.27237 | 3.26146 | 3.71762 | 3.67126 | 2.47081 | 3.26879 | 4.08933 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 241 | 3.41624 | 3.9134 | 4.58391 | 4.16764 | 2.00719 | 4.28964 | 3.70426 | 3.21172 | 4.01677 | 2.71346 | 3.87556 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 242 | 2.34081 | 1.8066 | 3.94181 | 3.45762 | 3.20828 | 3.32229 | 4.2184 | 3.00879 | 3.37351 | 2.78978 | 3.70097 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 243 | 3.01998 | 5.30594 | 2.30384 | 2.42731 | 4.70328 | 3.36933 | 4.01449 | 4.32714 | 3.00906 | 3.89198 | 4.77407 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 244 | 2.85299 | 4.67395 | 3.63506 | 3.3271 | 3.95006 | 3.53174 | 4.31756 | 3.34609 | 3.2219 | 2.4177 | 4.09253 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 245 | 2.63872 | 4.26914 | 3.6729 | 2.66729 | 3.38371 | 3.65952 | 3.92807 | 2.52854 | 3.03031 | 2.0617 | 2.45653 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 246 | 2.06644 | 4.57942 | 3.21268 | 2.65204 | 3.59365 | 3.33762 | 3.74312 | 3.14634 | 2.32144 | 2.81522 | 3.46208 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 247 | 2.42097 | 4.99986 | 2.57153 | 2.33872 | 4.29051 | 2.62131 | 3.62194 | 3.73959 | 2.2761 | 3.28171 | 3.5649 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 248 | 2.63485 | 5.03278 | 2.532 | 2.32444 | 4.33333 | 2.61312 | 3.43393 | 3.78927 | 2.37373 | 2.94474 | 3.74599 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 249 | 2.26615 | 4.88564 | 2.89599 | 2.38085 | 4.14454 | 2.44895 | 3.45952 | 3.57087 | 2.44748 | 3.15727 | 3.95984 |
| | 2.68618 | 4.42225 | 2.7752 | 2.73123 | 3.46354 | 2.40513 | 3.72495 | 3.29354 | 2.67741 | 2.69355 | 4.2469 |
| | 0.3617 | 3.4226 | 1.30608 | 0.52826 | 0.89069 | 0.48576 | 0.9551 | | | | |
| 250 | 2.54954 | 4.17252 | 2.92187 | 2.47432 | 3.85932 | 3.27263 | 3.62554 | 3.12385 | 2.38366 | 2.89437 | 3.72907 |
| | 2.68618 | 4.42225 | 2.77519 | 2.73123 | 3.46354 | 2.40513 | 3.72494 | 3.29354 | 2.67741 | 2.69355 | 4.2469 |
| | 0.02905 | 3.94901 | 4.67135 | 0.61958 | 0.77255 | 0.67539 | 0.71122 | | | | |
| 251 | 2.57457 | 4.62687 | 3.06534 | 2.23629 | 3.4396 | 3.30063 | 3.64972 | 3.22383 | 2.40089 | 2.7796 | 3.711 |
| | 2.68618 | 4.42225 | 2.77519 | 2.73123 | 3.46354 | 2.40513 | 3.72494 | 3.29354 | 2.67741 | 2.69355 | 4.2469 |
| | 0.027 | 4.02109 | 4.74344 | 0.61958 | 0.77255 | 0.54018 | 0.87381 | | | | |
| 252 | 2.60849 | 4.99454 | 2.75341 | 2.18415 | 4.29218 | 3.0117 | 3.07295 | 3.74488 | 2.2561 | 2.90066 | 4.04883 |
| | 2.68618 | 4.42225 | 2.77519 | 2.73123 | 3.46354 | 2.40513 | 3.72494 | 3.29354 | 2.67741 | 2.69355 | 4.2469 |
| | 0.02401 | 4.13724 | 4.85959 | 0.61958 | 0.77255 | 0.47497 | 0.9726 | | | | |
| 253 | 2.46135 | 4.13934 | 3.2243 | 2.66245 | 3.71593 | 3.12543 | 3.7403 | 2.98403 | 2.36968 | 2.26741 | 3.48037 |
| | 2.68618 | 4.42225 | 2.77519 | 2.73123 | 3.46354 | 2.40513 | 3.72494 | 3.29354 | 2.67741 | 2.69355 | 4.2469 |
| | 0.02213 | 4.21762 | 4.93996 | 0.61958 | 0.77255 | 0.53438 | 0.88197 | | | | |
| 254 | 2.50444 | 5.12456 | 2.32291 | 1.79401 | 4.43846 | 3.01478 | 3.63135 | 3.90857 | 2.30988 | 3.4177 | 3.71064 |
| | 2.68618 | 4.42225 | 2.77519 | 2.73123 | 3.46354 | 2.40513 | 3.72494 | 3.29354 | 2.67741 | 2.69355 | 4.2469 |
| | 0.02213 | 4.21762 | 4.93996 | 0.61958 | 0.77255 | 0.45652 | 1.00371 | | | | |
| 255 | 2.49388 | 4.93095 | 3.00082 | 2.43899 | 3.91192 | 3.45492 | 3.64128 | 3.438 | 1.79773 | 3.19888 | 3.7895 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 256 | 3.25318 | 4.5239 | 5.10916 | 4.56715 | 3.49459 | 4.6552 | 5.10831 | 1.24545 | 4.45727 | 1.68982 | 2.86955 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 257 | 2.6338 | 5.04188 | 3.10284 | 2.55441 | 4.42648 | 2.23944 | 3.68366 | 3.84084 | 1.79458 | 3.36504 | 4.16894 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 258 | 2.67312 | 5.09557 | 2.70212 | 2.23624 | 4.41629 | 3.12246 | 3.62298 | 3.87496 | 2.16191 | 3.38343 | 4.14836 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 259 | 2.84443 | 4.31866 | 4.21421 | 3.63569 | 2.80473 | 3.9502 | 4.22469 | 2.33451 | 3.27922 | 1.17312 | 3.13784 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 260 | 2.69899 | 5.03966 | 2.91348 | 2.31935 | 4.34581 | 2.52645 | 3.65636 | 3.78565 | 2.275 | 3.32512 | 4.11332 |
| | 2.68622 | 4.42229 | 2.77523 | 2.73127 | 3.46358 | 2.40516 | 3.72498 | 3.29358 | 2.67744 | 2.69359 | 4.24693 |
| | 0.24534 | 1.55746 | 4.97786 | 0.22027 | 1.62102 | 0.48576 | 0.9551 | | | | |
| 261 | 2.68464 | 4.96848 | 3.04303 | 2.46953 | 4.2532 | 3.48102 | 3.64351 | 3.67843 | 1.92496 | 3.23444 | 3.74123 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 262 | 2.82925 | 1.90123 | 4.5154 | 3.95678 | 3.20762 | 4.00292 | 4.46388 | 2.00496 | 3.82767 | 2.28387 | 3.36108 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 263 | 3.02 | 4.42773 | 4.55738 | 3.97126 | 2.60431 | 4.17447 | 4.44925 | 2.42728 | 3.82945 | 1.10478 | 2.29682 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 264 | 3.20722 | 4.49316 | 5.00708 | 4.47687 | 3.31092 | 4.59942 | 5.07613 | 1.60288 | 4.37679 | 2.11094 | 3.21966 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 265 | 2.47442 | 3.72768 | 3.50758 | 2.93968 | 3.46562 | 3.59791 | 3.85397 | 2.49965 | 2.89047 | 2.39448 | 3.43347 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 266 | 2.60251 | 4.42351 | 3.42569 | 2.90118 | 3.64053 | 1.66968 | 3.888 | 3.01906 | 2.84869 | 2.73324 | 3.20683 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 267 | 2.62169 | 3.46323 | 3.44614 | 2.88159 | 2.91217 | 3.1966 | 3.5939 | 2.88436 | 2.83697 | 2.59045 | 3.47903 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 268 | 2.65236 | 5.07623 | 2.38422 | 2.14728 | 4.0421 | 2.46953 | 3.62468 | 3.84771 | 2.38499 | 3.17843 | 4.13065 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 269 | 2.82989 | 5.15783 | 2.44532 | 2.24882 | 4.51176 | 1.49048 | 3.78835 | 3.96384 | 2.61007 | 3.51345 | 4.33114 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 270 | 3.45489 | 5.58801 | 0.49459 | 2.66181 | 5.02904 | 3.55014 | 4.33669 | 4.71613 | 3.50291 | 4.26044 | 5.2638 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 271 | 2.6365 | 4.48975 | 3.54692 | 3.11493 | 3.87196 | 3.44652 | 4.1206 | 2.83494 | 2.99698 | 2.66163 | 3.83607 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 272 | 2.73483 | 4.24792 | 4.06306 | 3.49098 | 3.27283 | 3.81333 | 4.1379 | 2.49748 | 3.37247 | 1.38006 | 2.63831 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 273 | 2.63386 | 4.09315 | 4.14883 | 3.56025 | 2.83115 | 3.74665 | 3.74659 | 2.04132 | 3.42641 | 2.0998 | 3.19065 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 274 | 3.45489 | 5.58801 | 0.49459 | 2.66181 | 5.02904 | 3.55014 | 4.33669 | 4.71613 | 3.50291 | 4.26044 | 5.2638 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 275 | 3.23995 | 5.28638 | 3.76625 | 3.02323 | 4.728 | 3.84715 | 3.73875 | 4.0651 | 1.77809 | 3.29347 | 4.39999 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 276 | 2.68753 | 4.87031 | 3.00963 | 2.53961 | 4.22381 | 3.44011 | 3.73761 | 3.4103 | 2.47454 | 3.23527 | 4.05648 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 277 | 2.6733 | 4.68298 | 3.21922 | 2.64183 | 3.88384 | 3.53798 | 3.72335 | 2.82769 | 2.179 | 2.80765 | 3.43324 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 278 | 2.55174 | 5.18765 | 2.3181 | 1.60966 | 4.5046 | 3.23667 | 3.4423 | 3.98017 | 2.44559 | 3.48195 | 4.24755 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 279 | 3.29516 | 4.62289 | 4.95445 | 4.39964 | 1.78595 | 4.51042 | 4.68391 | 2.31196 | 4.26 | 1.14468 | 3.03611 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 280 | 1.93349 | 4.37072 | 4.32671 | 3.8479 | 3.72065 | 3.88189 | 4.61677 | 2.14138 | 3.7436 | 2.33597 | 3.60497 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 281 | 2.75321 | 5.1783 | 2.83303 | 2.27749 | 4.53037 | 3.47744 | 3.63753 | 3.98048 | 1.73366 | 3.46015 | 4.23171 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 282 | 2.49895 | 4.13751 | 4.24952 | 3.65818 | 3.16064 | 3.81872 | 4.12462 | 2.27674 | 3.51633 | 1.68838 | 2.09863 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 283 | 3.49743 | 4.80833 | 5.10627 | 4.53987 | 3.15607 | 4.75636 | 5.05673 | 2.29267 | 4.37793 | 0.77362 | 2.16533 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 284 | 2.67425 | 5.04246 | 2.96181 | 1.78507 | 4.34741 | 3.45204 | 3.25784 | 3.46847 | 2.0028 | 3.32062 | 4.09834 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 285 | 2.18497 | 5.09173 | 2.73433 | 2.09101 | 4.40765 | 2.98645 | 3.62641 | 3.87259 | 2.04636 | 3.38583 | 4.14806 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 286 | 2.44001 | 4.00454 | 3.01117 | 2.44729 | 4.26774 | 3.46287 | 3.43871 | 3.69942 | 2.00307 | 3.25166 | 4.04275 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 287 | 3.21325 | 4.93276 | 3.96373 | 3.91957 | 5.02096 | 0.32494 | 4.99846 | 4.73153 | 4.15183 | 4.33001 | 5.32112 |
| | 2.68612 | 4.42226 | 2.77521 | 2.73125 | 3.46355 | 2.40508 | 3.72496 | 3.29355 | 2.67735 | 2.69356 | 4.24691 |
| | 0.04839 | 3.2103 | 4.97786 | 0.76165 | 0.62904 | 0.48576 | 0.9551 | | | | |
| 288 | 2.81074 | 4.42319 | 4.66205 | 4.14475 | 3.70592 | 4.29114 | 4.85958 | 1.8405 | 4.02996 | 2.19564 | 3.54246 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 289 | 2.51151 | 5.16672 | 2.39686 | 2.05227 | 4.50106 | 3.40534 | 2.94125 | 3.98238 | 2.27299 | 3.46563 | 4.21348 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 290 | 2.89766 | 4.47264 | 4.68 | 4.22123 | 3.73696 | 4.21922 | 4.95695 | 1.94721 | 4.07257 | 2.32057 | 3.32611 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 291 | 2.65493 | 4.7112 | 2.97929 | 2.10081 | 3.92515 | 3.49629 | 3.72875 | 3.15306 | 2.41153 | 2.95305 | 3.80241 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 292 | 1.93719 | 4.87093 | 2.99706 | 2.17255 | 4.14496 | 3.10869 | 3.668 | 3.56695 | 2.38983 | 3.15654 | 3.96244 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 293 | 2.64823 | 5.04328 | 2.8542 | 2.32909 | 4.35086 | 3.43433 | 2.9811 | 3.80385 | 2.02386 | 3.32758 | 3.57284 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 294 | 2.76882 | 4.1988 | 4.32404 | 3.74372 | 1.66172 | 3.88977 | 4.1087 | 2.41086 | 3.60436 | 1.91576 | 2.91596 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 295 | 2.56564 | 5.02622 | 2.32505 | 2.23525 | 4.32416 | 3.27898 | 3.619 | 3.77857 | 2.31036 | 3.31114 | 4.08034 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 296 | 2.6898 | 5.15584 | 2.16623 | 1.87611 | 4.47398 | 2.99689 | 3.64079 | 3.94745 | 2.25596 | 3.44884 | 4.20944 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 297 | 2.34498 | 4.95915 | 2.53276 | 2.29117 | 4.23019 | 2.09196 | 3.67539 | 3.66339 | 2.42365 | 3.2394 | 4.03998 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 298 | 2.70636 | 4.89473 | 2.44406 | 2.49039 | 4.38194 | 1.42345 | 3.60703 | 3.84057 | 2.76001 | 3.43492 | 4.25973 |
| | 2.68618 | 4.42225 | 2.77519 | 2.73123 | 3.46354 | 2.40513 | 3.72494 | 3.29354 | 2.67741 | 2.69355 | 4.2469 |
| | 0.04054 | 4.25552 | 3.6672 | 0.61958 | 0.77255 | 0.48576 | 0.9551 | | | | |
| 299 | 2.65614 | 3.18364 | 4.06911 | 3.49167 | 1.97653 | 3.74134 | 3.46093 | 2.58799 | 3.14746 | 2.321 | 3.25154 |
| | 2.68618 | 4.42225 | 2.77519 | 2.73123 | 3.46354 | 2.40513 | 3.72494 | 3.29354 | 2.67741 | 2.69355 | 4.2469 |
| | 0.02171 | 4.23668 | 4.95903 | 0.61958 | 0.77255 | 0.47063 | 0.97979 | | | | |
| 300 | 3.5141 | 5.23055 | 3.59545 | 3.41016 | 3.63357 | 3.83874 | 0.54184 | 4.27958 | 3.24679 | 3.71577 | 4.8026 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 301 | 2.13882 | 4.47679 | 3.34006 | 2.82079 | 3.86735 | 1.78241 | 3.88707 | 3.23094 | 2.79169 | 2.73054 | 3.51703 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 302 | 2.39981 | 3.78637 | 4.10096 | 3.27816 | 3.24279 | 3.75552 | 4.07271 | 2.01122 | 3.4022 | 2.301 | 2.63105 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 303 | 2.75206 | 5.19459 | 2.29843 | 1.44672 | 3.91789 | 3.39892 | 3.69215 | 3.96001 | 2.50524 | 3.48083 | 4.26301 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 304 | 2.61261 | 3.69779 | 3.67003 | 3.30824 | 2.75096 | 3.72571 | 4.03296 | 2.19251 | 3.41634 | 1.86586 | 3.03542 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 305 | 2.61251 | 4.09277 | 4.06076 | 3.47456 | 2.24559 | 3.03691 | 4.01288 | 2.35755 | 3.3562 | 2.18545 | 2.938 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 306 | 2.90848 | 5.41832 | 1.29325 | 1.96467 | 4.71447 | 3.37696 | 3.7858 | 4.19593 | 2.43441 | 3.70542 | 4.51235 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 307 | 2.4578 | 4.66658 | 3.07379 | 2.54401 | 4.0584 | 3.19558 | 3.8262 | 3.4527 | 2.67382 | 3.09889 | 3.94077 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 308 | 2.45461 | 5.01237 | 2.9391 | 2.27072 | 4.30765 | 3.43681 | 3.62961 | 3.75315 | 2.07456 | 2.98146 | 4.07153 |
| | 2.68619 | 4.42226 | 2.7752 | 2.73124 | 3.46339 | 2.40514 | 3.72495 | 3.29355 | 2.67734 | 2.69356 | 4.24691 |
| | 0.04727 | 3.23695 | 4.97786 | 0.6378 | 0.75174 | 0.48576 | 0.9551 | | | | |
| 309 | 2.7049 | 3.94849 | 3.05443 | 2.41168 | 4.30093 | 3.49162 | 3.39525 | 3.44696 | 1.62805 | 3.26986 | 4.06486 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 310 | 1.48885 | 4.62485 | 3.17131 | 2.68033 | 4.05066 | 3.04091 | 3.40451 | 3.4543 | 2.65644 | 3.09295 | 3.9287 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 311 | 2.49215 | 5.05184 | 2.76516 | 2.08438 | 4.0596 | 3.41875 | 3.26516 | 3.82102 | 1.93578 | 3.13912 | 4.10278 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 312 | 2.04528 | 4.60312 | 3.18634 | 2.2123 | 3.45106 | 3.50912 | 3.73276 | 2.93956 | 2.55287 | 2.83819 | 3.53467 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 313 | 3.03702 | 4.41534 | 4.68468 | 4.09869 | 2.27125 | 4.21502 | 4.4876 | 2.28531 | 3.95381 | 1.15138 | 2.37605 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 314 | 2.6119 | 3.63327 | 3.62377 | 3.37861 | 2.5622 | 3.49887 | 3.47755 | 2.42549 | 3.27565 | 1.96822 | 3.23485 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 315 | 2.28546 | 5.04925 | 2.56222 | 2.25888 | 4.35344 | 2.61914 | 3.61991 | 3.81188 | 2.23696 | 3.23931 | 3.77034 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 316 | 2.47485 | 4.83268 | 2.62191 | 2.37539 | 3.74481 | 3.45585 | 3.65933 | 3.32098 | 2.36128 | 2.99025 | 3.66378 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 317 | 2.84582 | 4.35412 | 4.78015 | 4.22282 | 3.50189 | 4.32789 | 4.74582 | 1.48701 | 4.10394 | 1.99785 | 2.82026 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 318 | 2.62206 | 5.18306 | 3.10741 | 2.36818 | 4.56173 | 3.37691 | 3.67375 | 3.96898 | 1.31727 | 3.45275 | 4.25939 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 319 | 2.70932 | 5.37526 | 1.70108 | 2.04734 | 4.68991 | 3.18323 | 3.73043 | 4.18785 | 2.21964 | 3.6687 | 4.44623 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 320 | 3.62982 | 4.93341 | 4.85945 | 4.50271 | 0.83157 | 4.53425 | 4.0598 | 2.82803 | 4.33088 | 1.89623 | 3.49824 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 321 | 3.20608 | 4.49457 | 5.03968 | 4.48609 | 3.10426 | 4.56281 | 4.97505 | 1.17306 | 4.36945 | 1.80697 | 2.84859 |
| | 2.68618 | 4.42225 | 2.77519 | 2.73123 | 3.46354 | 2.40513 | 3.72494 | 3.29354 | 2.67741 | 2.69355 | 4.2469 |
| | 0.0213 | 4.25552 | 4.97786 | 0.61958 | 0.77255 | 0.48576 | 0.9551 | | | | |
| 322 | 2.54797 | 4.44994 | 3.03683 | 2.77665 | 3.20753 | 3.55489 | 3.79261 | 2.81898 | 2.6619 | 2.3977 | 3.33032 |
| | 2.68618 | 4.42225 | 2.77519 | 2.73123 | 3.46354 | 2.40513 | 3.72494 | 3.29354 | 2.67741 | 2.69355 | 4.2469 |
| | 0.09568 | 4.25552 | 2.56321 | 0.61958 | 0.77255 | 0.48576 | 0.9551 | | | | |
| 323 | 2.61023 | 4.48456 | 2.85053 | 2.3202 | 4.11292 | 3.13072 | 3.63847 | 3.53709 | 2.42654 | 2.86095 | 3.92985 |
| | 2.68618 | 4.42225 | 2.77519 | 2.73123 | 3.46354 | 2.40513 | 3.72494 | 3.29354 | 2.67741 | 2.69355 | 4.2469 |
| | 0.11838 | 4.18277 | 2.33941 | 0.61958 | 0.77255 | 0.57559 | 0.82639 | | | | |
| 324 | 2.50346 | 3.4029 | 3.58408 | 3.01411 | 3.07703 | 3.31546 | 3.36398 | 2.61861 | 2.95275 | 2.26352 | 3.32498 |
| | 2.68618 | 4.42225 | 2.77519 | 2.73123 | 3.46354 | 2.40513 | 3.72494 | 3.29354 | 2.67741 | 2.69355 | 4.2469 |
| | 0.04406 | 4.08958 | 3.63611 | 0.61958 | 0.77255 | 0.67207 | 0.71468 | | | | |
| 325 | 2.42738 | 4.29616 | 2.8301 | 2.29943 | 4.05943 | 3.40536 | 3.61058 | 3.4806 | 2.27795 | 2.86682 | 3.88189 |
| | 2.68618 | 4.42225 | 2.77519 | 2.73123 | 3.46354 | 2.40513 | 3.72494 | 3.29354 | 2.67741 | 2.69355 | 4.2469 |
| | 0.11452 | 4.07119 | 2.39524 | 0.61958 | 0.77255 | 0.68908 | 0.69723 | | | | |
| 326 | 2.42393 | 4.73588 | 2.98431 | 2.43896 | 3.97462 | 3.40392 | 3.36831 | 3.25716 | 2.3385 | 2.99583 | 3.81855 |
| | 2.68591 | 4.42227 | 2.77528 | 2.73142 | 3.46372 | 2.40515 | 3.72484 | 3.29372 | 2.67741 | 2.69361 | 4.24532 |
| | 0.44569 | 1.02272 | * | 1.71775 | 0.1978 | 0 | * | | | | |

| HMM | N | P | Q | R | S | T | V | W | Y | |
|---|---|---|---|---|---|---|---|---|---|---|
| COMPO | 3.08332 | 3.16278 | 3.14933 | 2.95336 | 2.65169 | 2.88483 | 2.59999 | 4.67957 | 3.50072 | |
| | 2.90385 | 2.73746 | 3.18155 | 2.8983 | 2.3788 | 2.77503 | 2.98538 | 4.5849 | 3.61529 | |
| 1 | 2.88526 | 3.77642 | 2.57955 | 2.68167 | 2.4586 | 2.8237 | 3.21688 | 5.34464 | 3.99892 | 28 -- |
| | 2.90347 | 2.73739 | 3.18146 | 2.89801 | 2.37887 | 2.77519 | 2.98518 | 4.58477 | 3.61503 | |
| 2 | 2.70284 | 3.77638 | 2.72661 | 2.75666 | 2.59095 | 2.67365 | 3.19345 | 5.33953 | 3.98699 | 29 -- |
| | 2.90347 | 2.73739 | 3.18146 | 2.89801 | 2.37887 | 2.77519 | 2.98518 | 4.58477 | 3.61503 | |
| 3 | 2.87196 | 3.77565 | 2.39793 | 2.815 | 2.44517 | 2.82406 | 3.3054 | 5.41452 | 4.04333 | 30 -- |
| | 2.90347 | 2.73739 | 3.18146 | 2.89801 | 2.37887 | 2.77519 | 2.98518 | 4.58477 | 3.61503 | |
| 4 | 2.71561 | 3.83423 | 2.82061 | 2.93237 | 2.14826 | 2.68914 | 3.08385 | 5.2736 | 3.70913 | 31 -- |
| | 2.90347 | 2.73739 | 3.18146 | 2.89801 | 2.37887 | 2.77519 | 2.98518 | 4.58477 | 3.61503 | |
| 5 | 2.89654 | 3.20085 | 2.82089 | 2.74045 | 2.24849 | 2.66127 | 2.99154 | 4.57462 | 3.93097 | 32 -- |
| | 2.90347 | 2.73739 | 3.18146 | 2.89801 | 2.37887 | 2.77519 | 2.98518 | 4.58477 | 3.61503 | |
| 6 | 2.92122 | 3.28543 | 2.74721 | 2.86991 | 2.27246 | 2.50614 | 3.26999 | 5.40172 | 3.76473 | 33 -- |
| | 2.90347 | 2.73739 | 3.18146 | 2.89801 | 2.37887 | 2.77519 | 2.98518 | 4.58477 | 3.61503 | |
| 7 | 2.94258 | 2.67641 | 2.76522 | 2.76329 | 2.44021 | 2.84398 | 3.21693 | 5.3636 | 4.01506 | 34 -- |
| | 2.90347 | 2.73739 | 3.18146 | 2.89801 | 2.37887 | 2.77519 | 2.98518 | 4.58477 | 3.61503 | |
| 8 | 2.98477 | 2.35337 | 3.01053 | 3.08784 | 2.39118 | 2.58194 | 2.85901 | 5.13831 | 3.86638 | 35 -- |
| | 2.90347 | 2.73739 | 3.18146 | 2.89801 | 2.37887 | 2.77519 | 2.98518 | 4.58477 | 3.61503 | |
| 9 | 2.88639 | 2.42343 | 2.95167 | 3.03777 | 2.341 | 2.68781 | 2.89365 | 5.14493 | 3.8625 | 36 -- |
| | 2.90347 | 2.73739 | 3.18146 | 2.89801 | 2.37887 | 2.77519 | 2.98518 | 4.58477 | 3.61503 | |
| 10 | 2.68838 | 3.65725 | 2.96097 | 3.04353 | 2.36538 | 2.66847 | 2.67898 | 5.0936 | 3.81758 | 37 -- |
| | 2.90347 | 2.73739 | 3.18146 | 2.89801 | 2.37887 | 2.77519 | 2.98518 | 4.58477 | 3.61503 | |
| 11 | 3.3176 | 3.71261 | 3.16728 | 3.22162 | 2.40915 | 2.54589 | 2.42603 | 4.9238 | 3.68765 | 38 -- |
| | 2.90353 | 2.73746 | 3.18143 | 2.89807 | 2.37872 | 2.7751 | 2.98525 | 4.58483 | 3.6151 | |
| 12 | 2.60435 | 3.85767 | 2.72752 | 2.99753 | 2.6133 | 2.98557 | 3.55449 | 5.64272 | 4.23457 | 42 -- |
| | 2.90347 | 2.73739 | 3.18146 | 2.89801 | 2.37887 | 2.77519 | 2.98518 | 4.58477 | 3.61503 | |
| 13 | 3.28197 | 1.87242 | 3.16811 | 3.21784 | 2.47704 | 2.71377 | 2.79721 | 5.13677 | 3.88114 | 43 -- |
| | 2.90347 | 2.73739 | 3.18146 | 2.89801 | 2.37887 | 2.77519 | 2.98518 | 4.58477 | 3.61503 | |
| 14 | 2.84662 | 4.04913 | 3.23302 | 3.29664 | 2.92174 | 2.76367 | 2.78865 | 4.68319 | 1.71492 | 44 -- |
| | 2.90347 | 2.73739 | 3.18146 | 2.89801 | 2.37887 | 2.77519 | 2.98518 | 4.58477 | 3.61503 | |
| 15 | 2.84293 | 3.81574 | 2.59821 | 2.84826 | 2.62004 | 2.70016 | 3.43852 | 5.51887 | 4.12974 | 45 -- |
| | 2.90347 | 2.73739 | 3.18146 | 2.89801 | 2.37887 | 2.77519 | 2.98518 | 4.58477 | 3.61503 | |
| 16 | 3.46769 | 4.017 | 3.10192 | 3.19369 | 2.71875 | 2.84504 | 2.48514 | 4.78972 | 2.68075 | 46 -- |
| | 2.90347 | 2.73739 | 3.18146 | 2.89801 | 2.37887 | 2.77519 | 2.98518 | 4.58477 | 3.61503 | |
| 17 | 4.42048 | 4.60519 | 4.20951 | 4.19678 | 3.67927 | 3.35684 | 2.26569 | 5.04843 | 3.94788 | 47 -- |
| | 2.90347 | 2.73739 | 3.18146 | 2.89801 | 2.37887 | 2.77519 | 2.98518 | 4.58477 | 3.61503 | |
| 18 | 2.26103 | 3.84465 | 2.35167 | 2.72161 | 2.6768 | 2.94597 | 3.55173 | 5.59702 | 4.20735 | 48 -- |
| | 2.90347 | 2.73739 | 3.18146 | 2.89801 | 2.37887 | 2.77519 | 2.98518 | 4.58477 | 3.61503 | |
| 19 | 4.69285 | 4.81072 | 4.46598 | 4.46288 | 3.9533 | 3.51676 | 2.04961 | 5.25471 | 4.16887 | 49 -- |
| | 2.90347 | 2.73739 | 3.18146 | 2.89801 | 2.37887 | 2.77519 | 2.98518 | 4.58477 | 3.61503 | |
| 20 | 3.16016 | 3.96901 | 3.14643 | 2.91147 | 2.83107 | 2.30885 | 1.91662 | 4.96154 | 3.72256 | 50 -- |
| | 2.90347 | 2.73739 | 3.18146 | 2.89801 | 2.37887 | 2.77519 | 2.98518 | 4.58477 | 3.61503 | |
| 21 | 2.86197 | 3.35797 | 2.54337 | 2.77232 | 2.57082 | 2.85898 | 2.93104 | 5.26768 | 3.57153 | 51 -- |
| | 2.90347 | 2.73739 | 3.18146 | 2.89801 | 2.37887 | 2.77519 | 2.98518 | 4.58477 | 3.61503 | |
| 22 | 1.09296 | 3.95591 | 3.10731 | 3.39798 | 2.87684 | 3.00121 | 3.76878 | 5.92587 | 4.48837 | 52 -- |
| | 2.90347 | 2.73739 | 3.18146 | 2.89801 | 2.37887 | 2.77519 | 2.98518 | 4.58477 | 3.61503 | |
| 23 | 3.41693 | 1.03535 | 3.62679 | 3.62343 | 2.32477 | 2.9145 | 3.3638 | 5.8367 | 4.60374 | 53 -- |
| | 2.90347 | 2.73739 | 3.18146 | 2.89801 | 2.37887 | 2.77519 | 2.98518 | 4.58477 | 3.61503 | |
| 24 | 2.31166 | 3.97877 | 2.74169 | 3.54121 | 2.96954 | 3.38833 | 4.08478 | 6.1513 | 4.62343 | 54 -- |
| | 2.90347 | 2.73739 | 3.18146 | 2.89801 | 2.37887 | 2.77519 | 2.98518 | 4.58477 | 3.61503 | |
| 25 | 3.24288 | 3.91384 | 3.49669 | 3.68531 | 2.70827 | 3.04789 | 3.53763 | 5.92879 | 4.64662 | 55 -- |
| | 2.90341 | 2.7374 | 3.18147 | 2.89802 | 2.37888 | 2.7752 | 2.98519 | 4.58478 | 3.61504 | |
| 26 | 3.44969 | 3.8508 | 3.61161 | 3.6115 | 1.17415 | 1.65554 | 3.28065 | 5.76893 | 4.54493 | 57 -- |
| | 2.90347 | 2.73739 | 3.18146 | 2.89801 | 2.37887 | 2.77519 | 2.98518 | 4.58477 | 3.61503 | |
| 27 | 4.3877 | 4.58506 | 4.25882 | 4.2067 | 3.62501 | 3.26538 | 1.61066 | 5.10383 | 3.9568 | 58 -- |
| | 2.90347 | 2.73739 | 3.18146 | 2.89801 | 2.37887 | 2.77519 | 2.98518 | 4.58477 | 3.61503 | |
| 28 | 3.68528 | 4.05006 | 3.70235 | 3.66054 | 2.83531 | 1.18295 | 2.47321 | 5.29233 | 4.08047 | 59 -- |
| | 2.90347 | 2.73739 | 3.18146 | 2.89801 | 2.37887 | 2.77519 | 2.98518 | 4.58477 | 3.61503 | |
| 29 | 3.35021 | 4.08577 | 2.98303 | 1.07934 | 3.01372 | 3.17012 | 3.3723 | 5.43114 | 4.22778 | 60 -- |
| | 2.90347 | 2.73739 | 3.18146 | 2.89801 | 2.37887 | 2.77519 | 2.98518 | 4.58477 | 3.61503 | |
| 30 | 2.45453 | 3.01529 | 2.68525 | 2.72231 | 2.54764 | 2.85265 | 3.13332 | 5.39381 | 3.54934 | 61 -- |
| | 2.90347 | 2.73739 | 3.18146 | 2.89801 | 2.37887 | 2.77519 | 2.98518 | 4.58477 | 3.61503 | |
| 31 | 3.37178 | 3.54244 | 3.22706 | 3.12501 | 2.30812 | 2.59326 | 2.41278 | 4.87497 | 3.46753 | 62 -- |
| | 2.90347 | 2.73739 | 3.18146 | 2.89801 | 2.37887 | 2.77519 | 2.98518 | 4.58477 | 3.61503 | |
| 32 | 2.86463 | 3.26325 | 2.75733 | 2.87797 | 2.53839 | 2.52978 | 3.04053 | 5.40629 | 4.04675 | 63 -- |
| | 2.90347 | 2.73739 | 3.18146 | 2.89801 | 2.37887 | 2.77519 | 2.98518 | 4.58477 | 3.61503 | |
| 33 | 3.33944 | 4.00499 | 3.33155 | 3.22215 | 2.81222 | 2.72251 | 2.29236 | 4.22539 | 3.57862 | 64 -- |
| | 2.90349 | 2.73741 | 3.18148 | 2.89803 | 2.37889 | 2.77521 | 2.9852 | 4.58479 | 3.61473 | |
| 34 | 3.4287 | 0.97749 | 3.55495 | 3.4574 | 2.55956 | 3.02233 | 3.29902 | 5.63307 | 4.32568 | 66 -- |
| | 2.90347 | 2.73739 | 3.18146 | 2.89801 | 2.37887 | 2.77519 | 2.98518 | 4.58477 | 3.61503 | |
| 35 | 2.89322 | 3.87297 | 2.87907 | 2.76566 | 2.32019 | 2.42136 | 2.93327 | 5.22699 | 3.72907 | 67 -- |
| | 2.90347 | 2.73739 | 3.18146 | 2.89801 | 2.37887 | 2.77519 | 2.98518 | 4.58477 | 3.61503 | |
| 36 | 3.79101 | 4.20865 | 3.68813 | 3.66223 | 2.84069 | 2.38331 | 1.60478 | 4.93301 | 3.73293 | 68 -- |
| | 2.90347 | 2.73739 | 3.18146 | 2.89801 | 2.37887 | 2.77519 | 2.98518 | 4.58477 | 3.61503 | |
| 37 | 2.80195 | 2.31234 | 2.72429 | 2.9174 | 2.46453 | 2.87979 | 3.10901 | 5.41707 | 3.59287 | 69 -- |
| | 2.90347 | 2.73739 | 3.18146 | 2.89801 | 2.37887 | 2.77519 | 2.98518 | 4.58477 | 3.61503 | |
| 38 | 3.06737 | 2.59171 | 2.9065 | 3.00522 | 2.50811 | 2.64073 | 3.02249 | 5.2473 | 3.94336 | 70 -- |
| | 2.90347 | 2.73739 | 3.18146 | 2.89801 | 2.37887 | 2.77519 | 2.98518 | 4.58477 | 3.61503 | |
| 39 | 2.59889 | 3.49951 | 2.78908 | 2.91986 | 2.22144 | 2.21088 | 3.13513 | 5.45757 | 4.09404 | 71 -- |
| | 2.90347 | 2.73739 | 3.18146 | 2.89801 | 2.37887 | 2.77519 | 2.98518 | 4.58477 | 3.61503 | |
| 40 | 2.92785 | 2.04062 | 2.83067 | 2.99006 | 2.60202 | 2.93714 | 3.38754 | 5.52462 | 4.15156 | 72 -- |
| | 2.9034 | 2.73742 | 3.18149 | 2.89803 | 2.37889 | 2.77516 | 2.98521 | 4.58479 | 3.61505 | |
| 41 | 2.56911 | 3.81999 | 2.52532 | 2.90012 | 2.4293 | 2.62251 | 3.50604 | 5.58015 | 4.17583 | 74 -- |
| | 2.90347 | 2.73739 | 3.18146 | 2.89801 | 2.37887 | 2.77519 | 2.98518 | 4.58477 | 3.61503 | |
| 42 | 3.21273 | 2.46219 | 3.05297 | 3.12543 | 2.6968 | 2.67956 | 2.73819 | 4.42919 | 3.76111 | 75 -- |
| | 2.90347 | 2.73739 | 3.18146 | 2.89801 | 2.37887 | 2.77519 | 2.98518 | 4.58477 | 3.61503 | |
| 43 | 2.84648 | 3.85435 | 2.8428 | 2.95258 | 2.33051 | 2.3019 | 2.93967 | 5.27965 | 3.95863 | 76 -- |
| | 2.90347 | 2.73739 | 3.18146 | 2.89801 | 2.37887 | 2.77519 | 2.98518 | 4.58477 | 3.61503 | |
| 44 | 3.05565 | 2.52958 | 2.90885 | 2.98981 | 2.52861 | 2.44056 | 2.98637 | 5.21884 | 3.92272 | 77 -- |
| | 2.90347 | 2.73739 | 3.18146 | 2.89801 | 2.37887 | 2.77519 | 2.98518 | 4.58477 | 3.61503 | |
| 45 | 2.90966 | 3.58965 | 2.5829 | 2.87367 | 2.51928 | 2.63962 | 2.89759 | 5.38935 | 4.03054 | 78 -- |
| | 2.90347 | 2.73739 | 3.18146 | 2.89801 | 2.37887 | 2.77519 | 2.98518 | 4.58477 | 3.61503 | |
| 46 | 2.85028 | 3.83265 | 2.84622 | 2.94179 | 2.24496 | 2.60631 | 2.99409 | 5.20732 | 3.90003 | 79 -- |
| | 2.90352 | 2.73752 | 3.18113 | 2.89783 | 2.37895 | 2.77528 | 2.98514 | 4.58489 | 3.61515 | |
| 47 | 3.17979 | 2.56646 | 3.02797 | 2.86056 | 2.51903 | 2.75422 | 2.45651 | 5.078 | 3.81545 | 90 -- |
| | 2.90347 | 2.73739 | 3.18146 | 2.89801 | 2.37887 | 2.77519 | 2.98518 | 4.58477 | 3.61503 | |
| 48 | 3.55442 | 4.02663 | 3.49007 | 3.49924 | 2.50411 | 2.61715 | 1.66485 | 5.06123 | 3.84746 | 91 -- |
| | 2.90347 | 2.73739 | 3.18146 | 2.89801 | 2.37887 | 2.77519 | 2.98518 | 4.58477 | 3.61503 | |
| 49 | 3.57365 | 3.75431 | 3.45089 | 3.43793 | 2.84026 | 2.84365 | 2.03333 | 4.74524 | 3.53179 | 92 -- |
| | 2.90347 | 2.73739 | 3.18146 | 2.89801 | 2.37887 | 2.77519 | 2.98518 | 4.58477 | 3.61503 | |
| 50 | 3.36238 | 3.9572 | 3.24808 | 3.28933 | 1.67148 | 2.44429 | 2.68687 | 4.99283 | 3.74412 | 93 -- |
| | 2.90347 | 2.73739 | 3.18146 | 2.89801 | 2.37887 | 2.77519 | 2.98518 | 4.58477 | 3.61503 | |
| 51 | 3.22619 | 4.08215 | 2.87597 | 2.21843 | 2.68804 | 3.24395 | 3.70322 | 5.58826 | 4.37283 | 94 -- |
| | 2.90347 | 2.73739 | 3.18146 | 2.89801 | 2.37887 | 2.77519 | 2.98518 | 4.58477 | 3.61503 | |
| 52 | 3.23153 | 4.23051 | 3.58785 | 4.05877 | 3.38503 | 3.807 | 4.31921 | 6.11573 | 4.86017 | 95 -- |
| | 2.90347 | 2.73739 | 3.18146 | 2.89801 | 2.37887 | 2.77519 | 2.98518 | 4.58477 | 3.61503 | |
| 53 | 4.85534 | 4.99585 | 4.76643 | 4.71418 | 4.15094 | 3.54705 | 1.3399 | 5.68023 | 4.51296 | 96 -- |
| | 2.90347 | 2.73739 | 3.18146 | 2.89801 | 2.37887 | 2.77519 | 2.98518 | 4.58477 | 3.61503 | |
| 54 | 3.33015 | 1.65495 | 3.29233 | 3.30509 | 2.57981 | 2.02315 | 2.96046 | 5.38237 | 4.13085 | 97 -- |
| | 2.90347 | 2.73739 | 3.18146 | 2.89801 | 2.37887 | 2.77519 | 2.98518 | 4.58477 | 3.61503 | |
| 55 | 4.3535 | 4.59007 | 4.17656 | 4.1455 | 3.63988 | 3.32576 | 2.02158 | 5.05775 | 3.28703 | 98 -- |
| | 2.90347 | 2.73739 | 3.18146 | 2.89801 | 2.37887 | 2.77519 | 2.98518 | 4.58477 | 3.61503 | |
| 56 | 1.17295 | 3.95122 | 3.07197 | 3.43674 | 2.63178 | 3.26288 | 3.87679 | 5.99392 | 4.52402 | 99 -- |
| | 2.90347 | 2.73739 | 3.18146 | 2.89801 | 2.37887 | 2.77519 | 2.98518 | 4.58477 | 3.61503 | |
| 57 | 2.83035 | 2.57555 | 2.61299 | 2.86292 | 2.41236 | 2.77738 | 3.37675 | 5.47857 | 4.10149 | 100 -- |
| | 2.90347 | 2.73739 | 3.18146 | 2.89801 | 2.37887 | 2.77519 | 2.98518 | 4.58477 | 3.61503 | |
| 58 | 2.71628 | 3.82771 | 2.67225 | 2.90065 | 2.18354 | 2.44744 | 3.43486 | 5.5308 | 4.14267 | 101 -- |
| | 2.90347 | 2.73739 | 3.18146 | 2.89801 | 2.37887 | 2.77519 | 2.98518 | 4.58477 | 3.61503 | |
| 59 | 2.15446 | 3.8586 | 2.77846 | 2.81135 | 2.60548 | 2.49122 | 3.43224 | 5.51626 | 4.15469 | 102 -- |
| | 2.90347 | 2.73739 | 3.18146 | 2.89801 | 2.37887 | 2.77519 | 2.98518 | 4.58477 | 3.61503 | |
| 60 | 2.28079 | 3.49617 | 2.58314 | 2.71841 | 2.39751 | 2.9076 | 3.4994 | 5.56104 | 4.17059 | 103 -- |
| | 2.90347 | 2.73739 | 3.18146 | 2.89801 | 2.37887 | 2.77519 | 2.98518 | 4.58477 | 3.61503 | |
| 61 | 3.71802 | 4.15047 | 3.69748 | 3.6497 | 2.96409 | 1.21793 | 2.07913 | 5.38663 | 4.15931 | 104 -- |
| | 2.90347 | 2.73739 | 3.18146 | 2.89801 | 2.37887 | 2.77519 | 2.98518 | 4.58477 | 3.61503 | |
| 62 | 3.64365 | 4.14441 | 3.53448 | 3.31142 | 2.79036 | 2.97282 | 2.54734 | 2.09143 | 2.86733 | 105 -- |
| | 2.90347 | 2.73739 | 3.18146 | 2.89801 | 2.37887 | 2.77519 | 2.98518 | 4.58477 | 3.61503 | |
| 63 | 4.1695 | 4.44618 | 4.03353 | 3.99646 | 3.44567 | 3.16814 | 1.71352 | 4.99906 | 3.84857 | 106 -- |
| | 2.90347 | 2.73739 | 3.18146 | 2.89801 | 2.37887 | 2.77519 | 2.98518 | 4.58477 | 3.61503 | |
| 64 | 3.80428 | 4.38334 | 3.395 | 0.52404 | 3.57083 | 3.74393 | 4.02369 | 5.66284 | 4.58903 | 107 -- |
| | 2.90347 | 2.73739 | 3.18146 | 2.89801 | 2.37887 | 2.77519 | 2.98518 | 4.58477 | 3.61503 | |
| 65 | 4.85285 | 4.95471 | 4.618 | 4.61143 | 4.14693 | 3.63253 | 1.87296 | 5.44859 | 4.36767 | 108 -- |
| | 2.90347 | 2.73739 | 3.18146 | 2.89801 | 2.37887 | 2.77519 | 2.98518 | 4.58477 | 3.61503 | |
| 66 | 4.32199 | 4.97763 | 4.42893 | 4.52447 | 4.07852 | 4.24492 | 3.60237 | 3.68802 | 1.07985 | 109 -- |
| | 2.90347 | 2.73739 | 3.18146 | 2.89801 | 2.37887 | 2.77519 | 2.98518 | 4.58477 | 3.61503 | |
| 67 | 3.34726 | 4.00543 | 3.1601 | 2.06091 | 2.87905 | 2.90312 | 2.42108 | 5.00341 | 3.77164 | 110 -- |
| | 2.90347 | 2.73739 | 3.18146 | 2.89801 | 2.37887 | 2.77519 | 2.98518 | 4.58477 | 3.61503 | |
| 68 | 4.16117 | 0.34853 | 4.37081 | 4.1814 | 3.52713 | 3.81811 | 4.09794 | 5.89679 | 4.96637 | 111 -- |
| | 2.90353 | 2.73736 | 3.18153 | 2.89807 | 2.37886 | 2.77453 | 2.98525 | 4.58483 | 3.61509 | |
| 69 | 2.91922 | 3.62255 | 2.83304 | 2.36064 | 2.32803 | 2.49988 | 2.68205 | 5.27818 | 3.96021 | 122 -- |
| | 2.90347 | 2.73739 | 3.18146 | 2.89801 | 2.37887 | 2.77519 | 2.98518 | 4.58477 | 3.61503 | |
| 70 | 2.65101 | 3.62197 | 2.62581 | 2.75273 | 2.45298 | 2.35904 | 3.42085 | 5.50488 | 4.11715 | 123 -- |
| | 2.90348 | 2.73729 | 3.18158 | 2.89806 | 2.37889 | 2.77507 | 2.98525 | 4.58489 | 3.61515 | |
| 71 | 2.93938 | 2.97669 | 2.8543 | 2.96119 | 2.1677 | 2.64104 | 2.96092 | 5.25328 | 3.93857 | 129 -- |
| | 2.90347 | 2.73739 | 3.18146 | 2.89801 | 2.37887 | 2.77519 | 2.98518 | 4.58477 | 3.61503 | |
| 72 | 2.63519 | 2.50877 | 2.85154 | 2.96849 | 2.26531 | 2.86211 | 2.93905 | 5.37748 | 4.04387 | 130 -- |
| | 2.90347 | 2.73739 | 3.18146 | 2.89801 | 2.37887 | 2.77519 | 2.98518 | 4.58477 | 3.61503 | |
| 73 | 2.7498 | 3.04638 | 2.66461 | 2.87439 | 2.26911 | 2.72622 | 3.41714 | 5.51086 | 4.12418 | 131 -- |
| | 2.90347 | 2.73739 | 3.18146 | 2.89801 | 2.37887 | 2.77519 | 2.98518 | 4.58477 | 3.61503 | |
| 74 | 2.41749 | 3.82163 | 2.68319 | 2.68949 | 2.43711 | 2.60391 | 3.13389 | 5.44299 | 4.07585 | 132 -- |
| | 2.90347 | 2.7374 | 3.18147 | 2.89801 | 2.37887 | 2.7752 | 2.98519 | 4.58477 | 3.61503 | |
| 75 | 2.69287 | 3.51207 | 2.79214 | 2.94362 | 2.28079 | 2.15106 | 3.39419 | 5.51747 | 4.13972 | 134 -- |
| | 2.90347 | 2.73739 | 3.18146 | 2.89801 | 2.37887 | 2.77519 | 2.98518 | 4.58477 | 3.61503 | |
| 76 | 2.68228 | 3.81886 | 2.81125 | 2.92549 | 2.40989 | 2.52162 | 2.74556 | 5.33759 | 4.00304 | 135 -- |
| | 2.90347 | 2.73739 | 3.18146 | 2.89801 | 2.37887 | 2.77519 | 2.98518 | 4.58477 | 3.61503 | |
| 77 | 2.57096 | 3.80676 | 2.59612 | 2.73634 | 2.55036 | 2.76065 | 3.28037 | 5.40117 | 4.04464 | 136 -- |
| | 2.90347 | 2.73739 | 3.18146 | 2.89801 | 2.37887 | 2.77519 | 2.98518 | 4.58477 | 3.61503 | |
| 78 | 3.33015 | 4.16424 | 2.83579 | 1.61028 | 3.18173 | 3.34459 | 3.80083 | 5.56718 | 4.41071 | 137 -- |
| | 2.90347 | 2.73739 | 3.18146 | 2.89801 | 2.37887 | 2.77519 | 2.98518 | 4.58477 | 3.61503 | |
| 79 | 4.74816 | 4.91513 | 4.45747 | 4.36509 | 4.11077 | 3.78815 | 2.46198 | 5.28814 | 4.11463 | 138 -- |
| | 2.90347 | 2.73739 | 3.18146 | 2.89801 | 2.37887 | 2.77519 | 2.98518 | 4.58477 | 3.61503 | |
| 80 | 4.16117 | 0.34853 | 4.37081 | 4.1814 | 3.52713 | 3.81811 | 4.09794 | 5.89679 | 4.96637 | 139 -- |
| | 2.90347 | 2.73739 | 3.18146 | 2.89801 | 2.37887 | 2.77519 | 2.98518 | 4.58477 | 3.61503 | |
| 81 | 4.82118 | 4.93963 | 4.64161 | 4.62074 | 4.10683 | 3.57237 | 1.59668 | 5.49178 | 4.38566 | 140 -- |
| | 2.90347 | 2.73739 | 3.18146 | 2.89801 | 2.37887 | 2.77519 | 2.98518 | 4.58477 | 3.61503 | |
| 82 | 4.8407 | 4.97703 | 4.73975 | 4.69276 | 4.13067 | 3.53936 | 1.30674 | 5.63513 | 4.47926 | 141 -- |
| | 2.90347 | 2.73739 | 3.18146 | 2.89801 | 2.37887 | 2.77519 | 2.98518 | 4.58477 | 3.61503 | |
| 83 | 4.83709 | 4.97751 | 4.74739 | 4.6967 | 4.12724 | 3.53168 | 1.1862 | 5.65072 | 4.4873 | 142 -- |
| | 2.90347 | 2.73739 | 3.18146 | 2.89801 | 2.37887 | 2.77519 | 2.98518 | 4.58477 | 3.61503 | |
| 84 | 4.19844 | 4.8896 | 4.26857 | 4.34606 | 3.94889 | 4.12351 | 3.62249 | 3.69146 | 0.77075 | 143 -- |
| | 2.90347 | 2.73739 | 3.18146 | 2.89801 | 2.37887 | 2.77519 | 2.98518 | 4.58477 | 3.61503 | |
| 85 | 4.14156 | 4.57963 | 4.08777 | 4.09382 | 3.59024 | 3.47095 | 2.55469 | 4.13689 | 2.04898 | 144 -- |
| | 2.90347 | 2.73739 | 3.18146 | 2.89801 | 2.37887 | 2.77519 | 2.98518 | 4.58477 | 3.61503 | |
| 86 | 3.79703 | 4.41988 | 3.78638 | 3.48044 | 3.58665 | 3.83775 | 4.00762 | 5.08791 | 3.57711 | 145 -- |
| | 2.90347 | 2.73739 | 3.18146 | 2.89801 | 2.37887 | 2.77519 | 2.98518 | 4.58477 | 3.61503 | |
| 87 | 2.98299 | 3.87953 | 3.59841 | 3.68164 | 2.37665 | 2.97848 | 3.54566 | 5.94883 | 4.67727 | 146 -- |
| | 2.90347 | 2.73739 | 3.18146 | 2.89801 | 2.37887 | 2.77519 | 2.98518 | 4.58477 | 3.61503 | |
| 88 | 3.47564 | 3.88209 | 3.79973 | 3.83632 | 2.10584 | 2.9579 | 3.53501 | 5.98367 | 4.77258 | 147 -- |
| | 2.90347 | 2.73739 | 3.18146 | 2.89801 | 2.37887 | 2.77519 | 2.98518 | 4.58477 | 3.61503 | |
| 89 | 3.50276 | 3.91199 | 3.84058 | 3.85877 | 2.36157 | 3.00557 | 3.56988 | 5.98357 | 4.77492 | 148 -- |
| | 2.90347 | 2.73739 | 3.18146 | 2.89801 | 2.37887 | 2.77519 | 2.98518 | 4.58477 | 3.61503 | |
| 90 | 4.17955 | 4.68714 | 4.18386 | 4.21103 | 3.71458 | 3.67106 | 3.05343 | 3.41345 | 2.00158 | 149 -- |
| | 2.90347 | 2.73739 | 3.18146 | 2.89801 | 2.37887 | 2.77519 | 2.98518 | 4.58477 | 3.61503 | |
| 91 | 4.85527 | 5.00885 | 4.8066 | 4.73913 | 4.1517 | 3.53479 | 0.98865 | 5.76106 | 4.56361 | 150 -- |
| | 2.90347 | 2.73739 | 3.18146 | 2.89801 | 2.37887 | 2.77519 | 2.98518 | 4.58477 | 3.61503 | |
| 92 | 4.67795 | 4.77835 | 4.41049 | 4.41953 | 3.92694 | 3.52994 | 2.08409 | 5.16441 | 4.0988 | 151 -- |
| | 2.90347 | 2.73739 | 3.18146 | 2.89801 | 2.37887 | 2.77519 | 2.98518 | 4.58477 | 3.61503 | |
| 93 | 3.80438 | 4.12233 | 3.71233 | 3.6436 | 2.91929 | 2.85989 | 2.29233 | 4.64543 | 2.76597 | 152 -- |
| | 2.90347 | 2.73739 | 3.18146 | 2.89801 | 2.37887 | 2.77519 | 2.98518 | 4.58477 | 3.61503 | |
| 94 | 3.11761 | 3.549 | 3.00521 | 2.64849 | 1.3011 | 2.96976 | 3.31349 | 5.47545 | 4.16419 | 153 -- |
| | 2.90347 | 2.73739 | 3.18146 | 2.89801 | 2.37887 | 2.77519 | 2.98518 | 4.58477 | 3.61503 | |
| 95 | 3.55524 | 3.22825 | 3.60736 | 3.60762 | 2.7151 | 2.88976 | 1.92445 | 5.3993 | 4.18767 | 154 -- |
| | 2.90347 | 2.73739 | 3.18146 | 2.89801 | 2.37887 | 2.77519 | 2.98518 | 4.58477 | 3.61503 | |
| 96 | 2.81245 | 3.85588 | 2.86602 | 3.0359 | 2.37015 | 2.54389 | 3.41127 | 5.5624 | 4.18993 | 155 -- |
| | 2.90347 | 2.73739 | 3.18146 | 2.89801 | 2.37887 | 2.77519 | 2.98518 | 4.58477 | 3.61503 | |
| 97 | 3.00505 | 3.88038 | 2.90365 | 3.0026 | 2.01908 | 2.29191 | 2.98395 | 5.20707 | 3.90669 | 156 -- |
| | 2.90347 | 2.73739 | 3.18146 | 2.89801 | 2.37887 | 2.77519 | 2.98518 | 4.58477 | 3.61503 | |
| 98 | 3.10978 | 3.17225 | 2.7977 | 2.92585 | 2.58737 | 2.51353 | 2.64506 | 5.14038 | 3.85535 | 157 -- |
| | 2.90347 | 2.73739 | 3.18146 | 2.89801 | 2.37887 | 2.77519 | 2.98518 | 4.58477 | 3.61503 | |
| 99 | 3.13135 | 2.74761 | 3.3607 | 3.37423 | 2.91179 | 2.83996 | 2.40133 | 4.79664 | 3.38073 | 158 -- |
| | 2.90347 | 2.73739 | 3.18146 | 2.89801 | 2.37887 | 2.77519 | 2.98518 | 4.58477 | 3.61503 | |
| 100 | 3.38762 | 4.08133 | 3.49624 | 3.48704 | 2.98482 | 2.87964 | 2.34986 | 4.68835 | 3.18047 | 159 -- |
| | 2.90347 | 2.73739 | 3.18146 | 2.89801 | 2.37887 | 2.77519 | 2.98518 | 4.58477 | 3.61503 | |
| 101 | 2.6908 | 3.9553 | 2.81073 | 3.09238 | 2.8756 | 3.17848 | 3.6561 | 5.65299 | 4.21696 | 160 -- |
| | 2.90347 | 2.73739 | 3.18146 | 2.89801 | 2.37887 | 2.77519 | 2.98518 | 4.58477 | 3.61503 | |
| 102 | 2.60724 | 3.81636 | 2.64977 | 2.69099 | 2.55691 | 2.89531 | 3.36501 | 5.58279 | 4.17662 | 161 -- |
| | 2.90347 | 2.73739 | 3.18146 | 2.89801 | 2.37887 | 2.77519 | 2.98518 | 4.58477 | 3.61503 | |
| 103 | 3.38285 | 3.58741 | 3.24051 | 3.27861 | 2.63286 | 2.43026 | 2.56784 | 4.88648 | 3.65982 | 162 -- |
| | 2.90347 | 2.73739 | 3.18146 | 2.89801 | 2.37887 | 2.77519 | 2.98518 | 4.58477 | 3.61503 | |
| 104 | 4.41326 | 4.36592 | 4.69189 | 4.41124 | 3.37594 | 3.60566 | 3.39326 | 5.76248 | 4.73113 | 163 -- |
| | 2.90347 | 2.73739 | 3.18146 | 2.89801 | 2.37887 | 2.77519 | 2.98518 | 4.58477 | 3.61503 | |
| 105 | 2.74926 | 3.85078 | 2.82322 | 2.84007 | 2.14234 | 2.62463 | 3.04237 | 5.32817 | 3.99619 | 164 -- |
| | 2.90347 | 2.73739 | 3.18146 | 2.89801 | 2.37887 | 2.77519 | 2.98518 | 4.58477 | 3.61503 | |
| 106 | 2.54258 | 3.8109 | 2.56533 | 2.65835 | 2.39343 | 2.86594 | 3.29187 | 5.51768 | 3.78882 | 165 -- |
| | 2.90347 | 2.73739 | 3.18146 | 2.89801 | 2.37887 | 2.77519 | 2.98518 | 4.58477 | 3.61503 | |
| 107 | 3.70816 | 4.11671 | 3.58904 | 3.20257 | 3.0367 | 2.88616 | 2.17949 | 4.75507 | 3.56539 | 166 -- |
| | 2.90347 | 2.73739 | 3.18146 | 2.89801 | 2.37887 | 2.77519 | 2.98518 | 4.58477 | 3.61503 | |
| 108 | 3.50317 | 3.82571 | 3.74934 | 3.77795 | 1.83581 | 2.83574 | 3.334 | 5.85879 | 4.66988 | 167 -- |
| | 2.90347 | 2.73739 | 3.18146 | 2.89801 | 2.37887 | 2.77519 | 2.98518 | 4.58477 | 3.61503 | |
| 109 | 2.92361 | 3.82503 | 2.75196 | 2.67917 | 2.27334 | 2.58943 | 3.21968 | 5.47124 | 4.09825 | 168 -- |
| | 2.90347 | 2.73739 | 3.18146 | 2.89801 | 2.37887 | 2.77519 | 2.98518 | 4.58477 | 3.61503 | |
| 110 | 2.74215 | 3.81339 | 2.52207 | 2.76166 | 2.55736 | 2.80216 | 3.52093 | 5.57672 | 4.17124 | 169 -- |
| | 2.90347 | 2.73739 | 3.18146 | 2.89801 | 2.37887 | 2.77519 | 2.98518 | 4.58477 | 3.61503 | |
| 111 | 3.84841 | 4.19881 | 3.74635 | 3.69846 | 2.87972 | 2.82099 | 1.98318 | 4.80657 | 3.61938 | 170 -- |
| | 2.90347 | 2.73739 | 3.18146 | 2.89801 | 2.37887 | 2.77519 | 2.98518 | 4.58477 | 3.61503 | |
| 112 | 2.76246 | 2.12847 | 2.59942 | 2.91244 | 2.6841 | 2.78697 | 3.01012 | 5.36499 | 4.03038 | 171 -- |
| | 2.90347 | 2.73739 | 3.18146 | 2.89801 | 2.37887 | 2.77519 | 2.98518 | 4.58477 | 3.61503 | |
| 113 | 3.49762 | 3.92745 | 3.52287 | 3.53044 | 2.53195 | 2.4909 | 2.45812 | 5.38823 | 4.17148 | 172 -- |
| | 2.90347 | 2.73739 | 3.18146 | 2.89801 | 2.37887 | 2.77519 | 2.98518 | 4.58477 | 3.61503 | |
| 114 | 4.82865 | 4.96286 | 4.71561 | 4.67274 | 4.11561 | 3.53719 | 1.31626 | 5.60027 | 4.4533 | 173 -- |
| | 2.90347 | 2.73739 | 3.18146 | 2.89801 | 2.37887 | 2.77519 | 2.98518 | 4.58477 | 3.61503 | |
| 115 | 4.69765 | 4.90403 | 4.6244 | 4.55898 | 3.99966 | 3.48691 | 0.83238 | 5.65286 | 4.45953 | 174 -- |
| | 2.90347 | 2.73739 | 3.18146 | 2.89801 | 2.37887 | 2.77519 | 2.98518 | 4.58477 | 3.61503 | |
| 116 | 4.10739 | 4.45305 | 4.00681 | 3.97547 | 3.42443 | 3.15654 | 1.32925 | 5.13801 | 3.94577 | 175 -- |
| | 2.90347 | 2.73739 | 3.18146 | 2.89801 | 2.37887 | 2.77519 | 2.98518 | 4.58477 | 3.61503 | |
| 117 | 3.59408 | 3.96657 | 3.86868 | 3.8086 | 0.63275 | 3.06292 | 3.52165 | 5.8339 | 4.58201 | 176 -- |
| | 2.90347 | 2.73739 | 3.18146 | 2.89801 | 2.37887 | 2.77519 | 2.98518 | 4.58477 | 3.61503 | |
| 118 | 3.89844 | 3.42511 | 3.81453 | 3.76874 | 3.2146 | 3.07657 | 1.24673 | 5.12779 | 3.92641 | 177 -- |
| | 2.90347 | 2.73739 | 3.18146 | 2.89801 | 2.37887 | 2.77519 | 2.98518 | 4.58477 | 3.61503 | |
| 119 | 2.82335 | 3.91826 | 2.9346 | 3.26 | 2.4225 | 3.19029 | 3.85503 | 5.92312 | 4.45098 | 178 -- |
| | 2.90347 | 2.73739 | 3.18146 | 2.89801 | 2.37887 | 2.77519 | 2.98518 | 4.58477 | 3.61503 | |
| 120 | 4.22841 | 4.76709 | 4.30182 | 4.19058 | 3.93922 | 4.14006 | 3.70116 | 4.07146 | 0.50853 | 179 -- |
| | 2.90347 | 2.73739 | 3.18146 | 2.89801 | 2.37887 | 2.77519 | 2.98518 | 4.58477 | 3.61503 | |
| 121 | 3.80428 | 4.38334 | 3.395 | 0.52404 | 3.57083 | 3.74393 | 4.02369 | 5.66284 | 4.58903 | 180 -- |
| | 2.90347 | 2.73739 | 3.18146 | 2.89801 | 2.37887 | 2.77519 | 2.98518 | 4.58477 | 3.61503 | |
| 122 | 3.47596 | 4.39885 | 3.84479 | 3.64 | 3.36982 | 3.35845 | 2.62714 | 5.07894 | 3.73532 | 181 -- |
| | 2.90347 | 2.73739 | 3.18146 | 2.89801 | 2.37887 | 2.77519 | 2.98518 | 4.58477 | 3.61503 | |
| 123 | 3.63145 | 3.35531 | 3.86142 | 3.82119 | 2.55547 | 2.83994 | 3.09539 | 5.71638 | 4.54426 | 182 -- |
| | 2.90347 | 2.73739 | 3.18146 | 2.89801 | 2.37887 | 2.77519 | 2.98518 | 4.58477 | 3.61503 | |
| 124 | 4.16117 | 0.34853 | 4.37081 | 4.1814 | 3.52713 | 3.81811 | 4.09794 | 5.89679 | 4.96637 | 183 -- |
| | 2.90347 | 2.73739 | 3.18146 | 2.89801 | 2.37887 | 2.77519 | 2.98518 | 4.58477 | 3.61503 | |
| 125 | 2.97912 | 4.01507 | 3.13217 | 3.28924 | 2.97306 | 3.03134 | 3.73254 | 5.91984 | 4.52338 | 184 -- |
| | 2.90347 | 2.73739 | 3.18146 | 2.89801 | 2.37887 | 2.77519 | 2.98518 | 4.58477 | 3.61503 | |
| 126 | 2.3743 | 3.93517 | 2.90644 | 2.95964 | 2.83402 | 3.11751 | 3.60861 | 5.64573 | 4.25002 | 185 -- |
| | 2.90347 | 2.73739 | 3.18146 | 2.89801 | 2.37887 | 2.77519 | 2.98518 | 4.58477 | 3.61503 | |
| 127 | 3.43138 | 4.23481 | 2.91635 | 0.88496 | 3.31059 | 3.46196 | 3.87023 | 5.59281 | 4.47237 | 186 -- |
| | 2.90347 | 2.73739 | 3.18146 | 2.89801 | 2.37887 | 2.77519 | 2.98518 | 4.58477 | 3.61503 | |
| 128 | 4.70297 | 4.84509 | 4.50218 | 4.33521 | 4.10816 | 3.89905 | 2.70402 | 5.33025 | 4.13656 | 187 -- |
| | 2.90347 | 2.73739 | 3.18146 | 2.89801 | 2.37887 | 2.77519 | 2.98518 | 4.58477 | 3.61503 | |
| 129 | 4.16117 | 0.34853 | 4.37081 | 4.1814 | 3.52713 | 3.81811 | 4.09794 | 5.89679 | 4.96637 | 188 -- |
| | 2.90347 | 2.73739 | 3.18146 | 2.89801 | 2.37887 | 2.77519 | 2.98518 | 4.58477 | 3.61503 | |
| 130 | 3.76814 | 4.02005 | 4.06612 | 3.9693 | 2.7862 | 3.09112 | 3.25386 | 5.86855 | 4.71972 | 189 -- |
| | 2.90347 | 2.73739 | 3.18146 | 2.89801 | 2.37887 | 2.77519 | 2.98518 | 4.58477 | 3.61503 | |
| 131 | 3.39317 | 3.97023 | 3.09699 | 3.28335 | 2.7702 | 2.94935 | 2.90303 | 5.35783 | 4.10375 | 190 -- |
| | 2.90347 | 2.73739 | 3.18146 | 2.89801 | 2.37887 | 2.77519 | 2.98518 | 4.58477 | 3.61503 | |
| 132 | 4.26062 | 4.91982 | 4.34352 | 4.40766 | 4.00166 | 4.1738 | 3.56508 | 3.73496 | 0.65216 | 191 -- |
| | 2.90347 | 2.73739 | 3.18146 | 2.89801 | 2.37887 | 2.77519 | 2.98518 | 4.58477 | 3.61503 | |
| 133 | 2.80644 | 3.94187 | 2.65715 | 3.36015 | 2.89171 | 3.27395 | 3.9777 | 6.03531 | 4.53514 | 192 -- |
| | 2.90347 | 2.73739 | 3.18146 | 2.89801 | 2.37887 | 2.77519 | 2.98518 | 4.58477 | 3.61503 | |
| 134 | 3.23153 | 4.23051 | 3.58785 | 4.05877 | 3.38503 | 3.807 | 4.31921 | 6.11573 | 4.86017 | 193 -- |
| | 2.90347 | 2.73739 | 3.18146 | 2.89801 | 2.37887 | 2.77519 | 2.98518 | 4.58477 | 3.61503 | |
| 135 | 3.53526 | 3.84 | 3.68065 | 3.69839 | 2.40398 | 2.80578 | 2.81028 | 5.60012 | 4.41012 | 194 -- |
| | 2.90347 | 2.73739 | 3.18146 | 2.89801 | 2.37887 | 2.77519 | 2.98518 | 4.58477 | 3.61503 | |
| 136 | 3.50926 | 4.03937 | 3.37595 | 3.07922 | 2.93 | 2.61326 | 1.97187 | 4.81814 | 3.60728 | 195 -- |
| | 2.90347 | 2.73739 | 3.18146 | 2.89801 | 2.37887 | 2.77519 | 2.98518 | 4.58477 | 3.61503 | |
| 137 | 2.6935 | 3.91523 | 2.92601 | 3.04509 | 2.83069 | 3.18123 | 3.85035 | 5.91761 | 4.44826 | 196 -- |
| | 2.90347 | 2.73739 | 3.18146 | 2.89801 | 2.37887 | 2.77519 | 2.98518 | 4.58477 | 3.61503 | |
| 138 | 3.55846 | 3.84747 | 3.82739 | 3.8032 | 2.28623 | 2.64101 | 3.22675 | 5.84657 | 4.66418 | 197 -- |
| | 2.90347 | 2.73739 | 3.18146 | 2.89801 | 2.37887 | 2.77519 | 2.98518 | 4.58477 | 3.61503 | |
| 139 | 4.82529 | 4.94238 | 4.64265 | 4.62261 | 4.11168 | 3.57699 | 1.62526 | 5.49175 | 4.38705 | 198 -- |
| | 2.90347 | 2.73739 | 3.18146 | 2.89801 | 2.37887 | 2.77519 | 2.98518 | 4.58477 | 3.61503 | |
| 140 | 2.71222 | 3.84449 | 2.79741 | 2.8297 | 2.58989 | 2.7297 | 3.20049 | 5.35755 | 4.01653 | 199 -- |
| | 2.90347 | 2.73739 | 3.18146 | 2.89801 | 2.37887 | 2.77519 | 2.98518 | 4.58477 | 3.61503 | |
| 141 | 4.00615 | 4.4762 | 3.94211 | 3.89318 | 3.43946 | 3.39323 | 2.79067 | 1.19213 | 2.58759 | 200 -- |
| | 2.90347 | 2.73739 | 3.18146 | 2.89801 | 2.37887 | 2.77519 | 2.98518 | 4.58477 | 3.61503 | |
| 142 | 4.55145 | 4.78517 | 4.49632 | 4.43629 | 3.84074 | 3.37114 | 1.19156 | 5.5214 | 4.3256 | 201 -- |
| | 2.90347 | 2.73739 | 3.18146 | 2.89801 | 2.37887 | 2.77519 | 2.98518 | 4.58477 | 3.61503 | |
| 143 | 3.34396 | 4.18615 | 2.56635 | 1.23693 | 3.22682 | 3.38915 | 3.89554 | 5.5991 | 4.46463 | 202 -- |
| | 2.90347 | 2.73739 | 3.18146 | 2.89801 | 2.37887 | 2.77519 | 2.98518 | 4.58477 | 3.61503 | |
| 144 | 2.57803 | 3.37565 | 2.65536 | 2.9027 | 2.37923 | 2.42186 | 3.53077 | 5.59818 | 4.19026 | 203 -- |
| | 2.90347 | 2.73739 | 3.18146 | 2.89801 | 2.37887 | 2.77519 | 2.98518 | 4.58477 | 3.61503 | |
| 145 | 2.77551 | 3.83157 | 1.97277 | 2.86265 | 2.36563 | 2.69173 | 3.39663 | 5.49507 | 4.11945 | 204 -- |
| | 2.90347 | 2.73739 | 3.18146 | 2.89801 | 2.37887 | 2.77519 | 2.98518 | 4.58477 | 3.61503 | |
| 146 | 3.3476 | 3.95507 | 2.97916 | 3.21123 | 2.826 | 2.82194 | 2.30503 | 3.90281 | 3.62419 | 205 -- |
| | 2.90347 | 2.73739 | 3.18146 | 2.89801 | 2.37887 | 2.77519 | 2.98518 | 4.58477 | 3.61503 | |
| 147 | 2.92684 | 3.53415 | 2.79639 | 2.86504 | 2.6714 | 2.7148 | 2.75975 | 5.10163 | 3.81425 | 206 -- |
| | 2.90313 | 2.73737 | 3.18155 | 2.8982 | 2.37892 | 2.77538 | 2.98509 | 4.58502 | 3.61528 | |
| 148 | 2.86108 | 3.85204 | 2.83366 | 2.96774 | 2.41809 | 2.89927 | 3.27361 | 5.43288 | 4.08203 | 215 -- |
| | 2.90347 | 2.73739 | 3.18146 | 2.89801 | 2.37887 | 2.77519 | 2.98518 | 4.58477 | 3.61503 | |
| 149 | 2.827 | 3.9295 | 2.947 | 2.84094 | 2.85851 | 3.2174 | 3.8915 | 5.95037 | 4.47796 | 216 -- |
| | 2.90347 | 2.73739 | 3.18146 | 2.89801 | 2.37887 | 2.77519 | 2.98518 | 4.58477 | 3.61503 | |
| 150 | 2.86973 | 2.25357 | 2.54389 | 2.97094 | 2.69459 | 2.85852 | 3.58537 | 5.65728 | 4.24341 | 217 -- |
| | 2.90347 | 2.73739 | 3.18146 | 2.89801 | 2.37887 | 2.77519 | 2.98518 | 4.58477 | 3.61503 | |
| 151 | 4.04957 | 4.49307 | 3.98827 | 3.80764 | 3.47484 | 3.51075 | 2.50914 | 1.00434 | 2.78975 | 218 -- |
| | 2.90347 | 2.73739 | 3.18146 | 2.89801 | 2.37887 | 2.77519 | 2.98518 | 4.58477 | 3.61503 | |
| 152 | 4.72309 | 4.85061 | 4.4628 | 4.48098 | 4.02107 | 3.58102 | 1.88462 | 5.28186 | 4.19755 | 219 -- |
| | 2.90347 | 2.73739 | 3.18146 | 2.89801 | 2.37887 | 2.77519 | 2.98518 | 4.58477 | 3.61503 | |
| 153 | 2.83966 | 3.84698 | 2.74318 | 2.31801 | 2.40296 | 2.72285 | 3.24603 | 5.47504 | 4.12399 | 220 -- |
| | 2.90347 | 2.73739 | 3.18146 | 2.89801 | 2.37887 | 2.77519 | 2.98518 | 4.58477 | 3.61503 | |
| 154 | 2.47841 | 3.84817 | 2.80209 | 2.91069 | 2.48506 | 3.01269 | 3.6648 | 5.72456 | 4.28964 | 221 -- |
| | 2.90347 | 2.73739 | 3.18146 | 2.89801 | 2.37887 | 2.77519 | 2.98518 | 4.58477 | 3.61503 | |
| 155 | 3.50527 | 4.14049 | 3.37848 | 3.21929 | 3.03504 | 3.04768 | 2.76118 | 4.40663 | 1.67141 | 222 -- |
| | 2.90347 | 2.73739 | 3.18146 | 2.89801 | 2.37887 | 2.77519 | 2.98518 | 4.58477 | 3.61503 | |
| 156 | 3.46051 | 3.92853 | 3.45577 | 3.475 | 2.70269 | 2.85377 | 2.05286 | 5.27525 | 4.05253 | 223 -- |
| | 2.90347 | 2.73739 | 3.18146 | 2.89801 | 2.37887 | 2.77519 | 2.98518 | 4.58477 | 3.61503 | |
| 157 | 3.18455 | 4.1833 | 3.53233 | 3.99216 | 3.32886 | 3.74622 | 4.24748 | 6.06325 | 4.79998 | 224 -- |
| | 2.90347 | 2.73739 | 3.18146 | 2.89801 | 2.37887 | 2.77519 | 2.98518 | 4.58477 | 3.61503 | |
| 158 | 3.73503 | 3.52921 | 3.62282 | 3.59553 | 3.07632 | 2.93642 | 2.32618 | 4.62538 | 2.91699 | 225 -- |
| | 2.90347 | 2.73739 | 3.18146 | 2.89801 | 2.37887 | 2.77519 | 2.98518 | 4.58477 | 3.61503 | |
| 159 | 2.77504 | 3.86695 | 3.45173 | 3.48253 | 0.92277 | 2.95732 | 3.45348 | 5.76287 | 4.44925 | 226 -- |
| | 2.90347 | 2.73739 | 3.18146 | 2.89801 | 2.37887 | 2.77519 | 2.98518 | 4.58477 | 3.61503 | |
| 160 | 2.37252 | 3.96408 | 2.76789 | 1.79599 | 2.85932 | 2.93721 | 3.628 | 5.55554 | 4.27472 | 227 -- |
| | 2.90347 | 2.73739 | 3.18146 | 2.89801 | 2.37887 | 2.77519 | 2.98518 | 4.58477 | 3.61503 | |
| 161 | 3.86518 | 4.15703 | 3.87085 | 3.80476 | 2.84726 | 2.8073 | 1.56074 | 5.09092 | 3.89445 | 228 -- |
| | 2.90347 | 2.73739 | 3.18146 | 2.89801 | 2.37887 | 2.77519 | 2.98518 | 4.58477 | 3.61503 | |
| 162 | 4.22347 | 4.78698 | 4.25797 | 4.30799 | 3.83111 | 3.85336 | 2.8556 | 3.83491 | 1.53446 | 229 -- |
| | 2.90347 | 2.73739 | 3.18146 | 2.89801 | 2.37887 | 2.77519 | 2.98518 | 4.58477 | 3.61503 | |
| 163 | 4.80849 | 4.90642 | 4.54591 | 4.55791 | 4.09365 | 3.61491 | 1.88885 | 5.36168 | 4.31003 | 230 -- |
| | 2.90347 | 2.73739 | 3.18146 | 2.89801 | 2.37887 | 2.77519 | 2.98518 | 4.58477 | 3.61503 | |
| 164 | 3.98729 | 4.34003 | 3.84639 | 3.79801 | 3.30327 | 3.11266 | 2.30468 | 4.9072 | 3.39865 | 231 -- |
| | 2.90347 | 2.73739 | 3.18146 | 2.89801 | 2.37887 | 2.77519 | 2.98518 | 4.58477 | 3.61503 | |
| 165 | 4.13001 | 4.33412 | 4.44637 | 4.32905 | 3.39874 | 3.72363 | 4.18538 | 5.99711 | 5.14174 | 232 -- |
| | 2.90347 | 2.73739 | 3.18146 | 2.89801 | 2.37887 | 2.77519 | 2.98518 | 4.58477 | 3.61503 | |
| 166 | 3.03569 | 3.90075 | 3.00573 | 3.09204 | 1.84856 | 2.49828 | 2.87913 | 5.14368 | 3.86879 | 233 -- |
| | 2.90347 | 2.73739 | 3.18146 | 2.89801 | 2.37887 | 2.77519 | 2.98518 | 4.58477 | 3.61503 | |
| 167 | 3.02832 | 3.9191 | 3.05809 | 3.18797 | 1.20559 | 3.05132 | 3.45183 | 5.56968 | 4.18219 | 234 -- |
| | 2.90347 | 2.73739 | 3.18146 | 2.89801 | 2.37887 | 2.77519 | 2.98518 | 4.58477 | 3.61503 | |
| 168 | 2.63706 | 3.84577 | 3.36963 | 3.44049 | 2.09599 | 2.60546 | 3.27361 | 5.66066 | 4.40726 | 235 -- |
| | 2.90347 | 2.73739 | 3.18146 | 2.89801 | 2.37887 | 2.77519 | 2.98518 | 4.58477 | 3.61503 | |
| 169 | 4.13001 | 4.33412 | 4.44637 | 4.32905 | 3.39874 | 3.72363 | 4.18538 | 5.99711 | 5.14174 | 236 -- |
| | 2.90347 | 2.73739 | 3.18146 | 2.89801 | 2.37887 | 2.77519 | 2.98518 | 4.58477 | 3.61503 | |
| 170 | 3.5337 | 3.86418 | 3.82864 | 3.85176 | 2.57111 | 2.90314 | 3.40139 | 5.93785 | 4.76384 | 237 -- |
| | 2.90347 | 2.73739 | 3.18146 | 2.89801 | 2.37887 | 2.77519 | 2.98518 | 4.58477 | 3.61503 | |
| 171 | 1.14369 | 3.9346 | 3.26019 | 3.30428 | 2.7607 | 2.3136 | 3.4609 | 5.74715 | 4.40951 | 238 -- |
| | 2.90347 | 2.73739 | 3.18146 | 2.89801 | 2.37887 | 2.77519 | 2.98518 | 4.58477 | 3.61503 | |
| 172 | 4.79062 | 4.91387 | 4.55933 | 4.54474 | 4.08911 | 3.59769 | 2.02622 | 5.40471 | 4.3178 | 239 -- |
| | 2.90347 | 2.73739 | 3.18146 | 2.89801 | 2.37887 | 2.77519 | 2.98518 | 4.58477 | 3.61503 | |
| 173 | 3.55254 | 3.92217 | 3.62831 | 3.63291 | 2.4323 | 2.56229 | 2.06166 | 5.42804 | 4.22422 | 240 -- |
| | 2.90347 | 2.73739 | 3.18146 | 2.89801 | 2.37887 | 2.77519 | 2.98518 | 4.58477 | 3.61503 | |
| 174 | 4.25836 | 4.84403 | 4.30308 | 4.36634 | 3.90761 | 3.95172 | 3.28836 | 3.79313 | 1.01641 | 241 -- |
| | 2.90347 | 2.73739 | 3.18146 | 2.89801 | 2.37887 | 2.77519 | 2.98518 | 4.58477 | 3.61503 | |
| 175 | 2.32634 | 3.86247 | 2.48809 | 2.69167 | 2.68159 | 2.88789 | 3.22597 | 5.30912 | 3.57131 | 242 -- |
| | 2.90347 | 2.73739 | 3.18146 | 2.89801 | 2.37887 | 2.77519 | 2.98518 | 4.58477 | 3.61503 | |
| 176 | 3.70806 | 4.13653 | 3.67338 | 3.66534 | 2.96651 | 2.45344 | 1.58869 | 5.23813 | 4.02633 | 243 -- |
| | 2.90347 | 2.73739 | 3.18146 | 2.89801 | 2.37887 | 2.77519 | 2.98518 | 4.58477 | 3.61503 | |
| 177 | 3.09386 | 3.8353 | 3.58054 | 3.65727 | 2.38841 | 2.86886 | 3.38279 | 5.84237 | 4.61572 | 244 -- |
| | 2.90347 | 2.73739 | 3.18146 | 2.89801 | 2.37887 | 2.77519 | 2.98518 | 4.58477 | 3.61503 | |
| 178 | 4.84385 | 4.93546 | 4.49536 | 4.52685 | 4.16262 | 3.72765 | 2.26283 | 5.32102 | 4.30308 | 245 -- |
| | 2.90347 | 2.73739 | 3.18146 | 2.89801 | 2.37887 | 2.77519 | 2.98518 | 4.58477 | 3.61503 | |
| 179 | 2.95959 | 3.90852 | 2.73145 | 1.71155 | 2.75236 | 2.9299 | 3.19092 | 4.55388 | 4.02765 | 246 -- |
| | 2.90347 | 2.73739 | 3.18146 | 2.89801 | 2.37887 | 2.77519 | 2.98518 | 4.58477 | 3.61503 | |
| 180 | 3.45764 | 3.96581 | 3.39827 | 3.4257 | 2.32623 | 2.68907 | 2.05848 | 5.15342 | 3.93086 | 247 -- |
| | 2.90347 | 2.73739 | 3.18146 | 2.89801 | 2.37887 | 2.77519 | 2.98518 | 4.58477 | 3.61503 | |
| 181 | 3.49135 | 4.01152 | 3.21082 | 3.38793 | 2.78551 | 2.85216 | 2.31515 | 4.88732 | 3.67459 | 248 -- |
| | 2.90347 | 2.73739 | 3.18146 | 2.89801 | 2.37887 | 2.77519 | 2.98518 | 4.58477 | 3.61503 | |
| 182 | 2.59984 | 3.82397 | 2.74555 | 2.89028 | 2.57094 | 2.89663 | 3.30162 | 5.54537 | 4.15131 | 249 -- |
| | 2.90347 | 2.73739 | 3.18146 | 2.89801 | 2.37887 | 2.77519 | 2.98518 | 4.58477 | 3.61503 | |
| 183 | 3.10273 | 3.93899 | 3.06793 | 2.98479 | 2.64808 | 2.75893 | 2.46463 | 5.01643 | 3.76148 | 250 -- |
| | 2.90353 | 2.73746 | 3.18153 | 2.898 | 2.37887 | 2.77526 | 2.98489 | 4.58483 | 3.61509 | |
| 184 | 2.77556 | 3.9534 | 2.84007 | 3.52097 | 2.77605 | 3.36463 | 4.05969 | 6.13946 | 4.61155 | 253 -- |
| | 2.90347 | 2.73739 | 3.18146 | 2.89801 | 2.37887 | 2.77519 | 2.98518 | 4.58477 | 3.61503 | |
| 185 | 4.23309 | 4.51695 | 4.05418 | 4.04354 | 3.54514 | 2.79737 | 2.05308 | 5.05967 | 3.93294 | 254 -- |
| | 2.90347 | 2.73739 | 3.18146 | 2.89801 | 2.37887 | 2.77519 | 2.98518 | 4.58477 | 3.61503 | |
| 186 | 2.76217 | 3.82276 | 2.75088 | 2.91665 | 2.09891 | 2.81435 | 3.54307 | 5.61098 | 4.19982 | 255 -- |
| | 2.90347 | 2.73739 | 3.18146 | 2.89801 | 2.37887 | 2.77519 | 2.98518 | 4.58477 | 3.61503 | |
| 187 | 3.18428 | 1.33641 | 3.21308 | 3.22378 | 2.70037 | 2.97541 | 3.35691 | 5.64772 | 4.36442 | 256 -- |
| | 2.90347 | 2.73739 | 3.18146 | 2.89801 | 2.37887 | 2.77519 | 2.98518 | 4.58477 | 3.61503 | |
| 188 | 4.44297 | 4.64734 | 4.29787 | 4.25943 | 3.70513 | 3.32735 | 1.63114 | 5.19097 | 4.06177 | 257 -- |
| | 2.90347 | 2.73739 | 3.18146 | 2.89801 | 2.37887 | 2.77519 | 2.98518 | 4.58477 | 3.61503 | |
| 189 | 2.97283 | 3.8563 | 2.61019 | 2.44541 | 2.68096 | 2.77295 | 3.34473 | 5.43447 | 4.09447 | 258 -- |
| | 2.90347 | 2.73739 | 3.18146 | 2.89801 | 2.37887 | 2.77519 | 2.98518 | 4.58477 | 3.61503 | |
| 190 | 4.84266 | 4.98596 | 4.71018 | 4.66313 | 4.14762 | 3.57919 | 1.64071 | 5.61982 | 4.46683 | 259 -- |
| | 2.90347 | 2.73739 | 3.18146 | 2.89801 | 2.37887 | 2.77519 | 2.98518 | 4.58477 | 3.61503 | |
| 191 | 2.96886 | 3.84271 | 2.50606 | 2.23991 | 2.43867 | 2.75605 | 3.10587 | 5.38164 | 4.03615 | 260 -- |
| | 2.90347 | 2.73739 | 3.18146 | 2.89801 | 2.37887 | 2.77519 | 2.98518 | 4.58477 | 3.61503 | |
| 192 | 4.13001 | 4.33412 | 4.44637 | 4.32905 | 3.39874 | 3.72363 | 4.18538 | 5.99711 | 5.14174 | 261 -- |
| | 2.90347 | 2.73739 | 3.18146 | 2.89801 | 2.37887 | 2.77519 | 2.98518 | 4.58477 | 3.61503 | |
| 193 | 4.42883 | 4.64124 | 4.25288 | 4.22803 | 3.70233 | 3.35877 | 1.85988 | 5.16553 | 4.05112 | 262 -- |
| | 2.90347 | 2.73739 | 3.18146 | 2.89801 | 2.37887 | 2.77519 | 2.98518 | 4.58477 | 3.61503 | |
| 194 | 4.32006 | 4.60954 | 4.25312 | 4.18473 | 3.60893 | 3.2602 | 1.47614 | 5.33681 | 4.13282 | 263 -- |
| | 2.90347 | 2.73739 | 3.18146 | 2.89801 | 2.37887 | 2.77519 | 2.98518 | 4.58477 | 3.61503 | |
| 195 | 4.87099 | 4.92537 | 4.50327 | 4.55713 | 4.16435 | 3.72988 | 2.50069 | 5.24999 | 4.22787 | 264 -- |
| | 2.90347 | 2.73739 | 3.18146 | 2.89801 | 2.37887 | 2.77519 | 2.98518 | 4.58477 | 3.61503 | |
| 196 | 2.01295 | 3.83746 | 2.62923 | 2.78951 | 2.65108 | 2.88278 | 3.32791 | 5.44105 | 3.78625 | 265 -- |
| | 2.90347 | 2.73739 | 3.18146 | 2.89801 | 2.37887 | 2.77519 | 2.98518 | 4.58477 | 3.61503 | |
| 197 | 3.06148 | 3.91993 | 1.56025 | 2.78935 | 2.76841 | 2.9663 | 3.1073 | 5.3531 | 4.0176 | 266 -- |
| | 2.90347 | 2.73739 | 3.18146 | 2.89801 | 2.37887 | 2.77519 | 2.98518 | 4.58477 | 3.61503 | |
| 198 | 3.47759 | 1.07138 | 3.55227 | 3.47022 | 2.74934 | 2.99572 | 3.02021 | 5.57559 | 4.30789 | 267 -- |
| | 2.90347 | 2.73739 | 3.18146 | 2.89801 | 2.37887 | 2.77519 | 2.98518 | 4.58477 | 3.61503 | |
| 199 | 3.85272 | 4.36901 | 3.78968 | 3.79415 | 3.06756 | 3.25556 | 2.7829 | 4.11682 | 1.90503 | 268 -- |
| | 2.90347 | 2.73739 | 3.18146 | 2.89801 | 2.37887 | 2.77519 | 2.98518 | 4.58477 | 3.61503 | |
| 200 | 4.48245 | 4.84946 | 4.37958 | 4.39217 | 3.97301 | 3.81947 | 2.78356 | 4.34976 | 2.73477 | 269 -- |
| | 2.90347 | 2.73739 | 3.18146 | 2.89801 | 2.37887 | 2.77519 | 2.98518 | 4.58477 | 3.61503 | |
| 201 | 3.43781 | 3.83194 | 3.68626 | 3.73544 | 1.79077 | 2.55653 | 3.42345 | 5.92287 | 4.71349 | 270 -- |
| | 2.90347 | 2.73739 | 3.18146 | 2.89801 | 2.37887 | 2.77519 | 2.98518 | 4.58477 | 3.61503 | |
| 202 | 3.61161 | 3.9805 | 3.95199 | 3.93802 | 2.75595 | 3.10087 | 3.59477 | 5.99348 | 4.85068 | 271 -- |
| | 2.90347 | 2.73739 | 3.18146 | 2.89801 | 2.37887 | 2.77519 | 2.98518 | 4.58477 | 3.61503 | |
| 203 | 3.29571 | 3.96869 | 3.14178 | 3.19598 | 2.676 | 2.43226 | 1.94094 | 4.97522 | 3.73231 | 272 -- |
| | 2.90347 | 2.73739 | 3.18146 | 2.89801 | 2.37887 | 2.77519 | 2.98518 | 4.58477 | 3.61503 | |
| 204 | 2.8777 | 3.82831 | 2.55963 | 2.8794 | 2.64761 | 2.72971 | 3.54563 | 5.60351 | 4.19684 | 273 -- |
| | 2.90347 | 2.73739 | 3.18146 | 2.89801 | 2.37887 | 2.77519 | 2.98518 | 4.58477 | 3.61503 | |
| 205 | 3.17947 | 3.63101 | 2.88986 | 1.15319 | 2.81968 | 3.18288 | 3.59806 | 5.56228 | 4.322 | 274 -- |
| | 2.90347 | 2.73739 | 3.18146 | 2.89801 | 2.37887 | 2.77519 | 2.98518 | 4.58477 | 3.61503 | |
| 206 | 3.45519 | 3.9076 | 3.45303 | 3.46688 | 2.02051 | 1.35458 | 2.84596 | 5.3295 | 4.10639 | 275 -- |
| | 2.90347 | 2.73739 | 3.18146 | 2.89801 | 2.37887 | 2.77519 | 2.98518 | 4.58477 | 3.61503 | |
| 207 | 2.87525 | 3.0584 | 2.5371 | 2.73481 | 2.63606 | 2.90857 | 3.53788 | 5.59647 | 4.18833 | 276 -- |
| | 2.90347 | 2.73739 | 3.18146 | 2.89801 | 2.37887 | 2.77519 | 2.98518 | 4.58477 | 3.61503 | |
| 208 | 3.49912 | 3.84518 | 3.75548 | 3.75749 | 0.86738 | 2.87074 | 3.35269 | 5.89192 | 4.68098 | 277 -- |
| | 2.90347 | 2.73739 | 3.18146 | 2.89801 | 2.37887 | 2.77519 | 2.98518 | 4.58477 | 3.61503 | |
| 209 | 2.90712 | 4.0313 | 3.1536 | 3.5168 | 3.04275 | 3.44139 | 4.03108 | 6.0823 | 4.63717 | 278 -- |
| | 2.90347 | 2.73739 | 3.18146 | 2.89801 | 2.37887 | 2.77519 | 2.98518 | 4.58477 | 3.61503 | |
| 210 | 3.65396 | 4.10439 | 3.53411 | 3.51415 | 2.89227 | 2.72032 | 2.13945 | 4.79947 | 3.59983 | 279 -- |
| | 2.90347 | 2.73739 | 3.18146 | 2.89801 | 2.37887 | 2.77519 | 2.98518 | 4.58477 | 3.61503 | |
| 211 | 2.88129 | 4.01724 | 2.80596 | 1.40204 | 2.9345 | 3.14584 | 3.64213 | 5.55856 | 4.30545 | 280 -- |
| | 2.90347 | 2.73739 | 3.18146 | 2.89801 | 2.37887 | 2.77519 | 2.98518 | 4.58477 | 3.61503 | |
| 212 | 2.944 | 3.99632 | 3.21207 | 3.07928 | 2.47706 | 2.87363 | 2.62157 | 4.89026 | 3.40426 | 281 -- |
| | 2.90347 | 2.73739 | 3.18146 | 2.89801 | 2.37887 | 2.77519 | 2.98518 | 4.58477 | 3.61503 | |
| 213 | 3.29944 | 3.96475 | 3.14626 | 2.99158 | 2.82519 | 2.84993 | 2.19138 | 4.94948 | 3.70917 | 282 -- |
| | 2.90347 | 2.73739 | 3.18146 | 2.89801 | 2.37887 | 2.77519 | 2.98518 | 4.58477 | 3.61503 | |
| 214 | 1.73014 | 3.88825 | 2.89428 | 3.1513 | 2.65166 | 2.73562 | 3.67772 | 5.77869 | 4.34804 | 283 -- |
| | 2.90347 | 2.73739 | 3.18146 | 2.89801 | 2.37887 | 2.77519 | 2.98518 | 4.58477 | 3.61503 | |
| 215 | 2.97097 | 4.05638 | 3.29801 | 3.8004 | 3.07468 | 3.49776 | 4.13871 | 6.17808 | 4.76353 | 284 -- |
| | 2.90347 | 2.73739 | 3.18146 | 2.89801 | 2.37887 | 2.77519 | 2.98518 | 4.58477 | 3.61503 | |
| 216 | 2.92666 | 2.91335 | 2.44294 | 2.35575 | 2.4315 | 2.87572 | 3.38109 | 5.47602 | 3.88189 | 285 -- |
| | 2.90347 | 2.73739 | 3.18146 | 2.89801 | 2.37887 | 2.77519 | 2.98518 | 4.58477 | 3.61503 | |
| 217 | 3.44356 | 4.05419 | 3.44165 | 3.29071 | 2.84849 | 2.63023 | 1.70902 | 4.7806 | 3.57608 | 286 -- |
| | 2.90347 | 2.73739 | 3.18146 | 2.89801 | 2.37887 | 2.77519 | 2.98518 | 4.58477 | 3.61503 | |
| 218 | 4.30461 | 4.50902 | 4.14108 | 4.10077 | 3.54181 | 3.23769 | 2.15223 | 4.98317 | 3.85773 | 287 -- |
| | 2.90347 | 2.73739 | 3.18146 | 2.89801 | 2.37887 | 2.77519 | 2.98518 | 4.58477 | 3.61503 | |
| 219 | 3.38069 | 1.15025 | 3.43381 | 3.35959 | 2.46504 | 2.97792 | 3.17622 | 5.51871 | 4.21657 | 288 -- |
| | 2.90347 | 2.73739 | 3.18146 | 2.89801 | 2.37887 | 2.77519 | 2.98518 | 4.58477 | 3.61503 | |
| 220 | 3.07721 | 3.0092 | 3.2694 | 3.2637 | 2.66456 | 2.89078 | 2.57005 | 5.02262 | 3.78267 | 289 -- |
| | 2.90347 | 2.73739 | 3.18146 | 2.89801 | 2.37887 | 2.77519 | 2.98518 | 4.58477 | 3.61503 | |
| 221 | 3.21588 | 2.52613 | 2.94699 | 3.12992 | 2.58578 | 2.75312 | 2.67351 | 5.03407 | 3.77642 | 290 -- |
| | 2.90347 | 2.73739 | 3.18146 | 2.89801 | 2.37887 | 2.77519 | 2.98518 | 4.58477 | 3.61503 | |
| 222 | 3.14395 | 3.98051 | 3.25292 | 3.28556 | 2.76033 | 2.75863 | 1.98205 | 4.87558 | 3.67659 | 291 -- |
| | 2.90347 | 2.73739 | 3.18146 | 2.89801 | 2.37887 | 2.77519 | 2.98518 | 4.58477 | 3.61503 | |
| 223 | 2.18317 | 3.90854 | 3.02104 | 3.10187 | 2.49669 | 2.34772 | 2.74653 | 5.12823 | 3.85713 | 292 -- |
| | 2.90347 | 2.73739 | 3.18146 | 2.89801 | 2.37887 | 2.77519 | 2.98518 | 4.58477 | 3.61503 | |
| 224 | 2.88695 | 4.02477 | 3.15442 | 3.57919 | 3.03181 | 3.43913 | 4.05741 | 5.96487 | 4.4489 | 293 -- |
| | 2.90347 | 2.73739 | 3.18146 | 2.89801 | 2.37887 | 2.77519 | 2.98518 | 4.58477 | 3.61503 | |
| 225 | 3.65478 | 4.11341 | 3.52176 | 3.10264 | 3.02688 | 2.90444 | 2.04871 | 4.80614 | 3.61104 | 294 -- |
| | 2.90347 | 2.73739 | 3.18146 | 2.89801 | 2.37887 | 2.77519 | 2.98518 | 4.58477 | 3.61503 | |
| 226 | 4.0203 | 4.32364 | 3.88904 | 3.84436 | 3.29242 | 2.84047 | 2.27852 | 4.88107 | 3.71993 | 295 -- |
| | 2.90347 | 2.73739 | 3.18146 | 2.89801 | 2.37887 | 2.77519 | 2.98518 | 4.58477 | 3.61503 | |
| 227 | 4.07041 | 4.51704 | 4.01665 | 3.82098 | 3.5109 | 3.56822 | 2.6592 | 0.93931 | 2.74344 | 296 -- |
| | 2.90347 | 2.73739 | 3.18146 | 2.89801 | 2.37887 | 2.77519 | 2.98518 | 4.58477 | 3.61503 | |
| 228 | 2.74043 | 3.84877 | 2.69295 | 2.98051 | 2.17151 | 2.98395 | 3.6221 | 5.68425 | 4.26026 | 297 -- |
| | 2.90347 | 2.73739 | 3.18146 | 2.89801 | 2.37887 | 2.77519 | 2.98518 | 4.58477 | 3.61503 | |
| 229 | 4.06974 | 4.47111 | 3.90435 | 3.89941 | 3.46749 | 3.28148 | 2.36925 | 4.99959 | 3.75961 | 298 -- |
| | 2.90347 | 2.73739 | 3.18146 | 2.89801 | 2.37887 | 2.77519 | 2.98518 | 4.58477 | 3.61503 | |
| 230 | 3.53126 | 3.85527 | 3.6436 | 3.66186 | 1.67824 | 2.62186 | 2.8189 | 5.51449 | 4.31994 | 299 -- |
| | 2.90347 | 2.73739 | 3.18146 | 2.89801 | 2.37887 | 2.77519 | 2.98518 | 4.58477 | 3.61503 | |
| 231 | 4.70297 | 4.84509 | 4.50218 | 4.33521 | 4.10816 | 3.89905 | 2.70402 | 5.33025 | 4.13656 | 300 -- |
| | 2.90347 | 2.73739 | 3.18146 | 2.89801 | 2.37887 | 2.77519 | 2.98518 | 4.58477 | 3.61503 | |
| 232 | 4.16117 | 0.34853 | 4.37081 | 4.1814 | 3.52713 | 3.81811 | 4.09794 | 5.89679 | 4.96637 | 301 -- |
| | 2.90347 | 2.73739 | 3.18146 | 2.89801 | 2.37887 | 2.77519 | 2.98518 | 4.58477 | 3.61503 | |
| 233 | 3.08763 | 3.92642 | 3.00578 | 3.08654 | 2.76967 | 2.86943 | 1.99402 | 5.11237 | 3.8387 | 302 -- |
| | 2.90347 | 2.73739 | 3.18146 | 2.89801 | 2.37887 | 2.77519 | 2.98518 | 4.58477 | 3.61503 | |
| 234 | 3.20718 | 3.91966 | 3.4794 | 3.68802 | 2.53414 | 3.07678 | 3.60843 | 5.95082 | 4.64243 | 303 -- |
| | 2.90347 | 2.73739 | 3.18146 | 2.89801 | 2.37887 | 2.77519 | 2.98518 | 4.58477 | 3.61503 | |
| 235 | 3.57107 | 3.95413 | 3.58438 | 3.59272 | 2.13812 | 2.56649 | 1.71302 | 5.18442 | 3.98287 | 304 -- |
| | 2.90347 | 2.73739 | 3.18146 | 2.89801 | 2.37887 | 2.77519 | 2.98518 | 4.58477 | 3.61503 | |
| 236 | 2.5037 | 3.98042 | 3.12571 | 3.60425 | 2.95902 | 3.10524 | 4.01731 | 6.14562 | 4.63274 | 305 -- |
| | 2.90347 | 2.73739 | 3.18146 | 2.89801 | 2.37887 | 2.77519 | 2.98518 | 4.58477 | 3.61503 | |
| 237 | 3.33142 | 4.14725 | 2.64027 | 0.99486 | 3.1434 | 3.08656 | 3.69625 | 5.55454 | 4.37587 | 306 -- |
| | 2.90347 | 2.73739 | 3.18146 | 2.89801 | 2.37887 | 2.77519 | 2.98518 | 4.58477 | 3.61503 | |
| 238 | 2.69652 | 3.98805 | 3.1623 | 3.67603 | 2.71784 | 3.40211 | 4.06257 | 6.17855 | 4.65259 | 307 -- |
| | 2.90347 | 2.73739 | 3.18146 | 2.89801 | 2.37887 | 2.77519 | 2.98518 | 4.58477 | 3.61503 | |
| 239 | 3.79703 | 4.41988 | 3.78638 | 3.48044 | 3.58665 | 3.83775 | 4.00762 | 5.08791 | 3.57711 | 308 -- |
| | 2.90347 | 2.73739 | 3.18146 | 2.89801 | 2.37887 | 2.77519 | 2.98518 | 4.58477 | 3.61503 | |
| 240 | 2.98933 | 3.89375 | 2.8645 | 2.66573 | 2.74007 | 2.97191 | 2.71063 | 5.48831 | 4.14512 | 309 -- |
| | 2.90347 | 2.73739 | 3.18146 | 2.89801 | 2.37887 | 2.77519 | 2.98518 | 4.58477 | 3.61503 | |
| 241 | 4.08871 | 4.64221 | 4.09598 | 4.12045 | 3.6343 | 3.65785 | 3.08398 | 3.91121 | 0.85287 | 310 -- |
| | 2.90347 | 2.73739 | 3.18146 | 2.89801 | 2.37887 | 2.77519 | 2.98518 | 4.58477 | 3.61503 | |
| 242 | 3.59529 | 3.94484 | 3.63802 | 3.62731 | 1.72214 | 2.84419 | 2.7186 | 5.16428 | 3.9425 | 311 -- |
| | 2.90347 | 2.73739 | 3.18146 | 2.89801 | 2.37887 | 2.77519 | 2.98518 | 4.58477 | 3.61503 | |
| 243 | 0.92123 | 4.01695 | 3.2162 | 3.51804 | 2.98116 | 3.35957 | 3.89955 | 5.94846 | 4.50832 | 312 -- |
| | 2.90347 | 2.73739 | 3.18146 | 2.89801 | 2.37887 | 2.77519 | 2.98518 | 4.58477 | 3.61503 | |
| 244 | 3.63729 | 0.93137 | 3.64824 | 3.49941 | 3.03624 | 3.24099 | 3.122 | 5.44029 | 4.14682 | 313 -- |
| | 2.90347 | 2.73739 | 3.18146 | 2.89801 | 2.37887 | 2.77519 | 2.98518 | 4.58477 | 3.61503 | |
| 245 | 3.46225 | 4.03364 | 3.31958 | 3.21658 | 2.91809 | 2.41827 | 2.33006 | 4.86952 | 3.65313 | 314 -- |
| | 2.90347 | 2.73739 | 3.18146 | 2.89801 | 2.37887 | 2.77519 | 2.98518 | 4.58477 | 3.61503 | |
| 246 | 3.13012 | 3.90333 | 2.86653 | 2.93774 | 2.54333 | 2.76419 | 2.40077 | 5.12998 | 3.84971 | 315 -- |
| | 2.90347 | 2.73739 | 3.18146 | 2.89801 | 2.37887 | 2.77519 | 2.98518 | 4.58477 | 3.61503 | |
| 247 | 2.65165 | 3.81945 | 2.74001 | 2.73557 | 2.50587 | 2.8631 | 3.23909 | 5.46696 | 3.81282 | 316 -- |
| | 2.90347 | 2.73739 | 3.18146 | 2.89801 | 2.37887 | 2.77519 | 2.98518 | 4.58477 | 3.61503 | |
| 248 | 2.55415 | 3.61178 | 2.59243 | 2.78996 | 2.55147 | 2.79948 | 3.39944 | 5.49285 | 4.10988 | 317 -- |
| | 2.90347 | 2.73739 | 3.18146 | 2.89801 | 2.37887 | 2.77519 | 2.98518 | 4.58477 | 3.61503 | |
| 249 | 2.66634 | 3.41531 | 2.79728 | 2.91738 | 2.46457 | 2.86855 | 3.23119 | 4.22213 | 4.03642 | 318 -- |
| | 2.90347 | 2.7374 | 3.18147 | 2.89801 | 2.37887 | 2.7752 | 2.98511 | 4.58477 | 3.61503 | |
| 250 | 2.97532 | 3.41289 | 2.74765 | 2.68978 | 2.28116 | 2.7845 | 2.80195 | 5.17138 | 3.86328 | 320 -- |
| | 2.90347 | 2.73739 | 3.18146 | 2.89801 | 2.37887 | 2.77519 | 2.98518 | 4.58477 | 3.61503 | |
| 251 | 3.00997 | 3.82478 | 2.74314 | 2.7896 | 2.45999 | 2.80588 | 2.53965 | 5.1557 | 3.85547 | 321 -- |
| | 2.90347 | 2.73739 | 3.18146 | 2.89801 | 2.37887 | 2.77519 | 2.98518 | 4.58477 | 3.61503 | |
| 252 | 2.8122 | 3.7905 | 2.6251 | 2.62135 | 2.47224 | 2.83958 | 3.35964 | 5.4551 | 3.74485 | 322 -- |
| | 2.90347 | 2.73739 | 3.18146 | 2.89801 | 2.37887 | 2.77519 | 2.98518 | 4.58477 | 3.61503 | |
| 253 | 2.83295 | 3.58992 | 2.78824 | 3.05358 | 2.7367 | 2.84284 | 2.83694 | 5.09255 | 3.3308 | 323 -- |
| | 2.90347 | 2.73739 | 3.18146 | 2.89801 | 2.37887 | 2.77519 | 2.98518 | 4.58477 | 3.61503 | |
| 254 | 2.77944 | 3.81922 | 2.74459 | 2.90152 | 2.56399 | 2.90986 | 3.49974 | 5.57629 | 4.17314 | 324 -- |
| | 2.90347 | 2.73739 | 3.18146 | 2.89801 | 2.37887 | 2.77519 | 2.98518 | 4.58477 | 3.61503 | |
| 255 | 2.54247 | 3.84427 | 2.69556 | 2.5689 | 2.65794 | 2.87639 | 3.28082 | 5.40665 | 3.84179 | 325 -- |
| | 2.90347 | 2.73739 | 3.18146 | 2.89801 | 2.37887 | 2.77519 | 2.98518 | 4.58477 | 3.61503 | |
| 256 | 4.74398 | 4.88662 | 4.61038 | 4.5748 | 4.01958 | 3.49274 | 1.40449 | 5.47814 | 4.34912 | 326 -- |
| | 2.90347 | 2.73739 | 3.18146 | 2.89801 | 2.37887 | 2.77519 | 2.98518 | 4.58477 | 3.61503 | |
| 257 | 3.05039 | 3.92287 | 2.81208 | 2.10241 | 2.77657 | 2.87807 | 3.47322 | 5.51568 | 4.20875 | 327 -- |
| | 2.90347 | 2.73739 | 3.18146 | 2.89801 | 2.37887 | 2.77519 | 2.98518 | 4.58477 | 3.61503 | |
| 258 | 2.91438 | 3.83401 | 2.65614 | 1.93814 | 2.5552 | 2.90262 | 3.21183 | 5.53655 | 4.15574 | 328 -- |
| | 2.90347 | 2.73739 | 3.18146 | 2.89801 | 2.37887 | 2.77519 | 2.98518 | 4.58477 | 3.61503 | |
| 259 | 3.8897 | 4.28429 | 3.47181 | 3.69831 | 3.24204 | 3.07326 | 2.32841 | 4.8715 | 3.68322 | 329 -- |
| | 2.90347 | 2.73739 | 3.18146 | 2.89801 | 2.37887 | 2.77519 | 2.98518 | 4.58477 | 3.61503 | |
| 260 | 2.94878 | 2.34022 | 2.61667 | 2.58698 | 2.68977 | 2.8241 | 3.4139 | 5.50433 | 4.14518 | 330 -- |
| | 2.9035 | 2.73743 | 3.1815 | 2.89804 | 2.3789 | 2.77523 | 2.98522 | 4.58104 | 3.61507 | |
| 261 | 2.91846 | 3.26698 | 2.66368 | 2.06261 | 2.6113 | 2.83052 | 3.32774 | 4.33587 | 4.08982 | 332 -- |
| | 2.90347 | 2.73739 | 3.18146 | 2.89801 | 2.37887 | 2.77519 | 2.98518 | 4.58477 | 3.61503 | |
| 262 | 4.10273 | 4.38925 | 4.04113 | 3.98024 | 3.33621 | 2.92421 | 1.52938 | 5.06809 | 3.87243 | 333 -- |
| | 2.90347 | 2.73739 | 3.18146 | 2.89801 | 2.37887 | 2.77519 | 2.98518 | 4.58477 | 3.61503 | |
| 263 | 4.17895 | 4.46218 | 3.9882 | 3.50849 | 3.48131 | 3.24376 | 2.38294 | 4.93975 | 3.79486 | 334 -- |
| | 2.90347 | 2.73739 | 3.18146 | 2.89801 | 2.37887 | 2.77519 | 2.98518 | 4.58477 | 3.61503 | |
| 264 | 4.66922 | 4.86428 | 4.57938 | 4.53513 | 3.96412 | 3.45481 | 0.92447 | 5.52 | 4.33392 | 335 -- |
| | 2.90347 | 2.73739 | 3.18146 | 2.89801 | 2.37887 | 2.77519 | 2.98518 | 4.58477 | 3.61503 | |
| 265 | 2.88487 | 3.9778 | 3.1923 | 2.69826 | 2.68678 | 2.46972 | 2.38116 | 4.913 | 3.4294 | 336 -- |
| | 2.90347 | 2.73739 | 3.18146 | 2.89801 | 2.37887 | 2.77519 | 2.98518 | 4.58477 | 3.61503 | |
| 266 | 3.30168 | 3.9531 | 3.18156 | 3.21271 | 2.65066 | 2.87878 | 2.58741 | 5.07124 | 3.40935 | 337 -- |
| | 2.90347 | 2.73739 | 3.18146 | 2.89801 | 2.37887 | 2.77519 | 2.98518 | 4.58477 | 3.61503 | |
| 267 | 2.69055 | 3.96644 | 3.14739 | 2.93636 | 2.71216 | 2.85395 | 2.65451 | 4.9359 | 2.5173 | 338 -- |
| | 2.90347 | 2.73739 | 3.18146 | 2.89801 | 2.37887 | 2.77519 | 2.98518 | 4.58477 | 3.61503 | |
| 268 | 2.71543 | 3.82018 | 2.73692 | 2.61288 | 2.51777 | 2.88583 | 3.44807 | 5.53194 | 4.14028 | 339 -- |
| | 2.90347 | 2.73739 | 3.18146 | 2.89801 | 2.37887 | 2.77519 | 2.98518 | 4.58477 | 3.61503 | |
| 269 | 2.92877 | 3.91898 | 2.93968 | 2.62672 | 2.80567 | 3.09658 | 3.58288 | 5.70003 | 4.31784 | 340 -- |
| | 2.90347 | 2.73739 | 3.18146 | 2.89801 | 2.37887 | 2.77519 | 2.98518 | 4.58477 | 3.61503 | |
| 270 | 3.23153 | 4.23051 | 3.58785 | 4.05877 | 3.38503 | 3.807 | 4.31921 | 6.11573 | 4.86017 | 341 -- |
| | 2.90347 | 2.73739 | 3.18146 | 2.89801 | 2.37887 | 2.77519 | 2.98518 | 4.58477 | 3.61503 | |
| 271 | 3.4468 | 1.30454 | 3.39493 | 3.31617 | 2.82687 | 2.80687 | 2.7958 | 5.36469 | 4.09397 | 342 -- |
| | 2.90347 | 2.73739 | 3.18146 | 2.89801 | 2.37887 | 2.77519 | 2.98518 | 4.58477 | 3.61503 | |
| 272 | 3.75539 | 3.48523 | 3.62504 | 3.59527 | 2.9501 | 2.97448 | 2.17806 | 4.87076 | 3.68552 | 343 -- |
| | 2.90347 | 2.73739 | 3.18146 | 2.89801 | 2.37887 | 2.77519 | 2.98518 | 4.58477 | 3.61503 | |
| 273 | 3.74609 | 4.1125 | 3.64019 | 3.18714 | 3.03682 | 2.86575 | 1.74391 | 4.68996 | 3.25168 | 344 -- |
| | 2.90347 | 2.73739 | 3.18146 | 2.89801 | 2.37887 | 2.77519 | 2.98518 | 4.58477 | 3.61503 | |
| 274 | 3.23153 | 4.23051 | 3.58785 | 4.05877 | 3.38503 | 3.807 | 4.31921 | 6.11573 | 4.86017 | 345 -- |
| | 2.90347 | 2.73739 | 3.18146 | 2.89801 | 2.37887 | 2.77519 | 2.98518 | 4.58477 | 3.61503 | |
| 275 | 3.389 | 4.20134 | 2.87837 | 0.97353 | 3.2459 | 3.39214 | 3.76839 | 5.5477 | 4.40218 | 346 -- |
| | 2.90347 | 2.73739 | 3.18146 | 2.89801 | 2.37887 | 2.77519 | 2.98518 | 4.58477 | 3.61503 | |
| 276 | 2.91073 | 3.89154 | 1.55769 | 2.88255 | 2.46304 | 2.69593 | 3.29399 | 5.45123 | 4.12584 | 347 -- |
| | 2.90347 | 2.73739 | 3.18146 | 2.89801 | 2.37887 | 2.77519 | 2.98518 | 4.58477 | 3.61503 | |
| 277 | 3.12266 | 3.91936 | 2.70995 | 2.08216 | 2.76398 | 2.89886 | 2.6136 | 5.20313 | 3.92266 | 348 -- |
| | 2.90347 | 2.73739 | 3.18146 | 2.89801 | 2.37887 | 2.77519 | 2.98518 | 4.58477 | 3.61503 | |
| 278 | 2.87207 | 3.8426 | 2.77641 | 2.74598 | 2.67595 | 2.72565 | 3.56541 | 5.63559 | 4.22407 | 349 -- |
| | 2.90347 | 2.73739 | 3.18146 | 2.89801 | 2.37887 | 2.77519 | 2.98518 | 4.58477 | 3.61503 | |
| 279 | 4.552 | 4.74032 | 4.33421 | 4.338 | 3.85212 | 3.52054 | 1.97712 | 4.97337 | 3.70068 | 350 -- |
| | 2.90347 | 2.73739 | 3.18146 | 2.89801 | 2.37887 | 2.77519 | 2.98518 | 4.58477 | 3.61503 | |
| 280 | 4.04311 | 4.38632 | 4.03676 | 3.98298 | 3.26526 | 3.15902 | 1.13278 | 5.42087 | 4.20578 | 351 -- |
| | 2.90347 | 2.73739 | 3.18146 | 2.89801 | 2.37887 | 2.77519 | 2.98518 | 4.58477 | 3.61503 | |
| 281 | 2.57098 | 3.87941 | 2.74765 | 2.05076 | 2.72001 | 2.84902 | 3.57584 | 5.58202 | 4.22358 | 352 -- |
| | 2.90347 | 2.73739 | 3.18146 | 2.89801 | 2.37887 | 2.77519 | 2.98518 | 4.58477 | 3.61503 | |
| 282 | 3.83435 | 4.17303 | 3.71749 | 3.44045 | 3.11301 | 2.92441 | 2.26398 | 4.03334 | 3.56339 | 353 -- |
| | 2.90347 | 2.73739 | 3.18146 | 2.89801 | 2.37887 | 2.77519 | 2.98518 | 4.58477 | 3.61503 | |
| 283 | 4.79625 | 4.9058 | 4.45289 | 4.46832 | 4.1156 | 3.71584 | 2.31132 | 5.29224 | 4.26251 | 354 -- |
| | 2.90347 | 2.73739 | 3.18146 | 2.89801 | 2.37887 | 2.77519 | 2.98518 | 4.58477 | 3.61503 | |
| 284 | 2.94202 | 3.84477 | 2.63707 | 2.69451 | 2.66183 | 2.90137 | 3.10568 | 5.48944 | 4.12644 | 355 -- |
| | 2.90347 | 2.73739 | 3.18146 | 2.89801 | 2.37887 | 2.77519 | 2.98518 | 4.58477 | 3.61503 | |
| 285 | 2.79507 | 3.82214 | 2.73762 | 2.79451 | 2.56577 | 2.69653 | 3.46822 | 5.54614 | 4.15266 | 356 -- |
| | 2.90347 | 2.73739 | 3.18146 | 2.89801 | 2.37887 | 2.77519 | 2.98518 | 4.58477 | 3.61503 | |
| 286 | 2.60584 | 3.85391 | 2.76764 | 2.15057 | 2.53072 | 2.8974 | 3.33988 | 5.44131 | 4.09469 | 357 -- |
| | 2.90347 | 2.73739 | 3.18146 | 2.89801 | 2.37887 | 2.77519 | 2.98518 | 4.58477 | 3.61503 | |
| 287 | 4.13001 | 4.33412 | 4.44637 | 4.32905 | 3.39874 | 3.72363 | 4.18538 | 5.99711 | 5.14174 | 358 -- |
| | 2.90348 | 2.73741 | 3.18148 | 2.89802 | 2.37888 | 2.77521 | 2.9852 | 4.58478 | 3.61504 | |
| 288 | 4.36241 | 4.66366 | 4.31173 | 4.24713 | 3.65397 | 3.0981 | 0.9129 | 5.51418 | 4.30654 | 362 -- |
| | 2.90347 | 2.73739 | 3.18146 | 2.89801 | 2.37887 | 2.77519 | 2.98518 | 4.58477 | 3.61503 | |
| 289 | 2.6299 | 3.81865 | 2.38047 | 2.68704 | 2.6307 | 2.90437 | 3.55224 | 5.60138 | 4.1915 | 363 -- |
| | 2.90347 | 2.73739 | 3.18146 | 2.89801 | 2.37887 | 2.77519 | 2.98518 | 4.58477 | 3.61503 | |
| 290 | 4.39667 | 4.66484 | 4.38506 | 4.27796 | 3.62653 | 3.35899 | 0.81078 | 5.60905 | 4.38851 | 364 -- |
| | 2.90347 | 2.73739 | 3.18146 | 2.89801 | 2.37887 | 2.77519 | 2.98518 | 4.58477 | 3.61503 | |
| 291 | 3.06977 | 3.89719 | 2.91543 | 3.00163 | 2.73196 | 2.64931 | 1.99781 | 5.25273 | 3.94904 | 365 -- |
| | 2.90347 | 2.73739 | 3.18146 | 2.89801 | 2.37887 | 2.77519 | 2.98518 | 4.58477 | 3.61503 | |
| 292 | 2.98093 | 3.84902 | 2.81133 | 2.63405 | 2.56721 | 2.7822 | 2.95326 | 5.38468 | 4.04366 | 366 -- |
| | 2.90347 | 2.73739 | 3.18146 | 2.89801 | 2.37887 | 2.77519 | 2.98518 | 4.58477 | 3.61503 | |
| 293 | 2.80617 | 3.82557 | 2.65643 | 2.28622 | 2.51721 | 2.87727 | 3.16808 | 5.49435 | 4.11885 | 367 -- |
| | 2.90347 | 2.73739 | 3.18146 | 2.89801 | 2.37887 | 2.77519 | 2.98518 | 4.58477 | 3.61503 | |
| 294 | 3.89641 | 4.23778 | 3.78696 | 3.74151 | 3.01456 | 2.99923 | 2.27151 | 4.64053 | 3.04737 | 368 -- |
| | 2.90347 | 2.73739 | 3.18146 | 2.89801 | 2.37887 | 2.77519 | 2.98518 | 4.58477 | 3.61503 | |
| 295 | 2.78638 | 3.81717 | 2.66238 | 2.75489 | 2.46889 | 2.62557 | 2.90962 | 5.48798 | 3.65338 | 369 -- |
| | 2.90347 | 2.73739 | 3.18146 | 2.89801 | 2.37887 | 2.77519 | 2.98518 | 4.58477 | 3.61503 | |
| 296 | 2.8779 | 3.83106 | 2.65698 | 2.80591 | 2.65298 | 2.92588 | 3.01843 | 5.60076 | 4.19476 | 370 -- |
| | 2.90347 | 2.73739 | 3.18146 | 2.89801 | 2.37887 | 2.77519 | 2.98518 | 4.58477 | 3.61503 | |
| 297 | 2.94169 | 3.85121 | 2.8143 | 2.95598 | 2.67677 | 2.90977 | 3.15086 | 5.45641 | 3.65663 | 371 -- |
| | 2.90347 | 2.73739 | 3.18146 | 2.89801 | 2.37887 | 2.77519 | 2.98518 | 4.58477 | 3.61503 | |
| 298 | 2.99871 | 3.89494 | 3.04139 | 3.23675 | 2.49653 | 3.02301 | 3.24829 | 5.6479 | 4.28587 | 372 -- |
| | 2.90347 | 2.73739 | 3.18146 | 2.89801 | 2.37887 | 2.77519 | 2.98518 | 4.58477 | 3.61503 | |
| 299 | 3.6964 | 4.10981 | 3.59171 | 3.55629 | 3.02793 | 2.88812 | 2.23928 | 4.5871 | 2.60401 | 373 -- |
| | 2.90347 | 2.73739 | 3.18146 | 2.89801 | 2.37887 | 2.77519 | 2.98518 | 4.58477 | 3.61503 | |
| 300 | 3.79703 | 4.41988 | 3.78638 | 3.48044 | 3.58665 | 3.83775 | 4.00762 | 5.08791 | 3.57711 | 374 -- |
| | 2.90347 | 2.73739 | 3.18146 | 2.89801 | 2.37887 | 2.77519 | 2.98518 | 4.58477 | 3.61503 | |
| 301 | 3.04541 | 3.89626 | 3.12507 | 3.18587 | 2.58664 | 2.85626 | 2.80437 | 5.24199 | 3.97819 | 375 -- |
| | 2.90347 | 2.73739 | 3.18146 | 2.89801 | 2.37887 | 2.77519 | 2.98518 | 4.58477 | 3.61503 | |
| 302 | 3.73443 | 4.12588 | 3.62914 | 3.59162 | 2.7763 | 2.88073 | 1.80521 | 4.76515 | 3.57129 | 376 -- |
| | 2.90347 | 2.73739 | 3.18146 | 2.89801 | 2.37887 | 2.77519 | 2.98518 | 4.58477 | 3.61503 | |
| 303 | 2.87458 | 3.86163 | 2.65692 | 3.01054 | 2.71131 | 2.79001 | 3.56132 | 5.65054 | 4.23924 | 377 -- |
| | 2.90347 | 2.73739 | 3.18146 | 2.89801 | 2.37887 | 2.77519 | 2.98518 | 4.58477 | 3.61503 | |
| 304 | 3.72968 | 4.09384 | 3.62657 | 3.57749 | 3.01518 | 2.7385 | 2.14422 | 4.68749 | 2.9652 | 378 -- |
| | 2.90347 | 2.73739 | 3.18146 | 2.89801 | 2.37887 | 2.77519 | 2.98518 | 4.58477 | 3.61503 | |
| 305 | 3.68774 | 4.08206 | 3.57886 | 3.40275 | 2.763 | 2.84466 | 2.10693 | 4.7005 | 3.22265 | 379 -- |
| | 2.90347 | 2.73739 | 3.18146 | 2.89801 | 2.37887 | 2.77519 | 2.98518 | 4.58477 | 3.61503 | |
| 306 | 2.84039 | 3.91812 | 2.92771 | 3.21026 | 2.83117 | 3.16936 | 3.37129 | 5.8699 | 4.41766 | 380 -- |
| | 2.90347 | 2.73739 | 3.18146 | 2.89801 | 2.37887 | 2.77519 | 2.98518 | 4.58477 | 3.61503 | |
| 307 | 3.10827 | 1.72208 | 2.89443 | 3.09457 | 2.60755 | 2.91108 | 2.79239 | 5.37431 | 4.0777 | 381 -- |
| | 2.90347 | 2.73739 | 3.18146 | 2.89801 | 2.37887 | 2.77519 | 2.98518 | 4.58477 | 3.61503 | |
| 308 | 2.65587 | 3.83289 | 2.67303 | 2.74306 | 2.10359 | 2.81032 | 3.37697 | 5.47497 | 4.10635 | 382 -- |
| | 2.90347 | 2.7374 | 3.18147 | 2.89802 | 2.37888 | 2.7752 | 2.98519 | 4.58478 | 3.61504 | |
| 309 | 2.88917 | 3.87593 | 2.57634 | 2.42638 | 2.70625 | 2.92664 | 3.3701 | 5.44806 | 4.11455 | 385 -- |
| | 2.90347 | 2.73739 | 3.18146 | 2.89801 | 2.37887 | 2.77519 | 2.98518 | 4.58477 | 3.61503 | |
| 310 | 3.13176 | 3.8772 | 3.0175 | 2.85652 | 2.48078 | 2.68303 | 3.11756 | 5.36133 | 4.0705 | 386 -- |
| | 2.90347 | 2.73739 | 3.18146 | 2.89801 | 2.37887 | 2.77519 | 2.98518 | 4.58477 | 3.61503 | |
| 311 | 2.81549 | 3.81291 | 2.64312 | 2.84448 | 2.54176 | 2.79728 | 3.27095 | 5.50626 | 4.11911 | 387 -- |
| | 2.90347 | 2.73739 | 3.18146 | 2.89801 | 2.37887 | 2.77519 | 2.98518 | 4.58477 | 3.61503 | |
| 312 | 3.00338 | 3.70292 | 2.94366 | 3.03676 | 2.6418 | 2.72316 | 2.70038 | 5.14794 | 3.86175 | 388 -- |
| | 2.90347 | 2.73739 | 3.18146 | 2.89801 | 2.37887 | 2.77519 | 2.98518 | 4.58477 | 3.61503 | |
| 313 | 4.26075 | 4.49747 | 4.07809 | 4.05218 | 3.32305 | 3.26145 | 2.40478 | 4.9215 | 3.7577 | 389 -- |
| | 2.90347 | 2.73739 | 3.18146 | 2.89801 | 2.37887 | 2.77519 | 2.98518 | 4.58477 | 3.61503 | |
| 314 | 3.62934 | 4.06528 | 3.5133 | 3.49234 | 2.97307 | 2.6411 | 2.37715 | 4.72089 | 2.69149 | 390 -- |
| | 2.90347 | 2.73739 | 3.18146 | 2.89801 | 2.37887 | 2.77519 | 2.98518 | 4.58477 | 3.61503 | |
| 315 | 2.67215 | 3.81757 | 2.66453 | 2.86274 | 2.62285 | 2.76226 | 3.41816 | 5.50782 | 4.12161 | 391 -- |
| | 2.90347 | 2.73739 | 3.18146 | 2.89801 | 2.37887 | 2.77519 | 2.98518 | 4.58477 | 3.61503 | |
| 316 | 2.89859 | 3.84575 | 2.63854 | 2.83459 | 2.38629 | 2.77076 | 2.85238 | 5.33411 | 3.46915 | 392 -- |
| | 2.90347 | 2.73739 | 3.18146 | 2.89801 | 2.37887 | 2.77519 | 2.98518 | 4.58477 | 3.61503 | |
| 317 | 4.3899 | 4.634 | 4.30688 | 4.24796 | 3.66285 | 3.06959 | 1.30038 | 5.27231 | 4.09455 | 393 -- |
| | 2.90347 | 2.73739 | 3.18146 | 2.89801 | 2.37887 | 2.77519 | 2.98518 | 4.58477 | 3.61503 | |
| 318 | 3.06474 | 3.96574 | 2.79584 | 2.24886 | 2.85305 | 3.08196 | 3.60033 | 5.56411 | 4.2719 | 394 -- |
| | 2.90347 | 2.73739 | 3.18146 | 2.89801 | 2.37887 | 2.77519 | 2.98518 | 4.58477 | 3.61503 | |
| 319 | 2.26805 | 3.88228 | 2.85632 | 3.11139 | 2.65155 | 3.08567 | 3.75366 | 5.81284 | 4.36273 | 395 -- |
| | 2.90347 | 2.73739 | 3.18146 | 2.89801 | 2.37887 | 2.77519 | 2.98518 | 4.58477 | 3.61503 | |
| 320 | 4.42942 | 4.84922 | 4.38092 | 4.38584 | 3.958 | 3.88041 | 2.89332 | 4.22125 | 2.56544 | 396 -- |
| | 2.90347 | 2.73739 | 3.18146 | 2.89801 | 2.37887 | 2.77519 | 2.98518 | 4.58477 | 3.61503 | |
| 321 | 4.65005 | 4.80648 | 4.5078 | 4.47436 | 3.91515 | 3.44235 | 1.5474 | 5.35734 | 4.22496 | 397 -- |
| | 2.90347 | 2.73739 | 3.18146 | 2.89801 | 2.37887 | 2.77519 | 2.98518 | 4.58477 | 3.61503 | |
| 322 | 3.05966 | 3.93739 | 3.06446 | 2.73373 | 2.35106 | 2.85001 | 2.73416 | 4.38126 | 3.13613 | 398 -- |
| | 2.90347 | 2.73739 | 3.18146 | 2.89801 | 2.37887 | 2.77519 | 2.98518 | 4.58477 | 3.61503 | |
| 323 | 2.84932 | 3.82255 | 2.78012 | 2.70216 | 2.03782 | 2.58111 | 3.19901 | 5.35527 | 4.01084 | 399 -- |
| | 2.90347 | 2.73739 | 3.18146 | 2.89801 | 2.37887 | 2.77519 | 2.98518 | 4.58477 | 3.61503 | |
| 324 | 3.37707 | 3.95525 | 3.23876 | 3.26915 | 2.5015 | 2.4114 | 2.43034 | 4.80927 | 3.5886 | 400 -- |
| | 2.90347 | 2.73739 | 3.18146 | 2.89801 | 2.37887 | 2.77519 | 2.98518 | 4.58477 | 3.61503 | |
| 325 | 2.93089 | 3.32053 | 2.62015 | 2.86785 | 2.354 | 2.73771 | 3.03909 | 5.30984 | 3.96862 | 401 -- |
| | 2.90347 | 2.73739 | 3.18146 | 2.89801 | 2.37887 | 2.77519 | 2.98518 | 4.58477 | 3.61503 | |
| 326 | 2.69886 | 3.79927 | 2.6598 | 2.74064 | 2.24712 | 2.81046 | 2.87558 | 5.24658 | 3.92536 | 402 -- |
| | 2.90355 | 2.7376 | 3.18172 | 2.89834 | 2.37864 | 2.77524 | 2.98456 | 4.58511 | 3.61522 | |

What is claimed is:

1. A method of increasing in a crop plant at least one phenotype selected from the group consisting of: triple stress tolerance, drought stress tolerance, nitrogen stress tolerance, osmotic stress tolerance, ABA response, tiller number, yield and biomass, the method comprising increasing the expression of a carboxyl esterase in the crop plant.

2. The method of claim 1, wherein the crop plant is maize and the carboxyl esterase is a plant carboxyl esterase.

3. The method of claim 1, wherein the carboxyl esterase has at least 80% sequence identity, when compared to SEQ ID NO:18, 39, 43, 45, 47, 49, 51,55, 59, 61, 64, 65, 66, 95, 97, 101, 103, 107, 111, 113, 117, 119, 121, 123, 127, 129, 130, 131, 132, 135, 627 or 628.

4. (canceled)

5. A plant comprising in its genome a recombinant DNA construct comprising a polynucleotide operably linked to at least one heterologous regulatory element, wherein said polynucleotide encodes a polypeptide having an amino acid sequence of at least 80% sequence identity, when compared to SEQ ID NO:18, 39, 43, 45, 47, 49, 51, 55, 59, 61, 64, 65, 66, 95, 97, 101, 103, 107, 111, 113, 117, 119, 121, 123, 127, 129, 130, 131, 132, 135, 627 or 628, and wherein said plant exhibits an increase in yield, biomass, or both, when compared to a control plant not comprising said recombinant DNA construct.

6. The plant of claim 5, wherein said plant exhibits said increase in yield, biomass, or both when compared, under water limiting conditions, to said control plant not comprising said recombinant DNA construct.

7. The plant of claim 5, wherein said plant is selected from the group consisting of: Arabidopsis, maize, soybean, sunflower, sorghum, canola, wheat, alfalfa, cotton, rice, barley, millet, sugar cane and switchgrass.

8. Seed of the plant of claim 5, wherein said seed comprises in its genome a recombinant DNA construct comprising a polynucleotide operably linked to at least one heterologous regulatory element, wherein said polynucleotide encodes a polypeptide having an amino acid sequence of at least 80% sequence identity, when compared to SEQ ID NO:18, 39, 43, 45, 47, 49, 51, 55, 59, 61, 64, 65, 66, 95, 97, 101, 103, 107, 111, 113, 117, 119, 121, 123, 127, 129, 130, 131, 132, 135, 627 or 628, and wherein a plant produced from said seed exhibits an increase in at least one phenotype selected from the group consisting of: drought stress tolerance, triple stress tolerance, osmotic stress tolerance, nitrogen stress tolerance, tiller number, yield and biomass, when compared to a control plant not comprising said recombinant DNA construct.

9-14. (canceled)

15. An isolated polynucleotide comprising:
(a) a nucleotide sequence encoding a polypeptide with stress tolerance activity, wherein the stress is selected from a group consisting of drought stress, triple stress, nitrogen stress and osmotic stress, and wherein the polypeptide has an amino acid sequence of at least 95% sequence identity when compared to SEQ ID NO:18, 39, 43, 45, 47, 49, 51,55, 59, 61,64, 65, 66, 95, 97, 101, 103, 107, 111, 113, 117, 119, 121, 123, 127, 129, 130, 131, 132, 135, 627 or 628; or
(b) the full complement of the nucleotide sequence of (a), wherein the nucleotide is operably linked to a heterologous regulatory element.

16. The polynucleotide of claim 15, wherein the amino acid sequence of the polypeptide comprises SEQ ID NO:18, 39, 43, 45, 47, 49, 51, 55, 59, 61, 64, 65, 66, 95, 97, 101, 103, 107, 111, 113, 117, 119, 121, 123, 127, 129, 130, 131, 132, 135, 627 or 628.

17. The polynucleotide of claim 15 wherein the nucleotide sequence comprises SEQ ID NO:16, 17, 19, 38, 42, 44, 46, 48, 50, 54, 58, 60, 62, 63, 94, 96, 100, 102, 106, 110, 112, 116, 118, 120 or 122.

18. (canceled)

19. A plant comprising in its genome an endogenous polynucleotide operably linked to at least one heterologous regulatory element, wherein said endogenous polynucleotide encodes a polypeptide having an amino acid sequence of at least 80% sequence identity, when compared to SEQ ID NO:18, 39, 43, 45, 47, 49, 51, 55, 59, 61, 64, 65, 66, 95, 97, 101, 103, 107, 111, 113, 117, 119, 121, 123, 127, 129, 130, 131, 132, 135, 627 or 628, and wherein said plant exhibits at least one phenotype selected from the group consisting of increased triple stress tolerance, increased drought stress tolerance, increased nitrogen stress tolerance, increased osmotic stress tolerance, altered ABA response, altered root architecture, increased tiller number, when compared to a control plant not comprising the heterologous regulatory element.

20-28. (canceled)

29. The plant of claim 19, wherein the polynucleotide encodes a polypeptide having an amino acid sequence of at least 95% sequence identity, when compared to SEQ ID NO:18.

30. The plant of claim 19 is maize plant.

31. The plant of claim 19, wherein the heterologous nucleic acid is a constitutive plant promoter.
